(19) United States
(12) Patent Application Publication
(10) Pub. No.: US 2023/0374542 A1
(43) Pub. Date: Nov. 23, 2023

(54) THERAPEUTIC ADENO-ASSOCIATED VIRUS DELIVERY OF FUKUTIN RELATED PROTEIN (FKRP) FOR TREATING DYSTROGLYCANOPATHY. DISORDERS INCLUDING LIMB GIRDLE 21 (LGMD21)

(71) Applicants: Asklepios BioPharmaceutical, Inc., Research Triangle Park, NC (US); The University of North Carolina at Chapel Hill, Chapel Hill, NC (US)

(72) Inventors: Martin K. Childers, Research Triangle Park, NC (US); Jorge Omar Yanez-Cuna, Midlothian (GB); Juan Manuel Iglesias Gonzalez, Midlothian (GB); Sinclair Cooper, Midlothian (GB); Antonia Evripioti, Midlothian (GB); Michael L. Roberts, Midlothian (GB); Anna Tretiakova, Research Triangle Park, NC (US); Lester Suarez, Research Triangle Park, NC (US); Anh Nguyen, Research Triangle Park, NC (US); SiewHui Low, Research Triangle Park, NC (US); Xiao Xiao, Chapel Hill, NC (US)

(73) Assignees: Asklepios BioPharmaceutical, Inc., Research Triangle Park, NC (US); The University of North Carolina at Chapel Hill, Chapel Hill, NC (US)

(21) Appl. No.: 18/030,730

(22) PCT Filed: Oct. 6, 2021
(86) PCT No.: PCT/US2021/053768

§ 371 Date: Apr. 6, 2023

Related U.S. Application Data
(60) Provisional application No. 63/088,757, filed on Oct. 7, 2020.
(60) Provisional application No. 63/214,123, filed on Jun. 23, 2021.
(60) Provisional application No. 63/229,726, filed on Aug. 5, 2021.

(51) Int. Cl. C12N 15/86; A61P 21/00

(52) U.S. Cl. CPC C12N 15/86; A61P 21/00; C12N 2750/14143; C12N 2830/008

(57) ABSTRACT

Disclosed herein are various optimized nucleic acids encoding the fukutin-related protein (FKRP). Recombinant vectors comprising the optimized nucleic acid (e.g. operatively linked to a muscle specific promoter), such as recombinant adeno-associated virus vectors, for expressing the protein (e.g. in skeletal and cardiac muscle), and therapeutic compositions contains the vectors are also disclosed. Therapeutic methods of administration of the vectors to a subject for the treatment of a subject with a dystroglycanopathy disorder (e.g., limb-girdle muscular dystrophy 2I) are also disclosed.

### CROSS-REFERENCE TO RELATED APPLICATIONS

This application is a 35 U.S.C. § 371 National Phase Entry Application of International Application No. PCT/US2021/053768 filed Oct. 6, 2021, which designed the U.S., which claims benefit under 35 U.S.C. § 119(e) of U.S. Provisional Application No. 63/088,757 filed Oct. 7, 2020, U.S. Provisional Application No. 63/214,123 filed Jun. 23, 2021, and U.S. Provisional Application No. 63/229,726 filed Aug. 5, 2021, the contents of each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Nov. 24, 2021, is named 046192-098420WOPT_SL.txt and is 247,577 bytes in size.

### FIELD OF THE INVENTION

The present invention relates to the field of gene therapy and the treatment of dystroglycanopathy disorders.

### BACKGROUND OF THE INVENTION

Limb girdle muscular dystrophy, or LGMD, represents a broad class of over twenty rare genetically defined myopathies related to the weakness and atrophy of muscles that connect to the shoulder or hips, which are generally referred to as the limb girdles. These genetic myopathies are subdivided into LGMD1 and LGMD2 groups based on whether they are inherited as dominant or recessive diseases, respectively. Each of the LGMDs is caused by mutations in different genes.

Symptoms associated with LGMD2I often develop in late childhood when afflicted children begin to have difficulty running and walking. The symptoms and mobility issues gradually worsen overtime, with patients generally relying on a wheelchair between 23 and 26 years from onset. Shoulder and arm weakness can create challenges in holding, carrying and lifting objects and can result in the need for assistive devices. The disease may also cause difficulty breathing, cardiomyopathies and arrhythmias, and contraction-induced shear damage to the sarcolemma, the primary lesion leading to the LGMD2I phenotype. Dystroglycan is the central protein in the dystrophin-glycoprotein complex, or DGC, and its glycosylation is crucial for flexibly connecting structural elements of muscle cells to the structures that surround them, called the extra-cellular matrix, or ECM. FKRP attaches ribitol-5-P to the glycan sequence progressively adorning α-DG. A definitive study using a combination of high-performance liquid chromatography, or HPLC, mass spectroscopy and nuclear magnetic resonance, or NMR, demonstrated that fukutin-related protein (FKRP) is a transferase that inserts the second of two ribitol-5-phosphates into the glycan chain immediately preceding the ligand binding moiety of the glycan chain. Absence of any part of this glycan chain results in failure of α-DG to bind to its ECM targets, which leads to repeated stresses on the sarcolemma or cell membrane that are the hallmark of many LGMDs, including LGMD2I. Based on analyses of publicly available genome databases, it is estimated that 4.3 out of every million people suffer from LGMD2I. LGMD2I is most prevalent in Northern Europe due to a founder mutation effect, where a genetic alteration in the gene encoding FKRP is observed with high frequency in a group that is or was geographically or culturally isolated and one or more of the ancestors was a carrier of the altered gene.

Mutations in the gene encoding FKRP result in a wide spectrum of disease phenotypes including the mild limb-girdle muscular dystrophy 2I (LGMD2I), the severe Walker-Warburg syndrome, and muscle-eye-brain disease. Currently, no effective therapy is known for dystroglycanopathies involving a reduction in glycosylation of α-DG (Xu et al. Mol. Therapy 21:10doi:10.1038/mt.2013.156 (Jul. 2, 2013)). There are no approved therapies for LGMD2I and treatments are aimed at symptom management, including supportive care and assistive devices for mobility.

### SUMMARY OF THE INVENTION

Aspects of the invention relate to a recombinant adenovirus associated (AAV) vector comprising in its genome in the 5′ to 3′ direction a) a 5′ AAV inverted terminal repeat (ITR); b) a muscle specific promoter; c) an intron sequence; d) a nucleic acid encoding human fukutin-related protein (FKRP) which has a nucleotide sequence shown in SEQ ID NO: 2, and is operatively linked to the muscle specific promoter; e) a polyA signal sequence operatively linked to the nucleic acid encoding FKRP; f) a 3′ AAV ITR.

In some embodiments of the rAAV vector and methods recited herein, the 5′ITR is ITR2m.

In some embodiments of the rAAV vector and methods recited herein, the 3′ITR is ITR2.

In some embodiments of the rAAV vector and methods recited herein, the muscle-specific promoter is Syn100 (SEQ ID NO: 3).

In some embodiments of the rAAV vector and methods recited herein, the intron sequence is VH4-Ig-Intron 3 (SEQ ID NO: 4) or a derivative thereof.

In some embodiments of the rAAV vector and methods recited herein, the polyA signal sequence is SEQ ID NO: 5.

In some embodiments of the rAAV vector and methods recited herein, the muscle specific promoter, intron sequence, nucleic acid encoding FKRP, and polyA signal sequence are comprised within SEQ ID NO: 1.

In some embodiments of the rAAV vector and methods recited herein, the serotype is AAV9.

Aspects of the invention also relate to pharmaceutical compositions comprising the various embodiments of recombinant AAV vector described above and herein.

Aspects of the invention also relate to a method to treat a subject with a dystroglycanopathy disorder comprising systemically administering a therapeutically effective amount of the various embodiments of the recombinant AAV vector described herein, and/or the pharmaceutical composition described herein, to the subject, to thereby increase expression of functional FKRP in muscle tissue of the subject.

In some embodiments of the methods described herein, the dystroglycanopathy disorder is limb-girdle muscular dystrophy 2I.

In some embodiments of the methods described herein, a single dose is administered to the subject.

In some embodiments of the methods described herein, administration is by intravenous infusion.

In some embodiments of the methods described herein, the dose administered is from about 1E13 vg/kg to about 6E13 vg/kg (e.g. about 3E13 vg/kg).

In some embodiments of the methods described herein, one or more of the following occur in the subject following administration: a) functional glycosylation of α-DG is substantially increased in skeletal muscle and/or cardiac muscle of the subject; b) serum creatine kinase levels of the subject are substantially reduced; c) collagen deposition in skeletal muscle of the subject is substantially reduced; d) in vitro muscle force analysis of the subject's muscle tissue (e.g., soleus, diaphragm and/or EDL) is significantly increased; e) tidal volume of the subject is substantially increased; and/or f) the subject can run significantly further in a treadmill test.

In some embodiments of the methods described herein the subject is an adult, an adolescent, or an infant. In some embodiments of the methods described herein the subject is a male or a female.

Aspects of the invention also relate to a synthetic nucleic acid encoding human fukutin-related protein (FKRP), wherein: a) the nucleic acid has reduced CpG site content relative to the CpG site content of SEQ ID NO: 6; b) the GC content is reduced by greater than 10% relative to the GC content of SEQ ID NO:6; and/or c) the nucleic acid has at least 80% identity to SEQ ID NO: 2.

In some embodiments of the nucleic acid, rAAV vector and methods recited herein the coding sequence has at least 50% reduced CpG site content relative to the CpG site content of SEQ ID NO: 6.

In some embodiments of the nucleic acid, rAAV vector and methods recited herein the coding sequence has at least 75%, 80%, 85%, 90%, 95% reduced CpG site content relative to the CpG site content of SEQ ID NO: 6.

In some embodiments of the nucleic acid, rAAV vector and methods recited herein the coding sequence has 0% CpG site content.

In some embodiments of the nucleic acid, rAAV vector and methods recited herein the GC content is reduced by greater than 15% relative to the GC content of SEQ ID NO:6.

In some embodiments of the nucleic acid, rAAV vector and methods recited herein the nucleic acid has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 2.

In some embodiments of the nucleic acid, rAAV vector and methods recited herein, the nucleic acid has a sequence shown in SEQ ID NO: 2.

In some embodiments of the nucleic acid, rAAV vector and methods recited herein the synthetic nucleic acid is operably linked to a promoter.

In some embodiments of the nucleic acid, rAAV vector and methods recited herein the promoter is a muscle-specific promoter.

In some embodiments of the nucleic acid, rAAV vector and methods recited herein the promoter is a synthetic promoter.

In some embodiments of the nucleic acid, rAAV vector and methods recited herein the promoter is Syn100.

In some embodiments of the nucleic acid, rAAV vector and methods recited herein the promoter is selected from promoters listed in Tables 1-4.

In some embodiments of the nucleic acid, rAAV vector and methods recited herein the promoter is a creatine kinase (CK) promoter, a chicken R-actin promoter (CB).

In some embodiments of the nucleic acid, rAAV vector and methods recited herein the synthetic nucleic acid further comprises an enhancer sequence.

In some embodiments of the nucleic acid, rAAV vector and methods recited herein the enhancer sequence comprises a CMV enhancer, a muscle creatine kinase enhancer, and/or a myosin light chain enhancer.

Aspects of the invention also relate to a nucleic acid comprising: 5′ and 3′ AAV inverted terminal repeats (ITR); a coding sequence encoding human fukutin-related protein (FKRP) operatively linked to a muscle-specific promoter located between the 5′ITR and 3′ITR, wherein the coding sequence has: reduced CpG site content relative to the CpG site content of SEQ ID NO: 6; reduced GC content greater than 10% relative to the GC content of SEQ ID NO:6; and/or

at least 80% identity to SEQ ID NO: 2.

In some embodiments of the nucleic acid, rAAV vector and methods recited herein the nucleic acid further comprises an intron sequence located between the muscle-specific promoter and the coding sequence.

In some embodiments of the nucleic acid, rAAV vector and methods recited herein the intron sequence is VH4-Ig-Intron 3 (SEQ ID NO: 4) or a derivative thereof.

In some embodiments of the nucleic acid, rAAV vector and methods recited herein the nucleic acid further comprises at least one polyA signal sequence located downstream of the coding sequence.

In some embodiments of the nucleic acid, rAAV vector and methods recited herein the polyA signal sequence is SEQ ID NO: 5.

In some embodiments of the nucleic acid, rAAV vector and methods recited herein the 5′ITR is ITR2m.

In some embodiments of the nucleic acid, rAAV vector and methods recited herein the 3′ITR is ITR2.

In some embodiments of the nucleic acid, rAAV vector and methods recited herein the GC content of the coding sequence is reduced by greater than 15% relative to the GC content of SEQ ID NO:6.

In some embodiments of the nucleic acid, rAAV vector and methods recited herein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 2.

In some embodiments of the nucleic acid, rAAV vector and methods recited herein the coding sequence has at least 50% reduced CpG site content relative to the CpG site content of SEQ ID NO: 6.

In some embodiments of the nucleic acid, rAAV vector and methods recited herein the coding sequence has at least 75%, 80%, 85%, 90%, 95% reduced CpG site content relative to the CpG site content of SEQ ID NO: 6.

In some embodiments of the nucleic acid, rAAV vector and methods recited herein the coding sequence has 0% CpG site content.

In some embodiments of the nucleic acid, rAAV vector and methods recited herein the coding sequence is SEQ ID NO: 2.

Aspects of the invention also relate to a vector comprising the synthetic nucleic acids described above and herein.

In some embodiments of the nucleic acid, vectors and methods recited herein the vector is a viral vector.

In some embodiments of the nucleic acid, vector and methods recited herein the vector is a recombinant adeno-associated virus (AAV) vector.

In some embodiments of the nucleic acid, rAAV vector and methods recited herein the AAV vector is any serotype listed in Table 6 (e.g., AAV9).

Aspects of the invention also relate to a recombinant adenovirus associated (AAV) vector comprising in its genome: a) a 5′ AAV inverted terminal repeat (ITR) and a 3′ AAV ITR; b) located between the 5′ITR and 3′ITR, a nucleic acid encoding human fukutin-related protein (FKRP) which has: i) reduced CpG site content relative to the CpG site content of SEQ ID NO: 6; ii) reduced GC content greater than 10% relative to the GC content of SEQ ID NO:6; and/or iii) at least 80% identity to SEQ ID NO: 2, and is operatively linked to a muscle-specific promoter.

In some embodiments of the nucleic acid, rAAV vector and methods recited herein, the AAV genome comprises, in the 5′ to 3′ direction: the 5′ITR, the muscle-specific promoter, an intron sequence, the nucleic acid encoding FKRP; and the 3′ITR.

In some embodiments of the nucleic acid, rAAV vector and methods recited herein the muscle-specific promoter is selected from the group consisting of MCK promoter, dMCK promoter, tMCK promoter, enh358MCK promoter, CK6 promoter and Syn100 promoter, any promoter listed in Table 1-4 or 8-12, and derivatives thereof.

In some embodiments of the nucleic acid, rAAV vector and methods recited herein the nucleic acid encoding FKRP has reduced CpG site content relative to the CpG site content of SEQ ID NO: 6.

In some embodiments of the nucleic acid, rAAV vector and methods recited herein the nucleic acid encoding FKRP has at least 50% reduced CpG site content relative to the CpG site content of SEQ ID NO: 6.

In some embodiments of the nucleic acid, rAAV vector and methods recited herein the nucleic acid encoding FKRP has at least 75%, 80%, 85%, 90%, 95% reduced CpG site content relative to the CpG site content of SEQ ID NO: 6.

In some embodiments of the nucleic acid, rAAV vector and methods recited herein the nucleic acid encoding FKRP has 0% CpG site content.

In some embodiments of the nucleic acid, rAAV vector and methods recited herein, the nucleic acid encoding FKRP has reduced GC content greater than 10% relative to the GC content of SEQ ID NO:6.

In some embodiments of the nucleic acid, rAAV vector and methods recited herein the nucleic acid encoding FKRP has at least 80% identity to SEQ ID NO: 2.

In some embodiments of the nucleic acid, rAAV vector and methods recited herein the nucleic acid encoding FKRP has a sequence shown in SEQ ID NO: 2.

In some embodiments of the nucleic acid, rAAV vector and methods recited herein, the recombinant AAV vector further comprises at least one polyA signal sequence located 3′ of the nucleic acid encoding the FKRP polypeptide and 5′ of the 3′ITR sequence.

In some embodiments of the nucleic acid, rAAV vector and methods recited herein the polyA signal sequence is SEQ ID NO: 5.

In some embodiments of the nucleic acid, rAAV vector and methods recited herein the ITR comprises an insertion, deletion or substitution.

In some embodiments of the nucleic acid, rAAV vector and methods recited herein one or more CpG site sites in the ITR are removed.

In some embodiments of the nucleic acid, rAAV vector and methods recited herein the 5′ITR is ITR2m.

In some embodiments of the nucleic acid, rAAV vector and methods recited herein the 3′ITR is ITR2.

In some embodiments of the nucleic acid, rAAV vector and methods recited herein the intron sequence is VH4-Ig-Intron 3 (SEQ ID NO: 4) or a derivative thereof.

In some embodiments of the nucleic acid, rAAV vector and methods recited herein The recombinant AAV vector is a chimeric AAV vector, haploid AAV vector, a hybrid AAV vector or polyploid AAV vector.

In some embodiments of the nucleic acid, rAAV vector and methods recited herein the recombinant AAV vector is any AAV serotype listed in Table 6, e.g., AAV9.

In some embodiments of the nucleic acid, rAAV vector and methods recited herein the recombinant AAV vector comprises a capsid protein selected from Table 7 or any AAV serotype in the group consisting of those listed in Table 6, and combinations thereof.

Aspects of the invention also relate to a pharmaceutical composition comprising the recombinant AAV vector described above, and herein, in a pharmaceutically acceptable carrier.

Aspects of the invention also relate to a transformed cell comprising the nucleic acid described above, and herein and/or the vector described above and herein.

Aspects of the invention also relate to a transgenic animal comprising the nucleic acid described above and herein, and/or the vector (e.g., rAAV) described above and herein, and/or the transformed cell described above and herein.

Aspects of the invention also relate to a method of increasing glycosylation of α-dystroglycan (α-DG) in a subject in need thereof, comprising: administering to said subject a therapeutically effective amount of the nucleic acid described above and herein, the vector (e.g., rAAV) described above and herein, the pharmaceutical composition described above and herein, and/or the transformed cell described above and herein, wherein the synthetic nucleic acid is expressed in said subject, thereby producing human FKRP and increasing glycosylation of α-DG.

In some embodiments of the methods recited herein the subject has or is at risk for developing a dystroglycanopathy disorder.

Aspects of the invention also relate to a method of treating or a dystroglycanopathy disorder in a subject, comprising administering to the subject a therapeutically effective amount of the nucleic acid described above and herein, the vector (e.g., rAAV) described above and herein, the pharmaceutical composition described above and herein, and/or the transformed cell described above and herein, wherein the synthetic nucleic acid is expressed in said subject, thereby treating the dystroglycanopathy disorder in the subject.

In some embodiments of the methods recited herein the dystroglycanopathy disorder is associated with a FKRP anomaly.

In some embodiments of the methods recited herein the dystroglycanopathy disorder comprises a mutation in the nucleic acid encoding FKRP and/or a deficiency in glycosylation of α-dystroglycan (α-DG).

In some embodiments of the methods recited herein dystroglycanopathy disorder is limb-girdle muscular dystrophy 2I, congenital muscular dystrophy (CMD1C), Walker-Warburg syndrome, muscle-eye-brain disease, or any combination thereof.

Aspects of the invention also relate to a method to treat a subject with a dystroglycanopathy disorder comprising administering a therapeutically effective amount of any of the recombinant AAV vector, the rAAV genome, the nucleic acid sequence, and/or the pharmaceutical compositions, of any one of the previous claims to the subject, to thereby increase expression of functional FKRP in muscle tissue of the subject.

In some embodiments of the methods recited herein a single dose is administered to the subject.

In some embodiments of the methods recited herein, administration is systemic.

In some embodiments of the methods recited herein administration is by intravenous infusion.

In some embodiments of the methods recited herein functional glycosylation of α-DG is substantially increased in skeletal muscle and/or cardiac muscle of the subject following administration.

In some embodiments of the methods recited herein serum creatine kinase levels of the subject are substantially reduced following administration.

In some embodiments of the methods recited herein collagen deposition in skeletal muscle of the subject is substantially reduced following administration.

In some embodiments of the methods recited herein in vitro muscle force analysis of the subject's muscle tissue (e.g., soleus, diaphragm and/or EDL) is significantly increased.

In some embodiments of the methods recited herein tidal volume of the subject is substantially increased.

In some embodiments of the methods recited herein the subject can run significantly further in a treadmill test.

In some embodiments of the methods recited herein the subject is an adult.

In some embodiments of the methods recited herein the subject is a juvenile.

In some embodiments of the methods recited herein the subject is an infant.

In some embodiments of the methods recited herein the subject demonstrates significant disease pathology prior to administration.

In some embodiments of the methods recited herein the subject demonstrates no significant disease pathology prior to administration.

### BRIEF DESCRIPTION OF THE DRAWINGS

FIG. 1 is a summary of the dose finding and toxicology studies performed.

FIG. 2 shows representative levels of expression in diaphragm and quadriceps of recipient mice, compared to mice which had received empty vehicle.

FIG. 3 shows representative α-dystroglycan expression in normal BL6 mice (upper left photo) serving as a positive control, P448L mice that had received 1E14 vg/kg (upper right) and 3E13 vg/kg (lower left) AAV9-FKRP, and P448L mice that had received empty vehicle (lower right) serving as a negative control.

FIG. 4 shows representative immunofluorescence images of functional α-DG expression in cross-sectional areas of quadriceps muscle of P448L mice with DAPI. Upper left frame is α-dystroglycan in wild type BL6 mice. Upper right frame is α-dystroglycan in P448L mice administered 1E14 vg/kg. Lower left frame is α-dystroglycan in P448L mice administered 3E13 vg/kg. Lower right frame is P448L mice administered empty vehicle.

FIG. 5 shows representative images of picrosirius red staining for collagen deposition in cross-sections of quadriceps muscles (top). Male images are shown. Without treatment, P448 mice (center top) demonstrate large collagen deposition and irregular muscle fiber shape. These features progressively return to normal at different doses of AAV9-FKRP. (bottom) is a graphical representation of quantitative collagen deposition in the P448L mice. Collagen deposition is shown as a percentage of total area in quadriceps muscle. Male and female data are combined. Collagen deposition is reduced at all doses. ###Unpaired t-test, p<0.001, n≥13; *One-way ANOVA with Dunnett's Multiple Comparison Test, p<0.05, n≥12; **One-way ANOVA with Dunnett's Multiple Comparison Test, p<0.01, n≥12. The percent collagen was calculated as: (total area of red staining/total biopsy area)*100.

FIG. 6 shows two graphical representations of data obtained from investigation of serum creatine kinase levels in mice that had received various amounts of AAV9-FKRP. Left, results were obtained in male mice, right, results were obtained in female mice. BL6 vehicle recipient mice serve as the positive control, P448L mice serve as the negative control. P448L mice received 1E13, 3E13, 1E14 or 3E14 vg/kg AAV9-FKRP.

FIG. 7 shows two graphical representations of specific force response from isolated extensor digitorum longus muscle. Left—male responses, right—female responses. BL6 vehicle recipient mice serve as the positive control, P448L mice serve as the negative control. P448L mice received 1E13, 3E13, 1E14 or 3E14 vg/kg AAV9-FKRP. In most cases, restoration of skeletal muscle is achieved at a dose of 1E13. A threshold effect is observed beyond this dose. #BL6 different to P448L Vehicle (p<0.05), ##BL6 different to P448L Vehicle (p<0.01), ###BL6 different to P448L Vehicle (p<0.001), * Treatment group different to P448L Vehicle (p<0.05), ** Treatment group different to P448L Vehicle (p<0.01), *** Treatment group different to P448L Vehicle (p<0.001).

FIG. 8 shows two graphical representations of specific force response from isolated diaphragm muscle. Left—male responses, right—female responses. BL6 vehicle recipient mice serve as the positive control, P448L mice serve as the negative control. P448L mice received 1E13, 3E13, 1E14 or 3E14 vg/kg AAV9-FKRP. In most cases, restoration of skeletal muscle is achieved at a dose of 1E13. A threshold effect is observed beyond this dose. #BL6 different to P448L Vehicle (p<0.05), ##BL6 different to P448L Vehicle (p<0.01), ###BL6 different to P448L Vehicle (p<0.001), * Treatment group different to P448L Vehicle (p<0.05), ** Treatment group different to P448L Vehicle (p<0.01), *** Treatment group different to P448L Vehicle (p<0.001).

FIG. 9 shows two graphical representations of specific force from isolated soleus muscle. Left—male responses, right—female responses. BL6 vehicle recipient mice serve as the positive control, P448L mice serve as the negative control. P448L mice received 1E13, 3E13, 1E14 or 3E14 vg/kg AAV9-FKRP. In most cases, restoration of skeletal muscle is achieved at a dose of 1E13. A threshold effect is observed beyond this dose. #BL6 different to P448L Vehicle (p<0.05), ##BL6 different to P448L Vehicle (p<0.01), ###BL6 different to P448L Vehicle (p<0.001), * Treatment group different to P448L Vehicle (p<0.05), ** Treatment group different to P448L Vehicle (p<0.01), *** Treatment group different to P448L Vehicle (p<0.001).

FIG. 10 shows exhaustion treadmill distance in P448 mice. Total distance is restored in all doses except for the max dose at 3E14. #BL6 different to P448L Vehicle (p<0.05), ##BL6 different to P448L Vehicle (p<0.01), ###BL6 different to P448L Vehicle (p<0.001), * Treatment group different to P448L Vehicle (p<0.05), ** Treatment group different to P448L Vehicle (p<0.01), * Treatment group different to P448L Vehicle (p<0.001).

FIG. 11 shows running wheel distance in P448 mice. Total distance is restored in all doses except for the max dose at 3E14. #BL6 different to P448L Vehicle (p<0.05), ##BL6 different to P448L Vehicle (p<0.01), ###BL6 different to P448L Vehicle (p<0.001), * Treatment group different to P448L Vehicle (p<0.05), ** Treatment group different to P448L Vehicle (p<0.01), *** Treatment group different to P448L Vehicle (p<0.001).

FIG. 12 shows plethysmography studies in male (left) and female (right) recipient mice. BL6 vehicle recipient mice serve as the positive control, P448L mice serve as the negative control. P448L mice received 1E13, 3E13, 1E14 or 3E14 vg/kg AAV9-FKRP.

FIG. 13 is a schematic plasmid map of dsAAV-Syn100-FKRP. The nucleotide sequences of various components of the plasmid, including the ITRs (ITR2m, ITR2), promoter (Syn100), Intron (VH4-Ig-Intron3), optimized coding sequence for FKRP (Opti-hu-FKRP-CpG(−)), the polyA signal sequence (sPolyA), and various spacers are also shown. FIG. 13 discloses SEQ ID NO: 1.

FIG. 14 shows the nucleotide sequence (SEQ ID NO: 2) of a synthetic nucleic acid encoding the human FKRP protein of the plasmid of FIG. 13. The nucleic acid has 0% CpG sites.

FIG. 15 shows the nucleotide sequence (SEQ ID NO: 3) of the promoter (Syn100) of the plasmid of FIG. 13.

FIG. 16 shows the nucleotide sequence (SEQ ID NO: 4) of the intron (VH4-Ig-Intron 3) of the plasmid of FIG. 13.

FIG. 17 shows the nucleotide sequence (SEQ ID NO: 5) of the polyA signal sequence of the plasmid of FIG. 13.

FIG. 18 shows the nucleotide sequence (SEQ ID NO: 6) of the native nucleotide sequence encoding human FKRP.

FIG. 19 shows other nucleotide sequences of the plasmid of FIG. 13, the ITR2M sequence (SEQ ID NO: 7), the ITR2 sequence (SEQ ID NO: 8), spacer sequences (SEQ ID NOS: 9-13).

FIG. 20 shows the average activity of synthetic muscle-specific promoters according to some embodiments of this invention in H9C2 cell line differentiated into heart myotubes. The error bar is standard deviation. CBA and CK8 are control promoters.

FIG. 21 shows results from the short synthetic muscle specific promoters, that shows the average activity, normalized to the CBA control promoter, of 11 selected synthetic muscle-specific promoters SP0497, SP0500, SP0501, SP0506, SP0508, SP0510, SP0514, SP0519, SP0520, SP0521 and SP4169 in H9C2 cell line differentiated into heart myotubes. The error bar is standard deviation from triplicate. CBA and CK8 are control promoters.

FIG. 22 shows a bar graph of 48 hr and 72 hr human aortic smooth muscle cell (HA-VSMC or, HASMC) survival at the indicated MOI.

FIGS. 23A and 23B show FKRP in 48-hour post transduction HASMC cell lysate. (FIG. 23A) Protein expression of FKRP and GAPDH. (FIG. 23B) FKRP activity normalized to protein, and FKRP per vector genome (vg).

FIGS. 24A and 24B show FKRP in 72-hour post transduction HASMC cell lysate. (FIG. 24A) Protein expression of FKRP and GAPDH (FIG. 24B) FKRP activity normalized to protein, and FKRP per vector genome (vg).

FIG. 25 shows 72-hour HASMC cell survival at the indicated MOI.

FIGS. 26A and 26B show FKRP in 72-hour post transduction HASMC cell lysate. (FIG. 26A) Protein expression of FKRP and GAPDH. (FIG. 26B) FKRP activity normalized to protein, and FKRP per vector genome (vg).

The above described figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION OF THE INVENTION

Limb-girdle muscular dystrophy (LGMD) is a diverse group of disorders with many subtypes categorized by disease gene and inheritance. Multiple genetic mutations, which result in defects in either structural proteins or enzymes, have been identified as causing LGMD. Limb-girdle muscular dystrophy 2I (LGMD2I), also known in the art as muscular dystrophy limb-girdle; autosomal recessive 9; LGMDR9 muscular dystrophy; limb-girdle type 2I; muscular dystrophy-dystroglycanopathy limb-girdle; and FRKP-related limb-girdle, is a monogenic, ultra-rare orphan disease.

LGMD2I is classified as an autosomal recessive muscular dystrophy caused by mutations in the gene for fukutin-related protein (FKRP), needed for glycosylation of α-dystroglycan (α-DG). Without FKRP, impaired glycosylation of α-DG reduces binding to laminin in the extracellular matrix, thus allowing increased shear damage to the muscle cell sarcolemma, chronic inflammation, and breakdown of muscle fibers over time. LGMD2I is a slowly progressing disease with significant disability and early death in juveniles/adults. These patients are prone to cardiac fibrosis, respiratory complications, and dysphagia that may lead to early death. The founder L276I mutation (homozygous) represents approximately 70% of European cases. L276I heterozygotes (25%) have more severe phenotype (various mutations on 2nd allele).

Aspects of the invention relate to the development of nucleic acids encoding Fukutin-related protein for use in gene therapy for the treatment of diseases such as limb-girdle muscular dystrophy 2I.

As used herein, “FKRP” refers to fukutin-related protein. Nucleic acids, vectors, compositions and methods described herein are directed at increasing the level of FKRP in a cell (e.g., muscle cell). Such methods may, for example be beneficial to a subject having a deficiency in glycosylation α-dystroglycan (herein referred to as a dystroglycanopathy disorder).

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “hybridizing” means annealing to two at least partially complementary nucleotide sequences in a hybridization process. In order to allow hybridisation to occur complementary nucleic acid molecules are generally thermally or chemically denatured to melt a double strand into two single strands and/or to remove hairpins or other secondary structures from single-stranded nucleic acids. The stringency of hybridisation is influenced by conditions such as temperature, salt concentration and hybridisation buffer composition. Conventional hybridisation conditions are described in, for example, Sambrook (2001) Molecular Cloning: a laboratory manual, 3rd Edition Cold Spring Harbor Laboratory Press, CSH, New York, but the skilled craftsman will appreciate that numerous different hybridisation conditions can be designed in function of the known or the expected homology and/or length of the nucleic acid sequence. High stringency conditions for hybridisation include high temperature and/or low sodium/salt concentration (salts include sodium as for example in NaCl and Na-citrate) and/or the inclusion of formamide in the hybridisation buffer and/or lowering the concentration of compounds such as SDS (sodium dodecyl sulphate detergent) in the hybridisation buffer and/or exclusion of compounds such as dextran sulphate or polyethylene glycol (promoting molecular crowding) from the hybridisation buffer. By way of non-limiting example, representative salt and temperature conditions for stringent hybridization are: 1×SSC, 0.5% SDS at 65° C. The abbreviation SSC refers to a buffer used in nucleic acid hybridization solutions. One litre of a 20× (twenty times concentrate) stock SSC buffer solution (pH 7.0) contains 175.3 g sodium chloride and 88.2 g sodium citrate. A representative time period for achieving hybridisation is 12 hours.

The meaning of “consensus sequence” is well-known in the art. In the present application, the following notation is used for the consensus sequences, unless the context dictates otherwise. Considering the following exemplary DNA sequence:

A[CT]N{A}YR-A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof. The term “synthetic promoter” as used herein relates to a promoter that does not occur in nature.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridisation between nucleic acid strands.

A “spacer sequence” or “spacer” as used herein is a nucleic acid sequence that separates two functional nucleic acid sequences. It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another.

As used herein, the term “amino acid” encompasses any naturally occurring amino acid, modified forms thereof, and synthetic amino acids.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs) are for the expression of the exogenous gene in the target cell, and generally have a promoter sequence that drives expression of the exogenous gene/ORF. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

“Delivery vectors” are used to deliver their nucleic acid cargo into a cell, typically to express the nucleic acid in the cell. In one embodiment, delivery vectors of the present invention include, without limitation viral vectors. A variety of viral vectors are known in the art (e.g., those derived from herpesvirus, Epstein-Barr virus, retrovirus, baculovirus, adenovirus, or parvovirus such as adeno-associated virus). Non-viral delivery vectors are also known in the art and their use is also encompassed by the instant invention. In one embodiment, the viral vector is a recombinant adeno-associated virus (AAV). Such viral vectors comprise an AAV capsid and can package an AAV or rAAV genome or any other nucleic acid including viral nucleic acids. Alternatively, in some contexts, the term “vector,” “virus vector,” “delivery vector” (and similar terms) may be used to refer to the vector genome (e.g., vDNA) in the absence of the virion and/or to a viral capsid that acts as a transporter to deliver molecules tethered to the capsid or packaged within the capsid.

As used herein, the term “virus vector,” (e.g., AAV vector) “viral delivery vector” (and similar terms) in a specific embodiment generally refers to a virus particle that functions as a nucleic acid delivery vehicle, and which comprises the viral nucleic acid (i.e., the vector genome) packaged within the virion.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged.

A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base inverted terminal repeat(s) (ITR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two ITRs (e.g., AAV ITRs), which generally will be at the 5′ and 3′ ends of the heterologous nucleotide sequence(s), but need not be contiguous thereto. The ITRs can be the same or different from each other. The vector genome can also contain a single ITR at its 3′ or 5′ end.

As used herein, the terms “virus vector,” “viral vector”, “vector” or “gene delivery vector” refer to a virus (e.g., AAV) particle that functions as a nucleic acid delivery vehicle, and which comprises the vector genome (e.g., viral DNA [vDNA]) packaged within a virion.

As used herein, the term “viral vector” may refer to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring synthetic nucleic acids described herein into cells either in vitro or in vivo. Numerous forms of viral vectors are known in the art and provided herein.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered (see, e.g., Table 3). An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585. The capsid proteins can be naturally occurring or modified, as is well known in the art.

Further, the viral capsid or genomic elements can contain other modifications, including insertions, deletions and/or substitutions.

A “chimeric’ capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

As used herein, the term “haploid AAV” shall mean that AAV as described in PCT/US18/22725, which is incorporated herein.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are muscle-specific enhancers (often referred to as muscle-specific CREs, or muscle-specific CRE enhancers, or suchlike). In the present context, it is preferred that the CRE is located 1500 nucleotides or less from the transcription start site (TSS), more preferably 1000 nucleotides or less from the TSS, more preferably 500 nucleotides or less from the TSS, and suitably 250, 200, 150, or 100 nucleotides or less from the TSS. CREs of the present invention are preferably comparatively short in length, preferably 100 nucleotides or less in length, for example they may be 90, 80, 70, 60 nucleotides or less in length.

The term “cis-regulatory module” or “CRM” means a functional module made up of two or more CREs; in the present invention the CREs are typically liver-specific enhancers. Thus, in the present application a CRM typically comprises a plurality of muscle-specific enhancer CREs. Typically, the multiple CREs within the CRM act together (e.g. additively or synergistically) to enhance the transcription of a gene that the CRM is operably associated with. There is conservable scope to shuffle (i.e. reorder), invert (i.e. reverse orientation), and alter spacing in CREs within a CRM. Accordingly, functional variants of CRMs of the present invention include variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

The term “synthetic promoter” as used herein relates to a promoter that does not occur in nature. In the present context it typically comprises a synthetic CRE and/or CRM of the present invention operably linked to a minimal (or core) promoter or proximal promoter, e.g., a muscle-specific. The CREs and/or CRMs of the present invention serve to enhance muscle-specific transcription of a gene operably linked to the promoter. Parts of the synthetic promoter may be naturally occurring (e.g. the minimal promoter or one or more CREs in the promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters are discussed above, and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box).

As used herein, “proximal promoter” relates to the minimal promoter plus the proximal sequence upstream of the gene that tends to contain primary regulatory elements. It often extends approximately 250 base pairs upstream of the TSS, and includes specific TFBS. In the present case, the proximal promoter is suitably a naturally occurring proximal promoter (e.g., a liver-specific or CNS-specific) that can be combined with one or more CREs or CRMs of the present invention. However, the proximal promoter can be synthetic.

A “functional variant” of a cis-regulatory element, cis-regulatory module, promoter or other nucleic acid sequence in the context of the present invention is a variant of a reference sequence that retains the ability to function in the same way as the reference sequence, e.g. as a muscle-specific cis-regulatory enhancer element, muscle-specific cis-regulatory module or muscle-specific promoter. Alternative terms for such functional variants include “biological equivalents” or “equivalents”.

It will be appreciated that the ability of a given cis-regulatory element to function as a muscle-specific enhancer is determined principally by the ability of the sequence to bind the same muscle-specific TFs that bind to the reference sequence. Accordingly, in most cases, a functional variant of a cis-regulatory element will contain TFBS for the same TFs as the reference cis-regulatory element. It is preferred, but not essential, that the TFBS of a functional variant are in the same relative positions (i.e. order) as the reference cis-regulatory element. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, in the same orientation and on the same strand as the reference sequence. It will also be appreciated that the sequences lying between TFBS (referred to in some cases as spacer sequences, or suchlike) are of less consequence to the function of the cis-regulatory element. Such sequences can typically be varied considerably, and their lengths can be altered. However, in preferred embodiments the spacing (i.e. the distance between adjacent TFBS) is substantially the same (e.g. it does not vary by more than 20, preferably by not more than 10%, more preferably it is the same) in a functional variant as it is in the reference sequence. It will be apparent that in some cases a functional variant of a cis-regulatory enhancer element can be present in the reverse orientation, e.g. it can be the reverse complement of a cis-regulatory enhancer element as described above, or a variant thereof.

Levels of sequence identity between a functional variant and the reference sequence can also be an indicator or retained functionality. High levels of sequence identity in the TFBS of the cis-regulatory element is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

A “muscle specific promoter” is one that promotes substantially higher expression in muscle tissue than other tissues. Examples of muscle specific promoters include, without limitation, muscle creatine kinase (MCK) promoter, dMCK promoter, tMCK promoter, enh358MCK promoter, and the CK6 promoter (Wang et al. Gene Ther 15, 1489-1499 (2008)), and Syn100 promoter ((Qiao et al., Molecular Therapy Vol. 22 no. 11, p. 1890-1899 (2014)). Additional muscle-specific promoters are provided herein.

“Muscle-specific” or “muscle-specific expression” refers to the ability of a cis-regulatory element, cis-regulatory module or promoter to enhance or drive expression of a gene in muscle tissue (or in muscle-derived cells) in a preferential or predominant manner as compared to other tissues (e.g. spleen, liver, lung, blood, and brain). Expression of the gene can be in the form of mRNA or protein. In preferred embodiments, muscle-specific expression is such that there is negligible expression in other (i.e. non-muscle) tissues or cells, i.e. expression is highly muscle-specific. In some embodiments, a muscle specific promoter promotes expression in skeletal muscle and/or cardiac muscle. In some embodiments, the muscle specific promoter promotes 30% or more, 40% or more, 50% or more, 60% or more, 70% or more, 80% or more, 90% or more, 95% or more, 96% or more, 97% or more, 98% or more, 99% or more expression in muscle tissue than one or more other tissues. In some embodiments, the muscle specific promoter results in no significant or detectable expression in one or more non-muscle tissues.

The term “pharmaceutically acceptable” as used herein is consistent with the art and means compatible with the other ingredients of the pharmaceutical composition and not deleterious to the recipient thereof.

The term “effective amount” is synonymous with “therapeutically effective amount”, “effective dose”, or “therapeutically effective dose.” A “therapeutically effective” amount as used herein is an amount that is sufficient to provide some improvement or benefit to the subject. Alternatively stated, a “therapeutically effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject. In an embodiment, the effectiveness of a therapeutic compound disclosed herein to treat dystroglycanopathy disorders can be determined, without limitation, by observing an improvement in an individual based upon one or more clinical symptoms, and/or physiological indicators associated with the disorder. In an embodiment, an improvement in the symptoms associated with the disorder can be indicated by a reduced need for a concurrent therapy.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

### Nucleic Acids Encoding FKRP

One aspect of the invention relates to a synthetic nucleic acid encoding human fukutin related protein (FKRP). FKRP is one of the proteins identified to be in the DG glycosylation pathway. It is involved in the glycosylation of O-linked mannose in α-DG (Qiao et al., Molecular Therapy 22, pp 1890-1899 (2014)). Human FKRP is well characterized. Mutations in the gene encoding FKRP result in a wide spectrum of disease phenotypes including the mild limb-girdle muscular dystrophy 2I (LGMD2I), the severe Walker-Warburg syndrome, congenital muscle dystrophy type 1C (CMD1C), and muscle-eye-brain disease. Mutations in the FKRP gene can also result in a severe congenital muscular dystrophy-dystroglycanopathy with brain and eye anomalies (type A5; MDDGA5) and a congenital muscular dystrophy-dystroglycanopathy with or without impaired intellectual development (type B5; MDDGB5). Introduction of a functional FKRP gene into a subject with such a disease to thereby increase expression and functional FKRP levels in the muscle tissue of the subject will have therapeutic benefit to the subject. Optimization of the nucleic acid encoding the FKRP protein that is introduced into the subject maximizes expression to thereby increase the therapeutic benefit to the subject. Optimization includes, without limitation, reduction in the CpG sites, and overall reduction in GC content of the FKRP encoding nucleic acid.

In one embodiment, the subject has a mutation in the FKRP mutation that results in a FKRP deficiency. Exemplary FKRP mutations that result in an FKRP deficiency are described in, e.g., Liang, W-C; et al. Orphanet Journal of Rare Diseases (2020) 15:160.; Liu, W.; et al. bioRxiv preprint, doi: 10.1101/502708; posted Feb. 7, 2019.; Nallamilli, B.; et al. Annals of Clinical and Translational Neurology 2018; 5(12): 1574-1587.; Murphy, L. B.; et al. Annals of Clinical and Translational Neurology 2020; 7(5): 757-766, and are provided herein in Table 13.

TABLE 13
Exemplary FKRP mutations (e.g., as described in Murphy, L.B .; et al. Annals of Clinical and
Translational Neurology 2020; 7(5): 757-766)

| | Nucleotide | Effect on | Novel | Nucleotide | Effect on | Novel |
|---|---|---|---|---|---|---|
| No. of | change - | FKRP protein | mutation - | change - | FKRP protein | mutation - |
| patients | allele 1 | sequence 1 | allele 1 | allele 2 | sequence 2 | allele 2 |
| 206 | c.826C > A | p.Leu276Ile | No | c.826C > A | p.Leu276Ile | No |
| 1 | c.826C > A | p.Leu276Ile | No | c.826C > A | p.Leu276Ile | No |

| | | |
|---|---|---|
| c.390insTACC | p.Asp131TyrfsTer7 | Yes |

| | | | | | | |
|---|---|---|---|---|---|---|
| 4 | c.826C > A | p.Leu276Ile | No | c.586G > C | p.Gly196Arg | No |
| 4 | c.826C > A | p.Leu276Ile | No | c.1384C > T | p.Pro462Ser | No |
| 4 | c.826C > A | p.Leu276Ile | No | c.1073C > T | p.Pro358Leu | No |
| 4 | c.826C > A | p.Leu276Ile | No | c.919T > A | p.Tyr307Asn | No |
| 3 | c.826C > A | p.Leu276Ile | No | c.229C > T | p.Gln77* | Yes |
| 2 | c.826C > A | p.Leu276Ile | No | c.1187insA | p.Ala397Glyfs*67 | No |
| 2 | c.826C > A | p.Leu276Ile | No | c.1486T > A | p.*496Argext*21 | No |
| 2 | c.826C > A | p.Leu276Ile | No | c.962C > A | p.Ala321Glu | No |
| 2 | c.826C > A | p.Leu276Ile | No | c.1016G > A | p.Arg339His | No |
| 2 | c.826C > A | p.Leu276Ile | No | c.1088T > G | p.Val363Gly | Yes |
| 2 | c.826C > A | p.Leu276Ile | No | c.946C > T | p.Pro316Ser | No |
| 2 | c.826C > A | p.Leu276Ile | No | c.928G > T | p.Glu310* | No |
| 2 | c.826C > A | p.Leu276Ile | No | c.469G > C | p.Ala157Pro | No |
| 2 | c.826C > A | p.Leu276Ile | No | c.532T > G | p.Trp178Gly | Yes |
| 2 | c.826C > A | p.Leu276Ile | No | c.646C > T | p.Arg216Trp | Yes |
| 2 | c.826C > A | p.Leu276Ile | No | c.545A > G | p.Tyr182Cys | No |
| 2 | c.826C > A | p.Leu276Ile | No | c.217C > T | p.Gln73* | Yes |
| 1 | c.826C > A | p.Leu276Ile | No | c.1217A > C | p.Gln406Pro | Yes |
| 2 | c.826C > A | p.Leu276Ile | No | c.1054C > G | p.Arg352Gly | No |
| 1 | c.826C > A | p.Leu276Ile | No | c.1054C > T | p.Arg352Cys | No |
| 1 | c.826C > A | p.Leu276Ile | No | c.1054C > A | p.Arg352Ser | Yes |
| 1 | c.826C > A | p.Leu276Ile | No | c.673C > T | p.Gln225* | Yes |
| 1 | c.826C > A | p.Leu276Ile | No | Not | Not specified | — |
| | | | | specified | | |
| 1 | c.826C > A | p.Leu276Ile | No | c.1384C > T | p.Pro462Ser | No |

| | | |
|---|---|---|
| c.341C > G | p.Ala114Gly | No |

| | | | | | | |
|---|---|---|---|---|---|---|
| 1 | c.826C > A | p.Leu276Ile | No | c.341C > G | p.Ala114Gly | No |
| 1 | c.826C > A | p.Leu276Ile | No | c.135C > T | p.Ala45Ala | No |

| | | |
|---|---|---|
| c.341C > G | p.Ala114Gly | No |
| c.1486T > A | p.Stop496Arg | No |

| | | | | | | |
|---|---|---|---|---|---|---|
| 1 | c.826C > A | p.Leu276Ile | No | c.426_437del | p.Arg143_ | No |
| | | | | | Glu146del | |
| 1 | c.826C > A | p.Leu276Ile | No | c.1037C > T | p.Ser346Leu | Yes |
| 1 | c.826C > A | p.Leu276Ile | No | c.1381G > C | p.Ala461Pro | Yes |
| 1 | c.826C > A | p.Leu276Ile | No | c.948_949dupC | p.Cys317Serfs*112 | Yes |

| | | |
|---|---|---|
| c.1000_1017dup18 | p.Glu334_Arg339dup | No |

| | | | | | | |
|---|---|---|---|---|---|---|
| 1 | c.826C > A | p.Leu276Ile | No | c.934C > G | p.Arg312Gly | Yes |
| 1 | c.826C > A | p.Leu276Ile | No | c.430A > G | p.Met144Val | No |
| 1 | c.826C > A | p.Leu276Ile | No | c.362T > A | p.Val121Glu | No |
| 1 | c.826C > A | p.Leu276Ile | No | c.398C > A | p.Ala133Glu | Yes |
| 1 | c.826C > A | p.Leu276Ile | No | c.1268G > C | p.Arg423Pro | No |
| 1 | c.826C > A | p.Leu276Ile | No | c.391G > A | p.Asp131Asn | Yes |
| 1 | c.826C > A | p.Leu276Ile | No | c.88C > T | p.Gln30* | Yes |
| 1 | c.826C > A | p.Leu276Ile | No | c.214C > T | p.Gln72* | No |
| 1 | c.826C > A | p.Leu276Ile | No | c.534G > T | p.Trp178Cys | Yes |
| 1 | c.826C > A | p.Leu276Ile | No | c.1000G > T | p.Glu334* | Yes |
| 1 | c.826C > A | p.Leu276Ile | No | c.1433T > C | p.Ile478Thr | No |
| 1 | c.826C > A | p.Leu276Ile | No | c.605T > A | p.Leu202Gln | No |
| 1 | c.826C > A | p.Leu276Ile | No | c.620T > C | p.Leu207Pro | Yes |
| 1 | c.826C > A | p.Leu276Ile | No | c.872delA | p.Lys291Argfs*137 | No |
| 1 | c.826C > A | p.Leu276Ile | No | c.943C > T | p.Pro315Leu | Yes |
| 1 | c.826C > A | p.Leu276Ile | No | c.946C > G | p.Pro316Ala | Yes |

| | | |
|---|---|---|
| c.970G > C | p.Glu324Gln | Yes |

| | | | | | | |
|---|---|---|---|---|---|---|
| 1 | c.826C > A | p.Leu276Ile | No | c.836G > A | p.Trp279* | No |
| 1 | c.826C > A | p.Leu276Ile | No | c.1076G > C | p.Trp359Ser | Yes |
| 1 | c.826C > A | p.Leu276Ile | No | c.899T > C | p.Val300Ala | No |
| 1 | c.826C > A | p.Leu276Ile | No | c.1253G > A | p.Trp418* | No |
| 1 | c.826C > A | p.Leu276Ile | No | c.797insC | p.Ala267Glyfs*123 | Yes |
| 1 | c.826C > A | p.Leu276Ile | No | c.158_ | p.Glu55Cysfs*15 | No |
| | | | | 162dupTGCGG | | |
| 1 | c.826C > A | p.Leu276Ile | No | c.162_ | p.Phe56Glyfs*6 | No |
| | | | | 165dupGGAG | | |
| 1 | c.826C > A | p.Leu276Ile | No | c.1171G > A | p.Gly391Ser | No |
| 1 | c.826C > A | p.Leu276Ile | No | c.76_77delTG | p.Trp26Alafs*6 | No |
| 1 | c.826C > A | p.Leu276Ile | No | c.1115T > G | p.Val372Gly | No |
| 1 | c.826C > A | p.Leu276Ile | No | c.1141delG | p.Ala381Glnfs*47 | No |
| 1 | c.826C > A | p.Leu276Ile | No | c.264C > G | p.Tyr88* | No |
| 1 | c.826C > A | p.Leu276Ile | No | c.160C > G | p.Arg54Gly | Yes |
| 1 | c.826C > A | p.Leu276Ile | No | c.266C > T | p.Pro89Leu | No |
| 2 | c.1100T > C | p.Ile367Thr | No | c.1100T > C | p.Ile367Thr | No |
| 2 | c.1388A > G | p.Asn463Ser | Yes | c.162_ | p.Phe56Glyfs*6 | No |
| | | | | 165dupGGAG | | |
| 1 | c.1486T > A | p.*496Argext*21 | No | c.1486T > A | p.*496Argext*21 | No |
| 1 | c.1073C > T | p.Pro358Leu | No | c.1210C > T | p.Arg404Cys | No |
| 1 | c.265C > T | p.Pro89Ser | Yes | c.1433T > G | p.Ile478Ser | No |
| 1 | c.266C > T | p.Pro89Leu | No | c.1247A > G | p.Asp416Gly | Yes |
| 1 | c.430A > G | p.Met144Val | No | c.469G > C | p.Ala157Pro | No |
| 1 | c.520A > T | p.Ser174Cys | No | Not specified | Not specified | — |
| 1 | c.1343C > T | p.Pro448Leu | Yes | c.1387A > G | p.Gln460Glu | Yes |

In addition, known FKRP mutation are further described, e.g., on the world wide web uniprot.org/uniprot/Q9H9S5.

The CpG sites, or CG sites, are regions of DNA where a cytosine nucleotide is followed by a guanine nucleotide in the linear sequence of bases along its 5′→3′ direction. Deletion or reduction in the number of CpG sites can reduce the immunogenicity of an introduced coding sequence in a subject. This results from a reduction or complete inhibition in TLR-9 binding to the DNA sequence, which occurs at CpG sites. It is also well known that methylation of CpG motifs results in transcriptional silencing. Removal of CpG motifs in the sequence is expected to result in decreased TLR-9 recognition and/or decreased methylation and therefore decreased transgene silencing. In some embodiments, one or more CpG sites are omitted from the FKRP coding sequence. In some embodiments, 10, 20, 30, 40, 50, 60, 70, 80, 90, 95, 96, 97, 98, or 99% or all CpG sites are omitted from the FKRP coding sequence. In some embodiments, all CpG (or, 100% CpG) sites are omitted from the FKRP coding sequence. Removal or, depletion of the CpG sites is achieved by substitution with a different nucleotide, preserving the amino acid sequence of the protein which is encoded.

Another form of optimization of the FKRP coding sequence is a reduction in the overall GC content of the nucleic acid. This is accomplished by eliminating guanines and cytosines from the sequence and replacing them as needed to preserve the encoded amino acid sequence of the FKRP protein. Reduction in GC content can be quantitated by comparison to a FKRP coding sequence prior to the reduction (e.g., to native sequence SEQ ID NO: 6). In some embodiments, the overall GC content of the FKRP coding sequence is reduced by greater than 10% as compared to the native sequence (SEQ ID NO: 6). In some embodiments, the synthetic polynucleotide encoding a FKRP comprises, consists essentially of, or consists of a nucleotide sequence encoding FKRP, wherein the GC content is reduced by about 11% to about 15% compared to the GC content of SEQ ID NO: 6 (e.g., 10.5%, 11%, 11.5%, 12%, 12.5%, 13%, 13.5%, 14%, 14.5%, 15%, or any range or value therein). In some embodiments, the GC content is reduced by about 15% or more (e.g., 15%, 15.5%, 16%, 16.5%, 17%, 17.5%, 18%, 18.5%, 19%, 19.5%, 20% or more). In some embodiments, the GC content is reduced by about 20% to about 30% (e.g., 20%, 21%, 22%, 23%, 24%, 25%, 26%, 27%, 28%, 29% or 30%) as compared to the GC content of SEQ ID NO: 6. In some embodiments, the GC content is reduced by about 30-40% (e.g., 30%, 31%, 32%, 33%, 34%, 35%, 36%, 37%, 38%, 39% or 40%) as compared to the GC content of SEQ ID NO: 6. In some embodiments, the GC content is reduced by about 40-50%, about 50-60%, about 60-70% as compared to the GC content of SEQ ID NO: 6. The present inventors have surprisingly discovered that, contrary to what is commonly understood in the art of nucleic acid expression and protein production, wherein increasing GC content is understood to increase expression (Kudla et al. PLos Biology DOI: 10.1371/journal.pbio.0040180 (2006)), reducing the GC content of the polynucleotide encoding by greater than 10% that of SEQ ID NO: 6, increases expression of said polynucleotide as compared to the native polynucleotide encoding FKRP, and thereby increasing production of FKRP as compared to the native polynucleotide encoding FKRP.

As used herein, “coFKRP” means codon optimized FKRP including 0% CpG depleted FKRP.

In some embodiments, the synthetic nucleic acid has the nucleotide sequence set out in SEQ ID NO: 2. In some embodiments, the synthetic nucleic acid has a nucleotide sequence that has at least 70%, 75%, 80%, 81%, 82%, 83%, 84%, 85%, 86%, 87%, 88%, 89% or 90% identity to SEQ ID NO: 2. In some embodiments, the synthetic nucleic acid has a nucleotide sequence that has at least 91%, 92%, 93%, 94%, 95%, 96%, 97%, 98%, or 99% identity to SEQ ID NO: 2. In some embodiments, the synthetic nucleic acid has the indicated sequence identity to SEQ ID NO: 2, and further has the herein indicated reduced CpG sites (e.g., 0%) and/or reduced GC content (e.g., greater than 10%, or 15% or greater, relative to SEQ ID NO: 6) described herein.

In some embodiments, the synthetic nucleic acid has the nucleotide sequence set out in SEQ ID NO: 407. In some embodiments, the synthetic nucleic acid has a nucleotide sequence that has at least 70%, 75%, 80%, 81%, 82%, 83%, 84%, 85%, 86%, 87%, 88%, 89% or 90% identity to SEQ ID NO: 407. In some embodiments, the synthetic nucleic acid has a nucleotide sequence that has at least 91%, 92%, 93%, 94%, 95%, 96%, 97%, 98%, or 99% identity to SEQ ID NO: 407. In some embodiments, the synthetic nucleic acid has the indicated sequence identity to SEQ ID NO: 407, and further has the herein indicated reduced CpG sites (e.g., 0%) and/or reduced GC content (e.g., greater than 10%, or 15% or greater, relative to SEQ ID NO: 6) described herein.

In some embodiments, the synthetic nucleic acid encoding FKRP further comprises a promoter (e.g., a muscle-specific promoter). Preferably the FKRP is operatively linked to the promoter. In one embodiment, the muscle-specific promoter is Syn100. Various muscle-specific promoters (e.g., synthetic) for inclusion in the synthetic nucleic acids are described herein (e.g., those in Tables 1-4). In some embodiments, rAAV comprising SEQ ID NO: 2, further comprises a muscle specific promoter e.g., Syn100; or, a synthetic muscle specific promoter selected from the Tables 1-4, or fragments thereof, and/or, an enhancer, and/or cis-regulatory elements (CREs; see e.g., Tables 1-4), or any combination thereof, or, shortened muscle specific promoters selected from Table 8-12, or, fragments thereof, and/or, cis regulatory elements (CREs; see e.g., Tables 8-12), or any combination thereof. In some embodiments, rAAV comprising SEQ ID NO: 407, further comprises a muscle specific promoter e.g., Syn100; or, a synthetic muscle specific promoter selected from the Tables 1-4, or fragments thereof, and/or, an enhancer, and/or cis-regulatory elements (CREs; see e.g., Tables 1-4), or any combination thereof, or, shortened muscle specific promoters selected from Table 8-12, or, fragments thereof, and/or, cis regulatory elements (CREs; see e.g., Tables 8-12), or any combination thereof.

In some embodiments, the synthetic nucleic acid further comprises one or more additional regulatory components and/or components of a vector (e.g., a viral vector), as described herein. In some embodiments the additional regulatory component is an enhancer sequence (e.g., CMV enhancer, muscle creatine kinase enhancer, myosin light chain enhancer, etc., and combinations thereof). In some embodiments, the synthetic nucleic acid further comprises one or more AAV genome elements disclosed herein such as inverted terminal repeats. In some embodiments, the nucleic acid further comprises a 5′ and a 3′ AAV ITR.

### Vectors Comprising the FKRP Encoding Nucleic Acid

Another aspect of the invention relates to a vector comprising the synthetic nucleic acid encoding FKRP disclosed herein. Such vectors and compositions comprising the vectors are used for production of the synthetic nucleic acid, production of the vectors, and therapeutic use to increase the level of functional FKRP in a cell (e.g., muscle cells of a subject in need thereof). In various embodiments, the vector comprising the nucleic acid will, as appropriate, further comprise regulatory sequences operatively linked to the nucleic acid. Examples of such regulatory sequences are described herein.

In some embodiments, the vector (e.g., viral vector such as AAV) may further comprise a nucleic acid element that reduces expression in the liver. In representative embodiments, the vector further comprises a mir122 binding element. The mir122 sequence and its use to reduce expression in the liver is well known in the art (See, e.g., Qiao et al, Gene Therapy 18, 403-410 (April 2011) doi:10.1038/gt.2010.157).

In some embodiments, the vector is a non-viral vector such as a plasmid. Examples of non-viral vectors are provided herein. In some embodiments, the vector is a viral vector.

### Recombinant Viral Vectors and Production

In some embodiments of the invention, the vector is a DNA or RNA virus. Nonlimiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors produced may comprise the genome, in part or entirety, of any naturally occurring and/or recombinant viral vector nucleotide sequence (e.g., AAV, adeno virus, lentivirus, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

The viral vectors comprising the FKRP transgene cassette described herein can be produced by any means known in the art. Without limitation, one example of a method of producing viral particles is a method comprising (a) providing any of the stable cell line described herein, e.g., a cell line having stable expression of a heterologous toxic protein under the control of an inducible promoter, in a viral expression system; (b) culturing the cells under conditions in which at least one toxic protein is expressed, wherein the at least one toxic protein is operatively linked to at least one inducible promoter; (c) culturing the cells under conditions in which viral particles are produced; and (d) optionally isolating the viral particles.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Further, production of AAV vectors is further described, e.g., in U.S. Pat. No. 9,441,206, the contents of which is incorporated herein by reference in its entirety.

Viral vectors produced in a viral expression system can be released (i.e. set free from the cell that produced the vector) using any standard technique. For example, viral vectors can be released via mechanical methods, for example microfluidization, centrifugation, or sonication, or chemical methods, for example lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

Variant viral vector sequences can be used to produce viral vectors in the viral expression system described herein. For example, or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, adeno virus, lentivirus, etc.).

It is to be understood that a viral expression system will further be modified to include any necessary elements required to complement a given viral vector during its production using methods described herein. For example, in certain embodiment, the nucleic acid cassette is flanked by terminal repeat sequences. In one embodiment, for the production of rAAV vectors, the AAV expression system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

A viral expression system for manufacturing an AAV vector (e.g., an AAV expression system) could further comprise Replication (Rep) genes and/or Capsid (Cap) genes in trans, for example, under the control of an inducible promoter. Expression of Rep and Cap can be under the control of one inducible promoter, such that expression of these genes are turned “on” together, or under control of two separate inducible promoters that are turned “on” by distinct inducers. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties.

A viral expression system for manufacturing a lentivirus using methods described herein would further comprise long terminal repeats (LTRs) flanking the nucleic acid cassette. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase the host chromosome. LTRs and methods for manufacturing lentiviral vectors are further described, e.g., in U.S. Pat. Nos. 7,083,981B2; 6,207,455B1; 6,555,107B2; 8,349,606B2; 7,262,049B2; and U.S. Patent Application Numbers US20070025970A1; US20170067079A1; US20110028694A1; the contents of each are incorporated herein by reference in their entireties.

A viral expression system for manufacturing an adenovirus using methods described herein would further comprise identical Inverted Terminal Repeats (ITR) of approximately 90-140 base pairs (exact length depending on the serotype) flanking the nucleic acid cassette. The viral origins of replication are within the ITRs exactly at the genome ends. The adenovirus genome is a linear double-stranded DNA molecule of approximately 36000 base pairs. Often, adenoviral vectors used in gene therapy have a deletion in the E1 region, where novel genetic information can be introduced; the E1 deletion renders the recombinant virus replication defective. ITRs and methods for manufacturing adenovirus vectors are further described, e.g., in U.S. Pat. Nos. 7,510,875B2; 7,820,440B2; 7,749,493B2; 7,820,440B2; U.S. Ser. No. 10/041,049B2; International Patent Application Numbers WO2000070071A1; and U.S. Patent Application Numbers WO2000070071A1; US20030022356A1; US20080050770A1 the contents of each are incorporated herein by reference in their entireties.

In one embodiment, the viral expression system can be a host cell, such as a virus, a mammalian cell or an insect cell. Exemplary insect cells include but are not limited to Sf9, Sf21, Hi-5, and S2 insect cell lines. For example, a viral expression system for manufacturing an AAV vector could further comprise a baculovirus expression system, for example, if the viral expression system is an insect cell. The baculovirus expression system is designed for efficient large-scale viral production and expression of recombinant proteins from baculovirus-infected insect cells. Baculovirus expression systems are further described in, e.g., U.S. Pat. No. 6,919,085B2; 6,225,060B1; 5,194,376A; the contents of each are incorporated herein by reference in their entireties.

In another embodiment, the viral expression system is a cell-free system. Cell-free systems for viral vector production are further described in, for example, Cerqueira A., et al. Journal of Virology, 2016; Sheng J., et al. The Royal Society of Chemistry, 2017; and Svitkin Y. V., and Sonenberg N. Journal of Virology, 2003; the contents of which are incorporated herein by reference. In some embodiments the nucleic acid sequences disclosed herein is delivered via non-viral DNA constructs comprising at least one DD-ITR. The non-viral DNA constructs as described in WO 2019/246554 is incorporated herein by reference in its entirety.

rAAV Vectors and Production

Aspects of the invention relate to a recombinant AAV vector comprising the synthetic nucleic acid encoding FKRP described herein. In one embodiment, the rAAV vector (also referred to as a rAAV virion) as disclosed herein comprises a capsid protein, and a rAAV genome within the capsid protein. A rAAV capsid of the rAAV virion used in the vectors and methods described herein is any of those listed in Table 6, or any combination thereof. In one embodiment, the rAAV of the present invention comprises at least one capsid protein sequence from the capsid proteins of the AAV serotypes described in Table 6.

TABLE 6
Table 6: AAV Serotypes and exemplary published corresponding capsid sequence

| Serotype and where capsid sequence is | Serotype and where capsid sequence is |
|---|---|
| published | published |
| AAV3.3b (See SEQ ID NO: 72 in US20030138772) | AAV3-3 (See SEQ ID NO: 200 US20150315612) |
| AAV3-3 (See SEQ ID NO: 217 US20150315612) | AAV3a ((See SEQ ID NO: 5 in U.S. Pat. No. 6,156,303) |
| AAV3a (See SEQ ID NO: 9 in U.S. Pat. No. 6,156,303) | AAV3b (See SEQ ID NO: 6 in U.S. Pat. No. 6,156,303) |
| AAV3b (See SEQ ID NO: 10 in U.S. Pat. No. 6,156,303) | AAV3b (See SEQ ID NO: 1 in U.S. Pat. No. 6,156,303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO: 63 in |
| | US20030138772) and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in US20030138772) | AAV42.3b (See SEQ ID NO: 36 in US20030138772) |
| AAV42.3B (See SEQ ID NO: 107 in US20030138772) | AAV42.4 (See SEQ ID NO: 33 in US20030138772) |
| AAV42.4 (See SEQ ID NO: 88 in US20030138772) | AAV42.8 (See SEQ ID NO: 27 in US20030138772) |
| AAV42.8 (See SEQ ID NO: 85 in US20030138772) | AAV43.1 (See SEQ ID NO: 39 in US20030138772) |
| AAV43.1 (See SEQ ID NO: 92 in US20030138772) | AAV43.12 (See SEQ ID NO: 41 in US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO: 4 in US20030138772; |
| | US20150315612 SEQ |
| ID NO: 182 | AAV8 (See SEQ ID NO: 95 in |
| | US20030138772), US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | U.S. Pat. No. 7,198,951, or SEQ ID NO: 223 in |
| | US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in U.S. Pat. No. 7,198,951) |
| AAV9 (See SEQ ID NO: 9 in US20160017295) | AAV9 (See SEQ ID NO: 100 in |
| | US20030138772), U.S. Pat. No. 7,198,951 SEQ ID NO: 2 |
| | AAV9 (See SEQ ID NO: 3 in U.S. Pat. No. 7,198,951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in | AAVA3.3 (See SEQ ID NO: 57 in |
| US20030138772) | US20030138772) |
| AAVA3.3 (See SEQ ID NO: 66 in | AAVA3.4 (See SEQ ID NO: 54 in |
| US20030138772) | US20030138772) |
| AAVA3.4 (See SEQ ID NO: 68 in | AAVA3.5 (See SEQ ID NO: 55 in |
| US20030138772) | US20030138772) |
| AAVA3.5 (See SEQ ID NO: 69 in | AAVA3.7 (See SEQ ID NO: 56 in |
| US20030138772) | US20030138772) |
| AAVA3.7 (See SEQ ID NO: 67 in | AAV29. (See SEQ ID NO: 11 in (AAVbb. l) |
| US20030138772) | 161 US20030138772) |
| AAVC2 (See SEQ ID NO: 61 in | AAVCh.5 (See SEQ ID NO: 46 in |
| US20030138772) | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in |
| US20150315612) | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in | AAVCy.5Rl (See SEQ ID NO: in |
| US20150159173) | US20150159173 |
| AAVCy.5R2 (See SEQ ID NO: in | AAVCy.5R3 (See SEQ ID NO: in |
| US20150159173) | US20150159173 |
| AAVCy.5R4 (See SEQ ID NO: in | AAVDJ (See SEQ ID NO: 3 in |
| US20150159173) | US20140359799) and SEQ ID NO: 2 in |
| | U.S. Pat. No. 7,588,772) |
| | AAVDJ (See SEQ ID NO: 2 in |
| | US20140359799; and SEQ ID NO: 1 in |
| | U.S. Pat. No. 7,588,772) |
| | AAVDJ-8 (See SEQ ID NO: in |
| | U.S. Pat. No. 7,588,772; |
| | Grimm et al 2008 |
| AAVDJ-8 (See SEQ ID NO: in | AAVF5 (See SEQ ID NO: 110 in |
| U.S. Pat. No. 7,588,772; | US20030138772) |
| Grimm et al 2008 | |
| AAVH2 (See SEQ ID NO: 26 in US20030138772) | AAVH6 (See SEQ ID NO: 25 in |
| | US20030138772) |
| AAVhEl. l (See SEQ ID NO: 44 in | AAVhErl.14 (See SEQ ID NO: 46 in |
| U.S. Pat. No. 9,233,131) | U.S. Pat. No. 9,233,131) |
| AAVhErl.16 (See SEQ ID NO: 48 in | AAVhErl.18 (See SEQ ID NO: 49 in |
| U.S. Pat. No. 9,233,131) | U.S. Pat. No. 9,233,131) |
| AAVhErl.23 (AAVhEr2.29) (See SEQ ID NO: 53 | AAVhErl.35 (See SEQ ID NO: 50 in |
| in U.S. Pat. No. 9,233,131) | U.S. Pat. No. 9,233,131) |
| AAVhErl.36 (See SEQ ID NO: 52 in | AAVhErl.5 (See SEQ ID NO: 45 in |
| U.S. Pat. No. 9,233,131) | U.S. Pat. No. 9,233,131) |
| AAVhErl.7 (See SEQ ID NO: 51 in | AAVhErl.8 (See SEQ ID NO: 47 in |
| U.S. Pat. No. 9,233,131) | U.S. Pat. No. 9,233,131) |
| AAVhEr2.16 (See SEQ ID NO: 55 in | AAVhEr2.30 (See SEQ ID NO: 56 in |
| U.S. Pat. No. 9,233,131) | U.S. Pat. No. 9,233,131) |
| AAVhEr2.31 (See SEQ ID NO: 58 in | AAVhEr2.36 (See SEQ ID NO: 57 in |
| U.S. Pat. No. 9,233,131) | U.S. Pat. No. 9,233,131) |
| AAVhEr2.4 (See SEQ ID NO: 54 in | AAVhEr3.1 (See SEQ ID NO: 59 in |
| U.S. Pat. No. 9,233,131) | U.S. Pat. No. 9,233,131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 |
| US20150315612) | in US20150315612) |
| AAVhu.ll (AAV16.12) (See SEQ ID NO: 57 in | AAVhu.ll (AAV16.12) (See SEQ ID NO: 153 |
| US20150315612) | in US20150315612) |
| AAVhu.12 (See SEQ ID NO: 59 in | AAVhu.12 (See SEQ ID NO: 154 in |
| US20150315612) | US20150315612) |
| AAVhu.13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.l7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in | AAVhu.20 (See SEQ ID NO: 63 in |
| US20150315612) | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in | AAVhu.3 (See SEQ ID NO: 145 in |
| US20150315612) | US20150315612) |
| AAVhu.30 (See SEQ ID NO: 70 in | AAVhu.30 (See SEQ ID NO: 131 in |
| US20150315612) | US20150315612) |
| AAVhu.31 (See SEQ ID NO: 1 in | AAVhu.31 (See SEQ ID NO: 121 in |
| US20150315612) | US20150315612) |
| AAVhu.32 (See SEQ ID NO: 2 in | AAVhu.32 (See SEQ ID NO: 122 in |
| US20150315612) | US20150315612) |
| AAVhu.33 (See SEQ ID NO: 75 in | AAVhu.33 (See SEQ ID NO: 124 in |
| US20150315612) | US20150315612) |
| AAVhu.34 (See SEQ ID NO: 72 in | AAVhu.34 (See SEQ ID NO: 125 in |
| US20150315612) | US20150315612) |
| AAVhu.35 (See SEQ ID NO: 73 in | AAVhu.35 (See SEQ ID NO: 164 in |
| US20150315612) | US20150315612) |
| AAVhu.36 (See SEQ ID NO: 74 in | AAVhu.36 (See SEQ ID NO: 126 in |
| US20150315612) | US20150315612) |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in | AAVhu.40 (See SEQ ID NO: 87 in |
| US20150315612) | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO: 8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 |
| US20150315612) | in US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44Rl (See SEQ ID NO: in |
| US20150315612) | US20150159173 |
| AAVhu.44R2 (See SEQ ID NO: in | AAVhu.44R3 (See SEQ ID NO: in |
| US20150159173 | US20150159173 |
| AAVhu.45 (See SEQ ID NO: 76 in | AAVhu.45 (See SEQ ID NO: 127 in |
| US20150315612) | US20150315612) |
| AAVhu.46 (See SEQ ID NO: 82 in | AAVhu.46 (See SEQ ID NO: 159 in |
| US20150315612) | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 |
| US20150315612) | in US20150315612) |
| AAVhu.48Rl (See SEQ ID NO: in | AAVhu.48R2 (See SEQ ID NO: in |
| US20150159173 | US20150159173 |
| AAVhu.48R3 (See SEQ ID NO: in | AAVhu.49 (See SEQ ID NO: 209 in |
| US20150159173 | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in | AAVhu.51 (See SEQ ID NO: 208 in |
| US20150315612) | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in | AAVhu.53 (AAV145.1) (See SEQ ID NO: 176 |
| US20150159173) | in US20150315612) |
| AAVhu.54 (See SEQ ID NO: 188 in | AAVhu.54 (AAV145.5) (See SEQ ID NO: 177 |
| US20150315612) | in US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 |
| US20150315612) | in US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.61 (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in | AAVhu.7 (See SEQ ID NO: 150 in |
| US20150315612) | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in | AAVhu.8 (See SEQ ID NO: 12 in |
| US20150315612) | US20150315612) |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in |
| US20150376607) | US20150376607) |
| AAV-LK05 (See SEQ ID NO: 6 in | AAV-LK05 (See SEQ ID NO: 33 in |
| US20150376607) | US20150376607) |
| AAV-LK06 (See SEQ ID NO: 7 in | AAV-LK06 (See SEQ ID NO: 34 in |
| US20150376607) | US20150376607) |
| AAV-LK07 (See SEQ ID NO: 8 in | AAV-LK07 (See SEQ ID NO: 35 in |
| US20150376607) | US20150376607) |
| AAV-LK08 (See SEQ ID NO: 9 in | AAV-LK08 (See SEQ ID NO: 36 in |
| US20150376607) | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.l (See SEQ ID NO: 28 in | AAVpi.l (See SEQ ID NO: 93 in |
| US20150315612) | US20150315612; AAVpi.2 408, see SEQ ID |
| | NO: 30 in US20150315612) |
| AAVpi.2 (See SEQ ID NO: 95 in | AAVpi.3 (See SEQ ID NO: 29 in |
| US20150315612) | US20150315612) |
| AAVpi.3 (See SEQ ID NO: 94 in | AAVrh.10 (See SEQ ID NO: 9 in |
| US20150315612) | US20150159173) |
| AAVrh.10 (See SEQ ID NO: 25 in | AAV44.2 (See SEQ ID NO: 59 in |
| US20150159173) | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in | AAVrh.13 (See SEQ ID NO: 228 in |
| US20150159173) | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in | AAVrh.20 (See SEQ ID NO: 1 in |
| US20150315612) | US20150159173) |
| AAV42.10 (See SEQ ID NO: 106 in | AAVrh.21 (AAV42.10) (See SEQ ID NO: 35 |
| US20030138772) | in US20030138772) |
| AAV42.11 (See SEQ ID NO: 108 in | AAVrh.22 (AAV42.11) (See SEQ ID NO: 37 |
| US20030138772) | in US20030138772) |
| AAV42.12 (See SEQ ID NO: 113 in | AAVrh.23 (AAV42.12) (See SEQ ID NO: 58 |
| US20030138772) | in US20030138772) |
| AAV42.13 (See SEQ ID NO: 86 in | AAVrh.24 (AAV42.13) (See SEQ ID NO: 31 |
| US20030138772) | in US20030138772) |
| AAV42.15 (See SEQ ID NO: 84 in | AAVrh.25 (AAV42.15) (See SEQ ID NO: 28 |
| US20030138772) | in US20030138772) |
| AAVrh.2R (See SEQ ID NO: in US20150159173 | AAVrh.31 (AAV223.1) (See SEQ ID NO: 48 |
| | in US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 |
| US20150315612) | in US20150315612) |
| AAVrh.56 (See SEQ ID NO: 54 in | AAVrh.56 (See SEQ ID NO: 152 in |
| US20150315612) | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in | AAVrh.58 (See SEQ ID NO: 48 in |
| US20150315612) | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in | AAVrh.59 (See SEQ ID NO: 110 in |
| US20150315612) | US20150315612) |
| AAVrh.60 (See SEQ ID NO: 31 in | AAVrh.60 (See SEQ ID NO: 120 in |
| US20150315612) | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 |
| US20150315612) | in US20150315612) |
| AAVrh.64 (See SEQ ID NO: 15 in | AAVrh.64 (See SEQ ID NO: 43 in |
| US20150315612) | US20150159173 and SEQ ID NO: 99 in |
| | US20150315612) |
| | AAVrh.64 (See SEQ ID NO: 233 in |
| | US20150315612) |
| AAVRh.64Rl (See SEQ ID NO: in | AAVRh.64R2 (See SEQ ID NO: in |
| US20150159173 | US20150159173 |
| AAVrh.65 (See SEQ ID NO: 35 in | AAVrh.65 (See SEQ ID NO: 112 in |
| US20150315612) | US20150315612) |
| AAVrh.67 (See SEQ ID NO: 36 in | AAVrh.67 (See SEQ ID NO: 230 in |
| US20150315612) | US20150315612) |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in |
| US20150315612) | US20150315612) |
| AAVrh.69 (See SEQ ID NO: 39 in | AAVrh.69 (See SEQ ID NO: 119 in |
| US20150315612) | US20150315612) |
| AAVrh.70 (See SEQ ID NO: 20 in | AAVrh.70 (See SEQ ID NO: 98 in |
| US20150315612) | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in | AAVrh.74 (See SEQ ID NO: 6 in |
| US20150159173) | US20150159173) |
| AAVrh.8 (See SEQ ID NO: 41 in | AAVrh.8 (See SEQ ID NO: 235 in |
| US20150159173) | US20150315612) |
| AAVrh.8R (See SEQ ID NO: 9 in | AAVrh.8R A586R mutant (See SEQ ID NO: 10 |
| US20150159173, WO2015168666) | in WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in | caprine AAV (See SEQ ID NO: 4 in |
| U.S. Pat. No. 7,427,396) | U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-Pl (See SEQ ID NO: 29 in |
| WO2016065001) | WO2016065001) |
| AAV CHt-Pl (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | U.S. Pat. No. 8,734,809) |
| AAV CKd-1 (See SEQ ID NO: 131 in | AAV CKd-10 (See SEQ ID NO: 58 in |
| U.S. Pat. No. 8,734,809) | U.S. Pat. No. 8,734,809) |
| AAV CKd-10 (See SEQ ID NO: 132 in | AAV CKd-2 (See SEQ ID NO: 59 in |
| U.S. Pat. No. 8,734,809) | U.S. Pat. No. 8,734,809) |
| AAV CKd-2 (See SEQ ID NO: 133 in | AAV CKd-3 (See SEQ ID NO: 60 in |
| U.S. Pat. No. 8,734,809) | U.S. Pat. No. 8,734,809) |
| AAV CKd-3 (See SEQ ID NO: 134 in | AAV CKd-4 (See SEQ ID NO: 61 in |
| U.S. Pat. No. 8,734,809) | U.S. Pat. No. 8,734,809) |
| AAV CKd-4 (See SEQ ID NO: 135 in | AAV CKd-6 (See SEQ ID NO: 62 in |
| U.S. Pat. No. 8,734,809) | U.S. Pat. No. 8,734,809) |
| AAV CKd-6 (See SEQ ID NO: 136 in | AAV CKd-7 (See SEQ ID NO: 63 in |
| U.S. Pat. No. 8,734,809) | U.S. Pat. No. 8,734,809) |
| AAV CKd-7 (See SEQ ID NO: 137 in | AAV CKd-8 (See SEQ ID NO: 64 in |
| U.S. Pat. No. 8,734,809) | U.S. Pat. No. 8,734,809) |
| AAV CKd-8 (See SEQ ID NO: 138 in | AAV CKd-B 1 (See SEQ ID NO: 73 in |
| U.S. Pat. No. 8,734,809) | U.S. Pat. No. 8,734,809) |
| AAV CKd-B 1 (See SEQ ID NO: 147 in | AAV CKd-B2 (See SEQ ID NO: 74 in |
| U.S. Pat. No. 8,734,809) | U.S. Pat. No. 8,734,809) |
| AAV CKd-B2 (See SEQ ID NO: 148 in | AAV CKd-B3 (See SEQ ID NO: 75 in |
| U.S. Pat. No. 8,734,809) | U.S. Pat. No. 8,734,809) |
| AAV CKd-B3 (See SEQ ID NO: in | AAV CKd-B3 (See SEQ ID NO: 149 in |
| U.S. Pat. No. 8,734,809 | U.S. Pat. No. 8,734,809) |
| AAV CLv-1 (See SEQ ID NO: 65 in | AAV CLv-1 (See SEQ ID NO: 139 in |
| U.S. Pat. No. 8,734,809) | U.S. Pat. No. 8,734,809) |
| AAV CLvl-1 (See SEQ ID NO: 171 in | AAV Civ 1-10 (See SEQ ID NO: 178 in |
| U.S. Pat. No. 8,734,809) | U.S. Pat. No. 8,734,809) |
| AAV CLvl-2 (See SEQ ID NO: 172 in | AAV CLv-12 (See SEQ ID NO: 66 in |
| U.S. Pat. No. 8,734,809) | U.S. Pat. No. 8,734,809) |
| AAV CLv-12 (See SEQ ID NO: 140 in | AAV CLvl-3 (See SEQ ID NO: 173 in |
| U.S. Pat. No. 8,734,809) | U.S. Pat. No. 8,734,809) |
| AAV CLv-13 (See SEQ ID NO: 67 in | AAV CLv-13 (See SEQ ID NO: 141 in |
| U.S. Pat. No. 8,734,809) | U.S. Pat. No. 8,734,809) |
| AAV CLvl-4 (See SEQ ID NO: 174 in | AAV Civ 1-7 (See SEQ ID NO: 175 in |
| U.S. Pat. No. 8,734,809) | U.S. Pat. No. 8,734,809) |
| AAV Civ 1-8 (See SEQ ID NO: 176 in | AAV Civ 1-9 (See SEQ ID NO: 177 in |
| U.S. Pat. No. 8,734,809) | U.S. Pat. No. 8,734,809) |
| AAV CLv-2 (See SEQ ID NO: 68 in | AAV CLv-2 (See SEQ ID NO: 142 in |
| U.S. Pat. No. 8,734,809) | U.S. Pat. No. 8,734,809) |
| AAV CLv-3 (See SEQ ID NO: 69 in | AAV CLv-3 (See SEQ ID NO: 143 in |
| U.S. Pat. No. 8,734,809) | U.S. Pat. No. 8,734,809) |
| AAV CLv-4 (See SEQ ID NO: 70 in | AAV CLv-4 (See SEQ ID NO: 144 in |
| U.S. Pat. No. 8,734,809) | U.S. Pat. No. 8,734,809) |
| AAV CLv-6 (See SEQ ID NO: 71 in | AAV CLv-6 (See SEQ ID NO: 145 in |
| U.S. Pat. No. 8,734,809) | U.S. Pat. No. 8,734,809) |
| AAV CLv-8 (See SEQ ID NO: 72 in | AAV CLv-8 (See SEQ ID NO: 146 in |
| U.S. Pat. No. 8,734,809) | U.S. Pat. No. 8,734,809) |
| AAV CLv-Dl (See SEQ ID NO: 22 in | AAV CLv-Dl (See SEQ ID NO: 96 in |
| U.S. Pat. No. 8,734,809) | U.S. Pat. No. 8,734,809) |
| AAV CLv-D2 (See SEQ ID NO: 23 in | AAV CLv-D2 (See SEQ ID NO: 97 in |
| U.S. Pat. No. 8,734,809) | U.S. Pat. No. 8,734,809) |
| AAV CLv-D3 (See SEQ ID NO: 24 in | AAV CLv-D3 (See SEQ ID NO: 98 in |
| U.S. Pat. No. 8,734,809) | U.S. Pat. No. 8,734,809) |
| AAV CLv-D4 (See SEQ ID NO: 25 in | AAV CLv-D4 (See SEQ ID NO: 99 in |
| U.S. Pat. No. 8,734,809) | U.S. Pat. No. 8,734,809) |
| AAV CLv-D5 (See SEQ ID NO: 26 in | AAV CLv-D5 (See SEQ ID NO: 100 in |
| U.S. Pat. No. 8,734,809) | U.S. Pat. No. 8,734,809) |
| AAV CLv-D6 (See SEQ ID NO: 27 in | AAV CLv-D6 (See SEQ ID NO: 101 in |
| U.S. Pat. No. 8,734,809) | U.S. Pat. No. 8,734,809) |
| AAV CLv-D7 (See SEQ ID NO: 28 in | AAV CLv-D7 (See SEQ ID NO: 102 in |
| U.S. Pat. No. 8,734,809) | U.S. Pat. No. 8,734,809) |
| AAV CLv-D8 (See SEQ ID NO: 29 in | AAV CLv-D8 (See SEQ ID NO: 103 in |
| U.S. Pat. No. 8,734,809) | U.S. Pat. No. 8,734,809); |
| | AAV CLv-Kl 762, see SEQ ID |
| | NO: 18 in WO2016065001) |
| AAV CLv-Kl (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-Ml (See SEQ ID NO: 21 in |
| WO2016065001) | WO2016065001) |
| AAV CLv-Ml (See SEQ ID NO: 71 in | AAV CLv-Mll (See SEQ ID NO: 22 in |
| WO2016065001) | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in |
| WO2016065001) | U.S. Pat. No. 8,734,809) |
| AAV CLv-Rl (See SEQ ID NO: 104 in | AAV CLv-R2 (See SEQ ID NO: 31 in |
| U.S. Pat. No. 8,734,809) | U.S. Pat. No. 8,734,809) |
| AAV CLv-R2 (See SEQ ID NO: 105 in | AAV CLv-R3 (See SEQ ID NO: 32 in |
| U.S. Pat. No. 8,734,809) | U.S. Pat. No. 8,734,809) |
| AAV CLv-R3 (See SEQ ID NO: 106 in | AAV CLv-R4 (See SEQ ID NO: 33 in |
| U.S. Pat. No. 8,734,809) | U.S. Pat. No. 8,734,809) |
| AAV CLv-R4 (See SEQ ID NO: 107 in | AAV CLv-R5 (See SEQ ID NO: 34 in |
| U.S. Pat. No. 8,734,809) | U.S. Pat. No. 8,734,809) |
| AAV CLv-R5 (See SEQ ID NO: 108 in | AAV CLv-R6 (See SEQ ID NO: 35 in |
| U.S. Pat. No. 8,734,809) | U.S. Pat. No. 8,734,809) |
| AAV CLv-R6 (See SEQ ID NO: 109 in | AAV CLv-R7 (See SEQ ID NO: 110 in |
| U.S. Pat. No. 8,734,809); | U.S. Pat. No. 8,734,809) |
| AAV CLv-R7 802 (see SEQ ID NO: | |
| 36 in U.S. Pat. No. 8,734,809) | |
| AAV CLv-R8 (See SEQ ID NO: 37 in | AAV CLv-R8 (See SEQ ID NO: 111 in |
| U.S. Pat. No. 8,734,809) | U.S. Pat. No. 8,734,809) |
| AAV CLv-R9 (See SEQ ID NO: 38 in | AAV CLv-R9 (See SEQ ID NO: 112 in |
| U.S. Pat. No. 8,734,809) | U.S. Pat. No. 8,734,809) |
| AAV CSp-1 (See SEQ ID NO: 45 in | AAV CSp-1 (See SEQ ID NO: 119 in |
| U.S. Pat. No. 8,734,809) | U.S. Pat. No. 8,734,809) |
| AAV CSp-10 (See SEQ ID NO: 46 in | AAV CSp-10 (See SEQ ID NO: 120 in |
| U.S. Pat. No. 8,734,809) | U.S. Pat. No. 8,734,809) |
| AAV CSp-11 (See SEQ ID NO: 47 in | AAV CSp-11 (See SEQ ID NO: 121 in |
| U.S. Pat. No. 8,734,809) | U.S. Pat. No. 8,734,809) |
| AAV CSp-2 (See SEQ ID NO: 48 in | AAV CSp-2 (See SEQ ID NO: 122 in |
| U.S. Pat. No. 8,734,809) | U.S. Pat. No. 8,734,809) |
| AAV CSp-3 (See SEQ ID NO: 49 in | AAV CSp-3 (See SEQ ID NO: 123 in |
| U.S. Pat. No. 8,734,809) | U.S. Pat. No. 8,734,809) |
| AAV CSp-4 (See SEQ ID NO: 50 in | AAV CSp-4 (See SEQ ID NO: 124 in |
| U.S. Pat. No. 8,734,809) | U.S. Pat. No. 8,734,809) |
| AAV CSp-6 (See SEQ ID NO: 51 in | AAV CSp-6 (See SEQ ID NO: 125 in |
| U.S. Pat. No. 8,734,809) | U.S. Pat. No. 8,734,809) |
| AAV CSp-7 (See SEQ ID NO: 52 in | AAV CSp-7 (See SEQ ID NO: 126 in |
| U.S. Pat. No. 8,734,809) | U.S. Pat. No. 8,734,809) |
| AAV CSp-8 (See SEQ ID NO: 53 in | AAV CSp-8 (See SEQ ID NO: 127 in |
| U.S. Pat. No. 8,734,809) | U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| U.S. Pat. No. 8,734,809) | U.S. Pat. No. 8,734,809) |
| AAV.hu.48R3 (See SEQ ID NO: 183 in | AAV.VR-355 (See SEQ ID NO: 181 in |
| U.S. Pat. No. 8,734,809) | U.S. Pat. No. 8,734,809) |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

Table 7 describe exemplary chimeric or variant capsid proteins that can be used as the AAV capsid in the rAAV vectors and methods for producing the same as described herein, or with any combination with wild type capsid proteins and/or other chimeric or variant capsid proteins now known or later identified; reference described in Table 7 are incorporated herein by reference. In some embodiments, the rAAV vector is a chimeric vector, e.g., as disclosed in 9,012,224 and U.S. Pat. No. 7,892,809, which are incorporated herein in their entirety by reference. In some embodiments, the rAAV comprises at least one capsid from the chimeric or, variant capsids listed in Table 7.

In some embodiments, the rAAV vector is a polyploid rAAV vector, as disclosed in PCT/US2018/022725, or rational polyploid (or, haploid) rAAV vector, e.g., as disclosed in PCT/US2018/044632 filed on Jul. 31, 2018 and in U.S. Pat. No. 10,550,405, each of which are incorporated herein in their entirety by reference. In some embodiments, the rAAV vector is a rAAV3 vector, as disclosed in U.S. Pat. No. 9,012,224 and WO 2017/106236 which are incorporated herein in their entirety by reference.

TABLE 7
Exemplary chimeric and rAAV variant capsids

| Chimeric or | | Chimeric or variant | |
|---|---|---|---|
| variant capsid | reference | capsid | reference |
| LK03 and others | Lisowski et al. [REF 1] | AAV-leukemia | Michelfelder S |
| LK0-19 | | targeting | [REF 30] |
| AAV-DJ | Grimm et al., [REF 2] | AAV-tumor targeting | Muller O J, et al., |
| | | | [REF 31] |
| Olig001 | Powell S K et al., [REF 3] | AAV-tumor targeting | Grifman M et al., |
| | | | [REF 32] |
| rAAV2-retro | Tervo D et al., [REF 4] | AAV2 efficient | Girod et al., |
| | | targeting | [REF 33] |
| AAV-LiC | Marsic D et al., [REF 5] | AAVpo2.1, -po4, -poS, | Bello A, et al., |
| | | and -po6). | [REF 34] |
| (AAV-Keral, AAV- | Sallach et al., [REF 6] | AAV rh and AAV Hu | Gao G, et al., |
| Kera2, and AAV- | | | [REF 35] |
| Kera3) | | | |
| AAV 7m8 | Dalkara et al., [REF 7] | AAV-Go.1 | Arbetman A E et al., |
| | | | [REF 36] |
| (AAV1.9 | Asuri P et al., [REF 8] | AAV-mo.1 | Lochrie M A et al., |
| | | | [REF 37] |
| AAV r3.45 | Jang J H et al., [REF 9] | BAAV | Schmidt M, et al., |
| | | | [REF 38] |
| AAV clone 32 and | Gray S J, et al., [REF 10] | AAAV | Bossis I et al., |
| 83) | | | [REF 39] |
| AAV-U87R7-C5 | Maguire et al., [REF 11] | AAV variants | Chen C L et al., |
| | | | [REF 40] |
| AAV ShH13, AAV | Koerber et al., [REF 12] | AAV8 K137R | Sen D et al., |
| ShH19, AAV Ll-12 | | | [REF 41] |
| AAV HAE-1, AAV | Li W et al., [REF 13] | AAV2 Y | Li B, et al., |
| HAE-2 | | | [REF 42] |
| AAV variant ShH10 | Klimczak et al., [REF 14] | AAV2 | Gabriel N et al., |
| | | | [REF 43] |
| AAV2.5T | Excoffon et al., [REF 15] | AAV Anc80L65 | Zinn E, et al., |
| | | | [REF 44] |
| AAV LS1-4, AAV | Sellner L et al., [REF 16] | AAV2G9 | Shen S et al., |
| Lsm | | | [REF 45] |
| AAV1289 | Li W, et al., [REF 17] | AAV2 265 insertion- | Li C, et al., |
| | | AAV2/265D | [REF 46] |
| AAVHSC 1-17 | Charbel Issa P et al., [REF | AAV2.5 | Bowles D E, et al., |
| | 18] | | [REF 47] |
| AAV2 Rec 1-4 | Huang W, et al., [REF 19] | AAV3 SASTG | Messina E L et al., |
| | | | [REF 48] and |
| | | | [REF 55]. |
| | | | (Piacentio et al., |
| | | | (Hum Gen Ther, 2012, |
| | | | 23: 635-646)) |
| AAV8BP2 | Cronin T, et al., [REF 20] | AAV2i8 | Asokan A et al., |
| | | | [REF 49] |
| AAV-B1 | Choudhury S R, et al., | AAV8G9 | Vance M, et al., |
| | [REF 21] | | [REF 50] |
| AAV-PHP.B | Deverman B E, et al., [REF | AAV2 tyrosine | Zhong L et al., |
| | 22] | mutants AAV2 Y-F | [REF 51] |
| AAV9.45, | Pulicherla N[REF 23], et | AAV8 Y-F and AAV9 | Petrs-Silva H et al., |
| AAV9.61, AAV9.47 | al., | Y-F | [REF 52] |
| AAVM41 | Yang L et al., [REF 24] | AAV6 Y-F | Qiao C et al., |
| | | | [REF 53] |
| AAV2 displayed | Korbelin J et al. [REF 25], | (AAV6.2) PCT | Carlon M, et al., |
| peptides) | | Publication No. | [REF 54] |
| | | WO2013158879A1 | |
| | | (lysine mutants) | |
| AAV2-GMN | Geoghegan J C [REF 26] | | |
| AAV9-peptide | Varadi K, et al., [REF 27] | | |
| displayed | | | |
| AAV8 and AAV9 | Michelfelder et al., [REF 28] | | |
| peptide displayed | | | |
| AAV2-muscle | Yu C Y et al., [REF 29] | | |
| targeting peptide | | | |

In one embodiment, the rAAV vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. patent application Ser. No. 11/486,254 and the other biological sequence files are to be read in a similar manner): 11486254.raw, 11932017.raw, 12172121.raw, 12302206.raw, 12308959.raw, 12679144.raw, 13036343.raw, 13121532.raw, 13172915.raw, 13583920.raw, 13668120.raw, 13673351.raw, 13679684.raw, 14006954.raw, 14149953.raw, 14192101.raw, 14194538.raw, 14225821.raw, 14468108.raw, 14516544.raw, 14603469.raw, 14680836.raw, 14695644.raw, 14878703.raw, 14956934.raw, 15191357.raw, 15284164.raw, 15368570.raw, 15371188.raw, 15493744.raw, 15503120.raw, 15660906.raw, and 15675677.raw.

In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference.

In some embodiments, an rAAV vector genome is single stranded or a monomeric duplex as described in U.S. Pat. No. 8,784,799, which is incorporated herein by reference.

As a further embodiment, the AAV capsid proteins and virus capsids of this invention can be polyploid (also referred to as haploid) in that they can comprise different combinations of VP1, VP2 and VP3 AAV serotypes in a single AAV capsid as described in PCT/US18/22725, which is incorporated by reference.

In one embodiment, the capsid can be any capsid, but preferably a capsid that is muscle tropic, e.g., a rational haploid capsid designed to be preferentially skeletal muscle-specific and/or cardiac muscle-specific.

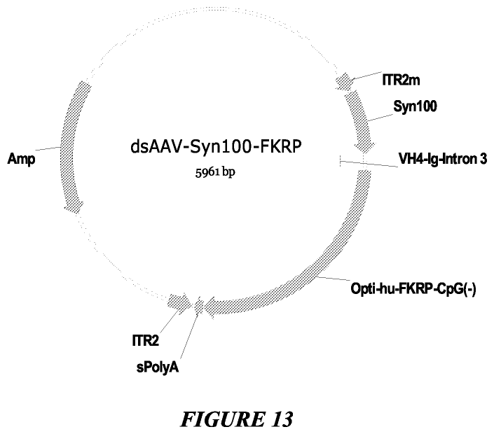

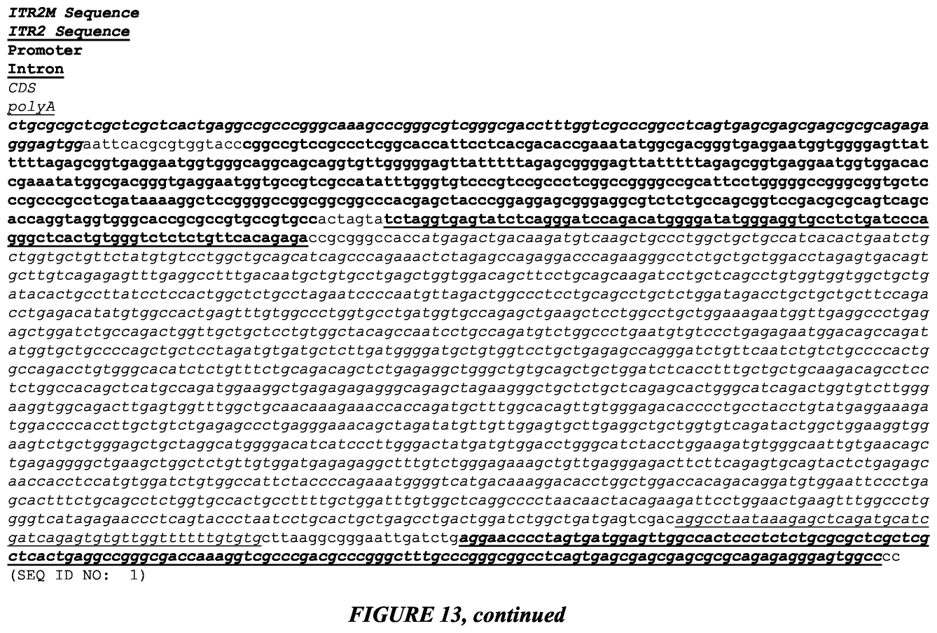

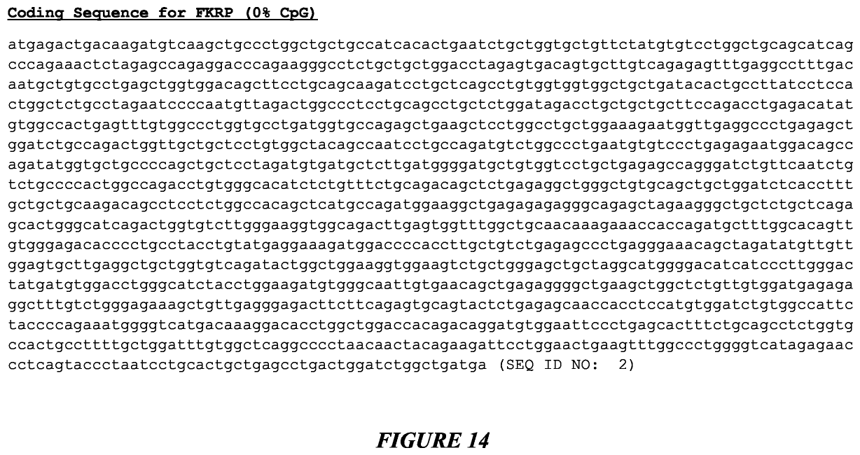

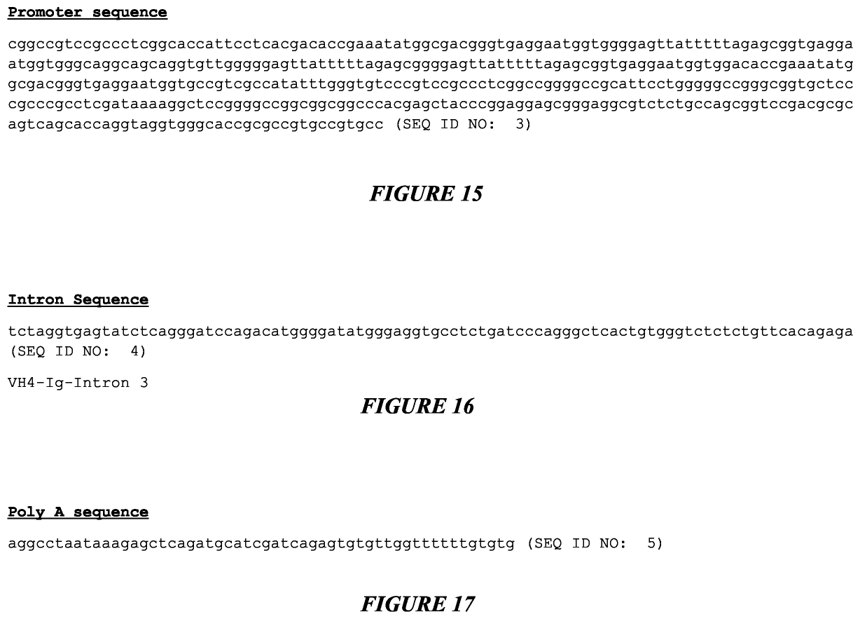

In one embodiment, the nucleic acid used to manufacture rAAV that lacks bacterial sequence has the nucleotide sequence set out in SEQ ID NO: 406. In one embodiment, the the nucleic acid used to manufacture rAAV that lacks bacterial sequence has the nucleotide sequence has at least 70%, 75%, 80%, 81%, 82%, 83%, 84%, 85%, 86%, 87%, 88%, 89% or 90% identity to SEQ ID NO: 406. In some embodiments, the rAAV of the invention is manufactured from plasmid DNA template e.g., as shown in FIG. 13. In some embodiments, the rAAV of the invention is manufactured from close ended linear duplexed DNA e.g., as set forth in SEQ ID NO: 406.

In order to facilitate their introduction into a cell, an rAAV vector genome useful in the invention are recombinant nucleic acid constructs that include (1) a heterologous sequence to be expressed (in one embodiment, a polynucleotide encoding a FKRP polypeptide) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid.

Optimized rAAV Vector Genome

In some embodiments of the methods and compositions as disclosed herein, an optimized rAAV vector genome is created from any of the elements disclosed herein and in any combination, including nucleic acid sequences encoding a promoter, an ITR, a poly-A tail, elements capable of increasing or decreasing expression of a heterologous gene, and in one embodiment, a nucleic acid sequence that is codon optimized for expression of FKRP in vivo and optionally, one or more element to reduce immunogenicity. Such an optimized rAAV vector genome can be used with any AAV capsid that has tropism for the tissue and cells, e.g., the skeletal and cardiac muscle, in which the rAAV vector genome is to be transduced and expressed.

### Recombinant AAV Vector Production

The recombinant AAV vectors described herein may be produced by any method known in the art. Without limitation, one example of such a method to produce adeno-associate virus (AAV) particles comprises (a) providing the any of the stable cells described herein, e.g., a cell line having stable expression of at least one heterologous toxic protein required for AAV vector production, such as rep or cap, under the control of an inducible promoter, in an AAV expression system, (b) culturing the cells under conditions in which the at least one toxic protein is expressed, (c) culturing the cells under conditions in which AAV particles are produced, and (d) optionally isolating the AAV particles.

In one embodiment, the step of culturing the cells under conditions in which AAV particles are produced occurs only after the toxic protein is sufficiently expressed. For example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more hours after the cell is contacted with the inducer or applying suitable inducing conditions to the cell. As used herein, “sufficient expression” refers to the level of expression a protein required for proper function, e.g., the level of rep protein needed in the cell to induce replication.

If a cell comprises more than one distinct inducible promoter, the more than one inducible promoters can be induced to drive expression on the protein at substantially the same time, or at different times. Alternatively, if a cell comprises more than one distinct inducible promoter, the more than one inducible promoters can be induced to drive expression on the protein induced for the same period of time, or for different periods of time. In one embodiment, the cells are cultured with at least two inducers at substantially the same time, and for the same duration. In one embodiment, culturing with a first inducer is occurring when culturing with a second inducer begins, such that there is overlap in terms of culturing. This is sometimes referred to herein as “simultaneous” or “concurrent culturing.” In other embodiments, culturing with the first inducer ends prior to culturing with the second inducer beginning. When culturing occurs at substantially the same time or simultaneously, the first and second inducer can be provided in the same culture medium. Alternatively, when culturing occurs at substantially the same time or simultaneously, the first and second inducer can be provided in different culture mediums.

In one embodiment, the cells are cultured in suspension. In another embodiment, the cells are cultured in animal component-free conditions. The animal component-free medium can be any animal component-free medium (e.g., serum-free medium) compatible with a given cell line, for example, HEK293 cells. Examples include, without limitation, SFM4Transfx-293 (Hyclone), Ex-Cell 293 (JRH Biosciences), LC-SFM (Invitrogen), and Pro293-S (Lonza).

Conditions sufficient for the replication and packaging of the AAV particles can be, e.g., the presence of AAV sequences sufficient for replication of an AAV template and encapsidation into AAV capsids (e.g., AAV rep sequences and AAV cap sequences) and helper sequences from adenovirus and/or herpesvirus. In particular embodiments, the AAV template comprises two AAV ITR sequences, which are located 5′ and 3′ to the heterologous nucleic acid sequence, although they need not be directly contiguous thereto.

In some embodiments, the AAV template comprises an ITR that is not resolved by Rep to make duplexed AAV vectors as described in international patent publication WO 01/92551 and U.S. Pat. No. 8,784,799.

The AAV template and AAV rep and/or cap sequences are provided under conditions such that virus vector comprising the AAV template packaged within the AAV capsid is produced in the cell. The method can further comprise the step of collecting the virus vector from the culture. In one embodiment, the virus vector can be collected by lysing the cells, e.g., after removing the cells from the culture medium, e.g., by pelleting the cells. In another embodiment, the virus vector can be collected from the medium in which the cells are cultured, e.g., to isolate vectors that are secreted from the cells. Some or all of the medium can be removed from the culture one time or more than one time, e.g., at regular intervals during the culturing step for collection of rAAV (such as every 12, 18, 24, or 36 hours, or longer extended time that is compatible with cell viability and vector production), e.g., beginning about 48 hours post-transfection. After removal of the medium, fresh medium, with or without additional nutrient supplements, can be added to the culture. In one embodiment, the cells can be cultured in a perfusion system such that medium constantly flows over the cells and is collected for isolation of secreted rAAV. Collection of rAAV from the medium can continue for as long as the transfected cells remain viable, e.g., 48, 72, 96, or 120 hours or longer post-transfection, or in the case of the use of an inducible promoter to express a toxic protein, e.g., 48, 72, 96, or 120 hours or longer post-induction. In certain embodiments, the collection of secreted rAAV is carried out with serotypes of AAV (such as AAV8 and AAV9), which do not bind or only loosely bind to the producer cells. In other embodiments, the collection of secreted rAAV is carried out with heparin binding serotypes of AAV (e.g., AAV2) that have been modified so as to not bind to the cells in which they are produced. Examples of suitable modifications, as well as rAAV collection techniques, are disclosed in U.S. Publication No. 2009/0275107, which is incorporated by reference herein in its entirety.

In the event that a stable cell line does not stably or transiently express rep or cap, these sequences are to be provided to the AAV expression system. AAV rep and cap sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV rep/cap sequences will not be flanked by the TRs, to prevent rescue and/or packaging maintain of these sequences.

The AAV template can be provided to the cell using any method known in the art. For example, the template can be supplied by a non-viral (e.g., plasmid) or viral vector. In particular embodiments, the AAV template is supplied by a herpesvirus or adenovirus vector (e.g., inserted into the Ela or E3 regions of a deleted adenovirus). As another illustration, Palombo et al., J. Virol. 72:5025 (1998), describes a baculovirus vector carrying a reporter gene flanked by the AAV TRs. EBV vectors may also be employed to deliver the template, as described above with respect to the rep/cap genes.

In another representative embodiment, the AAV template is provided by a replicating rAAV virus. In still other embodiments, an AAV provirus comprising the AAV template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., Nature Med. 3:1295 (1997), and U.S. Pat. Nos. 6,040,183 and 6,093,570, which is incorporated herein by reference.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV cap and rep sequences and the helper virus sequences (e.g., adenovirus sequences) on a single helper construct. In one embodiment, expression of at least one gene product encoded by the single helper construct is controlled by an inducible promoter. This helper construct may be a non-viral or viral construct. As one nonlimiting illustration, the helper construct can be a hybrid adenovirus or hybrid herpesvirus comprising the AAV rep and/or cap genes.

In one particular embodiment, the AAV rep and/or cap sequences and the adenovirus helper sequences are supplied by a single adenovirus helper vector. This vector can further comprise the AAV template. The AAV rep and/or cap sequences and/or the AAV template can be inserted into a deleted region (e.g., the E1 a or E3 regions) of the adenovirus. In one embodiment, expression of at least one gene product encoded by the AAV template is controlled by an inducible promoter.

In a further embodiment, the AAV rep and/or cap sequences and the adenovirus helper sequences are supplied by a single adenovirus helper vector. According to this embodiment, the AAV template can be provided as a plasmid template.

In another illustrative embodiment, the AAV rep and/or cap sequences and adenovirus helper sequences are provided by a single adenovirus helper vector, and the AAV template is integrated into the cell as a provirus. Alternatively, the AAV template is provided by an EBV vector that is maintained within the cell as an extrachromosomal element (e.g., as an EBV based nuclear episome).

Use of the inducible and repressible promoters described herein can be used to achieve temporal regulation of any of the toxic proteins required for viral vector production, for example, rep and cap. In one embodiment, inducible and/or repressible promoters provide for careful fine tuning of expression of a toxic protein, such that one can tailor the start and stop of the expression to achieve the desired level of expression, and at the desired timing during production.

In a further exemplary embodiment, the AAV rep and/or cap sequences and adenovirus helper sequences are provided by a single adenovirus helper. The AAV template can be provided as a separate replicating viral vector. For example, the AAV template can be provided by an AAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep and/or cap sequences and, if present, the AAV template are embedded in the adenovirus backbone and are flanked by the 5′ and 3′ cis sequences, so that these sequences may be packaged into adenovirus capsids. As described above, the adenovirus helper sequences and the AAV rep and/or cap sequences are generally not flanked by TRs so that these sequences are not packaged into the AAV virions. Zhang et al., Gene Ther. 18:704 ((2001)) describe a chimeric helper comprising both adenovirus and the AAV rep and/or cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., Gene Ther. 6:986 (1999) and WO 00/17377).

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. Gene Ther. 6:973 (1999)). Deleted replication-defective helper viruses can be used so that any contaminating helper virus is not replication competent. As a further alternative, an adenovirus helper lacking late gene expression may be employed, as only adenovirus early gene expression is required to mediate packaging of AAV. Adenovirus mutants defective for late gene expression are known in the art (e.g., ts100K and ts149 adenovirus mutants).

In various embodiments, the method of producing the AAV viral vector of the invention is completely scalable, so it can be carried out in any desired volume of culture medium, e.g., from 10 ml (e.g., in shaker flasks) to 10 L, 50 L, 100 L, or more (e.g., in bioreactors such as wave bioreactor systems and stirred tanks). In one embodiment, the rAAV is produced using closed ended linear duplexed nucleic acid. In other embodiments, the rAAV is produced using other forms of nucleic acid e.g, plasmid DNA.

The method is suitable for production of all serotypes and chimeras of AAV, e.g., AAV1, AAV2, AAV3, AAV4, AAV5, AAV6, AAV7, AAV8, AAV9, AAV10, AAV11, AAV12, AAV13, and any chimeras thereof.

In certain embodiments, the method provides at least about 1×10⁴ vector genome-containing particles per cell prior to purification, e.g., at least about 2×10⁴, 3×10⁴, 4×10⁴, 5×10⁴, 6×10⁴, 7×10⁴, 8×10⁴, 9×10⁴, or 1×10⁵ or more vector genome-containing particles per cell prior to purification. In other embodiments, the method provides at least about 1×10¹² purified vector genome-containing particles per liter of cell culture, e.g., at least about 5×10¹², 1×10¹³, 5×10¹³, or 1×10¹⁴ or more purified vector genome-containing particles per liter of cell culture.

rAAV Genome Elements

As disclosed herein, aspects of the invention relate to a rAAV vector comprising a synthetic nucleic acid encoding FKRP. The rAAV vector comprises a capsid, and within its capsid, a nucleotide sequence referred to as the “rAAV vector genome”. The rAAV vector genome (also referred to as “rAAV genome) includes multiple elements, including, but not limited to two inverted terminal repeats (ITRs, e.g., the 5′-ITR and the 3′-ITR). Typically, located between the ITRs are additional elements, including one or more of the following: a promoter (e.g., a muscle-specific promotor) operatively linked to the synthetic nucleic acid encoding FKRP (as the heterologous gene), and a polyA signal sequence operatively linked to the synthetic nucleic acid. Typically, the polyA signal sequence is functionally located downstream of the coding sequence. In some embodiments, the polyA signal has the nucleic acid sequence shown in SEQ ID NO: 5. Other polyA signal sequences that can be used include, without limitation, bGH, hGH, SV40early, SV40late, synthetic polyA, rBG polyA, TK polyA, bovine growth hormone, rabbit β-globin, and SV40 polyA signal. Additional examples of polyA signal sequences are provided herein.

In some embodiments, the heterologous nucleic acid sequence can further comprise one or more additional elements (e.g., regulatory elements, spacer elements) such as an intron sequence and a poly A signal sequence. In some embodiments, the intron sequence is located between the promoter and the nucleic acid encoding FKRP and is operatively positioned to facilitate expression (e.g., in a subject). In some embodiments, the intron sequence is VH4-Ig-Intron 3 (SEQ ID NO: 4). Examples of other possible intron sequences include, without limitation, Chimeric pro mega intron, cmv intron, chimeric chicken beta actin-human globin intron, mvm intron, human ubB intron, human UbC intron, human beta globin IVS2. Additional intron sequences are provided herein.

Each of the elements in the rAAV genome are discussed herein.

### Intron Sequence

In some embodiments, the rAAV genotype comprises an intron sequence located 3′ of the promoter sequence. Intron sequences serve to increase one or more of: mRNA stability, mRNA transport out of nucleus and/or expression and/or regulation of the expressed FKRP protein product. One of skill in the art will appreciate that the nucleotide sequence of an intron can be modified or adapted while substantially preserving the functionality. Such derivatives of intron sequences are also encompassed in the various embodiments of the invention described herein.

In some embodiments, the intron sequence is a MVM intron sequence or a nucleic acid sequence having at least 80%, 85%, 90%, 95%, 96%, 97%, 98% or 99% nucleotide sequence identity thereto.

In some embodiments, the intron sequence is a HBB2 intron sequence, or a nucleic acid sequence having at least 80%, 85%, 90%, 95%, 96%, 97%, 98% or 99% nucleotide sequence identity thereto.

In some embodiments, the intron sequence is selected in the group consisting of a human beta globin b2 (or HBB2) intron, a FIX intron, a chicken beta-globin intron, and a SV40 intron. In some embodiments, the intron is optionally a modified intron such as a modified HBB2 intron (see, e.g., SEQ ID NO: 17 in of WO2018046774A1): a modified FIX intron (see., e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, the contents of which are incorporated herein by reference in their entirety.

### Poly-A Signal Sequence

In some embodiments, an rAAV vector genome containing the synthetic nucleic acid encoding FKRP includes at least one polyA signal sequence. Typically, such sequences are located 3′ and downstream from a coding sequence. In some embodiments, a spacer sequence is located between the coding sequence and the polyA signal sequence. In some embodiments, the polyA signal is 3′ of a stability sequence or CS sequence as defined herein. Any polyA signal sequence can be used, including but not limited to hGH poly A, synpA polyA and the like (e.g. SEQ ID NO: 5). In some embodiments, the polyA is a synthetic polyA signal sequence. In some embodiments, the rAAV vector genome comprises two polyA signal sequences, e.g., SEQ ID NO: 5 and another polyA sequence, where a spacer nucleic acid sequence is located between the two poly A signal sequences. In some embodiments, the rAAV genome comprises 3′ of the nucleic acid encoding FKRP, or alternatively, 3′ of the CS sequence the following elements; a first polyA signal sequence, a spacer nucleic acid sequence (of between 100-400 bp, or about 250 bp), a second poly A signal sequence, a spacer nucleic acid sequence, and the 3′ ITR. In some embodiments, the first and second poly A sequence is SEQ ID NO: 5, and in some embodiments, the first and second poly A sequences are a synthetic poly A sequence. In some embodiments, the first poly A sequence is a SEQ ID NO: 5 and the second poly A sequence is a synthetic sequence, or vice versa—that is, in alternative embodiments, the first poly A sequence is a synthetic poly A sequence and the second poly A sequence is SEQ ID NO: 5. An exemplary poly A signal sequence is, for example, SEQ ID NO: 5, or a poly A signal sequence having at least 80%, 85%, 90%, 95%, 96%, 97%, 98% or 99% nucleotide sequence identity to SEQ ID NO: 5.

In some embodiments, a polyA tail is engineered to stabilize the FKRP RNA transcript that is transcribed from an rAAV vector genome. In alternative embodiments, the poly-A signal sequence can be engineered to include elements in the RNA transcript that are destabilizing.

In an embodiment, a polyA signal sequence can engineer destabilizing elements by altering the length of the poly-A tail. In an embodiment, the poly-A tail can be lengthened or shortened. In a further embodiment, the 3′ untranslated region that lies between the FKRP coding sequences and the poly-A sequences can be lengthened or shortened to alter the expression levels of the FKRP or alter the final polypeptide that is produced.

In another embodiment, a destabilizing element is a microRNA (miRNA) that has the ability to silence (repress translation and promote degradation) the RNA transcripts the miRNA binds to that encode a heterologous gene. Modulation of the expression of the FKRP transgene can be undertaken by modifying, adding or deleting seed regions within the poly-A tail to which the miRNA bind. In an embodiment, addition or deletion of seed regions within the poly-A tail can increase or decrease expression of the FKRP. In a further embodiment, such increase or decrease in expression resultant from the addition or deletion of seed regions is dependent on the cell type transduced by the AAV containing an rAAV vector genome. For instance, seed regions specific for miRNA expressed in muscle and cardiac cells, but not found in liver cells, can be used to allow for production of the FKRP in liver cells, but not muscle cells or cardiac cells.

In another embodiment, seed regions can also be engineered into the 3′ untranslated regions located between the FKRP transgene and the poly-A tail. In a further embodiment, the destabilizing agent can be an siRNA. The coding region of the siRNA can be included in an rAAV vector genome and is generally located downstream, 3′ of the poly-A tail. In an embodiment, the expression of a FKRP transgene can be undertaken by inclusion of the coding region for an siRNA in the rAAV cassette, for instance, downstream, 3′ of the poly-A sequence. In a further embodiment, the promoter to induce expression of the siRNA can be tissue specific, such that the siRNA is silenced in tissues where expression of the FKRP transgene is not desired and siRNA expression does not occur in tissues where expression of the FKRP transgene is desired.

### Spacer Elements

In some embodiments, one or more spacer elements or sequences are located within the AAV genome sequence. In some embodiments, the spacer element comprises one or more nucleic acids encoding a spacer of at least 1 amino acid. In some embodiments, the spacer element(s) does not serve to encode any amino acids.

In all aspects of the methods and compositions as disclosed herein, the rAAV genome may also comprise one or more a stuffer or spacer DNA nucleic sequences located between the various components described herein (see FIGS. 13 and 19). In some embodiments, a spacer sequence is located between the 5′ ITR and the promoter (e.g., SEQ ID NO: 9). In some embodiments, a spacer sequence is located between the promoter and the intron (e.g., SEQ ID NO: 10). In some embodiments, a spacer sequence is located between intron and the FKRP coding sequence (e.g., SEQ ID NO: 11). In some embodiments, a spacer sequence is located between and the FKRP coding sequence and the polyA signal sequence (e.g., SEQ ID NO: 12). In some embodiments, a spacer sequence is located between the polyA signal sequence and the 3′ ITR (e.g. SEQ ID NO: 13). An exemplary stuffer DNA sequence is SEQ ID NO: 13, or a nucleic acid sequence having at least 80%, 85%, 90%, 95%, 96%, 97%, 98% or 99% nucleotide sequence identity thereto. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence comprise SEQ ID NO: 9-13 or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical thereto.

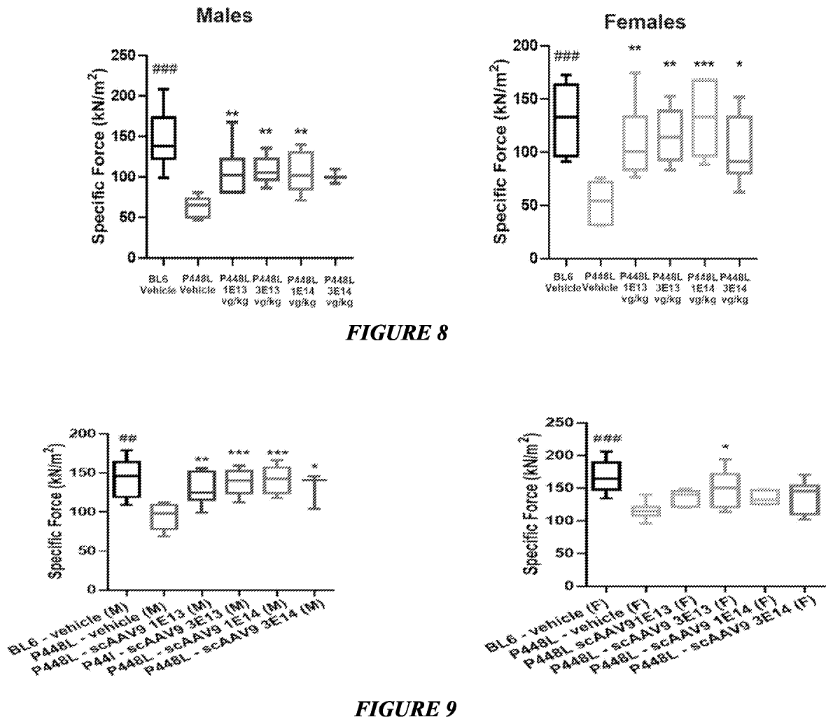

The stuffer sequence can be located 3′ of the poly A signal sequence, for example, and is located 5‘ of the’3 ITR sequence. In some embodiments, the stuffer DNA sequence comprises a synthetic polyadenylation signal in the reverse orientation. In some embodiments, a stuffer nucleic acid sequence (also referred to as a “spacer” nucleic acid fragment, see FIGS. 13 and 19) can be located between the poly A sequence and the 3′ ITR (i.e., a stuffer nucleic acid sequence is located 3′ of the polyA sequence and 5′ of the 3′ ITR) (see, e.g., FIG. 8-10). Such a stuffer nucleic acid sequence can be about 30 bp, 50 pb, 75 bp, 100 bp, 150 bp, 200 bp, 250 bp, 300 bp or longer than 300 bp.

### AAV ITRs

The rAAV genome as disclosed here comprises AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference. In some embodiments, one of the ITRs has a mutation that allows the formation of self-complementary AAV vectors, discussed further below. In some embodiments, the rAAVs of the present invention comprise self-complementary genomes as disclosed in International Patent application WO2001092551; U.S. Pat. Nos. 7,465,583, 7,790,154, 8,361,457, 8,784,799; all of which are incorporated herein by reference in their entirety.

The AAV ITRs for use in the rAAV genome as disclosed herein may be of any serotype suitable for a particular application. In some embodiments, the rAAV vector genome is flanked by AAV ITRs. In some embodiments, an ITR comprises a full length ITR sequence, an ITR with sequences comprising CpG sites/motifs/islands removed, an ITR with sequences comprising CpG sequences added, a truncated ITR sequence, an ITR sequence with one or more deletions within an ITR, an ITR sequence with one or more additions within an ITR, or a combination comprising any portion of the aforementioned ITRs linked together to form a hybrid ITR.

In order to facilitate long term expression, in an embodiment, the synthetic nucleic acid encoding FKRP is interposed between AAV inverted terminal repeats (ITRs) (e.g., the first or 5′ and second 3′ AAV ITRs). AAV ITRs are found at both ends of a WT rAAV vector genome, and serve as the origin and primer of DNA replication. ITRs are required in cis for AAV DNA replication as well as for rescue, or excision, from prokaryotic plasmids. In an embodiment, the AAV ITR sequences that are contained within the nucleic acid of the rAAV genome can be derived from any AAV serotype (e.g. 1, 2, 3, 3b, 4, 5, 6, 7, 8, 9, and 10, any serotypes shown in Table 6) or can be derived from more than one serotype, including combining portions of two or more AAV serotypes to construct an ITR. In an embodiment, for use in the rAAV vector, including an rAAV vector genome, the first and second ITRs should include at least the minimum portions of a WT or engineered ITR that are necessary for packaging and replication.

In some embodiments, the rAAV vector genome comprises at least one AAV ITR, wherein said ITR comprises, consists essentially of, or consists of; (a) an AAV rep binding element; (b) an AAV terminal resolution sequence; and (c) an AAV RBE (Rep binding element); wherein said ITR does not comprise any other AAV ITR sequences. In another embodiment, elements (a), (b), and (c) are from an AAV2 ITR and the ITR does not comprise any other AAV2 ITR sequences. In a further embodiment, elements (a), (b) and (c) are from any AAV ITR, including but not limited to AAV2, AAV8 and AAV9. In some embodiments, the polynucleotide comprises two synthetic ITRs, which may be the same or different.

In some embodiments, the polynucleotide in the rAAV vector, including an rAAV vector genome comprises two ITRs, which may be the same or different. The three elements in the ITR have been determined to be sufficient for ITR function. This minimal functional ITR can be used in all aspects of AAV vector production and transduction. Additional deletions may define an even smaller minimal functional ITR. The shorter length advantageously permits the packaging and transduction of larger transgenic cassettes.

In some embodiments, each of the elements that are present in a synthetic ITR can be the exact sequence as exists in a naturally occurring AAV ITR (the WT sequence) or can differ slightly (e.g., differ by addition, deletion, and/or substitution of 1, 2, 3, 4, 5 or more nucleotides) so long as the functioning of the elements of the AAV ITR continue to function at a level sufficient to are not substantially different from the functioning of these same elements as they exist in a naturally occurring AAV ITR.

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

In some embodiments, the rAAV vector, including an rAAV vector genome as described herein comprises a polynucleotide comprising at least one synthetic AAV ITR, wherein one or more CpG sites/motifs (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG sites can reduce the immunogenicity of the rAAV vector. This results from a reduction or complete inhibition in TLR-9 binding to the rAAV vector DNA sequence, which occurs at CpG site. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG site are deleted and/or substituted. In an embodiment, AAV ITR2 is known to contain 16 CpG sites of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs. The phrase “deleted and/or substituted” as used herein means that one or both nucleotides in the CpG motif is deleted, substituted with a different nucleotide, or any combination of deletions and substitutions.

In some embodiments, the synthetic ITR comprises, consists essentially of, or consists of one of the nucleotide sequences listed below. In other embodiments, the synthetic ITR comprises, consist essentially of, or consist of a nucleotide sequence that is at least 80% identical, e.g., at least 85%, 90%, 95%, 96%, 97%, 98%, or 99% identical to one of the nucleotide sequences listed below.

MH-257
(SEQ ID NO: 36)
AGGAACCCCTAGTGATGGAGTTGGCCACTCCCTCTCTGCGCGCTCGCTC
GCTCACTGAGGCAATTTGATAAAAATCGTCAAATTATAAACAGGCTTTG
CCTGTTTAGCCTCAGTGAGCGAGCGAGCGCGCAGAGAGGGAGTGGCCAA
CTCCATCACTAGGGGTTCCT
MH-258
(SEQ ID NO: 37)
AGGAACCCCTAGTGATGGAGTTGGCCACTCCCTCTCTGCGCGCTCGCTC
GCTCACTGAGGGATAAAAATCCAGGCTTTGCCTGCCTCAGTGAGCGAGC
GAGCGCGCAGAGAGGGAGTGGCCAACTCCATCACTAGGGGTTCCT
MH Delta 258
(SEQ ID NO: 38)
AGGAACCCCTAGTGATGGAGTTGGCCACTCCCTCTCTGCGCGCTCGCTC
GCTCACTGAGGGATAAAAATCCAGGCTTTGCCTGCCTCAGTGAGCGAGC
GAGCGCGCAGAGAGGGAGTGGCCAACTCCATCACTAGGGGTTCCT
MH Telomere-1 ITR
(SEQ ID NO: 39)
AGGAACCCCTAGTGATGGAGTTGGCCACTCCCTCTCTGGGATTGGGATT
GCGCGCTCGCTCGCGGGATTGGGATTGGGATTGGGATTGGGATTGGGAT
TGATAAAAATCAATCCCAATCCCAATCCCAATCCCAATCCCAATCCCGC
GAGCGAGCGCGCAATCCCAATCCCAGAGAGGGAGTGGCCAACTCCATCA
CTAGGGGTTCCT
MH Telomere-2 ITR
(SEQ ID NO: 409)
AGGAACCCCTAGTGATGGAGTTGGCCACTCCCTCTCTGCGCGCTCGCTC
GCTCGGGATTGGGATTGGGATTGGGATTGGGATTGGGATTGATAAAAAT
CAATCCCAATCCCAATCCCAATCCCAATCCCAATCCCGCGAGCGAGCGC
GCAGGAGAGGGAGTGGCCAACTCCATCACTAGGGGTTCCTAAGCTTATT
ATA
MH PolII 258 ITR
(SEQ ID NO: 410)
AGGAACCCCTAGTGATGGAGTTGGCCACTCCCTCTCTGCGCGCTCGCTC
GCTCACTGAGGGCGCCTATAAAGATAAAAATCCAGGCTTTGCCTGCCTC
AGTTAGCGAGCGAGCGCGCAGAGAGGGAGTGGCCAACTCCATCACTAGG
GGTTCCT
MH 258 Delta D conservative
(SEQ ID NO: 411)
CTAGTGATGGAGTTGGCCACTCCCTCTCTGCGCGCTCGCTCGCTCACTG
AGGGATAAAAATCCAGGCTTTGCCTGCCTCAGTGAGCGAGCGAGCGCGC
AGAGAGGGAGTGGCCAACTCCATCACTAG

In certain embodiments, a rAAV vector genome as described herein comprises a synthetic ITR that is capable of producing AAV virus particles that can transduce host cells. Such ITRs can be used, for example, for viral delivery of heterologous nucleic acids. Examples of such ITRs include MH-257, MH-258, and MH Delta 258 listed above.

In other embodiments, a rAAV vector genome as described herein containing a synthetic ITR is not capable of producing AAV virus particles. Such ITRs can be used, for example, for non-viral transfer of heterologous nucleic acids. Examples of such ITRs include MH Telomere-1, MH Telomere-2, and MH Pol II 258 listed above.

In some embodiments, an rAAV vector genome as described herein comprising the synthetic ITR of the invention further comprises a second ITR which may be the same as or different from the first ITR. In some embodiments, one of the ITRs (e.g., the 5′ITR) cannot be resolved by the Rep protein, i.e., promoting the formation of a double stranded viral DNA. Such ITRs are described in U.S. Pat. No. 8,784,799, the contents of which are incorporated herein by reference. The presence of such an ITR results in the production of single chain viral DNA.

In some embodiments, the second ITR is ITR2m (SEQ ID NO: 7). In some embodiments, the 5′ ITR is ITR2m (SEQ ID NO: 7) and the 3′ ITR is ITR2 (SEQ ID NO: 8). In some embodiments, the 5′ ITR is ITR2 (SEQ ID NO: 8) and the 3′ ITR is ITR2m (SEQ ID NO: 7).

In an embodiment, an rAAV vector genome comprises a polynucleotide comprising a synthetic ITR of the invention. In a further embodiment, the viral vector can be a parvovirus vector, e.g., an AAV vector. In another embodiment, a recombinant parvovirus particle (e.g., a recombinant AAV particle) comprises a synthetic ITR.

In some embodiments, the rAAV vector comprises nucleic acid that is devoid of bacterial sequence, and/or, lacks alternative open reading frames, and/or, lacks CpGs from the coding sequence, and/or, has double stranded RNA blocker. In some embodiments, the recombinant AAV of the invention is generated from closed ended linear duplexed DNA template. In some embodiments, the recombinant AAV of the invention is generated from plasmid DNA template.

Another embodiment of the invention relates to a method of increasing the transgenic DNA packaging capacity of an AAV capsid, comprising generating an rAAV vector genome that contains the synthetic nucleic acid encoding FKRP and further contains at least one synthetic AAV ITR, wherein said ITR comprises: (a) an AAV rep binding element; (b) an AAV terminal resolution sequence; and (c) an AAV RBE element; wherein said ITR does not comprise any other AAV ITR sequences. Such rAAV vectors are encompassed by the invention.

A further embodiment of the invention relates to a method of altering the cellular response to infection by an rAAV vector genome, comprising generating an rAAV vector genome that contains the synthetic nucleic acid encoding FKRP and further contains at least one synthetic ITR, wherein the nucleotide sequence of one or more transcription factor binding sites in said ITR is deleted and/or substituted, and further wherein an rAAV vector genome comprises at least one synthetic ITR that produces an altered cellular response to infection. Such rAAV vectors are encompassed by the invention.

An additional embodiment of the invention relates to a method of altering the cellular response to infection by an rAAV vector genome, comprising generating an rAAV vector genome that contains the synthetic nucleic acid encoding FKRP and further contains at least one synthetic ITR, wherein one or more CpG motifs in said ITR are deleted and/or substituted, wherein the vector comprising at least one synthetic ITR produces an altered cellular response to infection.

### Muscle Specific Promoters

In some embodiments, the promoter used in the compositions and methods of the invention is a synthetic muscle-specific promoter active in both skeletal and cardiac muscle. Examples of muscle-specific promoters active in both skeletal and cardiac muscle include those shown in Table 1 below, e.g. SP0010, SP0020, SP0033, SP0038, SP0040, SP0042, SP0051, SP0057, SP0058, SP0061, SP0062, SP0064, SP0065, SP0066, SP0068, SP0070, SP0071, SP0076, SP0132, SP0133, SP0134, SP0136, SP0146, SP0147, SP0148, SP0150, SP0153, SP0155, SP0156, SP0157, SP0158, SP0159, SP0160, SP0161, SP0162, SP0163, SP0164, SP0165, SP0166, SP0169, SP0170, SP0171, SP0173, SP0228, SP0229, SP0230, SP0231, SP0232, SP0257, SP0262, SP0264, SP0265, SP0266, SP0267, SP0268, SP0270, SP0271, SP0279, SP0286, SP0305, SP0306, SP0307, SP0309, SP0310, SP0311, SP0312, SP0313, SP0314, SP0315, SP0316, SP0320, SP0322, SP0323, SP0324, SP0325, SP0326, SP0327, SP0328, SP0329, SP0330, SP0331, SP0332, SP0333, SP0334, SP0335, SP0336, SP0337, SP0338, SP0339, SP0340, SP0341, SP0343, SP0345, SP0346, SP0347, SP0348, SP0349, SP0350, SP0351, SP0352, SP0353, SP0354, SP0355, SP0356, SP0358, SP0359, SP0361, SP0362, SP0363, SP0364, SP0365, SP0366, SP0367, SP0368, SP0369, SP0370, SP0371, SP0372, SP0373, SP0374, SP0375, SP0376, SP0377, SP0378, SP0379, SP0380, SP0381, SP0382, SKM 14, SKM_18, SKM_20, SP0357, SP0437-SP0445, SP0447 and SP0453-SP0471, SP0473-474. Examples of preferred synthetic muscle-specific promoters which are active in both skeletal and cardiac muscles are SP0057, SP0134, SP0173, SP0279, SP0286, SP0310, SP0316, SP0320 and SP0326.

### SP0057 and Variants Thereof

In some embodiments, the promoter is a synthetic muscle-specific promoter comprising a combination of the cis-regulatory elements CRE0029 and CRE0071, or functional variants thereof. Typically, the CREs are operably linked to a promoter element. In some preferred embodiments, the muscle-specific promoter comprises said CREs, or functional variants thereof, in the order CRE0029, CRE0071, and then the promoter element (order is given in an upstream to downstream direction, as is conventional in the art). In some preferred embodiments, the muscle-specific promoter comprises said CREs, or functional variants thereof, in the order CRE0071, CRE0029 and then the promoter element.

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0029, CRE0071 and CRE0070, or functional variants thereof.

CRE0029 has a sequence according to SEQ ID NO: 206, shown in the herein provided tables. Functional variants thereof may have a sequence that is at least 60%, 65%, 70%, 75%, 80%, 85%, 90%, 95%, 96%, 97%, 98%, or 99% identical thereto.

Functional variants of CRE0029 are regulatory elements with sequences which vary from CRE0029, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0029 can be viewed as a CRE which, when substituted in place of CRE0029 in a promoter, substantially retains its activity. For example, a muscle-specific promoter which comprises a functional variant of CRE0029 substituted in place of CRE0029 preferably retains 80% of its activity, more preferably 90% of its activity, more preferably 95% of its activity, and yet more preferably 100% of its activity. For example, considering promoter SP0057 as an example, CRE0029 in SP0057 can be replaced with a functional variant of CRE0029, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0029 or functional variant thereof can be provided on either strand of a double stranded polynucleotide and can be provided in either orientation. As such, complementary and reverse complementary sequences of SEQ ID NO: 206 or a functional variant thereof fall within the scope of the invention. Single stranded nucleic acids comprising the sequence according to SEQ ID NO: 206 or a functional variant thereof also fall within the scope of the invention.

CRE0071 has a sequence according to SEQ ID NO: 216, shown in the herein provided tables. Functional variants thereof may have a sequence that is at least 60%, 65%, 70%, 75%, 80%, 85%, 90%, 95%, 96%, 97%, 98%, or 99% identical thereto.

Functional variants of CRE0071 are regulatory elements with sequences which vary from CRE0071, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0071 can be viewed as a CRE which, when substituted in place of CRE0071 in a promoter, substantially retains its activity. For example, a muscle-specific promoter which comprises a functional variant of CRE0029 substituted in place of CRE0071 preferably retains 80% of its activity, more preferably 90% of its activity, more preferably 95% of its activity, and yet more preferably 100% of its activity. For example, considering promoter SP0057 as an example, CRE0071 in SP0057 can be replaced with a functional variant of CRE0071, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0071 or functional variant thereof can be provided on either strand of a double stranded polynucleotide and can be provided in either orientation. As such, complementary and reverse complementary sequences of SEQ ID NO: 216 or a functional variant thereof fall within the scope of the invention. Single stranded nucleic acids comprising the sequence according to SEQ ID NO: 216 or a functional variant thereof also fall within the scope of the invention.

The sequence of CRE0070 (SEQ ID NO: 42) and variants thereof are set out elsewhere herein.

In some embodiments the muscle-specific promoter comprises a sequence according to SEQ ID NO: 87, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 87, shown in the table provided herein, is referred to as SP0057. The SP0057 promoter is particularly preferred in some embodiments. This promoter has been found to be very specific for muscle, which is advantageous in some circumstances.

### SP0134 and Variants Thereof

In some embodiments, the promoter is a synthetic muscle-specific promoter comprising a combination of the cis-regulatory elements CRE0020 and CRE0071, or functional variants thereof. Typically, the CREs are operably linked to a promoter element. In some preferred embodiments, the muscle-specific promoter comprises said CREs, or functional variants thereof, in the order CRE0020, CRE0071, and then the promoter element (order is given in an upstream to downstream direction, as is conventional in the art). In some embodiments, the muscle-specific promoter comprises said CREs, or functional variants thereof, in the order CRE0071, CRE0020 and then the promoter element

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0020, CRE0071 and CRE0070, or functional variants thereof.

CRE0020 has a sequence according to SEQ ID NO: 203, shown in the herein provided table. Functional variants thereof may have a sequence that is at least 60%, 65%, 70%, 75%, 80%, 85%, 90%, 95%, 96%, 97%, 98%, or 99% identical thereto.

Functional variants of CRE0020 are regulatory elements with sequences which vary from CRE0020, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0020 can be viewed as a CRE which, when substituted in place of CRE0020 in a promoter, substantially retains its activity. For example, a skeletal muscle-specific promoter which comprises a functional variant of CRE0020 substituted in place of CRE0020 preferably retains 80% of its activity, more preferably 90% of its activity, more preferably 95% of its activity, and yet more preferably 100% of its activity. For example, considering promoter SP0227 as an example, CRE0020 in SP0227 can be replaced with a functional variant of CRE0020, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0020 or functional variant thereof can be provided on either strand of a double stranded polynucleotide and can be provided in either orientation. As such, complementary and reverse complementary sequences of SEQ ID NO: 203 or a functional variant thereof fall within the scope of the invention. Single stranded nucleic acids comprising the sequence according to SEQ ID NO: 203 or a functional variant thereof also fall within the scope of the invention.

In some embodiments, the CRE0020 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides.

The sequence of CRE0071 and variants thereof are set out above.

The sequence of CRE0070 and variants thereof are set out elsewhere herein.

In some embodiments the muscle-specific promoter comprises a sequence according to the indicated sequences in the tables provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 100, shown in the table provided herein, is referred to as SP0134. The SP0134 promoter is particularly preferred in some embodiments. This promoter has been found to be very specific for muscle, which is advantageous in some circumstances.

### SP0173 and Variants Thereof

In some embodiments, the promoter is a synthetic muscle-specific promoter comprising a combination of muscle specific proximal promoter CRE0010 and cis-regulatory element CRE0035, or functional variants thereof. Typically, muscle specific proximal promoter CRE0010 and cis-regulatory element CRE0035 are operably linked to a further promoter element. In some preferred embodiments, the synthetic muscle-specific promoter comprises said proximal promoter and CRE, or functional variants thereof, in the order CRE0010, CRE0035 and then the further promoter element (order is given in an upstream to downstream direction, as is conventional in the art). In some embodiments, the synthetic muscle-specific promoter comprises said proximal promoter and CRE, or functional variants thereof, in the order CRE0035, CRE0010 and then the further promoter element.

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is SKM_18 or a functional variant thereof. SKM_18 is a muscle-specific proximal promoter.

Thus, in one embodiment the promoter comprises the following regulatory elements: CRE0010, CRE0035 and SKM 18, or functional variants thereof.

CRE0010 has a sequence according to SEQ ID NO: 264. Functional variants thereof may have a sequence that is at least 60%, 65%, 70%, 75%, 80%, 85%, 90%, 95%, 96%, 97%, 98%, or 99% identical thereto.

As discussed above, functional variants of CRE0010 substantially retain the ability of CRE0010 to act as a muscle-specific promoter element. For example, when a functional variant of CRE0010 is substituted into muscle-specific promoter SP0320, the modified promoter retains at least 80% of its activity, more preferably at least 90% of its activity, more preferably at least 95% of its activity, and yet more preferably 100% of the activity of SP0320. Suitably the functional variant of CRE0010 comprises a sequence which is at least 70%, 80%, 90%, 95% or 99% identity to SEQ ID NO: 264, shown in the table provided herein.

In some preferred embodiments, a promoter element comprising or consisting of CRE0010 or a functional variant thereof has a length of 400 or fewer nucleotides, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

CRE0035 has a sequence according to SEQ ID NO: 208, shown in the table provided herein. Functional variants thereof may have a sequence that is at least 60%, 65%, 70%, 75%, 80%, 85%, 90%, 95%, 96%, 97%, 98%, or 99% identical thereto.

Functional variants of CRE0035 are regulatory elements with sequences which vary from CRE0035, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0035 can be viewed as a CRE which, when substituted in place of CRE0035 in a promoter, substantially retains its activity. For example, a muscle-specific promoter which comprises a functional variant of CRE0035 substituted in place of CRE0035 preferably retains 80% of its activity, more preferably 90% of its activity, more preferably 95% of its activity, and yet more preferably 100% of its activity. For example, considering promoter SP0173 as an example, CRE0035 in SP0173 can be replaced with a functional variant of CRE0035, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0035 or functional variant thereof can be provided on either strand of a double stranded polynucleotide and can be provided in either orientation. As such, complementary and reverse complementary sequences of SEQ ID NO: 208 or a functional variant thereof fall within the scope of the invention. Single stranded nucleic acids comprising the sequence according to SEQ ID NO: 208 or a functional variant thereof also fall within the scope of the invention.

The sequence of SKM_18 and variants thereof are set out elsewhere herein.

In some embodiments the muscle-specific promoter comprises a sequence according to SEQ ID NO: 122, shown on the table provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 122 is referred to as SP0173. The SP0173 promoter is particularly preferred in some embodiments. This promoter has been found to be very specific for muscle, which is advantageous in some circumstances.

### SP0279 and Variants Thereof

In some embodiments, the promoter is a synthetic muscle-specific promoter comprising a combination of the cis-regulatory elements CRE0020 and CRE0071, or functional variants thereof. Typically, the CREs are operably linked to a promoter element. In some preferred embodiments, the muscle-specific promoter comprises said CREs, or functional variants thereof, in the order CRE0020, CRE0071, and then the promoter element (order is given in an upstream to downstream direction, as is conventional in the art). In some preferred embodiments, the muscle-specific promoter comprises said CREs, or functional variants thereof, in the order CRE0071, CRE0020 and then the promoter element. In some preferred embodiments, the muscle-specific promoter comprises said CREs, or functional variants thereof, in the order CRE0020, CRE0071, the promoter element and the CMV-IE 5′UTR and Intron (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0020, CRE0071, CRE0070 and CMV-IE 5′UTR and intron, or functional variants thereof.

The sequence of CRE0020 and variants thereof are set out above.

The sequence of CRE0071 and variants thereof are set out above.

The sequence of CRE0070 and variants thereof are set out elsewhere herein.

The sequence of CMV-IE 5′UTR and intron and variants thereof are set out elsewhere herein.

In some embodiments the muscle-specific promoter comprises a sequence according to SEQ ID NO: 137, shown on the table provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 137 is referred to as SP0279. The SP0279 promoter is particularly preferred in some embodiments. This promoter has been found to be very specific for muscle, which is advantageous in some circumstances.

### SP0286 and Variants Thereof

In some embodiments, the promoter is a synthetic muscle-specific promoter comprising CRE0071 operably linked to a promoter element. In some preferred embodiments, the synthetic muscle-specific promoter comprises CRE0071 immediately upstream of the promoter element. In some preferred embodiments, the synthetic muscle-specific promoter comprises CRE0071 immediately upstream of the promoter element and CMV-IE 5′UTR and intron.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is CRE0070 or functional variant thereof. CRE0070 is a muscle-specific proximal promoter.

In some embodiments the synthetic muscle-specific promoter comprises the following elements (or functional variants thereof): CRE0071, CRE0070 and then CMV-IE 5′UTR and intron.

The sequence of CRE0071 and variants thereof are set out above.

The sequence of CRE0070 and variants thereof are set out elsewhere herein.

The sequence of CMV-IE 5′UTR and intron and variants thereof are set out elsewhere herein.

In some embodiments the muscle-specific promoter comprises a sequence according to SEQ ID NO: 138, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 138 is referred to as SP0286. The SP0286 promoter is particularly preferred in some embodiments. This promoter has been found to be very specific for muscle, which is advantageous in some circumstances.

### SP0310 and Variants Thereof

In some embodiments, the promoter is a synthetic muscle-specific promoter comprising CRE0035 operably linked to a promoter element. In some preferred embodiments, the synthetic muscle-specific promoter comprises CRE0035 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0035 and then SKM_18.

The sequence of CRE0035 and variants thereof are set out above.

The sequence of SKM_18 and variants thereof are set out elsewhere herein.

In some embodiments the muscle-specific promoter comprises a sequence according to SEQ ID NO: 143, shown in the table provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 143 is referred to as SP0310. The SP0310 promoter is particularly preferred in some embodiments. This promoter has been found to be very specific for muscle, which is advantageous in some circumstances.

### SP0316 and Variants Thereof

In some embodiments, the promoter is a synthetic muscle-specific promoter comprising CRE0050 operably linked to a promoter element. In some preferred embodiments, the synthetic muscle-specific promoter comprises CRE0050 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0050 and then SKM_18.

CRE0050 has a sequence according to SEQ ID NO: 211. Functional variants thereof may have a sequence that is at least 60%, 65%, 70%, 75%, 80%, 85%, 90%, 95%, 96%, 97%, 98%, or 99% identical thereto.

Functional variants of CRE0050 are regulatory elements with sequences which vary from CRE0050, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0050 can be viewed as a CRE which, when substituted in place of CRE0050 in a promoter, substantially retains its activity. For example, a muscle-specific promoter which comprises a functional variant of CRE0035 substituted in place of CRE0050 preferably retains 80% of its activity, more preferably 90% of its activity, more preferably 95% of its activity, and yet more preferably 100% of its activity. For example, considering promoter SP0316 as an example, CRE0050 in SP0316 can be replaced with a functional variant of CRE0050, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0050 or functional variant thereof can be provided on either strand of a double stranded polynucleotide and can be provided in either orientation. As such, complementary and reverse complementary sequences of SEQ ID NO: 211 or a functional variant thereof fall within the scope of the invention. Single stranded nucleic acids comprising the sequence according to SEQ ID NO: 211 or a functional variant thereof also fall within the scope of the invention.

The sequence of SKM_18 and variants thereof are set out elsewhere herein.

In some embodiments the muscle-specific promoter comprises a sequence according to SEQ ID NO: 149, shown in the table provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 149 is referred to as SP0316. The SP0316 promoter is particularly preferred in some embodiments. This promoter has been found to be very specific for muscle, which is advantageous in some circumstances.

### SP0320 and Variants Thereof

In some embodiments, the promoter is a synthetic muscle-specific promoter comprising a combination of muscle specific proximal promoter CRE0010 and cis-regulatory element CRE0035, or functional variants thereof. Typically, muscle specific proximal promoter CRE0010 and cis-regulatory element CRE0035 are operably linked to a further promoter element. In some preferred embodiments, the synthetic muscle-specific promoter comprises said proximal promoter and CRE, or functional variants thereof, in the order CRE0010, CRE0035 and then the further promoter element (order is given in an upstream to downstream direction, as is conventional in the art). In some embodiments, the synthetic muscle-specific promoter comprises said proximal promoter and CRE, or functional variants thereof, in the order CRE0035, CRE0010 and then the further promoter element. In some preferred embodiments, the synthetic muscle-specific promoter comprises said proximal promoter and CRE, or functional variants thereof, in the order CRE0010, CRE0035, the further promoter element followed by the CMV-IE 5′UTR and Intron.

The further promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is SKM_18 or a functional variant thereof. SKM_18 is a muscle-specific proximal promoter.

Thus, in one embodiment the promoter comprises the following regulatory elements: CRE0010, CRE0035, SKM_18 and CMV-IE 5′UTR and intron, or functional variants thereof.

The sequence of CRE0010 and variants thereof are set out above.

The sequence of CRE0035 and variants thereof are set out above.

The sequence of SKM_18 and variants thereof are set out elsewhere herein.

The sequence of the CMV-IE 5′UTR and intron and variants thereof are set out elsewhere herein.

In some embodiments the muscle-specific promoter comprises a sequence according to SEQ ID NO: 150, shown in the table provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 150 is referred to as SP0320. The SP0320 promoter is particularly preferred in some embodiments. This promoter has been found to be very specific for muscle, which is advantageous in some circumstances.

### SP0326 and Variants Thereof

In some embodiments, the promoter is a synthetic muscle-specific promoter comprising CRE0071 operably linked to a promoter element. In some preferred embodiments, the synthetic muscle-specific promoter comprises CRE0071 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0071 and then SKM_18.

The sequence of CRE0071 and variants thereof are set out above.

The sequence of SKM_18 and variants thereof are set out elsewhere herein.

In some embodiments the muscle-specific promoter comprises a sequence according to SEQ ID NO: 155, shown in the table provided herein, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 155 is referred to as SP0326. The SP0326 promoter is particularly preferred in some embodiments. This promoter has been found to be very specific for muscle, which is advantageous in some circumstances.

TABLE 1
Muscle-specific promoters active in cardiac and skeletal muscle

| NAME | SEQUENCE | LENGTH |
|---|---|---|
| SP0010 | GTTTCTTAGCAGCTGCTGCTGTGTCCAAGGCTTGGAATTGCTG | 298 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | C (SEQ ID NO: 80) | |
| SP0020 | GGGCCCCACAGCAGCTGGGGGCATTTATGGGCCTTCCTATAA | 354 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCCACCGCCTGCTG | |
| | CCACGGCCGGCCGTATAAATAGAGGCGAGGAGCAGCTGGGC | |
| | TCTCTTGGCAGTCACC (SEQ ID NO: 81) | |
| SP0033 | GCCACTACGGGTCTAGGCTGCCCATGTAAGGAGGCAAGGCCT | 270 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCCACCGCCTGCTGCCAC | |
| | GGCCGGCCGTATAAATAGAGGCGAGGAGCAGCTGGGCTCTCT | |
| | TGGCAGTCACCGCCACC (SEQ ID NO: 82) | |
| SP0038 | TAAGTCCGGGCAGGGTCCTGTCCATAAAAGGCTTTTCCCGGG | 286 |
| | CCGGCTCCCCGCCGGCAGCGTGCCCCGCCCCGGCCCGCTCCA | |
| | TCTCCAAAGCATGCAGAGAATGTCTCGGCAGCCCCGGTAGAC | |
| | TGCTCCAACTTGGTGTCTTTCCCCAAATATGGAGCCTGTGTGG | |
| | AGTCACTGGGGGAGCCGGGGGTGGGGAGCGGAGCCGGCTTC | |
| | CTCTAGCCACCGCCTGCTGCCACGGCCGGCCGTATAAATAGA | |
| | GGCGAGGAGCAGCTGGGCTCTCTTGGCAGTCACC (SEQ ID | |
| | NO: 83) | |
| SP0040 | CTGAGATTTTCCTAGCATTTTGTGTTTCATGACTAAATATGGT | 315 |
| | TTGTGTTTCAAGACCAATGAGCTGGGAACTGTACTGTTCTTTC | |
| | CCCTCCCATCAACTCATTTTTGGCACAAGACGCACTCTAGTCA | |
| | GTTGGAGCAAATCCCCTGACCCGGGTGCAGTTCCAAAAGCAG | |
| | ACACTCGAGCGTGTTTTACCTAATTAGGAAATGCTTTGCTCCA | |
| | AACCGAACTGCTCATTCAGGTTAGAGAGGAGCCACCGCCTGC | |
| | TGCCACGGCCGGCCGTATAAATAGAGGCGAGGAGCAGCTGG | |
| | GCTCTCTTGGCAGTCACC (SEQ ID NO: 84) | |
| SP0042 | CTGAGATTTTCCTAGCATTTTGTGTTTCATGACTAAATATGGT | 421 |
| | TTGTGTTTCAAGACCAATGAGCTGGGAACTGTACTGTTCTTTC | |
| | CCCTCCCATCAACTCATTTTTGGCACAAGACGCACTCTAGTCA | |
| | GTTGGAGCAAATCCCCTGACCCGGGTGCAGTTCCAAAAGCAG | |
| | ACACTCGAGCGTGTTTTACCTAATTAGGAAATGCTTTGCTCCA | |
| | AACCGAACTGCTCATTCAGGTTAGAGAGGAGAGGTCCCTATA | |
| | TGGTTGTGTTAGAGTGAACGGCCAGCTTCAGCCCGTCTTTGCT | |
| | CCTTGTTTGGGAAGCGAGTGGGAGGGGATCAGAGCAAGGGG | |
| | CTATATAACCCTTCAGCGTTCAGCCTCCCGGGACACCACCCA | |
| | CCCAGAGTGGAGAAGCCCAGCCAGTCGCTGTCAGCCACC | |
| | ((SEQ ID NO: 85) | |
| SP0051 | GGGCCCCACAGCAGCTGGGGGCATTTATGGGCCTTCCTATAA | 524 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTTTCTCCTCTATAA | |
| | ATACCCGCTCTGGTATTTGGGGTTGGCAGCTGTTGCTGCCAGG | |
| | GAGATGGTTGGGTTGACGGGATCTTGCAGCTGTCAGGGGAGG | |
| | GGAGGCGGGGGCTGATGTCAGGAGGGATACAAATAGTGCCG | |
| | ACGGCTGGGGGCCCTGTCTCCCCTCGCCGCATCCACTCTCCGG | |
| | CCGGCCGCCTGCCCGCCGCCTCCTCCGTGCGCCCGCCAGCCTC | |
| | GCCCGCGCCGTCACC (SEQ ID NO: 86) | |
| SP0057 | CTCTGTCTCCTCAGGTGCCTGGCTCCCAGTCCCCAGAACGCCT | 601 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCCACCGCGGTGGCGGCCGTCCGCCCTC | |
| | GGCACCATCCTCACGACACCCAAATATGGCGACGGGTGAGGA | |
| | ATGGTGGGGAGTTATTTTTAGAGCGGTGAGGAAGGTGGGCAG | |
| | GCAGCAGGTGTTGGCGCTCTAAAAATAACTCCCGGGAGTTAT | |
| | TTTTAGAGCGGAGGAATGGTGGACACCCAAATATGGCGACGG | |
| | TTCCTCACCCGTCGCCATATTTGGGTGTCCGCCCTCGGCCGGG | |
| | GCCGCATTCCTGGGGGCCGGGCGGTGCTCCCGCCCGCCTCGA | |
| | TAAAAGGCTCCGGGGCCGGCGGCGGCCCACGAGCTACCCGG | |
| | AGGAGCGGGAGGCGCCAAGCTCTAGAACTAGTGGATCCCGC | |
| | GGCCGCCACC (SEQ ID NO: 87) | |
| SP0058 | CCTTGCCTGACTATTGGCAGGCGGACCTGGTGGTCAGACCTC | 531 |
| | AGTGATCCTCAGGGACCAGTGAATATTTCAGGCTGGGGCTGA | |
| | GCATCACCTGCTCCCTTGGCCCCACTTATAGGGCAAAGGGGA | |
| | GTCTACCAGCCTACTCACTGATGACAAACTGGAAAAGTTTGT | |
| | CCTGTCTCTGCTCTGGCCCCACCTCGCCCTCTCCCCTACTTGG | |
| | AAGTTCCTTTCCTGAACCACTGACTGCCAAAGCTTGAGGGAT | |
| | TAAATAAATCATCTGGCCCAAACTCGGGGGCCAGGCACTGGC | |
| | GCTGACGCAGGCTAGCAGGGCGCCACTGGCTGGTCCCCACCC | |
| | ACCTCGGTGGGTTGGGGGATGGGCGCACCAGCCCCTCCTGGG | |
| | TGAGCCCTAGCCTGGGGCTTCCTATTTCGGGAGCCGGGGGCG | |
| | TGGGCCACGTCTCCTCATGTGATGCGAGGGCTATTTAAAGCG | |
| | GCAGCCCGGGCAGGGAGCCGCCGTCGGAGCCCTTGCACGCCT | |
| | GCTCTCTTGTAGCTGCGGCCGCCACC (SEQ ID NO: 88) | |
| SP0061 | CCTTGCCTGACTATTGGCAGGCGGACCTGGTGGTCAGACCTC | 528 |
| | AGTGATCCTCAGGGACCAGTGAATATTTCAGGCTGGGGCTGA | |
| | GCATCACCTGCTCCCTTGGCCCCACTTATAGGGCAAAGGGGA | |
| | GTCTACCAGCCTACTCACTGATGACAAACTGGAAAAGTTTGT | |
| | CCTGTCTCTGCTCTGGCCCCACCTCGCCCTCTCCCCTACTTGG | |
| | AAGTTCCTTTCCTGAACCACTGACTGCCAAAGCTTGAGGGAT | |
| | TAAATAAATCATCTGGCCCAAATAAATACCCGCTCTGGTATTT | |
| | GGGGTTCTCCTCTATAAATACCCGCTCTGGTATTTGGGGTTGG | |
| | CAGCTGTTGCGGGATCTTGCAGCTGTCAGGGGAGGGGAGGCG | |
| | GGGGCTGATGTCAGGAGGGATACAAATAGTGCCGACGGCTG | |
| | GGGGCCCTGTCTCCCCTCGCCGCATCCACTCTCCGGCCGGCCG | |
| | CCTGCCCGCCGCCTCCTCCGTGCGCCCGCCAGCCTCGCCCGCG | |
| | CCGTCACCGCGGCCGCCACC (SEQ ID NO: 89) | |
| SP0062 | CTGTGTGTTTCTGTGGCTGAGTCAGATGGAGGAGTCCTCATGT | 454 |
| | TTCACTGCTTAGCAGTTTTTGTCCTTCCTAGTACCCGTTCCCA | |
| | GCCCACAAGATGCAGAAAGAGCTGTTGCTAGCGTGAGTTATT | |
| | TTTGTCAGCTGAGTCACCACGCCAGAAAGCAAGAAATGACCC | |
| | GCTTTATGTCTGCTCTGAGGAGCTGGAACCATAAATACCCGC | |
| | TCTGGTATTTGGGGTTCTCCTCTATAAATACCCGCTCTGGTAT | |
| | TTGGGGTTGGCAGCTGTTGCGGGATCTTGCAGCTGTCAGGGG | |
| | AGGGGAGGCGGGGGCTGATGTCAGGAGGGATACAAATAGTG | |
| | CCGACGGCTGGGGGCCCTGTCTCCCCTCGCCGCATCCACTCTC | |
| | CGGCCGGCCGCCTGCCCGCCGCCTCCTCCGTGCGCCCGCCAG | |
| | CCTCGCCCGCGCCGTCACCGCGGCCGCCACC (SEQ ID NO: 90) | |
| SP0064 | TACATCATTTACCTAGAAAAGAGGACAGCTGTCCTTTCCCAA | 484 |
| | AGCTCCGGTGACCCTGCCCCGCCCAGTGTGACTAGCCCAGGT | |
| | TGGTGATTCTGATCTGTTGCCAAACCAAACTGGCTCCCCGGG | |
| | GAGCCATTTGGTAATGTTCCCTGGAGTCATTTCCTTGCGAAGC | |
| | ATTCCTTTTCGGTGAGAGGACATTTTTTTCATCCCTGATAAAC | |
| | AACCACAGCCTGCGCCAGATAAATACCCGCTCTGGTATTTGG | |
| | GGTTCTCCTCTATAAATACCCGCTCTGGTATTTGGGGTTGGCA | |
| | GCTGTTGCGGGATCTTGCAGCTGTCAGGGGAGGGGAGGCGGG | |
| | GGCTGATGTCAGGAGGGATACAAATAGTGCCGACGGCTGGG | |
| | GGCCCTGTCTCCCCTCGCCGCATCCACTCTCCGGCCGGCCGCC | |
| | TGCCCGCCGCCTCCTCCGTGCGCCCGCCAGCCTCGCCCGCGCC | |
| | GTCACCGCGGCCGCCACC (SEQ ID NO: 91) | |
| SP0065 | TAAGTGTGATGCACAGTGCTTGCATTTTCTTGATACGTTAGTC | 465 |
| | ATATGAGAGCTGACAAAGAAGGAAAAAGAGCAGCGATGTGG | |
| | TGCAATATTAACAGGCAGCTGTCCCCTGGCTTCCCGATACGT | |
| | GGGATGACTCGCATTGCTGAGCGGTGTGGTCACTGCCAAAGG | |
| | AATGACCCTCTCACATTTCTTCCTGATTCGCATACGCCGCGGC | |
| | ATAAATACCCGCTCTGGTATTTGGGGTTCTCCTCTATAAATAC | |
| | CCGCTCTGGTATTTGGGGTTGGCAGCTGTTGCGGGATCTTGCA | |
| | GCTGTCAGGGGAGGGGAGGCGGGGGCTGATGTCAGGAGGGA | |
| | TACAAATAGTGCCGACGGCTGGGGGCCCTGTCTCCCCTCGCC | |
| | GCATCCACTCTCCGGCCGGCCGCCTGCCCGCCGCCTCCTCCGT | |
| | GCGCCCGCCAGCCTCGCCCGCGCCGTCACCGCGGCCGCCACC | |
| | (SEQ ID NO: 92) | |
| SP0066 | CTCTGTCTCCTCAGGTGCCTGGCTCCCAGTCCCCAGAACGCCT | 484 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCATAAATACCCGCTCTGGTATTTGGGG | |
| | TTCTCCTCTATAAATACCCGCTCTGGTATTTGGGGTTGGCAGC | |
| | TGTTGCGGGATCTTGCAGCTGTCAGGGGAGGGGAGGCGGGG | |
| | GCTGATGTCAGGAGGGATACAAATAGTGCCGACGGCTGGGG | |
| | GCCCTGTCTCCCCTCGCCGCATCCACTCTCCGGCCGGCCGCCT | |
| | GCCCGCCGCCTCCTCCGTGCGCCCGCCAGCCTCGCCCGCGCC | |
| | GTCACCGCGGCCGCCACC (SEQ ID NO: 93) | |
| SP0068 | GCCACTACGGGTCTAGGCTGCCCATGTAAGGAGGCAAGGCCT | 448 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGATAAATACCCGCTCTGG | |
| | TATTTGGGGTTCTCCTCTATAAATACCCGCTCTGGTATTTGGG | |
| | GTTGGCAGCTGTTGCGGGATCTTGCAGCTGTCAGGGGAGGGG | |
| | AGGCGGGGGCTGATGTCAGGAGGGATACAAATAGTGCCGAC | |
| | GGCTGGGGGCCCTGTCTCCCCTCGCCGCATCCACTCTCCGGCC | |
| | GGCCGCCTGCCCGCCGCCTCCTCCGTGCGCCCGCCAGCCTCG | |
| | CCCGCGCCGTCACCGCGGCCGCCACC (SEQ ID NO: 94) | |
| SP0070 | CTGTGTGTTTCTGTGGCTGAGTCAGATGGAGGAGTCCTCATGT | 444 |
| | TTCACTGCTTAGCAGTTTTTGTCCTTCCTAGTACCCGTTCCCA | |
| | GCCCACAAGATGCAGAAAGAGCTGTTGCTAGCGTGAGTTATT | |
| | TTTGTCAGCTGAGTCACCACGCCAGAAAGCAAGAAATGACCC | |
| | GCTTTATGTCTGCTCTGAGGAGCTGGAACCATTTTTAAAGACT | |
| | GAGGAATTAGGCACCTGTCATTTTTGCCAGCTGGTGTAGATG | |
| | TTAAAAATTACTGTCACTCTTCCGCCTGCTACTTTATTTTGCA | |
| | CCTGCTGTTACTTGAGTTACAGGCATTTCACACATGGTAATTT | |
| | AATAAGGTTAGTTCCCATGACACACCGCCTGCTGCCACGGCC | |
| | GGCCGTATAAATAGAGGCGAGGAGCAGCTGGGCTCTCTTGGC | |
| | AGTCACCGCGGCCGCCACC (SEQ ID NO: 95) | |
| SP0071 | GCGCCCTGATGAATATGCATCGCGGCGCGCCCGCCCCCGGCT | 404 |
| | CCTCCTTTCGGTTTCCTTCCCGCCGCCAGGCGGAAGCGAAGA | |
| | GCCGCGCTTCCCGCGCGCCCAGGCCGGCCGTGGTAGGGTGGG | |
| | GCGGGGCGGGCCGCGAGCCGGAGAAAGAGAAAGCATTTTTA | |
| | AAGACTGAGGAATTAGGCACCTGTCATTTTTGCCAGCTGGTG | |
| | TAGATGTTAAAAATTACTGTCACTCTTCCGCCTGCTACTTTAT | |
| | TTTGCACCTGCTGTTACTTGAGTTACAGGCATTTCACACATGG | |
| | TAATTTAATAAGGTTAGTTCCCATGACACACCGCCTGCTGCCA | |
| | CGGCCGGCCGTATAAATAGAGGCGAGGAGCAGCTGGGCTCTC | |
| | TTGGCAGTCACCGCGGCCGCCACC (SEQ ID NO: 96) | |
| SP0076 | GCCACTACGGGTCTAGGCTGCCCATGTAAGGAGGCAAGGCCT | 438 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGATTTTTAAAGACTGAGG | |
| | AATTAGGCACCTGTCATTTTTGCCAGCTGGTGTAGATGTTAAA | |
| | AATTACTGTCACTCTTCCGCCTGCTACTTTATTTTGCACCTGCT | |
| | GTTACTTGAGTTACAGGCATTTCACACATGGTAATTTAATAAG | |
| | GTTAGTTCCCATGACACACCGCCTGCTGCCACGGCCGGCCGT | |
| | ATAAATAGAGGCGAGGAGCAGCTGGGCTCTCTTGGCAGTCAC | |
| | CGCGGCCGCCACC (SEQ ID NO: 97) | |
| SP0132 | GGGCCCCACAGCAGCTGGGGGCATTTATGGGCCTTCCTATAA | 538 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTATAAATACCCGCT | |
| | CTGGTATTTGGGGTTCTCCTCTATAAATACCCGCTCTGGTATT | |
| | TGGGGTTGGCAGCTGTTGCGGGATCTTGCAGCTGTCAGGGGA | |
| | GGGGAGGCGGGGGCTGATGTCAGGAGGGATACAAATAGTGC | |
| | CGACGGCTGGGGGCCCTGTCTCCCCTCGCCGCATCCACTCTCC | |
| | GGCCGGCCGCCTGCCCGCCGCCTCCTCCGTGCGCCCGCCAGC | |
| | CTCGCCCGCGCCGTCACCGCGGCCGCCACC (SEQ ID NO: 98) | |
| SP0133 | GGGCCCCACAGCAGCTGGGGGCATTTATGGGCCTTCCTATAA | 528 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTATTTTTAAAGACT | |
| | GAGGAATTAGGCACCTGTCATTTTTGCCAGCTGGTGTAGATG | |
| | TTAAAAATTACTGTCACTCTTCCGCCTGCTACTTTATTTTGCA | |
| | CCTGCTGTTACTTGAGTTACAGGCATTTCACACATGGTAATTT | |
| | AATAAGGTTAGTTCCCATGACACACCGCCTGCTGCCACGGCC | |
| | GGCCGTATAAATAGAGGCGAGGAGCAGCTGGGCTCTCTTGGC | |
| | AGTCACCGCGGCCGCCACC (SEQ ID NO: 99) | |
| SP0134 | GGGCCCCACAGCAGCTGGGGGCATTTATGGGCCTTCCTATAA | 655 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCGGCCGGGGCCGCATTCCTGGGGGCCGGGCGGTGCT | |
| | CCCGCCCGCCTCGATAAAAGGCTCCGGGGCCGGCGGCGGCCC | |
| | ACGAGCTACCCGGAGGAGCGGGAGGCGCCAAGCTCTAGAAC | |
| | TAGTGGATCCCGCGGCCGCCACC (SEQ ID NO: 100) | |
| SP0136 | GGGCCCCACAGCAGCTGGGGGCATTTATGGGCCTTCCTATAA | 588 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTGTTTCTTAGCAGC | |
| | TGCTGCTGTGTCCAAGGCTTGGAATTGCTGTGGTGAATCTAA | |
| | AACTGTCTCAGTAGTGGTGAGCTGACCTCACCCAAGTTCAAA | |
| | GCCCTACTCTGCCTGATCCTTTTTTCCTGAGCCTCAGAGCTAA | |
| | AATGCCCCCGAGCTCTTTCCTATTGGCTGGAAAGACGAATTG | |
| | AAGTTCCCTTGCCCATGTTAGGAGGTGTACGCCTCCTGAACTA | |
| | AAGATAGAAACAGCTGGCCCTTCCAGGCAGCTAAAAGCCTCC | |
| | AGACTAAGAGGTGTTCCCCATTCGGGCGGCCGCCACC (SEQ | |
| | ID NO: 101) | |
| SP0146 | CTAGACTAGCATGCTGCCCATGTAAGGAGGCAAGGCCTGGGG | 660 |
| | ACACCCGAGATGCCTGGTTATAATTAACCCAGACATGTGGCT | |
| | GCCCCCCCCCCCCCAACACCTGCTGCCTCTAAAAATAACCCT | |
| | GCATGCCATGTTCCCGGCGAAGGGCCAGCTGTCCCCCGCCAG | |
| | CTAGACTCAGCACTTAGTTTAGGAACCAGTGAGCAAGTCAGC | |
| | CCTTGGGGCAGCCCATACAAGGCCATGGGGCTGGGCAAGCTG | |
| | CACGCCTGGGTCCGGGGTGGGCACGGTGCCCGGGCAACGAG | |
| | CTGAAAGCTCATCTGCTCTCAGGGGCCCCTCCCTGGGGACAG | |
| | CCCCTCCTGGCTAGTCACACCCTGTAGGCTCCTCTATATAACC | |
| | CAGGGGCACAGGGGCTGCCCTCATTCTACCACCACCTCCACA | |
| | GCACAGACAGACACTCAGGAGCCAGCCAGCCAGGTAGGGAC | |
| | TGTACTAGCAGCTACAATCCAGCTACCATTCTGCTTTTATTTT | |
| | ATGGTTGGGATAAGGCTGGATTATTCTGAGTCCAAGCTAGGC | |
| | CCTTTTGCTAATCATGTTCATACCTCTTATCTTCCTCCCACAGC | |
| | TCCTGGGCAACGTGCTGGTCTGTGTGCTGGCCCATCACTTTGG | |
| | CAAAGAATTGCGATCGCCTCTAGAACC (SEQ ID NO: 102) | |
| SP0147 | GGGCCCCACAGCAGCTGGGGGCATTTATGGGCCTTCCTATAA | 806 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCATGTTCCCGGCG | |
| | AAGGGCCAGCTGTCCCCCGCCAGCTAGACTCAGCACTTAGTT | |
| | TAGGAACCAGTGAGCAAGTCAGCCCTTGGGGCAGCCCATACA | |
| | AGGCCATGGGGCTGGGCAAGCTGCACGCCTGGGTCCGGGGTG | |
| | GGCACGGTGCCCGGGCAACGAGCTGAAAGCTCATCTGCTCTC | |
| | AGGGGCCCCTCCCTGGGGACAGCCCCTCCTGGCTAGTCACAC | |
| | CCTGTAGGCTCCTCTATATAACCCAGGGGCACAGGGGCTGCC | |
| | CTCATTCTACCACCACCTCCACAGCACAGACAGACACTCAGG | |
| | AGCCAGCCAGCCAGGTAGGGACTGTACTAGCAGCTACAATCC | |
| | AGCTACCATTCTGCTTTTATTTTATGGTTGGGATAAGGCTGGA | |
| | TTATTCTGAGTCCAAGCTAGGCCCTTTTGCTAATCATGTTCAT | |
| | ACCTCTTATCTTCCTCCCACAGCTCCTGGGCAACGTGCTGGTC | |
| | TGTGTGCTGGCCCATCACTTTGGCAAAGAATTGCGATCGCCA | |
| | CC (SEQ ID NO: 103) | |
| SP0148 | GGGCCCCACAGCAGCTGGGGGCATTTATGGGCCTTCCTATAA | 938 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCAATTCTCATGTT | |
| | TGACAGCTTATCATCGCAGATCCGTATGGTGCACTCTCAGTAC | |
| | AATCTGCTCTGATGCCGCATAGTTAAGCCAGTATCTGCTCCCT | |
| | GCTTGTGTGTTGGAGGTCGCTGAGTAGTGCGCGAGCAAAATT | |
| | TAAGCTACAACAAGGCAAGGCTTGACCGACAATTGCATGAAG | |
| | AATCTGCTTAGGGTTAGGCGTTTTGCGCTGCTTCGCGATGTAC | |
| | GGGCCAGATATACGCGTATCTGAGGGGACTAGGGTGTGTTTA | |
| | GGCGAAAAGCGGGGCTTCGGTTGTACGCGGTTAGGAGTCCCC | |
| | TCAGGATATAGTAGTTTCGCTTTTGCATAGGGAGGGGGAAAT | |
| | GTAGTCTTATGCAATACTCTTGTAGTCTTGCAACATGGTAACG | |
| | ATGAGTTAGCAACATGCCTTACAAGGAGAGAAAAAGCACCG | |
| | TGCATGCCGATTGGTGGAAGTAAGGTGGTACGATCGTGCCTT | |
| | ATTAGGAAGGCAACAGACGGGTCTGACATGGATTGGACGAA | |
| | CCACTGAATTCCGCATTGCAGAGATATTGTATTTAAGTGCCTA | |
| | GCTCGATACAATAAACGCCATTTGACCATTCACCACATTGGT | |
| | GTGCACCTCCAAGCTGGGTACCGCGGGCCCGGGATCCACCGG | |
| | TCGCCACC (SEQ ID NO: 104) | |
| SP0150 | GCGCCCTGATGAATATGCATCGCGGCGCGCCCGCCCCCGGCT | 814 |
| | CCTCCTTTCGGTTTCCTTCCCGCCGCCAGGCGGAAGCGAAGA | |
| | GCCGCGCTTCCCGCGCGCCCAGGCCGGCCGTGGTAGGGTGGG | |
| | GCGGGGCGGGCCGCGAGCCGGAGAAAGAGAAAGCCAATTCT | |
| | CATGTTTGACAGCTTATCATCGCAGATCCGTATGGTGCACTCT | |
| | CAGTACAATCTGCTCTGATGCCGCATAGTTAAGCCAGTATCT | |
| | GCTCCCTGCTTGTGTGTTGGAGGTCGCTGAGTAGTGCGCGAG | |
| | CAAAATTTAAGCTACAACAAGGCAAGGCTTGACCGACAATTG | |
| | CATGAAGAATCTGCTTAGGGTTAGGCGTTTTGCGCTGCTTCGC | |
| | GATGTACGGGCCAGATATACGCGTATCTGAGGGGACTAGGGT | |
| | GTGTTTAGGCGAAAAGCGGGGCTTCGGTTGTACGCGGTTAGG | |
| | AGTCCCCTCAGGATATAGTAGTTTCGCTTTTGCATAGGGAGG | |
| | GGGAAATGTAGTCTTATGCAATACTCTTGTAGTCTTGCAACAT | |
| | GGTAACGATGAGTTAGCAACATGCCTTACAAGGAGAGAAAA | |
| | AGCACCGTGCATGCCGATTGGTGGAAGTAAGGTGGTACGATC | |
| | GTGCCTTATTAGGAAGGCAACAGACGGGTCTGACATGGATTG | |
| | GACGAACCACTGAATTCCGCATTGCAGAGATATTGTATTTAA | |
| | GTGCCTAGCTCGATACAATAAACGCCATTTGACCATTCACCA | |
| | CATTGGTGTGCACCTCCAAGCTGGGTACCGCGGGCCCGGGAT | |
| | CCACCGGTCGCCACC (SEQ ID NO: 105) | |
| SP0153 | GCCACTACGGGTCTAGGCTGCCCATGTAAGGAGGCAAGGCCT | 418 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCCCGGCAGACGCTCCTT | |
| | ATACGGCCCGGCCTCGCTCACCTGGGCCGCGGCCAGGAGCGC | |
| | CTTCTTTGGGCAGCGCCGGGCCGGGGCCGCGCCGGGCCCGAC | |
| | ACCCAAATATGGCGACGGCCGGGGCCGCATTCCTGGGGGCCG | |
| | GGCGGCGCTCCCGCCCGCCTCGATAAAAGGCTCCGGGGCCGG | |
| | CGGCGGCCCACGAGCTACCCGGAGGAGCGGGAGGCCACC | |
| | (SEQ ID NO: 106) | |
| SP0155 | GCCACTACGGGTCTAGGCTGCCCATGTAAGGAGGCAAGGCCT | 508 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTTCTCCTCTATAAATACC | |
| | CGCTCTGGTATTTGGGGTTGGCAGCTGTTGTTCTCCTCTATAA | |
| | ATACCCGCTCTGGTATTTGGGGTTGGCAGCTGTTGCCCGGCA | |
| | GACGCTCCTTATACGGCCCGGCCTCGCTCACCTGGGCCGCGG | |
| | CCAGGAGCGCCTTCTTTGGGCAGCGCCGGGCCGGGGCCGCGC | |
| | CGGGCCCGACACCCAAATATGGCGACGGCCGGGGCCGCATTC | |
| | CTGGGGGCCGGGCGGCGCTCCCGCCCGCCTCGATAAAAGGCT | |
| | CCGGGGCCGGCGGCGGCCCACGAGCTACCCGGAGGAGCGGG | |
| | AG (SEQ ID NO: 107) | |
| SP0156 | GCCACTACGGGTCTAGGCTGCCCATGTAAGGAGGCAAGGCCT | 718 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGGGGCCCCACAGCAGCTG | |
| | GGGGCATTTATGGGCCTTCCTATAAACTTCTGAGAGGGTAAC | |
| | TTTATCCTGCTTCTTTCAGCCAAGTATCCTCCTCCAGCAGCTG | |
| | GTCACAAAGCTGGTTAATCTCCCAGAGTGCTCAGCTTAAAAC | |
| | CCGTGACTCACAGCACAGCCAGTGTGGGGGAGGGGGTGGCT | |
| | GCCTCCAATACGTGGCGCCCAGAGTCAGCTGTTCTGGGGCCT | |
| | TCTCTGGTTTCTCCAACTGAGTCCTGAGGTTTGGGGCCTTGTC | |
| | TTCCTTCCTGGAGTTTCTCCTCTATAAATACCCGCTCTGGTATT | |
| | TGGGGTTGGCAGCTGTTGCTGCCAGGGAGATGGTTGGGTTGA | |
| | CGGGATCTTGCAGCTGTCAGGGGAGGGGAGGCGGGGGCTGA | |
| | TGTCAGGAGGGATACAAATAGTGCCGACGGCTGGGGGCCCTG | |
| | TCTCCCCTCGCCGCATCCACTCTCCGGCCGGCCGCCTGCCCGC | |
| | CGCCTCCTCCGTGCGCCCGCCAGCCTCGCCCGCGCCGTCACC | |
| | (SEQ ID NO: 108) | |
| SP0157 | CTAGACTAGCATGCTGCCCATGTAAGGAGGCAAGGCCTGGGG | 202 |
| | ACACCCGAGATGCCTGGTTATAATTAACCCAGACATGTGGCT | |
| | GCCCCCCCCCCCCCAACACCTGCTGCCTCTAAAAATAACCCT | |
| | GCATGCCCACCGCCTGCTGCCACGGCCGGCCGTATAAATAGA | |
| | GGCGAGGAGCAGCTGGGCTCTCTTGGCAGTCACC (SEQ ID | |
| | NO: 109) | |
| SP0158 | GGGCCCCACAGCAGCTGGGGGCATTTATGGGCCTTCCTATAA | 705 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCTGAGATTTTCCT | |
| | AGCATTTTGTGTTTCATGACTAAATATGGTTTGTGTTTCAAGA | |
| | CCAATGAGCTGGGAACTGTACTGTTCTTTCCCCTCCCATCAAC | |
| | TCATTTTTGGCACAAGACGCACTCTAGTCAGTTGGAGCAAAT | |
| | CCCCTGACCCGGGTGCAGTTCCAAAAGCAGACACTCGAGCGT | |
| | GTTTTACCTAATTAGGAAATGCTTTGCTCCAAACCGAACTGCT | |
| | CATTCAGGTTAGAGAGGAGAGGTCCCTATATGGTTGTGTTAG | |
| | AGTGAACGGCCAGCTTCAGCCCGTCTTTGCTCCTTGTTTGGGA | |
| | AGCGAGTGGGAGGGGATCAGAGCAAGGGGCTATATAACCCT | |
| | TCAGCGTTCAGCCTCCCGGGACACCACCCACCCAGAGTGGAG | |
| | AAGCCCAGCCAGTCGCTGTCAGCCACC (SEQ ID NO: 110) | |
| SP0159 | GCCACTACGGGTCTAGGCTGCCCATGTAAGGAGGCAAGGCCT | 615 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCTGAGATTTTCCTAGCA | |
| | TTTTGTGTTTCATGACTAAATATGGTTTGTGTTTCAAGACCAA | |
| | TGAGCTGGGAACTGTACTGTTCTTTCCCCTCCCATCAACTCAT | |
| | TTTTGGCACAAGACGCACTCTAGTCAGTTGGAGCAAATCCCC | |
| | TGACCCGGGTGCAGTTCCAAAAGCAGACACTCGAGCGTGTTT | |
| | TACCTAATTAGGAAATGCTTTGCTCCAAACCGAACTGCTCATT | |
| | CAGGTTAGAGAGGAGAGGTCCCTATATGGTTGTGTTAGAGTG | |
| | AACGGCCAGCTTCAGCCCGTCTTTGCTCCTTGTTTGGGAAGCG | |
| | AGTGGGAGGGGATCAGAGCAAGGGGCTATATAACCCTTCAG | |
| | CGTTCAGCCTCCCGGGACACCACCCACCCAGAGTGGAGAAGC | |
| | CCAGCCAGTCGCTGTCAGCCACC (SEQ ID NO: 111) | |
| SP0160 | GCCACTACGGGTCTAGGCTGCCCATGTAAGGAGGCAAGGCCT | 586 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTAAGTCCGGGCAGGGTC | |
| | CTGTCCATAAAAGGCTTTTCCCGGGCCGGCTCCCCGCCGGCA | |
| | GCGTGCCCCGCCCCGGCCCGCTCCATCTCCAAAGCATGCAGA | |
| | GAATGTCTCGGCAGCCCCGGTAGACTGCTCCAACTTGGTGTC | |
| | TTTCCCCAAATATGGAGCCTGTGTGGAGTCACTGGGGGAGCC | |
| | GGGGGTGGGGAGCGGAGCCGGCTTCCTCTAGAGGTCCCTATA | |
| | TGGTTGTGTTAGAGTGAACGGCCAGCTTCAGCCCGTCTTTGCT | |
| | CCTTGTTTGGGAAGCGAGTGGGAGGGGATCAGAGCAAGGGG | |
| | CTATATAACCCTTCAGCGTTCAGCCTCCCGGGACACCACCCA | |
| | CCCAGAGTGGAGAAGCCCAGCCAGTCGCTGTCAGCCACC | |
| | (SEQ ID NO: 112) | |
| SP0161 | GGGCCCCACAGCAGCTGGGGGCATTTATGGGCCTTCCTATAA | 740 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCTGAGATTTTCCT | |
| | AGCATTTTGTGTTTCATGACTAAATATGGTTTGTGTTTCAAGA | |
| | CCAATGAGCTGGGAACTGTACTGTTCTTTCCCCTCCCATCAAC | |
| | TCATTTTTGGCACAAGACGCACTCTAGTCAGTIGGAGCAAAT | |
| | CCCCTGACCCGGGTGCAGTTCCAAAAGCAGACACTCGAGCGT | |
| | GTTTTACCTAATTAGGAAATGCTTTGCTCCAAACCGAACTGCT | |
| | CATTCAGGTTAGAGAGGAGCTGAGTCCTTTTGCATACATTTTT | |
| | CAAATGATAACTCACTCTACCCACCCCCCTTCCCTACCCCCAA | |
| | GGCGATTTATTGAAAAAACCACCTTATATGGTAATATTGCTA | |
| | ACACACCGTCAGCTGGCCTTTTTAGGGACTTTGTTTAAAGAA | |
| | GATCCGCCTCTGGGGTTTTATATTGCTCTGGTATTCATGCCAA | |
| | AGACACACCAGGCCACC (SEQ ID NO: 113) | |
| SP0162 | GCCACTACGGGTCTAGGCTGCCCATGTAAGGAGGCAAGGCCT | 650 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCTGAGATTTTCCTAGCA | |
| | TTTTGTGTTTCATGACTAAATATGGTTTGTGTTTCAAGACCAA | |
| | TGAGCTGGGAACTGTACTGTTCTTTCCCCTCCCATCAACTCAT | |
| | TTTTGGCACAAGACGCACTCTAGTCAGTTGGAGCAAATCCCC | |
| | TGACCCGGGTGCAGTTCCAAAAGCAGACACTCGAGCGTGTTT | |
| | TACCTAATTAGGAAATGCTTTGCTCCAAACCGAACTGCTCATT | |
| | CAGGTTAGAGAGGAGCTGAGTCCTTTTGCATACATTTTTCAA | |
| | ATGATAACTCACTCTACCCACCCCCCTTCCCTACCCCCAAGGC | |
| | GATTTATTGAAAAAACCACCTTATATGGTAATATTGCTAACA | |
| | CACCGTCAGCTGGCCTTTTTAGGGACTTTGTTTAAAGAAGATC | |
| | CGCCTCTGGGGTTTTATATTGCTCTGGTATTCATGCCAAAGAC | |
| | ACACCAGGCCACC (SEQ ID NO: 114) | |
| SP0163 | GCCACTACGGGTCTAGGCTGCCCATGTAAGGAGGCAAGGCCT | 621 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTAAGTCCGGGCAGGGTC | |
| | CTGTCCATAAAAGGCTTTTCCCGGGCCGGCTCCCCGCCGGCA | |
| | GCGTGCCCCGCCCCGGCCCGCTCCATCTCCAAAGCATGCAGA | |
| | GAATGTCTCGGCAGCCCCGGTAGACTGCTCCAACTTGGTGTC | |
| | TTTCCCCAAATATGGAGCCTGTGTGGAGTCACTGGGGGAGCC | |
| | GGGGGTGGGGAGCGGAGCCGGCTTCCTCTAGCTGAGTCCTTT | |
| | TGCATACATTTTTCAAATGATAACTCACTCTACCCACCCCCCT | |
| | TCCCTACCCCCAAGGCGATTTATTGAAAAAACCACCTTATAT | |
| | GGTAATATTGCTAACACACCGTCAGCTGGCCTTTTTAGGGACT | |
| | TTGTTTAAAGAAGATCCGCCTCTGGGGTTTTATATTGCTCTGG | |
| | TATTCATGCCAAAGACACACCAGGCCACC (SEQ ID NO: 115) | |
| SP0164 | CCCACCCATGCCTCCTCAGGTACCCCCTGCCCCCCACAGCTCC | 764 |
| | TCTCCTGTGCCTTGTTTCCCAGCCATGCGTTCTCCTCTATAAAT | |
| | ACCCGCTCTGGTATTTGGGGTTGGCAGCTGTTGCTGCCAGGG | |
| | AGATGGTTGGGTTGACATGCGGCTCCTGACAAAACACAAACC | |
| | CCTGGTGTGTGTGGGCGTGGGTGGTGTGAGTAGGGGGATGAA | |
| | TCAGGGAGGGGGCGGGGGGGGCCCCACAGCAGCTGGGGGCA | |
| | TTTATGGGCCTTCCTATAAACTTCTGAGAGGGTAACTTTATCC | |
| | TGCTTCTTTCAGCCAAGTATCCTCCTCCAGCAGCTGGTCACAA | |
| | AGCTGGTTAATCTCCCAGAGTGCTCAGCTTAAAACCCGTGAC | |
| | TCACAGCACAGCCAGTGTGGGGGAGGGGGTGGCTGCCTCCAA | |
| | TACGTGGCGCCCAGAGTCAGCTGTTCTGGGGCCTTCTCTGGTT | |
| | TCTCCAACTGAGTCCTGAGGTTTGGGGCCTTGTCTTCCTTCCT | |
| | GGAGTGACTCAGGGGCGCAGGCCTCTTGCGGGGGAGCTGGCC | |
| | TCCCCGCCCCCACGGCCACGGGCCGCCCTTTCCTGGCAGGAC | |
| | AGCGGGATCTTGCAGCTGTCAGGGGAGGGGAGGCGGGGGCT | |
| | GATGTCAGGAGGGATACAAATAGTGCCGACGGCTGGGGGCC | |
| | CTGTCTCCCCTCGCCGCATCCACTCTCCGGCCGGCCGCCTGCC | |
| | CGCCGCCTCCTCCGTGCGCCCGCCAGCCTCGCCCGCGCCGTC | |
| | ACC (SEQ ID NO: 116) | |
| SP0165 | CCCACCCATGCCTCCTCAGGTACCCCCTGCCCCCCACAGCTCC | 480 |
| | TCTCCTGTGCCTTGTTTCCCAGCCATGCGTTCTCCTCTATAAAT | |
| | ACCCGCTCTGGTATTTGGGGTTGGCAGCTGTTGCTGCCAGGG | |
| | AGATGGTTGGGTTGACATGCGGCTCCTGACAAAACACAAACC | |
| | CCTGGTGTGTGTGGGCGTGGGTGGTGTGAGTAGGGGGATGAA | |
| | TCAGGGAGGGGGCGGGGGGACTCAGGGGCGCAGGCCTCTTG | |
| | CGGGGGAGCTGGCCTCCCCGCCCCCACGGCCACGGGCCGCCC | |
| | TTTCCTGGCAGGACAGCGGGATCTTGCAGCTGTCAGGGGAGG | |
| | GGAGGCGGGGGCTGATGTCAGGAGGGATACAAATAGTGCCG | |
| | ACGGCTGGGGGCCCTGTCTCCCCTCGCCGCATCCACTCTCCGG | |
| | CCGGCCGCCTGCCCGCCGCCTCCTCCGTGCGCCCGCCAGCCTC | |
| | GCCCGCGCCGTCACC (SEQ ID NO: 117) | |
| SP0166 | CACCGCGGTGGCGGCCGTCCGCCCTCGGCACCATCCTCACGA | 894 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCGGCCGGGGCCCAATTCTCATGT | |
| | TTGACAGCTTATCATCGCAGATCCGTATGGTGCACTCTCAGTA | |
| | CAATCTGCTCTGATGCCGCATAGTTAAGCCAGTATCTGCTCCC | |
| | TGCTTGTGTGTTGGAGGTCGCTGAGTAGTGCGCGAGCAAAAT | |
| | TTAAGCTACAACAAGGCAAGGCTTGACCGACAATTGCATGAA | |
| | GAATCTGCTTAGGGTTAGGCGTTTTGCGCTGCTTCGCGATGTA | |
| | CGGGCCAGATATACGCGTATCTGAGGGGACTAGGGTGTGTTT | |
| | AGGCGAAAAGCGGGGCTTCGGTTGTACGCGGTTAGGAGTCCC | |
| | CTCAGGATATAGTAGTTTCGCTTTTGCATAGGGAGGGGGAAA | |
| | TGTAGTCTTATGCAATACTCTTGTAGTCTTGCAACATGGTAAC | |
| | GATGAGTTAGCAACATGCCTTACAAGGAGAGAAAAAGCACC | |
| | GTGCATGCCGATTGGTGGAAGTAAGGTGGTACGATCGTGCCT | |
| | TATTAGGAAGGCAACAGACGGGTCTGACATGGATTGGACGA | |
| | ACCACTGAATTCCGCATTGCAGAGATATTGTATTTAAGTGCCT | |
| | AGCTCGATACAATAAACGCCATTTGACCATTCACCACATTGG | |
| | TGTGCACCTCCAAGCTGGGTACCGCGGGCCCGGGATCCACCG | |
| | GTCGCCACC (SEQ ID NO: 118) | |
| SP0169 | ATAAATACCCGCTCTGGTATTTGGGGTTCTCCTCTATAAATAC | 248 |
| | CCGCTCTGGTATTTGGGGTTGGCAGCTGTTGCGGGATCTTGCA | |
| | GCTGTCAGGGGAGGGGAGGCGGGGGCTGATGTCAGGAGGGA | |
| | TACAAATAGTGCCGACGGCTGGGGGCCCTGTCTCCCCTCGCC | |
| | GCATCCACTCTCCGGCCGGCCGCCTGCCCGCCGCCTCCTCCGT | |
| | GCGCCCGCCAGCCTCGCCCGCGCCGTCACCGCCACC (SEQ ID | |
| | NO: 119) | |
| SP0170 | CACCGCGGTGGCGGCCGTCCGCCCTCGGCACCATCCTCACGA | 482 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCGGCCGGGGCCATAAATACCCG | |
| | CTCTGGTATTTGGGGTTCTCCTCTATAAATACCCGCTCTGGTA | |
| | TTTGGGGTTGGCAGCTGTTGCGGGATCTTGCAGCTGTCAGGG | |
| | GAGGGGAGGCGGGGGCTGATGTCAGGAGGGATACAAATAGT | |
| | GCCGACGGCTGGGGGCCCTGTCTCCCCTCGCCGCATCCACTCT | |
| | CCGGCCGGCCGCCTGCCCGCCGCCTCCTCCGTGCGCCCGCCA | |
| | GCCTCGCCCGCGCCGTCACC (SEQ ID NO: 120) | |
| SP0171 | GTTTCTTAGCAGCTGCTGCTGTGTCCAAGGCTTGGAATTGCTG | 534 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGATAAA | |
| | TACCCGCTCTGGTATTTGGGGTTCTCCTCTATAAATACCCGCT | |
| | CTGGTATTTGGGGTTGGCAGCTGTTGCGGGATCTTGCAGCTGT | |
| | CAGGGGAGGGGAGGCGGGGGCTGATGTCAGGAGGGATACAA | |
| | ATAGTGCCGACGGCTGGGGGCCCTGTCTCCCCTCGCCGCATC | |
| | CACTCTCCGGCCGGCCGCCTGCCCGCCGCCTCCTCCGTGCGCC | |
| | CGCCAGCCTCGCCCGCGCCGTCACC (SEQ ID NO: 121) | |
| SP0173 | GTTTCTTAGCAGCTGCTGCTGTGTCCAAGGCTTGGAATTGCTG | 728 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | TACGGGTCTAGGCTGCCCATGTAAGGAGGCAAGGCCTGGGGA | |
| | CACCCGAGATGCCTGGTTATAATTAACCCAGACATGTGGCTG | |
| | CCCCCCCCCCCCAACACCTGCTGCCTGAGCCTCACCCCCACCC | |
| | CGGTGCCTGGGTCTTAGGCTCTGTACACCATGGAGGAGAAGC | |
| | TCGCTCTAAAAATAACCCTGATAAATACCCGCTCTGGTATTTG | |
| | GGGTTCTCCTCTATAAATACCCGCTCTGGTATTTGGGGTTGGC | |
| | AGCTGTTGCGGGATCTTGCAGCTGTCAGGGGAGGGGAGGCGG | |
| | GGGCTGATGTCAGGAGGGATACAAATAGTGCCGACGGCTGG | |
| | GGGCCCTGTCTCCCCTCGCCGCATCCACTCTCCGGCCGGCCGC | |
| | CTGCCCGCCGCCTCCTCCGTGCGCCCGCCAGCCTCGCCCGCGC | |
| | CGTCACC (SEQ ID NO: 122) | |
| SP0228 | GGGCCCCACAGCAGCTGGGGGCATTTATGGGCCTTCCTATAA | 885 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCTCTGTCTCCTCA | |
| | GGTGCCTGGCTCCCAGTCCCCAGAACGCCTCTCCTGTACCTTG | |
| | CTTCCTAGCTGGGCCTTTCCTTCTCCTCTATAAATACCAGCTC | |
| | TGGTATTTCGCCTTGGCAGCTGTTGCTGCTAGGGAGACGGCT | |
| | GGCTTGACATGCATCTCCTGACAAAACACAAACCCGTGGTGT | |
| | GAGTGGGTGTGGGCGGTGTGAGTAGGGGGATGAATCAGAGA | |
| | GGGGGCCACCGCGGTGGCGGCCGTCCGCCCTCGGCACCATCC | |
| | TCACGACACCCAAATATGGCGACGGGTGAGGAATGGTGGGG | |
| | AGTTATTTTTAGAGCGGTGAGGAAGGTGGGCAGGCAGCAGGT | |
| | GTTGGCGCTCTAAAAATAACTCCCGGGAGTTATTTTTAGAGC | |
| | GGAGGAATGGTGGACACCCAAATATGGCGACGGTTCCTCACC | |
| | CGTCGCCATATTTGGGTGTCCGCCCTCGGCCGGGGCCGCATTC | |
| | CTGGGGGCCGGGCGGTGCTCCCGCCCGCCTCGATAAAAGGCT | |
| | CCGGGGCCGGCGGCGGCCCACGAGCTACCCGGAGGAGCGGG | |
| | AGGCGCCAAGCTCTAGAACTAGTGGATCCCGCGGCCGCCACC | |
| | (SEQ ID NO: 123) | |
| SP0229 | GGGCCCCACAGCAGCTGGGGGCATTTATGGGCCTTCCTATAA | 1003 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCTCTGTCTCCTCA | |
| | GGTGCCTGGCTCCCAGTCCCCAGAACGCCTCTCCTGTACCTTG | |
| | CTTCCTAGCTGGGCCTTTCCTTCTCCTCTATAAATACCAGCTC | |
| | TGGTATTTCGCCTTGGCAGCTGTTGCTGCTAGGGAGACGGCT | |
| | GGCTTGACATGCATCTCCTGACAAAACACAAACCCGTGGTGT | |
| | GAGTGGGTGTGGGCGGTGTGAGTAGGGGGATGAATCAGAGA | |
| | GGGGGCCACCGCGGTGGCGGCCGTCCGCCCTCGGCACCATCC | |
| | TCACGACACCCAAATATGGCGACGGGTGAGGAATGGTGGGG | |
| | AGTTATTTTTAGAGCGGTGAGGAAGGTGGGCAGGCAGCAGGT | |
| | GTTGGCGCTCTAAAAATAACTCCCGGGAGTTATTTTTAGAGC | |
| | GGAGGAATGGTGGACACCCAAATATGGCGACGGTTCCTCACC | |
| | CGTCGCCATATTTGGGTGTCCGCCCTCGGCCGATAAATACCC | |
| | GCTCTGGTATTTGGGGTTCTCCTCTATAAATACCCGCTCTGGT | |
| | ATTTGGGGTTGGCAGCTGTTGCGGGATCTTGCAGCTGTCAGG | |
| | GGAGGGGAGGCGGGGGCTGATGTCAGGAGGGATACAAATAG | |
| | TGCCGACGGCTGGGGGCCCTGTCTCCCCTCGCCGCATCCACTC | |
| | TCCGGCCGGCCGCCTGCCCGCCGCCTCCTCCGTGCGCCCGCC | |
| | AGCCTCGCCCGCGCCGTCACCGCGGCCGCCACC (SEQ ID NO: | |
| | 124) | |
| SP0230 | GGGCCCCACAGCAGCTGGGGGCATTTATGGGCCTTCCTATAA | 953 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTATCAAGCTTGGTA | |
| | CGGGCCCCACAGCAGCTGGGGGCATTTATGGGCCTTCCTATA | |
| | AACTTCTGAGAGGGTAACTTTATCCTGCTTCTTTCAGCCAAGT | |
| | ATCCTCCTCCAGCAGCTGGTCACAAAGCTGGTTAATCTCCCA | |
| | GAGTGCTCAGCTTAAAACCCGTGACTCACAGCACAGCCAGTG | |
| | TGGGGGAGGGGGTGGCTGCCTCCAATACGTGGCGCCCAGAGT | |
| | CAGCTGTTCTGGGGCCTTCTCTGGTTTCTCCAACTGAGTCCTG | |
| | AGGTTTGGGGCCTTGTCTTCCTTCCTGGAGTCACCGCGGTGGC | |
| | GGCCGTCCGCCCTCGGCACCATCCTCACGACACCCAAATATG | |
| | GCGACGGGTGAGGAATGGTGGGGAGTTATTTTTAGAGCGGTG | |
| | AGGAAGGTGGGCAGGCAGCAGGTGTTGGCGCTCTAAAAATA | |
| | ACTCCCGGGAGTTATTTTTAGAGCGGAGGAATGGTGGACACC | |
| | CAAATATGGCGACGGTTCCTCACCCGTCGCCATATTTGGGTGT | |
| | CCGCCCTCGGCCGGGGCCGCATTCCTGGGGGCCGGGCGGTGC | |
| | TCCCGCCCGCCTCGATAAAAGGCTCCGGGGCCGGCGGCGGCC | |
| | CACGAGCTACCCGGAGGAGCGGGAGGCGCCAAGCTCTAGAA | |
| | CTAGTGGATCCCGCGGCCGCCACC (SEQ ID NO: 125) | |
| SP0231 | GGGCCCCACAGCAGCTGGGGGCATTTATGGGCCTTCCTATAA | 773 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCGGCCGATAAATACCCGCTCTGGTATTTGGGGTTCTC | |
| | CTCTATAAATACCCGCTCTGGTATTTGGGGTTGGCAGCTGTTG | |
| | CGGGATCTTGCAGCTGTCAGGGGAGGGGAGGCGGGGGCTGA | |
| | TGTCAGGAGGGATACAAATAGTGCCGACGGCTGGGGGCCCTG | |
| | TCTCCCCTCGCCGCATCCACTCTCCGGCCGGCCGCCTGCCCGC | |
| | CGCCTCCTCCGTGCGCCCGCCAGCCTCGCCCGCGCCGTCACC | |
| | GCGGCCGCCACC (SEQ ID NO: 126) | |
| SP0232 | GCCACTACGGGTCTAGGCTGCCCATGTAAGGAGGCAAGGCCT | 683 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCACCGCGGTGGCGGCCG | |
| | TCCGCCCTCGGCACCATCCTCACGACACCCAAATATGGCGAC | |
| | GGGTGAGGAATGGTGGGGAGTTATTTTTAGAGCGGTGAGGAA | |
| | GGTGGGCAGGCAGCAGGTGTTGGCGCTCTAAAAATAACTCCC | |
| | GGGAGTTATTTTTAGAGCGGAGGAATGGTGGACACCCAAATA | |
| | TGGCGACGGTTCCTCACCCGTCGCCATATTTGGGTGTCCGCCC | |
| | TCGGCCGATAAATACCCGCTCTGGTATTTGGGGTTCTCCTCTA | |
| | TAAATACCCGCTCTGGTATTTGGGGTTGGCAGCTGTTGCGGG | |
| | ATCTTGCAGCTGTCAGGGGAGGGGAGGCGGGGGCTGATGTCA | |
| | GGAGGGATACAAATAGTGCCGACGGCTGGGGGCCCTGTCTCC | |
| | CCTCGCCGCATCCACTCTCCGGCCGGCCGCCTGCCCGCCGCCT | |
| | CCTCCGTGCGCCCGCCAGCCTCGCCCGCGCCGTCACCGCGGC | |
| | CGCCACC (SEQ ID NO: 127) | |
| SP0257 | GTTTCTTAGCAGCTGCTGCTGTGTCCAAGGCTTGGAATTGCTG | 710 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | TACGGGTCTAGGCTGCCCATGTAAGGAGGCAAGGCCTGGGGA | |
| | CACCCGAGATGCCTGGTTATAATTAACCCAGACATGTGGCTG | |
| | CCCCCCCCCCCCAACACCTGCTGCCTGAGCCTCACCCCCACCC | |
| | CGGTGCCTGGGTCTTAGGCTCTGTACACCATGGAGGAGAAGC | |
| | TCGCTCTAAAAATAACCCTGCCCGGCAGACGCTCCTTATACG | |
| | GCCCGGCCTCGCTCACCTGGGCCGCGGCCAGGAGCGCCTTCT | |
| | TTGGGCAGCGCCGGGCCGGGGCCGCGCCGGGCCCGACACCC | |
| | AAATATGGCGACGGCCGGGGCCGCATTCCTGGGGGCCGGGC | |
| | GGCGCTCCCGCCCGCCTCGATAAAAGGCTCCGGGGCCGGCGG | |
| | CGGCCCACGAGCTACCCGGAGGAGCGGGAGGCCACC (SEQ ID | |
| | NO: 128) | |
| SP0262 | GTTTCTTAGCAGCTGCTGCTGTGTCCAAGGCTTGGAATTGCTG | 943 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | TACGGGTCTAGGCTGCCCATGTAAGGAGGCAAGGCCTGGGGA | |
| | CACCCGAGATGCCTGGTTATAATTAACCCAGACATGTGGCTG | |
| | CCCCCCCCCCCCAACACCTGCTGCCTGAGCCTCACCCCCACCC | |
| | CGGTGCCTGGGTCTTAGGCTCTGTACACCATGGAGGAGAAGC | |
| | TCGCTCTAAAAATAACCCTGCCAGCTGCCTGCCCCCTGCCTGG | |
| | CACAGCCCGTACCTGGCCGCACGCTCCCTCACAGGTGAAGCT | |
| | CGAAAACTCCGTCCCCGTAAGGAGCCCCGCTGCCCCCCGAGG | |
| | CCTCCTCCCTCACGCCTCGCTGCGCTCCCGGCTCCCGCACGGC | |
| | CCTGGGAGAGGCCCCCACCGCTTCGTCCTTAACGGGCCCGGC | |
| | GGTGCCGGGGGATTATTTCGGCCCCGGCCCCGGGGGGGCCCG | |
| | GCAGACGCTCCTTATACGGCCCGGCCTCGCTCACCTGGGCCG | |
| | CGGCCAGGAGCGCCTTCTTTGGGCAGCGCCGGGCCGGGGCCG | |
| | CGCCGGGCCCGACACCCAAATATGGCGACGGCCGGGGCCGC | |
| | ATTCCTGGGGGCCGGGCGGCGCTCCCGCCCGCCTCGATAAAA | |
| | GGCTCCGGGGCCGGCGGCGGCCCACGAGCTACCCGGAGGAG | |
| | CGGGAGGCGGCCACC (SEQ ID NO: 129) | |
| SP0264 | GCCACTACGGGTCTAGGCTGCCCATGTAAGGAGGCAAGGCCT | 724 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGGCCGCGAAGACCGGAA | |
| | GCTGGGGCGGCCCCGGGCCGCGCGCGCTGGGCCTGGGAGGC | |
| | GAAACTCAGCTTCCTTCGTTTCCGACTTTTCCATCCGCGTCCT | |
| | CCACTTCCCCGTTCCGCCCTCCCCCATTGCCAACATTCTGGCT | |
| | GAGTCACGGCGCCCCAGAGCGCGCCAGGCTGGGGGAAAGGA | |
| | GCAGAAGGGAGGGCCCTAGCGACCCGCGGGATGTGGTCCGA | |
| | GTCACGTCCGAGGGGGGTGGGGAGGGATCGTGTTCTCGGCGC | |
| | CCGCCCCTTCCTAGCGCGGCCTCTGGGCTGCGCCTCTCGGGG | |
| | GCGGCCCGTAGCCCAGTCCGTCGCCTGCCATTGGACGCCGCC | |
| | CGCTCCTCGTAAAGGAAAAAGCTCGGCGGAGGGCGGAGTGG | |
| | TGCCTTTAAAAGGCCGGGCGCCGCCTTCCGCCTGCCCGCCTCC | |
| | TGCGCCGCCCCTTCCGAGGCTAAATCGGCTGCGTTCCTCTCGG | |
| | AACGCGCCGCAGAAGGGGTCCTGGTGACGAGTCCCGCGTTCT | |
| | CTCCGCCACC (SEQ ID NO: 130) | |
| SP0265 | GTTTCTTAGCAGCTGCTGCTGTGTCCAAGGCTTGGAATTGCTG | 822 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCGC | |
| | GAAGACCGGAAGCTGGGGCGGCCCCGGGCCGCGCGCGCTGG | |
| | GCCTGGGAGGCGAAACTCAGCTTCCTTCGTTTCCGACTTTTCC | |
| | ATCCGCGTCCTCCACTTCCCCGTTCCGCCCTCCCCCATTGCCA | |
| | ACATTCTGGCTGAGTCACGGCGCCCCAGAGCGCGCCAGGCTG | |
| | GGGGAAAGGAGCAGAAGGGAGGGCCCTAGCGACCCGCGGGA | |
| | TGTGGTCCGAGTCACGTCCGAGGGGGGTGGGGAGGGATCGTG | |
| | TTCTCGGCGCCCGCCCCTTCCTAGCGCGGCCTCTGGGCTGCGC | |
| | CTCTCGGGGGCGGCCCGTAGCCCAGTCCGTCGCCTGCCATTG | |
| | GACGCCGCCCGCTCCTCGTAAAGGAAAAAGCTCGGCGGAGG | |
| | GCGGAGTGGTGCCTTTAAAAGGCCGGGCGCCGCCTTCCGCCT | |
| | GCCCGCCTCCTGCGCCGCCCCTTCCGAGGCTAAATCGGCTGC | |
| | GTTCCTCTCGGAACGCGCCGCAGAAGGGGTCCTGGTGACGAG | |
| | TCCCGCGTTCTCTCCGCCACC (SEQ ID NO: 131) | |
| SP0266 | GTTTCTTAGCAGCTGCTGCTGTGTCCAAGGCTTGGAATTGCTG | 1016 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | TACGGGTCTAGGCTGCCCATGTAAGGAGGCAAGGCCTGGGGA | |
| | CACCCGAGATGCCTGGTTATAATTAACCCAGACATGTGGCTG | |
| | CCCCCCCCCCCCAACACCTGCTGCCTGAGCCTCACCCCCACCC | |
| | CGGTGCCTGGGTCTTAGGCTCTGTACACCATGGAGGAGAAGC | |
| | TCGCTCTAAAAATAACCCTGGCCGCGAAGACCGGAAGCTGGG | |
| | GCGGCCCCGGGCCGCGCGCGCTGGGCCTGGGAGGCGAAACT | |
| | CAGCTTCCTTCGTTTCCGACTTTTCCATCCGCGTCCTCCACTTC | |
| | CCCGTTCCGCCCTCCCCCATTGCCAACATTCTGGCTGAGTCAC | |
| | GGCGCCCCAGAGCGCGCCAGGCTGGGGGAAAGGAGCAGAAG | |
| | GGAGGGCCCTAGCGACCCGCGGGATGTGGTCCGAGTCACGTC | |
| | CGAGGGGGGTGGGGAGGGATCGTGTTCTCGGCGCCCGCCCCT | |
| | TCCTAGCGCGGCCTCTGGGCTGCGCCTCTCGGGGGCGGCCCG | |
| | TAGCCCAGTCCGTCGCCTGCCATTGGACGCCGCCCGCTCCTCG | |
| | TAAAGGAAAAAGCTCGGCGGAGGGCGGAGTGGTGCCTTTAA | |
| | AAGGCCGGGCGCCGCCTTCCGCCTGCCCGCCTCCTGCGCCGC | |
| | CCCTTCCGAGGCTAAATCGGCTGCGTTCCTCTCGGAACGCGC | |
| | CGCAGAAGGGGTCCTGGTGACGAGTCCCGCGTTCTCTCCGCC | |
| | ACC (SEQ ID NO: 132) | |
| SP0267 | CCCTTCAGATTAAAAATAACTGAGGTAAGGGCCTGGGTAGGG | 560 |
| | GAGGTGGTGTGAGACGCTCCTGTCTCTCCTCTATCTGCCCATC | |
| | GGCCCTTTGGGGAGGAGGAATGTGCCCAAGGACTAAAAAAA | |
| | GGCCATGGAGCCAGAGGGGCGAGGGCAACAGACCTTTCATG | |
| | GGCAAACCTTGGGGCCCTGCTGCACCGCGGTGGCGGCCGTCC | |
| | GCCCTCGGCACCATCCTCACGACACCCAAATATGGCGACGGG | |
| | TGAGGAATGGTGGGGAGTTATTTTTAGAGCGGTGAGGAAGGT | |
| | GGGCAGGCAGCAGGTGTTGGCGCTCTAAAAATAACTCCCGGG | |
| | AGTTATTTTTAGAGCGGAGGAATGGTGGACACCCAAATATGG | |
| | CGACGGTTCCTCACCCGTCGCCATATTTGGGTGTCCGCCCTCG | |
| | GCCGGGGCCGCATTCCTGGGGGCCGGGCGGTGCTCCCGCCCG | |
| | CCTCGATAAAAGGCTCCGGGGCCGGCGGCGGCCCACGAGCTA | |
| | CCCGGAGGAGCGGGAGGCGCCAAGCTCTAGAACTAGTGGAT | |
| | CCCGCGGCCGCCACC (SEQ ID NO: 133) | |
| SP0268 | GCCACTACGGGTCTAGGCTGCCCATGTAAGGAGGCAAGGCCT | 728 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGGTTTCTTAGCAGCTGCT | |
| | GCTGTGTCCAAGGCTTGGAATTGCTGTGGTGAATCTAAAACT | |
| | GTCTCAGTAGTGGTGAGCTGACCTCACCCAAGTTCAAAGCCC | |
| | TACTCTGCCTGATCCTTTTTTCCTGAGCCTCAGAGCTAAAATG | |
| | CCCCCGAGCTCTTTCCTATTGGCTGGAAAGACGAATTGAAGT | |
| | TCCCTTGCCCATGTTAGGAGGTGTACGCCTCCTGAACTAAAG | |
| | ATAGAAACAGCTGGCCCTTCCAGGCAGCTAAAAGCCTCCAGA | |
| | CTAAGAGGTGTTCCCCATTCGGATAAATACCCGCTCTGGTATT | |
| | TGGGGTTCTCCTCTATAAATACCCGCTCTGGTATTTGGGGTTG | |
| | GCAGCTGTTGCGGGATCTTGCAGCTGTCAGGGGAGGGGAGGC | |
| | GGGGGCTGATGTCAGGAGGGATACAAATAGTGCCGACGGCT | |
| | GGGGGCCCTGTCTCCCCTCGCCGCATCCACTCTCCGGCCGGCC | |
| | GCCTGCCCGCCGCCTCCTCCGTGCGCCCGCCAGCCTCGCCCGC | |
| | GCCGTCACC (SEQ ID NO: 134) | |
| SP0270 | GCCACTACGGGTCTAGGCTGCCCATGTAAGGAGGCAAGGCCT | 562 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTCAAAGCCCTACTCTGC | |
| | CTGATCCTTTTTTCCTGAGCCTCAGAGCTAAAATGCCCCCGAG | |
| | CTCTTTCCTATTGGCTGGAAAGACGAATTGAAGTTCCCTTGCC | |
| | CATGTTAGGAGGTGTACGCCTCCTGAACTAAAGATAGAAACA | |
| | GCTGGCCCTTCCAGGCAGCTAAAAGCCTCCAGACTAAGAGGT | |
| | GTTCCCCATTCGGCGGGATCTTGCAGCTGTCAGGGGAGGGGA | |
| | GGCGGGGGCTGATGTCAGGAGGGATACAAATAGTGCCGACG | |
| | GCTGGGGGCCCTGTCTCCCCTCGCCGCATCCACTCTCCGGCCG | |
| | GCCGCCTGCCCGCCGCCTCCTCCGTGCGCCCGCCAGCCTCGCC | |
| | CGCGCCGTCACC (SEQ ID NO: 135) | |
| SP0271 | GCCACTACGGGTCTAGGCTGCCCATGTAAGGAGGCAAGGCCT | 451 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTCAAAGCCCTACTCTGC | |
| | CTGATCCTTTTTTCCTGAGCCTCAGAGCTAAAATGCCCCCGAG | |
| | CTCTTTCCTATTGGCTGGAAAGACGAATTGAAGTTCCCTTGCC | |
| | CATGTTAGGAGGTGTACGCCTCCTGAACTAAAGATAGAAACA | |
| | GCTGGCCCTTCCAGGCAGCTAAAAGCCTCCAGACTAAGAGGT | |
| | GTTCCCCATTCGGCAGCCAGACTCCTTGAAATACCCTTTCAGT | |
| | AATCATTCAACCAACGCTTCCGCCACC (SEQ ID NO: 136) | |
| SP0279 | GGGCCCCACAGCAGCTGGGGGCATTTATGGGCCTTCCTATAA | 883 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCGGCCGGGGCCGCATTCCTGGGGGCCGGGCGGTGCT | |
| | CCCGCCCGCCTCGATAAAAGGCTCCGGGGCCGGCGGCGGCCC | |
| | ACTCAGATCGCCTGGAGACGCCATCCACGCTGTTTTGACCTCC | |
| | ATAGAAGACACCGGGACCGATCCAGCCTCCGCGGCCGGGAA | |
| | CGGTGCATTGGAACGCGGATTCCCCGTGCCAAGAGTGACGTA | |
| | AGTACCGCCTATAGACTCTATAGGCACACCCCTTTGGCTCTTA | |
| | TGCATGAACGGTGGAGGGCAGTGTAGTCTGAGCAGTACTCGT | |
| | TGCTGCCGCGCGCGCCACCAGACATAATAGCTGACAGACTAA | |
| | CAGACTGTTCCTTTCCATGGGTCTTTTCTGCAGGCCACC (SEQ | |
| | ID NO: 137) | |
| SP0286 | CACCGCGGTGGCGGCCGTCCGCCCTCGGCACCATCCTCACGA | 616 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCGGCCGGGGCCGCATTCCTGGG | |
| | GGCCGGGCGGTGCTCCCGCCCGCCTCGATAAAAGGCTCCGGG | |
| | GCCGGCGGCGGCCCACTCAGATCGCCTGGAGACGCCATCCAC | |
| | GCTGTTTTGACCTCCATAGAAGACACCGGGACCGATCCAGCC | |
| | TCCGCGGCCGGGAACGGTGCATTGGAACGCGGATTCCCCGTG | |
| | CCAAGAGTGACGTAAGTACCGCCTATAGACTCTATAGGCACA | |
| | CCCCTTTGGCTCTTATGCATGAACGGTGGAGGGCAGTGTAGT | |
| | CTGAGCAGTACTCGTTGCTGCCGCGCGCGCCACCAGACATAA | |
| | TAGCTGACAGACTAACAGACTGTTCCTTTCCATGGGTCTTTTC | |
| | TGCAGTCACCGTCCTTGACACGGCCACC (SEQ ID NO: 138) | |
| SP0305 | GTTTCTTAGCAGCTGCTGCTGTGTCCAAGGCTTGGAATTGCTG | 562 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | TACGGGTCTAGGCTGCCCATGTAAGGAGGCAAGGCCTGGGGA | |
| | CACCCGAGATGCCTGGTTATAATTAACCCAGACATGTGGCTG | |
| | CCCCCCCCCCCCAACACCTGCTGCCTGAGCCTCACCCCCACCC | |
| | CGGTGCCTGGGTCTTAGGCTCTGTACACCATGGAGGAGAAGC | |
| | TCGCTCTAAAAATAACCCTGCCACCGCCTGCTGCCACGGCCG | |
| | GCCGTATAAATAGAGGCGAGGAGCAGCTGGGCTCTCTTGGCA | |
| | GTCACCGCCACC (SEQ ID NO: 139) | |
| SP0306 | CTCTGTCTCCTCAGGTGCCTGGCTCCCAGTCCCCAGAACGCCT | 50 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCGCCACTACGGGTCTAGGCTGCCCATG | |
| | TAAGGAGGCAAGGCCTGGGGACACCCGAGATGCCTGGTTATA | |
| | ATTAACCCAGACATGTGGCTGCCCCCCCCCCCCAACACCTGC | |
| | TGCCTGAGCCTCACCCCCACCCCGGTGCCTGGGTCTTAGGCTC | |
| | TGTACACCATGGAGGAGAAGCTCGCTCTAAAAATAACCCTGC | |
| | CACCGCCTGCTGCCACGGCCGGCCGTATAAATAGAGGCGAGG | |
| | AGCAGCTGGGCTCTCTTGGCAGTCACCGCCACC (SEQ ID NO: | |
| | 140) | |
| SP0307 | GGGCCCCACAGCAGCTGGGGGCATTTATGGGCCTTCCTATAA | 554 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTGCCACTACGGGTC | |
| | TAGGCTGCCCATGTAAGGAGGCAAGGCCTGGGGACACCCGA | |
| | GATGCCTGGTTATAATTAACCCAGACATGTGGCTGCCCCCCC | |
| | CCCCCAACACCTGCTGCCTGAGCCTCACCCCCACCCCGGTGC | |
| | CTGGGTCTTAGGCTCTGTACACCATGGAGGAGAAGCTCGCTC | |
| | TAAAAATAACCCTGCCACCGCCTGCTGCCACGGCCGGCCGTA | |
| | TAAATAGAGGCGAGGAGCAGCTGGGCTCTCTTGGCAGTCACC | |
| | GCCACC (SEQ ID NO: 141) | |
| SP0309 | GCCACTACGGGTCTAGGCTGCCCATGTAAGGAGGCAAGGCCT | 636 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGGCCACTACGGGTCTAGG | |
| | CTGCCCATGTAAGGAGGCAAGGCCTGGGGACACCCGAGATG | |
| | CCTGGTTATAATTAACCCAGACATGTGGCTGCCCCCCCCCCCC | |
| | AACACCTGCTGCCTGAGCCTCACCCCCACCCCGGTGCCTGGG | |
| | TCTTAGGCTCTGTACACCATGGAGGAGAAGCTCGCTCTAAAA | |
| | ATAACCCTGATAAATACCCGCTCTGGTATTTGGGGTTCTCCTC | |
| | TATAAATACCCGCTCTGGTATTTGGGGTTGGCAGCTGTTGCGG | |
| | GATCTTGCAGCTGTCAGGGGAGGGGAGGCGGGGGCTGATGTC | |
| | AGGAGGGATACAAATAGTGCCGACGGCTGGGGGCCCTGTCTC | |
| | CCCTCGCCGCATCCACTCTCCGGCCGGCCGCCTGCCCGCCGCC | |
| | TCCTCCGTGCGCCCGCCAGCCTCGCCCGCGCCGTCACCGCCA | |
| | CC (SEQ ID NO: 142) | |
| SP0310 | GCCACTACGGGTCTAGGCTGCCCATGTAAGGAGGCAAGGCCT | 441 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGATAAATACCCGCTCTGG | |
| | TATTTGGGGTTCTCCTCTATAAATACCCGCTCTGGTATTTGGG | |
| | GTTGGCAGCTGTTGCGGGATCTTGCAGCTGTCAGGGGAGGGG | |
| | AGGCGGGGGCTGATGTCAGGAGGGATACAAATAGTGCCGAC | |
| | GGCTGGGGGCCCTGTCTCCCCTCGCCGCATCCACTCTCCGGCC | |
| | GGCCGCCTGCCCGCCGCCTCCTCCGTGCGCCCGCCAGCCTCG | |
| | CCCGCGCCGTCACCGCCACC (SEQ ID NO: 143) | |
| SP0311 | GCCACTACGGGTCTAGGCTGCCCATGTAAGGAGGCAAGGCCT | 318 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTTCTCCTCTATAAATACC | |
| | CGCTCTGGTATTTGGGGTTGGCAGCTGTTGCCACCGCCTGCTG | |
| | CCACGGCCGGCCGTATAAATAGAGGCGAGGAGCAGCTGGGC | |
| | TCTCTTGGCAGTCACCGCCACC (SEQ ID NO: 144) | |
| SP0312 | CCCACCCATGCCTCCTCAGGTACCCCCTGCCCCCCACAGCTCC | 501 |
| | TCTCCTGTGCCTTGTTTCCCAGCCATGCGTTCTCCTCTATAAAT | |
| | ACCCGCTCTGGTATTTGGGGTTGGCAGCTGTTGCTGCCAGGG | |
| | AGATGGTTGGGTTGACATGCGGCTCCTGACAAAACACAAACC | |
| | CCTGGTGTGTGTGGGCGTGGGTGGTGTGAGTAGGGGGATGAA | |
| | TCAGGGAGGGGGCGGGGGGCCACTACGGGTCTAGGCTGCCC | |
| | ATGTAAGGAGGCAAGGCCTGGGGACACCCGAGATGCCTGGTT | |
| | ATAATTAACCCAGACATGTGGCTGCCCCCCCCCCCCAACACC | |
| | TGCTGCCTGAGCCTCACCCCCACCCCGGTGCCTGGGTCTTAGG | |
| | CTCTGTACACCATGGAGGAGAAGCTCGCTCTAAAAATAACCC | |
| | TGCCACCGCCTGCTGCCACGGCCGGCCGTATAAATAGAGGCG | |
| | AGGAGCAGCTGGGCTCTCTTGGCAGTCACCGCCACC (SEQ ID | |
| | NO: 145) | |
| SP0313 | GCCACTACGGGTCTAGGCTGCCCATGTAAGGAGGCAAGGCCT | 395 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCCCCTGCCCCCCACAGC | |
| | TCCTCTCCTGTGCCTTGTTTCCCAGCCATGCGTTCTCCTCTATA | |
| | AATACCCGCTCTGGTATTTGGGGTTGGCAGCTGTTGCTGCCAG | |
| | GGAGATGGTTGGGTTGACATGCCACCGCCTGCTGCCACGGCC | |
| | GGCCGTATAAATAGAGGCGAGGAGCAGCTGGGCTCTCTTGGC | |
| | AGTCACCGCCACC ((SEQ ID NO: 146) | |
| SP0314 | GCCACTACGGGTCTAGGCTGCCCATGTAAGGAGGCAAGGCCT | 334 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCTCTATAAATACCCGCT | |
| | CTGGTATTTGGGGTTCTCTATAAATACCCGCTCTGGTATTTGG | |
| | GGTTCCACCGCCTGCTGCCACGGCCGGCCGTATAAATAGAGG | |
| | CGAGGAGCAGCTGGGCTCTCTTGGCAGTCACCGCCACC (SEQ | |
| | ID NO: 147) | |
| SP0315 | CTAGACTAGCATGCTGCCCATGTAAGGAGGCAAGGCCTGGGG | 204 |
| | ACACCCGAGATGCCTGGTTATAATTAACCCAGACATGTGGCT | |
| | GCCCCCCCCCCCCCAACACCTGCTGCCTCTAAAAATAACCCT | |
| | GCCCACCGCCTGCTGCCACGGCCGGCCGTATAAATAGAGGCG | |
| | AGGAGCAGCTGGGCTCTCTTGGCAGTCACCGCCACC (SEQ ID | |
| | NO: 148) | |
| SP0316 | CTAGACTAGCATGCTGCCCATGTAAGGAGGCAAGGCCTGGGG | 376 |
| | ACACCCGAGATGCCTGGTTATAATTAACCCAGACATGTGGCT | |
| | GCCCCCCCCCCCCCAACACCTGCTGCCTCTAAAAATAACCCT | |
| | GCATAAATACCCGCTCTGGTATTTGGGGTTCTCCTCTATAAAT | |
| | ACCCGCTCTGGTATTTGGGGTTGGCAGCTGTTGCGGGATCTTG | |
| | CAGCTGTCAGGGGAGGGGAGGCGGGGGCTGATGTCAGGAGG | |
| | GATACAAATAGTGCCGACGGCTGGGGGCCCTGTCTCCCCTCG | |
| | CCGCATCCACTCTCCGGCCGGCCGCCTGCCCGCCGCCTCCTCC | |
| | GTGCGCCCGCCAGCCTCGCCCGCGCCGTCACCGCCACC ((SEQ | |
| | ID NO: 149) | |
| SP0320 | GTTTCTTAGCAGCTGCTGCTGTGTCCAAGGCTTGGAATTGCTG | 944 |
| | TGGTGAATCTAAAACTGTCTCAGTAGTGGTGAGCTGACCTCA | |
| | CCCAAGTTCAAAGCCCTACTCTGCCTGATCCTTTTTTCCTGAG | |
| | CCTCAGAGCTAAAATGCCCCCGAGCTCTTTCCTATTGGCTGGA | |
| | AAGACGAATTGAAGTTCCCTTGCCCATGTTAGGAGGTGTACG | |
| | CCTCCTGAACTAAAGATAGAAACAGCTGGCCCTTCCAGGCAG | |
| | CTAAAAGCCTCCAGACTAAGAGGTGTTCCCCATTCGGGCCAC | |
| | TACGGGTCTAGGCTGCCCATGTAAGGAGGCAAGGCCTGGGGA | |
| | CACCCGAGATGCCTGGTTATAATTAACCCAGACATGTGGCTG | |
| | CCCCCCCCCCCCAACACCTGCTGCCTGAGCCTCACCCCCACCC | |
| | CGGTGCCTGGGTCTTAGGCTCTGTACACCATGGAGGAGAAGC | |
| | TCGCTCTAAAAATAACCCTGATAAATACCCGCTCTGGTATTTG | |
| | GGGTTCTCCTCTATAAATACCCGCTCTGGTATTTGGGGTTGGC | |
| | AGCTGTTGCGGGATCTTGCAGCTGTCAGGGGAGGGGAGGCGG | |
| | GGGCTGATGTCAGGAGGGATACAAATAGTGCCGACGGCTGG | |
| | GGGCCCTGTCTCCCCTCGCTCAGATCGCCTGGAGACGCCATC | |
| | CACGCTGTTTTGACCTCCATAGAAGACACCGGGACCGATCCA | |
| | GCCTCCGCGGCCGGGAACGGTGCATTGGAACGCGGATTCCCC | |
| | GTGCCAAGAGTGACGTAAGTACCGCCTATAGACTCTATAGGC | |
| | ACACCCCTTTGGCTCTTATGCATGAACGGTGGAGGGCAGTGT | |
| | AGTCTGAGCAGTACTCGTTGCTGCCGCGCGCGCCACCAGACA | |
| | TAATAGCTGACAGACTAACAGACTGTTCCTTTCCATGGGTCTT | |
| | TTCTGCAGGCCACC (SEQ ID NO: 150) | |
| SP0322 | AGACTGGGGCAGGTGCAGGCTGGATTGGGTTTCCAGAGGCTA | 661 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCC | |
| | ACCGCGGTGGCGGCCGTCCGCCCTCGGCACCATCCTCACGAC | |
| | ACCCAAATATGGCGACGGGTGAGGAATGGTGGGGAGTTATTT | |
| | TTAGAGCGGTGAGGAAGGTGGGCAGGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTCGGCCGGGGCCATAAATACCCGC | |
| | TCTGGTATTTGGGGTTCTCCTCTATAAATACCCGCTCTGGTAT | |
| | TTGGGGTTGGCAGCTGTTGCGGGATCTTGCAGCTGTCAGGGG | |
| | AGGGGAGGCGGGGGCTGATGTCAGGAGGGATACAAATAGTG | |
| | CCGACGGCTGGGGGCCCTGTCTCCCCTCGCCGCATCCACTCTC | |
| | CGGCCGGCCGCCTGCCCGCCGCCTCCTCCGTGCGCCCGCCAG | |
| | CCTCGCCCGCGCCGTCACCGCGGCCGCCACC (SEQ ID NO: | |
| | 151) | |
| SP0323 | AGACTGGGGCAGGTGCAGGCTGGATTGGGTTTCCAGAGGCTA | 613 |
| | TATATATAAAGGCTGCCGGGAGCCCACATTCCTTTCCAGAGG | |
| | CCAGCTCTCCATTTATAGCCCCTGGGCAGAGCAGCCACCGCG | |
| | GTGGCGGCCGTCCGCCCTCGGCACCATCCTCACGACACCCAA | |
| | ATATGGCGACGGGTGAGGAATGGTGGGGAGTTATTTTTAGAG | |
| | CGGTGAGGAAGGTGGGCAGGCAGCAGGTGTTGGCGCTCTAA | |
| | AAATAACTCCCGGGAGTTATTTTTAGAGCGGAGGAATGGTGG | |
| | ACACCCAAATATGGCGACGGTTCCTCACCCGTCGCCATATTT | |
| | GGGTGTCCGCCCTCGGCCGGGGCCATAAATACCCGCTCTGGT | |
| | ATTTGGGGTTCTCCTCTATAAATACCCGCTCTGGTATTTGGGG | |
| | TTGGCAGCTGTTGCGGGATCTTGCAGCTGTCAGGGGAGGGGA | |
| | GGCGGGGGCTGATGTCAGGAGGGATACAAATAGTGCCGACG | |
| | GCTGGGGGCCCTGTCTCCCCTCGCCGCATCCACTCTCCGGCCG | |
| | GCCGCCTGCCCGCCGCCTCCTCCGTGCGCCCGCCAGCCTCGCC | |
| | CGCGCCGTCACCGCGGCCGCCACC (SEQ ID NO: 152) | |
| SP0324 | AGACTGGGGCAGGTGCAGGCTGGATTGGGTTTCCAGAGGCTA | 407 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCT | |
| | TCTCCTCTATAAATACCCGCTCTGGTATTTGGGGTTGGCAGCT | |
| | GTTGCTGCCAGGGAGATGGTTGGGTTGACGGGATCTTGCAGC | |
| | TGTCAGGGGAGGGGAGGCGGGGGCTGATGTCAGGAGGGATA | |
| | CAAATAGTGCCGACGGCTGGGGGCCCTGTCTCCCCTCGCCGC | |
| | ATCCACTCTCCGGCCGGCCGCCTGCCCGCCGCCTCCTCCGTGC | |
| | GCCCGCCAGCCTCGCCCGCGCCGTCACC (SEQ ID NO: 153) | |
| SP0325 | AGACTGGGGCAGGTGCAGGCTGGATTGGGTTTCCAGAGGCTA | 409 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCA | |
| | TAAATACCCGCTCTGGTATTTGGGGTTCTCCTCTATAAATACC | |
| | CGCTCTGGTATTTGGGGTTGGCAGCTGTTGCGGGATCTTGCAG | |
| | CTGTCAGGGGAGGGGAGGCGGGGGCTGATGTCAGGAGGGAT | |
| | ACAAATAGTGCCGACGGCTGGGGGCCCTGTCTCCCCTCGCCG | |
| | CATCCACTCTCCGGCCGGCCGCCTGCCCGCCGCCTCCTCCGTG | |
| | CGCCCGCCAGCCTCGCCCGCGCCGTCACC (SEQ ID NO: 154) | |
| SP0326 | CACCGCGGTGGCGGCCGTCCGCCCTCGGCACCATCCTCACGA | 483 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTATAAATACCCGCTCTGGTATTTG | |
| | GGGTTCTCCTCTATAAATACCCGCTCTGGTATTTGGGGTTGGC | |
| | AGCTGTTGCGGGATCTTGCAGCTGTCAGGGGAGGGGAGGCGG | |
| | GGGCTGATGTCAGGAGGGATACAAATAGTGCCGACGGCTGG | |
| | GGGCCCTGTCTCCCCTCGCCGCATCCACTCTCCGGCCGGCCGC | |
| | CTGCCCGCCGCCTCCTCCGTGCGCCCGCCAGCCTCGCCCGCGC | |
| | CGTCACCGCGGCCGCCACC (SEQ ID NO: 155) | |
| SP0327 | AGACTGGGGCAGGTGCAGGCTGGATTGGGTTTCCAGAGGCTA | 538 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCC | |
| | ACCGCGGTGGCGGCCGTCCGCCCTCGGCACCATCCTCACGAC | |
| | ACCCAAATATGGCGACGGGTGAGGAATGGTGGGGAGTTATTT | |
| | TTAGAGCGGTGAGGAAGGTGGGCAGGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTCGGCCGGGGCCGCATTCCTGGGG | |
| | GCCGGGCGGTGCTCCCGCCCGCCTCGATAAAAGGCTCCGGGG | |
| | CCGGCGGCGGCCCACGAGCTACCCGGAGGAGCGGGAGGCGC | |
| | CAAGCTCTAGAACTAGTGGATCCCGCGGCCGCCACC (SEQ ID | |
| | NO: 156) | |
| SP0328 | GGGCCCCACAGCAGCTGGGGGCATTTATGGGCCTTCCTATAA | 822 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTAGACTGGGGCAG | |
| | GTGCAGGCTGGATTGGGTTTCCAGAGGCTATATATATAAAGG | |
| | CTGCCGGGAGCCCCAGGGCCGCTCCCTGAGGGCACAACACTG | |
| | TGGGGGCCCAGCCAGGCCCACATTCCTTTCCAGAGGCCAGCT | |
| | CTCCATTTATAGCCCCTGGGCAGAGCAGCCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCGGCCGGGGCCGCATTCCTGGGGGCCGGGCGGTGCT | |
| | CCCGCCCGCCTCGATAAAAGGCTCCGGGGCCGGCGGCGGCCC | |
| | ACGAGCTACCCGGAGGAGCGGGAGGCGCCAAGCTCTAGAAC | |
| | TAGTGGATCCCGCGGCCGCCACC (SEQ ID NO: 157) | |
| SP0329 | ACACCCAAATATGGCGACGGGTGAGGAATGGTGGGGAGTTA | 324 |
| | TTTTTAGAGCGGTGAGGAAGGTGGGCAGGCAGCAGGTGTTGG | |
| | CGCTCTAAAAATAACTCCCGGGAGTTATTTTTAGAGCGGAGG | |
| | AATGGTGGACACCCAAATATGGCGACGGTTCCTCACCCGTCG | |
| | CCATATTTGGGTGTCCGCCCTCGGCCGGGGCCGCATTCCTGG | |
| | GGGCCGGGCGGTGCTCCCGCCCGCCTCGATAAAAGGCTCCGG | |
| | GGCCGGCGGCGGCCCACGAGCTACCCGGAGGAGCGGGAGGC | |
| | GCCAAGCTCTAGAACTAGTGGATCCCGCCACC (SEQ ID NO: | |
| | 158) | |
| SP0330 | CACCGCGGTGGCGGCCGTCCGCCCTCGGCACCATCCTCACGA | 365 |
| | CACCCAAATATGGCGACGGGTGAGGAATGGTGGGGAGTTATT | |
| | TTTAGAGCGTAAACGAGCTATTAGTTGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTCGGCCGGGGCCGCATTCCTGGGG | |
| | GCCGGGCGGTGCTCCCGCCCGCCTCGATAAAAGGCTCCGGGG | |
| | CCGGCGGCGGCCCACGAGCTACCCGGAGGAGCGGGAGGCGC | |
| | CAAGCTCTAGAACTAGTGGATCCCGCCACC (SEQ ID NO: 159) | |
| SP0331 | CACCGCGGTGGCGGCCGTCCGCCCTCGGCACCATCCTCACGA | 365 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGAGGTA | |
| | AACGAGCTATTAGTTATGAGGTCCGTAGATTGAACCCGTCGC | |
| | CATATTTGGGTGTCCGCCCTCGGCCGGGGCCGCATTCCTGGG | |
| | GGCCGGGCGGTGCTCCCGCCCGCCTCGATAAAAGGCTCCGGG | |
| | GCCGGCGGCGGCCCACGAGCTACCCGGAGGAGCGGGAGGCG | |
| | CCAAGCTCTAGAACTAGTGGATCCCGCCACC (SEQ ID NO: | |
| | 160) | |
| SP0332 | GCCACTACGGGTCTAGGCTGCCCATGTAAGGAGGCAAGGCCT | 565 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCACCGCGGTGGCGGCCG | |
| | TCCGCCCTCGGCACCATCCTCACGACACCCAAATATGGCGAC | |
| | GGGTGAGGAATGGTGGGGAGTTATTTTTAGAGCGGTGAGGAA | |
| | GGTGGGCAGGCAGCAGGTGTTGGCGCTCTAAAAATAACTCCC | |
| | GGGAGTTATTTTTAGAGCGGAGGAATGGTGGACACCCAAATA | |
| | TGGCGACGGTTCCTCACCCGTCGCCATATTTGGGTGTCCGCCC | |
| | TCGGCCGGGGCCGCATTCCTGGGGGCCGGGCGGTGCTCCCGC | |
| | CCGCCTCGATAAAAGGCTCCGGGGCCGGCGGCGGCCCACGA | |
| | GCTACCCGGAGGAGCGGGAGGCGCCAAGCTCTAGAACTAGT | |
| | GGATCCCGCGGCCGCCACC (SEQ ID NO: 161) | |
| SP0333 | GCCACTACGGGTCTAGGCTGCCCATGTAAGGAGGCAAGGCCT | 543 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGGTTTCTTAGCAGCTGCT | |
| | GCTGTGTCCAAGGCTTGGAATTGCTGTGGTGAATCTAAAACT | |
| | GTCTCAGTAGTGGTGAGCTGACCTCACCCAAGTTCAAAGCCC | |
| | TACTCTGCCTGATCCTTTTTTCCTGAGCCTCAGAGCTAAAATG | |
| | CCCCCGAGCTCTTTCCTATTGGCTGGAAAGACGAATTGAAGT | |
| | TCCCTTGCCCATGTTAGGAGGTGTACGCCTCCTGAACTAAAG | |
| | ATAGAAACAGCTGGCCCTTCCAGGCAGCTAAAAGCCTCCAGA | |
| | CTAAGAGGTGTTCCCCATTCGGCAGCCAGACTCCTTGAAATA | |
| | CCCTTTCAGTAATCATTCAACCAACGCTTCCGCCACC (SEQ ID | |
| | NO: 162) | |
| SP0334 | GCCACTACGGGTCTAGGCTGCCCATGTAAGGAGGCAAGGCCT | 362 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCGGGATCTTGCAGCTGT | |
| | CAGGGGAGGGGAGGCGGGGGCTGATGTCAGGAGGGATACAA | |
| | ATAGTGCCGACGGCTGGGGGCCCTGTCTCCCCTCGCCGCATC | |
| | CACTCTCCGGCCGGCCGCCTGCCCGCCGCCTCCTCCGTGCGCC | |
| | CGCCAGCCTCGCCCGCGCCGTCACC (SEQ ID NO: 163) | |
| SP0335 | GCCACTACGGGTCTAGGCTGCCCATGTAAGGAGGCAAGGCCT | 715 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTCAAAGCCCTACTCTGC | |
| | CTGATCCTTTTTTCCTGAGCCTCAGAGCTAAAATGCCCCCGAG | |
| | CTCTTTCCTATTGGCTGGAAAGACGAATTGAAGTTCCCTTGCC | |
| | CATGTTAGGAGGTGTACGCCTCCTGAACTAAAGATAGAAACA | |
| | GCTGGCCCTTCCAGGCAGCTAAAAGCCTCCAGACTAAGAGGT | |
| | GTTCCCCATTCGGCCATGTTCCCGGCGAAGGGCCAGCTGTCC | |
| | CCCGCCAGCTAGACTCAGCACTTAGTTTAGGAACCAGTGAGC | |
| | AAGTCAGCCCTTGGGGCAGCCCATACAAGGCCATGGGGCTGG | |
| | GCAAGCTGCACGCCTGGGTCCGGGGTGGGCACGGTGCCCGGG | |
| | CAACGAGCTGAAAGCTCATCTACTCTCAGGGGCCCCTCCCTG | |
| | GGGACAGCCCCTCCTGGCTAGTCACACCCTGTAGGCTCCTCT | |
| | ATATAACCCAGGGGCACAGGGGCTGCCCCCGGGTCACCACCA | |
| | CCTCCACAGCACAGACAGACACTCAGGAGCCAGCGCCACC | |
| | (SEQ ID NO: 164) | |
| SP0336 | TCAAAGCCCTACTCTGCCTGATCCTTTTTTCCTGAGCCTCAGA | 521 |
| | GCTAAAATGCCCCCGAGCTCTTTCCTATTGGCTGGAAAGACG | |
| | AATTGAAGTTCCCTTGCCCATGTTAGGAGGTGTACGCCTCCTG | |
| | AACTAAAGATAGAAACAGCTGGCCCTTCCAGGCAGCTAAAA | |
| | GCCTCCAGACTAAGAGGTGTTCCCCATTCGGCCATGTTCCCG | |
| | GCGAAGGGCCAGCTGTCCCCCGCCAGCTAGACTCAGCACTTA | |
| | GTTTAGGAACCAGTGAGCAAGTCAGCCCTTGGGGCAGCCCAT | |
| | ACAAGGCCATGGGGCTGGGCAAGCTGCACGCCTGGGTCCGG | |
| | GGTGGGCACGGTGCCCGGGCAACGAGCTGAAAGCTCATCTAC | |
| | TCTCAGGGGCCCCTCCCTGGGGACAGCCCCTCCTGGCTAGTC | |
| | ACACCCTGTAGGCTCCTCTATATAACCCAGGGGCACAGGGGC | |
| | TGCCCCCGGGTCACCACCACCTCCACAGCACAGACAGACACT | |
| | CAGGAGCCAGCGCCACC (SEQ ID NO: 165) | |
| SP0337 | GCCACTACGGGTCTAGGCTGCCCATGTAAGGAGGCAAGGCCT | 618 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTCAAAGCCCTACTCTGC | |
| | CTGATCCTTTTTTCCTGAGCCTCAGAGCTAAAATGCCCCCGAG | |
| | CTCTTTCCTATTGGCTGGAAAGACGAATTGAAGTTCCCTTGCC | |
| | CATGTTAGGAGGTGTACGCCTCCTGAACTAAAGATAGAAACA | |
| | GCTGGCCCTTCCAGGCAGCTAAAAGCCTCCAGACTAAGAGGT | |
| | GTTCCCCATTCGGCCCGGCAGACGCTCCTTATACGGCCCGGC | |
| | CTCGCTCACCTGGGCCGCGGCCAGGAGCGCCTTCTTTGGGCA | |
| | GCGCCGGGCCGGGGCCGCGCCGGGCCCGACACCCAAATATG | |
| | GCGACGGCCGGGGCCGCATTCCTGGGGGCCGGGCGGCGCTCC | |
| | CGCCCGCCTCGATAAAAGGCTCCGGGGCCGGCGGCGGCCCAC | |
| | GAGCTACCCGGAGGAGCGGGAGGCCACC (SEQ ID NO: 166) | |
| SP0338 | AGACTGGGGCAGGTGCAGGCTGGATTGGGTTTCCAGAGGCTA | 729 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCG | |
| | CCACTACGGGTCTAGGCTGCCCATGTAAGGAGGCAAGGCCTG | |
| | GGGACACCCGAGATGCCTGGTTATAATTAACCCAGACATGTG | |
| | GCTGCCCCCCCCCCCCAACACCTGCTGCCTGAGCCTCACCCCC | |
| | ACCCCGGTGCCTGGGTCTTAGGCTCTGTACACCATGGAGGAG | |
| | AAGCTCGCTCTAAAAATAACCCTGTCAAAGCCCTACTCTGCC | |
| | TGATCCTTTTTTCCTGAGCCTCAGAGCTAAAATGCCCCCGAGC | |
| | TCTTTCCTATTGGCTGGAAAGACGAATTGAAGTTCCCTTGCCC | |
| | ATGTTAGGAGGTGTACGCCTCCTGAACTAAAGATAGAAACAG | |
| | CTGGCCCTTCCAGGCAGCTAAAAGCCTCCAGACTAAGAGGTG | |
| | TTCCCCATTCGGCGGGATCTTGCAGCTGTCAGGGGAGGGGAG | |
| | GCGGGGGCTGATGTCAGGAGGGATACAAATAGTGCCGACGG | |
| | CTGGGGGCCCTGTCTCCCCTCGCCGCATCCACTCTCCGGCCGG | |
| | CCGCCTGCCCGCCGCCTCCTCCGTGCGCCCGCCAGCCTCGCCC | |
| | GCGCCGTCACC (SEQ ID NO: 167) | |
| SP0339 | GCCACTACGGGTCTAGGCTGCCCATGTAAGGAGGCAAGGCCT | 610 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTTCTCCTCTATAAATACC | |
| | CGCTCTGGTATTTGGGGTTGGCAGCTGTTGTCAAAGCCCTACT | |
| | CTGCCTGATCCTTTTTTCCTGAGCCTCAGAGCTAAAATGCCCC | |
| | CGAGCTCTTTCCTATTGGCTGGAAAGACGAATTGAAGTTCCCT | |
| | TGCCCATGTTAGGAGGTGTACGCCTCCTGAACTAAAGATAGA | |
| | AACAGCTGGCCCTTCCAGGCAGCTAAAAGCCTCCAGACTAAG | |
| | AGGTGTTCCCCATTCGGCGGGATCTTGCAGCTGTCAGGGGAG | |
| | GGGAGGCGGGGGCTGATGTCAGGAGGGATACAAATAGTGCC | |
| | GACGGCTGGGGGCCCTGTCTCCCCTCGCCGCATCCACTCTCCG | |
| | GCCGGCCGCCTGCCCGCCGCCTCCTCCGTGCGCCCGCCAGCC | |
| | TCGCCCGCGCCGTCACC (SEQ ID NO: 168) | |
| SP0340 | GCCACTACGGGTCTAGGCTGCCCATGTAAGGAGGCAAGGCCT | 654 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCCCGGCAGACGCTCCTT | |
| | ATACGGCCCGGCCTCGCTCACCTGGGCCGCGGCCAGGAGCGC | |
| | CTTCTTTGGGCAGCGCCGGGCCGGGGCCGCGCCGGGCCCGAC | |
| | ACCCAAATATGGCGACGGCCGGGGCCGCATTCCTGGGGGCCG | |
| | GGCGGCGCTCCCGCCCGCCTCGATAAAAGGCTCCGGGGCCGG | |
| | CGGCGGCCCACGAGCTACCCGGAGGAGCGGGAGATAAATAC | |
| | CCGCTCTGGTATTTGGGGTTCTCCTCTATAAATACCCGCTCTG | |
| | GTATTTGGGGTTGGCAGCTGTTGCGGGATCTTGCAGCTGTCA | |
| | GGGGAGGGGAGGCGGGGGCTGATGTCAGGAGGGATACAAAT | |
| | AGTGCCGACGGCTGGGGGCCCTGTCTCCCCTCGCCGCATCCA | |
| | CTCTCCGGCCGGCCGCCTGCCCGCCGCCTCCTCCGTGCGCCCG | |
| | CCAGCCTCGCCCGCGCCGTCACC (SEQ ID NO: 169) | |
| SP0341 | GCCACTACGGGTCTAGGCTGCCCATGTAAGGAGGCAAGGCCT | 924 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGTCAAAGCCCTACTCTGC | |
| | CTGATCCTTTTTTCCTGAGCCTCAGAGCTAAAATGCCCCCGAG | |
| | CTCTTTCCTATTGGCTGGAAAGACGAATTGAAGTTCCCTTGCC | |
| | CATGTTAGGAGGTGTACGCCTCCTGAACTAAAGATAGAAACA | |
| | GCTGGCCCTTCCAGGCAGCTAAAAGCCTCCAGACTAAGAGGT | |
| | GTTCCCCATTCGGGCCGCGAAGACCGGAAGCTGGGGCGGCCC | |
| | CGGGCCGCGCGCGCTGGGCCTGGGAGGCGAAACTCAGCTTCC | |
| | TTCGTTTCCGACTTTTCCATCCGCGTCCTCCACTTCCCCGTTCC | |
| | GCCCTCCCCCATTGCCAACATTCTGGCTGAGTCACGGCGCCCC | |
| | AGAGCGCGCCAGGCTGGGGGAAAGGAGCAGAAGGGAGGGCC | |
| | CTAGCGACCCGCGGGATGTGGTCCGAGTCACGTCCGAGGGGG | |
| | GTGGGGAGGGATCGTGTTCTCGGCGCCCGCCCCTTCCTAGCG | |
| | CGGCCTCTGGGCTGCGCCTCTCGGGGGCGGCCCGTAGCCCAG | |
| | TCCGTCGCCTGCCATTGGACGCCGCCCGCTCCTCGTAAAGGA | |
| | AAAAGCTCGGCGGAGGGCGGAGTGGTGCCTTTAAAAGGCCG | |
| | GGCGCCGCCTTCCGCCTGCCCGCCTCCTGCGCCGCCCCTTCCG | |
| | AGGCTAAATCGGCTGCGTTCCTCTCGGAACGCGCCGCAGAAG | |
| | GGGTCCTGGTGACGAGTCCCGCGTTCTCTCCGCCACC (SEQ ID | |
| | NO: 170) | |
| SP0343 | GCCACTACGGGTCTAGGCTGCCCATGTAAGGAGGCAAGGCCT | 652 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGATAAATACCCGCTCTGG | |
| | TATTTGGGGTTCTCCTCTATAAATACCCGCTCTGGTATTTGGG | |
| | GTTGGCAGCTGTTGCGGGATCTTGCAGCTGTCAGGGGAGGGG | |
| | AGGCGGGGGCTGATGTCAGGAGGGATACAAATAGTGCCGAC | |
| | GGCTGGGGGCCCTGTCTCCCCTCGCTCAGATCGCCTGGAGAC | |
| | GCCATCCACGCTGTTTTGACCTCCATAGAAGACACCGGGACC | |
| | GATCCAGCCTCCGCGGCCGGGAACGGTGCATTGGAACGCGGA | |
| | TTCCCCGTGCCAAGAGTGACGTAAGTACCGCCTATAGACTCT | |
| | ATAGGCACACCCCTTTGGCTCTTATGCATGAACGGTGGAGGG | |
| | CAGTGTAGTCTGAGCAGTACTCGTTGCTGCCGCGCGCGCCAC | |
| | CAGACATAATAGCTGACAGACTAACAGACTGTTCCTTTCCAT | |
| | GGGTCTTTTCTGCAGGCCACC (SEQ ID NO: 171) | |
| SP0345 | GGGCCCCACAGCAGCTGGGGGCATTTATGGGCCTTCCTATAA | 693 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCGGGATCTTGCAGCTGTCAGGGGAGGGGAGGCGGG | |
| | GGCTGATGTCAGGAGGGATACAAATAGTGCCGACGGCTGGG | |
| | GGCCCTGTCTCCCCTCGCCGCATCCACTCTCCGGCCGGCCGCC | |
| | TGCCCGCCGCCTCCTCCGTGCGCCCGCCAGCCTCGCCCGCGCC | |
| | GTCACCGCGGCCGCCACC (SEQ ID NO: 172) | |
| SP0346 | AGACTGGGGCAGGTGCAGGCTGGATTGGGTTTCCAGAGGCTA | 576 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCC | |
| | ACCGCGGTGGCGGCCGTCCGCCCTCGGCACCATCCTCACGAC | |
| | ACCCAAATATGGCGACGGGTGAGGAATGGTGGGGAGTTATTT | |
| | TTAGAGCGGTGAGGAAGGTGGGCAGGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTCGGGATCTTGCAGCTGTCAGGGG | |
| | AGGGGAGGCGGGGGCTGATGTCAGGAGGGATACAAATAGTG | |
| | CCGACGGCTGGGGGCCCTGTCTCCCCTCGCCGCATCCACTCTC | |
| | CGGCCGGCCGCCTGCCCGCCGCCTCCTCCGTGCGCCCGCCAG | |
| | CCTCGCCCGCGCCGTCACCGCGGCCGCCACC (SEQ ID NO: | |
| | 173) | |
| SP0347 | CTCTGTCTCCTCAGGTGCCTGGCTCCCAGTCCCCAGAACGCCT | 606 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCCTAGACTAGCATGCTGCCCATGTAAG | |
| | GAGGCAAGGCCTGGGGACACCCGAGATGCCTGGTTATAATTA | |
| | ACCCAGACATGTGGCTGCCCCCCCCCCCCCAACACCTGCTGC | |
| | CTCTAAAAATAACCCTGCATAAATACCCGCTCTGGTATTTGG | |
| | GGTTCTCCTCTATAAATACCCGCTCTGGTATTTGGGGTTGGCA | |
| | GCTGTTGCGGGATCTTGCAGCTGTCAGGGGAGGGGAGGCGGG | |
| | GGCTGATGTCAGGAGGGATACAAATAGTGCCGACGGCTGGG | |
| | GGCCCTGTCTCCCCTCGCCGCATCCACTCTCCGGCCGGCCGCC | |
| | TGCCCGCCGCCTCCTCCGTGCGCCCGCCAGCCTCGCCCGCGCC | |
| | GTCACCGCCACC ((SEQ ID NO: 174) | |
| SP0348 | CTCTGTCTCCTCAGGTGCCTGGCTGCTTCCTAGCTGGGCCTTT | 575 |
| | CCTTCTCCTCTATAAATACCAGCTCTGGTATTTCGCCTTGGCA | |
| | GCTGTTGCTGCTAGGGAGACGGCTGGCTTGACATGCATCTCC | |
| | TGACAAAACACAAACCCGTGGTGTGAGTGGGTGTGGGCGGTG | |
| | TGAGTAGGGGGATGAATCAGAGAGGGGGCCTAGACTAGCAT | |
| | GCTGCCCATGTAAGGAGGCAAGGCCTGGGGACACCCGAGAT | |
| | GCCTGGTTATAATTAACCCAGACATGTGGCTGCCCCCCCCCCC | |
| | CCAACACCTGCTGCCTCTAAAAATAACCCTGCATAAATACCC | |
| | GCTCTGGTATTTGGGGTTCTCCTCTATAAATACCCGCTCTGGT | |
| | ATTTGGGGTTGGCAGCTGTTGCGGGATCTTGCAGCTGTCAGG | |
| | GGAGGGGAGGCGGGGGCTGATGTCAGGAGGGATACAAATAG | |
| | TGCCGACGGCTGGGGGCCCTGTCTCCCCTCGCCGCATCCACTC | |
| | TCCGGCCGGCCGCCTGCCCGCCGCCTCCTCCGTGCGCCCGCC | |
| | AGCCTCGCCCGCGCCGTCACCGCCACC (SEQ ID NO: 175) | |
| SP0349 | CTCTGTCTCCTCAGGTGCCTGGCTCCCAGTCCCCAGAACGCCT | 907 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCGCCACTACGGGTCTAGGCTGCCCATG | |
| | TAAGGAGGCAAGGCCTGGGGACACCCGAGATGCCTGGTTATA | |
| | ATTAACCCAGACATGTGGCTGCCCCCCCCCCCCAACACCTGC | |
| | TGCCTGAGCCTCACCCCCACCCCGGTGCCTGGGTCTTAGGCTC | |
| | TGTACACCATGGAGGAGAAGCTCGCTCTAAAAATAACCCTGC | |
| | ACCGCGGTGGCGGCCGTCCGCCCTCGGCACCATCCTCACGAC | |
| | ACCCAAATATGGCGACGGGTGAGGAATGGTGGGGAGTTATTT | |
| | TTAGAGCGGTGAGGAAGGTGGGCAGGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTATAAATACCCGCTCTGGTATTTGG | |
| | GGTTCTCCTCTATAAATACCCGCTCTGGTATTTGGGGTTGGCA | |
| | GCTGTTGCGGGATCTTGCAGCTGTCAGGGGAGGGGAGGCGGG | |
| | GGCTGATGTCAGGAGGGATACAAATAGTGCCGACGGCTGGG | |
| | GGCCCTGTCTCCCCTCGCCGCATCCACTCTCCGGCCGGCCGCC | |
| | TGCCCGCCGCCTCCTCCGTGCGCCCGCCAGCCTCGCCCGCGCC | |
| | GTCACCGCGGCCGCCACC (SEQ ID NO: 176) | |
| SP0350 | GGGCCCCACAGCAGCTGGGGGCATTTATGGGCCTTCCTATAA | 727 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTTTCTCCTCTATAA | |
| | ATACCCGCTCTGGTATTTGGGGTTGGCAGCTGTTGCTGCCAGG | |
| | GAGATGGTTGGGTTGACACCGCGGTGGCGGCCGTCCGCCCTC | |
| | GGCACCATCCTCACGACACCCAAATATGGCGACGGGTGAGGA | |
| | ATGGTGGGGAGTTATTTTTAGAGCGGTGAGGAAGGTGGGCAG | |
| | GCAGCAGGTGTTGGCGCTCTAAAAATAACTCCCGGGAGTTAT | |
| | TTTTAGAGCGGAGGAATGGTGGACACCCAAATATGGCGACGG | |
| | TTCCTCACCCGTCGCCATATTTGGGTGTCCGCCCTCGGCCGGG | |
| | GCCGCATTCCTGGGGGCCGGGCGGTGCTCCCGCCCGCCTCGA | |
| | TAAAAGGCTCCGGGGCCGGCGGCGGCCCACGAGCTACCCGG | |
| | AGGAGCGGGAGGCGCCAAGCTCTAGAACTAGTGGATCCCGC | |
| | GGCCGCCACC (SEQ ID NO: 177) | |
| SP0351 | CACCGCGGTGGCGGCCGTCCGCCCTCGGCACCATCCTCACGA | 365 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGAGCTC | |
| | TATAAATACCCGCTCTGGTATTTGGGGTTTTGAACCCGTCGCC | |
| | ATATTTGGGTGTCCGCCCTCGGCCGGGGCCGCATTCCTGGGG | |
| | GCCGGGCGGTGCTCCCGCCCGCCTCGATAAAAGGCTCCGGGG | |
| | CCGGCGGCGGCCCACGAGCTACCCGGAGGAGCGGGAGGCGC | |
| | CAAGCTCTAGAACTAGTGGATCCCGCCACC (SEQ ID NO: 178) | |
| SP0352 | CACCGCGGTGGCGGCCGTCCGCCCTCGGCACCATCCTCACGA | 365 |
| | CACCCAAATATGGCGACGGGTGAGGAATGGTGGGGAGCTATT | |
| | TTTAGAGCGGTGAGGAAGGTGGGCAGGCAGCAGGTGTTGGC | |
| | GCTCTAAAAATAGCTCCCGGGAGCTATTTTTAGAGCGGAGGA | |
| | ATGGTGGACACCCAAATATGGCGACGGTTCCTCACCCGTCGC | |
| | CATATTTGGGTGTCCGCCCTCGGCCGGGGCCGCATTCCTGGG | |
| | GGCCGGGCGGTGCTCCCGCCCGCCTCGATAAAAGGCTCCGGG | |
| | GCCGGCGGCGGCCCACGAGCTACCCGGAGGAGCGGGAGGCG | |
| | CCAAGCTCTAGAACTAGTGGATCCCGCCACC (SEQ ID NO: | |
| | 179) | |
| SP0353 | TCCCTAACCTCCTGCTTGCGAGGCCTCTCTCTGGCCTCTGAGA | 568 |
| | GGGTCAGTGTCCTGCCCCAACCCATGAGATGACAGACTATAA | |
| | TAGCCACAGGATTAACATAGCAGGCATTGTCTTTCTCTGACTA | |
| | TAGGGTGGGTATTATGTGTTCATCAACCATCCTAAAAATACC | |
| | CGGTAAACAGGTGCAGCCCCTGTGGCTCCAGTCCCCTGGGAT | |
| | CTGTTGGCTTCTGGCTGGAGATGAAGATTAGGGCAGAGGAGA | |
| | GGTGAATTAGTCTCACTGAGTTCCAGGCATGAGACTCGGGTG | |
| | TCCTTTGGAACCTGGGAAATCTAGATTCCAGGAAACCCATCT | |
| | GGAGGGCCCGGCAGACGCTCCTTATACGGCCCGGCCTCGCTC | |
| | ACCTGGGCCGCGGCCAGGAGCGCCTTCTTTGGGCAGCGCCGG | |
| | GCCGGGGCCGCGCCGGGCCCGACACCCAAATATGGCGACGG | |
| | CCGGGGCCGCATTCCTGGGGGCCGGGCGGCGCTCCCGCCCGC | |
| | CTCGATAAAAGGCTCCGGGGCCGGCGGCGGCCCACGAGCTAC | |
| | CCGGAGGAGCGGGAGGCCACC (SEQ ID NO: 180) | |
| SP0354 | CCATCCTAAAAATACCCGGTAAACAGGTGCAGCCCCTGTGGC | 376 |
| | TCCAGTCCCCTGGGATCTGTTGGCTTCTGGCTGGAGATGAAG | |
| | ATTAGGGCAGAGGAGAGGTGAATTAGTCTCACTGAGTTCCAG | |
| | GCATGAGACTCGGGTGTCCTTTGGAACCCGGCAGACGCTCCT | |
| | TATACGGCCCGGCCTCGCTCACCTGGGCCGCGGCCAGGAGCG | |
| | CCTTCTTTGGGCAGCGCCGGGCCGGGGCCGCGCCGGGCCCGA | |
| | CACCCAAATATGGCGACGGCCGGGGCCGCATTCCTGGGGGCC | |
| | GGGCGGCGCTCCCGCCCGCCTCGATAAAAGGCTCCGGGGCCG | |
| | GCGGCGGCCCACGAGCTACCCGGAGGAGCGGGAGGCCACC | |
| | (SEQ ID NO: 181) | |
| SP0355 | AGGGTCAGTGTCCTGCCCCAACCCATGAGATGACAGACTATA | 296 |
| | ATAGCCACAGGATTAACATAGCAGGCATTGCCCGGCAGACGC | |
| | TCCTTATACGGCCCGGCCTCGCTCACCTGGGCCGCGGCCAGG | |
| | AGCGCCTTCTTTGGGCAGCGCCGGGCCGGGGCCGCGCCGGGC | |
| | CCGACACCCAAATATGGCGACGGCCGGGGCCGCATTCCTGGG | |
| | GGCCGGGCGGCGCTCCCGCCCGCCTCGATAAAAGGCTCCGGG | |
| | GCCGGCGGCGGCCCACGAGCTACCCGGAGGAGCGGGAGGCC | |
| | ACC (SEQ ID NO: 182) | |
| SP0356 | CTGAGGGGTGTCAGAGCACAGGCTGAGGCCTCTTGCCTGACG | 654 |
| | TGGGACCCCTTGGTCTGGCATTTGTCAGTGAGGCAGGCTGGG | |
| | GGCAGGCCCCGGAGCTTGGCAGGAGGTGTAAACCGGCCTTGG | |
| | AAGGTAGGGCCCCACAATGGGGACAGTTGGATCTCTGAGGG | |
| | AGACAGGGAGGCATGATCACTGCCAAATGCCCACCAAGGAC | |
| | AAGGCACATCCCAGGGAGACAGACGCAGACCTGGTGCCCTCT | |
| | GGACACTGGCATTCCTGGAGGCTGATGATGGACAGATGGGCC | |
| | TGGAGGTGGCTCTTCGCCAGCTGGTGTTTCCTTTGGACTTCCT | |
| | CAGTGTCTTTGGAGAAGCAGAGCCCTAAGAATAAGCAGCTGC | |
| | CCATAAAATCTAATACCAGCCAAGCATCTCAGGAATTCATGG | |
| | ATTGTCTCCATCCCGGCAGACGCTCCTTATACGGCCCGGCCTC | |
| | GCTCACCTGGGCCGCGGCCAGGAGCGCCTTCTTTGGGCAGCG | |
| | CCGGGCCGGGGCCGCGCCGGGCCCGACACCCAAATATGGCG | |
| | ACGGCCGGGGCCGCATTCCTGGGGGCCGGGCGGCGCTCCCGC | |
| | CCGCCTCGATAAAAGGCTCCGGGGCCGGCGGCGGCCCACGA | |
| | GCTACCCGGAGGAGCGGGAGGCCACC (SEQ ID NO: 183) | |
| SP0358 | TTCTGAGTCCTCTAAGGTCCCTCACTCCCAACTCAGCCCCATG | 659 |
| | TCCTGTCAATTCCCACTCAGTGTCTGATCTCCTTCTCCTCACCT | |
| | TTCCCATCTCCCGTTTGACCCAAGCTTCCTGAGCTCTCCTCCC | |
| | ATTCCCCTTTTTGGAGTCCTCCTCCTCTCCCAGAACCCAGTAA | |
| | TAAGTGGGCTCCTCCCTGGCCTGGACCCCCGTGGTAACCCTAT | |
| | AAGGCGAGGCAGCTGCTGTCTGAGGCAGGGAGGGGCTGGTG | |
| | TGGGAGGCTAAGGGCAGCTGCTAAGTTTAGGGTGGCTCCTTC | |
| | TCTCTTCTTAGAGACAACAGGTGGCTGGGGCCTCAGTGCCCA | |
| | GAAAAGAAAATGTCTTAGAGGTATCGGCATGGGCCTGGAGG | |
| | AGGGGGGACAGGGCAGGGGGAGGCATCTTCCTCAGGACATC | |
| | GGGTCCTAGAGGCCCGGCAGACGCTCCTTATACGGCCCGGCC | |
| | TCGCTCACCTGGGCCGCGGCCAGGAGCGCCTTCTTTGGGCAG | |
| | CGCCGGGCCGGGGCCGCGCCGGGCCCGACACCCAAATATGG | |
| | CGACGGCCGGGGCCGCATTCCTGGGGGCCGGGCGGCGCTCCC | |
| | GCCCGCCTCGATAAAAGGCTCCGGGGCCGGCGGCGGCCCACG | |
| | AGCTACCCGGAGGAGCGGGAGGCCACC (SEQ ID NO: 184) | |
| SP0359 | CCTCCCTGGCCTGGACCCCCGTGGTAACCCTATAAGGCGAGG | 332 |
| | CAGCTGCTGTCTGAGGCAGGGAGGGGCTGGTGTGGGAGGCTA | |
| | AGGGCAGCTGCTAAGTTTAGGGTGCCCGGCAGACGCTCCTTA | |
| | TACGGCCCGGCCTCGCTCACCTGGGCCGCGGCCAGGAGCGCC | |
| | TTCTTTGGGCAGCGCCGGGCCGGGGCCGCGCCGGGCCCGACA | |
| | CCCAAATATGGCGACGGCCGGGGCCGCATTCCTGGGGGCCGG | |
| | GCGGCGCTCCCGCCCGCCTCGATAAAAGGCTCCGGGGCCGGC | |
| | GGCGGCCCACGAGCTACCCGGAGGAGCGGGAGGCCACC | |
| | ((SEQ ID NO: 185) | |
| SP0361 | CACCGCGGTGGCGGCCGTCCGCCCTCGGCACCATCCTCACGA | 483 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGAGCTC | |
| | TATAAATACCCGCTCTGGTATTTGGGGTTTTGAACCCGTCGCC | |
| | ATATTTGGGTGTCCGCCCTATAAATACCCGCTCTGGTATTTGG | |
| | GGTTCTCCTCTATAAATACCCGCTCTGGTATTTGGGGTTGGCA | |
| | GCTGTTGCGGGATCTTGCAGCTGTCAGGGGAGGGGAGGCGGG | |
| | GGCTGATGTCAGGAGGGATACAAATAGTGCCGACGGCTGGG | |
| | GGCCCTGTCTCCCCTCGCCGCATCCACTCTCCGGCCGGCCGCC | |
| | TGCCCGCCGCCTCCTCCGTGCGCCCGCCAGCCTCGCCCGCGCC | |
| | GTCACCGCGGCCGCCACC (SEQ ID NO: 186) | |
| SP0362 | AGACTGGGGCAGGTGCAGGCTGGATTGGGTTTCCAGAGGCTA | 535 |
| | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCA | |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGAGCTC | |
| | TATAAATACCCGCTCTGGTATTTGGGGTTTTGAACCCGTCGCC | |
| | ATATTTGGGTGTCCGCCCTCGGGATCTTGCAGCTGTCAGGGG | |
| | AGGGGAGGCGGGGGCTGATGTCAGGAGGGATACAAATAGTG | |
| | CCGACGGCTGGGGGCCCTGTCTCCCCTCGCCGCATCCACTCTC | |
| | CGGCCGGCCGCCTGCCCGCCGCCTCCTCCGTGCGCCCGCCAG | |
| | CCTCGCCCGCGCCGTCACCGCGGCCGCCACC (SEQ ID NO: | |
| | 187) | |
| SP0363 | CTCTGTCTCCTCAGGTGCCTGGCTCCCAGTCCCCAGAACGCCT | 598 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCACACCCAAATATGGCGACGGGTGAG | |
| | GAATGGTGGGGAGTTATTTTTAGAGCGGTGAGGAAGGTGGGC | |
| | AGGCAGCAGGTGTTGGCGCTCTAAAAATAACTCCCGGGAGTT | |
| | ATTTTTAGAGCGAGCTCTATAAATACCCGCTCTGGTATTTGGG | |
| | GTTTTGAACCCGTCGCCATATTTGGGTGTCCGCCCTCGGGATC | |
| | TTGCAGCTGTCAGGGGAGGGGAGGCGGGGGCTGATGTCAGG | |
| | AGGGATACAAATAGTGCCGACGGCTGGGGGCCCTGTCTCCCC | |
| | TCGCCGCATCCACTCTCCGGCCGGCCGCCTGCCCGCCGCCTCC | |
| | TCCGTGCGCCCGCCAGCCTCGCCCGCGCCGTCACCGCGGCCG | |
| | CCACC (SEQ ID NO: 188) | |
| SP0364 | CTCTGTCTCCTCAGGTGCCTGGCTCCCAGTCCCCAGAACGCCT | 683 |
| | CTCCTGTACCTTGCTTCCTAGCTGGGCCTTTCCTTCTCCTCTAT | |
| | AAATACCAGCTCTGGTATTTCGCCTTGGCAGCTGTTGCTGCTA | |
| | GGGAGACGGCTGGCTTGACATGCATCTCCTGACAAAACACAA | |
| | ACCCGTGGTGTGAGTGGGTGTGGGCGGTGTGAGTAGGGGGAT | |
| | GAATCAGAGAGGGGGCCACCGCGGTGGCGGCCGTCCGCCCTC | |
| | GGCACCATCCTCACGACACCCAAATATGGCGACGGGTGAGGA | |
| | ATGGTGGGGAGTTATTTTTAGAGCGGTGAGGAAGGTGGGCAG | |
| | GCAGCAGGTGTTGGCGCTCTAAAAATAACTCCCGGGAGTTAT | |
| | TTTTAGAGCGGAGGAATGGTGGACACCCAAATATGGCGACGG | |
| | TTCCTCACCCGTCGCCATATTTGGGTGTCCGCCCTCCCGGCAG | |
| | ACGCTCCTTATACGGCCCGGCCTCGCTCACCTGGGCCGCGGC | |
| | CAGGAGCGCCTTCTTTGGGCAGCGCCGGGCCGGGGCCGCGCC | |
| | GGGCCCGACACCCAAATATGGCGACGGCCGGGGCCGCATTCC | |
| | TGGGGGCCGGGCGGCGCTCCCGCCCGCCTCGATAAAAGGCTC | |
| | CGGGGCCGGCGGCGGCCCACGAGCTACCCGGAGGAGCGGGA | |
| | GGCCACC (SEQ ID NO: 189) | |
| SP0365 | CACCGCGGTGGCGGCCGTCCGCCCTCGGCACCATCCTCACGA | 453 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCCCGGCAGACGCTCCTTATACGG | |
| | CCCGGCCTCGCTCACCTGGGCCGCGGCCAGGAGCGCCTTCTT | |
| | TGGGCAGCGCCGGGCCGGGGCCGCGCCGGGCCCGACACCCA | |
| | AATATGGCGACGGCCGGGGCCGCATTCCTGGGGGCCGGGCG | |
| | GCGCTCCCGCCCGCCTCGATAAAAGGCTCCGGGGCCGGCGGC | |
| | GGCCCACGAGCTACCCGGAGGAGCGGGAGGCCACC (SEQ ID | |
| | NO: 190) | |
| SP0366 | CCTCCCTGGCCTGGACCCCCGTGGTAACCCTATAAGGCGAGG | 591 |
| | CAGCTGCTGTCTGAGGCAGGGAGGGGCTGGTGTGGGAGGCTA | |
| | AGGGCAGCTGCTAAGTTTAGGGTGCACCGCGGTGGCGGCCGT | |
| | CCGCCCTCGGCACCATCCTCACGACACCCAAATATGGCGACG | |
| | GGTGAGGAATGGTGGGGAGTTATTTTTAGAGCGGTGAGGAAG | |
| | GTGGGCAGGCAGCAGGTGTTGGCGCTCTAAAAATAACTCCCG | |
| | GGAGTTATTTTTAGAGCGGAGGAATGGTGGACACCCAAATAT | |
| | GGCGACGGTTCCTCACCCGTCGCCATATTTGGGTGTCCGCCCT | |
| | ATAAATACCCGCTCTGGTATTTGGGGTTCTCCTCTATAAATAC | |
| | CCGCTCTGGTATTTGGGGTTGGCAGCTGTTGCGGGATCTTGCA | |
| | GCTGTCAGGGGAGGGGAGGCGGGGGCTGATGTCAGGAGGGA | |
| | TACAAATAGTGCCGACGGCTGGGGGCCCTGTCTCCCCTCGCC | |
| | GCATCCACTCTCCGGCCGGCCGCCTGCCCGCCGCCTCCTCCGT | |
| | GCGCCCGCCAGCCTCGCCCGCGCCGTCACCGCGGCCGCCACC | |
| | (SEQ ID NO: 191) | |
| SP0367 | CCTCCCTGGCCTGGACCCCCGTGGTAACCCTATAAGGCGAGG | 429 |
| | CAGCTGCTGTCTGAGGCAGGGAGGGGCTGGTGTGGGAGGCTA | |
| | AGGGCAGCTGCTAAGTTTAGGGTGCCATGTTCCCGGCGAAGG | |
| | GCCAGCTGTCCCCCGCCAGCTAGACTCAGCACTTAGTTTAGG | |
| | AACCAGTGAGCAAGTCAGCCCTTGGGGCAGCCCATACAAGGC | |
| | CATGGGGCTGGGCAAGCTGCACGCCTGGGTCCGGGGTGGGCA | |
| | CGGTGCCCGGGCAACGAGCTGAAAGCTCATCTACTCTCAGGG | |
| | GCCCCTCCCTGGGGACAGCCCCTCCTGGCTAGTCACACCCTGT | |
| | AGGCTCCTCTATATAACCCAGGGGCACAGGGGCTGCCCCCGG | |
| | GTCACCACCACCTCCACAGCACAGACAGACACTCAGGAGCCA | |
| | GCGCCACC (SEQ ID NO: 192) | |
| SP0368 | CCTCCCTGGCCTGGACCCCCGTGGTAACCCTATAAGGCGAGG | 550 |
| | CAGCTGCTGTCTGAGGCAGGGAGGGGCTGGTGTGGGAGGCTA | |
| | AGGGCAGCTGCTAAGTTTAGGGTGGCCACTACGGGTCTAGGC | |
| | TGCCCATGTAAGGAGGCAAGGCCTGGGGACACCCGAGATGC | |
| | CTGGTTATAATTAACCCAGACATGTGGCTGCCCCCCCCCCCCA | |
| | ACACCTGCTGCCTGAGCCTCACCCCCACCCCGGTGCCTGGGT | |
| | CTTAGGCTCTGTACACCATGGAGGAGAAGCTCGCTCTAAAAA | |
| | TAACCCTGATAAATACCCGCTCTGGTATTTGGGGTTCTCCTCT | |
| | ATAAATACCCGCTCTGGTATTTGGGGTTGGCAGCTGTTGCGG | |
| | GATCTTGCAGCTGTCAGGGGAGGGGAGGCGGGGGCTGATGTC | |
| | AGGAGGGATACAAATAGTGCCGACGGCTGGGGGCCCTGTCTC | |
| | CCCTCGCCGCATCCACTCTCCGGCCGGCCGCCTGCCCGCCGCC | |
| | TCCTCCGTGCGCCCGCCAGCCTCGCCCGCGCCGTCACCGCCA | |
| | CC (SEQ ID NO: 193) | |
| SP0369 | CGACACCCAAATATGGCGACGGGTGAGGAATGGTGGGGAGT | 388 |
| | TATTTTTAGAGCGGTGAGGAAGGTGGGCAGGCAGCAGGTGTT | |
| | GGCGCTCTAAAAATAACTCCCGGGAGTTATTTTTAGAGCGGA | |
| | GCGACACCCAAATATGGCGACGGGTGAGGAATGGTGGGGAG | |
| | TTATTTTTAGAGCGGTGAGGAAGGTGGGCAGGCAGCAGGTGT | |
| | TGGCGCTCTAAAAATAACTCCCGGGAGTTATTTTTAGAGCGG | |
| | AGCGGCCGGGGCCGCATTCCTGGGGGCCGGGCGGTGCTCCCG | |
| | CCCGCCTCGATAAAAGGCTCCGGGGCCGGCGGCGGCCCACGA | |
| | GCTACCCGGAGGAGCGGGAGGCGCCAAGCTCTAGAACTAGT | |
| | GGATCCCGCCACC (SEQ ID NO: 194) | |
| SP0370 | CGACACCCAAATATGGCGACGGGTGAGGAATGGTGGGGAGT | 514 |
| | TATTTTTAGAGCGGTGAGGAAGGTGGGCAGGCAGCAGGTGTT | |
| | GGCGCTCTAAAAATAACTCCCGGGAGTTATTTTTAGAGCGGA | |
| | GCGACACCCAAATATGGCGACGGGTGAGGAATGGTGGGGAG | |
| | TTATTTTTAGAGCGGTGAGGAAGGTGGGCAGGCAGCAGGTGT | |
| | TGGCGCTCTAAAAATAACTCCCGGGAGTTATTTTTAGAGCGG | |
| | AGCGACACCCAAATATGGCGACGGGTGAGGAATGGTGGGGA | |
| | GTTATTTTTAGAGCGGTGAGGAAGGTGGGCAGGCAGCAGGTG | |
| | TTGGCGCTCTAAAAATAACTCCCGGGAGTTATTTTTAGAGCG | |
| | GAGCGGCCGGGGCCGCATTCCTGGGGGCCGGGCGGTGCTCCC | |
| | GCCCGCCTCGATAAAAGGCTCCGGGGCCGGCGGCGGCCCACG | |
| | AGCTACCCGGAGGAGCGGGAGGCGCCAAGCTCTAGAACTAG | |
| | TGGATCCCGCCACC (SEQ ID NO: 195) | |
| SP0371 | TAAGGCGAGGCAGCTGCTGTCTGAGGCAGGACACCCAAATAT | 354 |
| | GGCGACGGGTGAGGAATGGTGGGGAGTTATTTTTAGAGCGGT | |
| | GAGGAAGGTGGGCAGGCAGCAGGTGTTGGCGCTCTAAAAAT | |
| | AACTCCCGGGAGTTATTTTTAGAGCGGAGGAATGGTGGACAC | |
| | CCAAATATGGCGACGGTTCCTCACCCGTCGCCATATTTGGGT | |
| | GTCCGCCCTCGGCCGGGGCCGCATTCCTGGGGGCCGGGCGGT | |
| | GCTCCCGCCCGCCTCGATAAAAGGCTCCGGGGCCGGCGGCGG | |
| | CCCACGAGCTACCCGGAGGAGCGGGAGGCGCCAAGCTCTAG | |
| | AACTAGTGGATCCCGCCACC (SEQ ID NO: 196) | |
| SP0372 | AGGCTAAGGGCAGCTGCTAAGTTTAGGGTGACACCCAAATAT | 354 |
| | GGCGACGGGTGAGGAATGGTGGGGAGTTATTTTTAGAGCGGT | |
| | GAGGAAGGTGGGCAGGCAGCAGGTGTTGGCGCTCTAAAAAT | |
| | AACTCCCGGGAGTTATTTTTAGAGCGGAGGAATGGTGGACAC | |
| | CCAAATATGGCGACGGTTCCTCACCCGTCGCCATATTTGGGT | |
| | GTCCGCCCTCGGCCGGGGCCGCATTCCTGGGGGCCGGGCGGT | |
| | GCTCCCGCCCGCCTCGATAAAAGGCTCCGGGGCCGGCGGCGG | |
| | CCCACGAGCTACCCGGAGGAGCGGGAGGCGCCAAGCTCTAG | |
| | AACTAGTGGATCCCGCCACC (SEQ ID NO: 197) | |
| SP0373 | TAAGGCGAGGCAGCTGCTGTCTGAGGCAGGACACCCAAATAT | 362 |
| | GGCGACGGGTGAGGAATGGTGGGGAGTTATTTTTAGAGCGGT | |
| | GAGGAAGGTGGGCAGGCAGCAGGTGTTGGCGCTCTAAAAAT | |
| | AACTCCCGGGAGTTATTTTTAGAGCGCTCTAAGGTCCCTCACT | |
| | CCCAACTCAGCCCCATGTCCTGTCAATTCACCCGTCGCCATAT | |
| | TTGGGTGTCCGCCCTCGGCCGGGGCCGCATTCCTGGGGGCCG | |
| | GGCGGTGCTCCCGCCCGCCTCGATAAAAGGCTCCGGGGCCGG | |
| | CGGCGGCCCACGAGCTACCCGGAGGAGCGGGAGGCGCCAAG | |
| | CTCTAGAACTAGTGGATCCCGCCACC (SEQ ID NO: 198) | |
| SP0374 | CTCTAAGGTCCCTCACTCCCAACTCAGCCCCATGTCCTGTCAA | 362 |
| | TTCGACACCCAAATATGGCGACGGGTGAGGAATGGTGGGGA | |
| | GTTATTTTTAGAGCGGTGAGGAAGGTGGGCAGGCAGCAGGTG | |
| | TTGGCGCTCTAAAAATAACTCCCGGGAGTTATTTTTAGAGCGT | |
| | AAGGCGAGGCAGCTGCTGTCTGAGGCAGACCCGTCGCCATAT | |
| | TTGGGTGTCCGCCCTCGGCCGGGGCCGCATTCCTGGGGGCCG | |
| | GGCGGTGCTCCCGCCCGCCTCGATAAAAGGCTCCGGGGCCGG | |
| | CGGCGGCCCACGAGCTACCCGGAGGAGCGGGAGGCGCCAAG | |
| | CTCTAGAACTAGTGGATCCCGCCACC (SEQ ID NO: 199) | |
| SP0375 | TAAGGCGAGGCAGCTGCTGTCTGAGGCAGAGGCTAAGGGCA | 376 |
| | GCTGCTAAGTTTAGGGTCTCTAAGGTCCCTCACTCCCAACTCA | |
| | GCCCCATGTCCTGTCAATTCCGACACCCAAATATGGCGACGG | |
| | GTGAGGAATGGTGGGGAGTTATTTTTAGAGCAGGCAGCAGGT | |
| | GTTGGCGCTCTAAAAATAACTCCCGGGAGTTATTTTTAGAGC | |
| | GACCCGTCGCCATATTTGGGTGTCCGCCCTCGGCCGGGGCCG | |
| | CATTCCTGGGGGCCGGGCGGTGCTCCCGCCCGCCTCGATAAA | |
| | AGGCTCCGGGGCCGGCGGCGGCCCACGAGCTACCCGGAGGA | |
| | GCGGGAGGCGCCAAGCTCTAGAACTAGTGGATCCCGCCACC | |
| | (SEQ ID NO: 200) | |
| SP0376 | GCCACTACGGGTCTAGGCTGCCCATGTAAGGAGGCAAGGCCT | 434 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGATAAATACCCGCTCTGG | |
| | TATTTGGGGTACTAAAAATAGAACGACTATTTTTAGGCTTTTC | |
| | TGGCAGCTGGCCCGGGATCTTGCAGCTGTCAGGGGAGGGGAG | |
| | GCGGGGGCTGATGTCAGGAGGGATACAAATAGTGCCGACGG | |
| | CTGGGGGCCCTGTCTCCCCTCGCCGCATCCACTCTCCGGCCGG | |
| | CCGCCTGCCCGCCGCCTCCTCCGTGCGCCCGCCAGCCTCGCCC | |
| | GCGCCGTCACC (SEQ ID NO: 290) | |
| SP0377 | GCCACTACGGGTCTAGGCTGCCCATGTAAGGAGGCAAGGCCT | 436 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGATAAATACCCGCTCTGG | |
| | TATTTGGGGCGAGGTACTATAAATACCCTTAGAGGTATTTTAT | |
| | CTTGGCAGCTAGGTCGGGATCTTGCAGCTGTCAGGGGAGGGG | |
| | AGGCGGGGGCTGATGTCAGGAGGGATACAAATAGTGCCGAC | |
| | GGCTGGGGGCCCTGTCTCCCCTCGCCGCATCCACTCTCCGGCC | |
| | GGCCGCCTGCCCGCCGCCTCCTCCGTGCGCCCGCCAGCCTCG | |
| | CCCGCGCCGTCACC (SEQ ID NO: 291) | |
| SP0378 | GGGCCCCACAGCAGCTGGGGGCATTTATGGGCCTTCCTATAA | 522 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTTACTAAAAATAGA | |
| | ACGACTATTTTTAGGCTTTTCTGGCAGCTGGCCCTGCCAGACA | |
| | GAGTTCCTCAGTAACGGGATCTTGCAGCTGTCAGGGGAGGGG | |
| | AGGCGGGGGCTGATGTCAGGAGGGATACAAATAGTGCCGAC | |
| | GGCTGGGGGCCCTGTCTCCCCTCGCCGCATCCACTCTCCGGCC | |
| | GGCCGCCTGCCCGCCGCCTCCTCCGTGCGCCCGCCAGCCTCG | |
| | CCCGCGCCGTCACC (SEQ ID NO: 292) | |
| SP0379 | GGGCCCCACAGCAGCTGGGGGCATTTATGGGCCTTCCTATAA | 524 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCGAGGTACTATAA | |
| | ATACCCTTAGAGGTATTTTATCTTGGCAGCTAGGTCTGCCAGA | |
| | CAGAGTTCCTCAGTAACGGGATCTTGCAGCTGTCAGGGGAGG | |
| | GGAGGCGGGGGCTGATGTCAGGAGGGATACAAATAGTGCCG | |
| | ACGGCTGGGGGCCCTGTCTCCCCTCGCCGCATCCACTCTCCGG | |
| | CCGGCCGCCTGCCCGCCGCCTCCTCCGTGCGCCCGCCAGCCTC | |
| | GCCCGCGCCGTCACC (SEQ ID NO: 293) | |
| SP0380 | GGGCCCCACAGCAGCTGGGGGCATTTATGGGCCTTCCTATAA | 522 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTTACTAAAAATAGA | |
| | ACGACTATTTTTAGGCTTTTCTGGCAGCTGGCCCTGCCAGACA | |
| | GATAAACGAGCTATCGGGATCTTGCAGCTGTCAGGGGAGGGG | |
| | AGGCGGGGGCTGATGTCAGGAGGGATACAAATAGTGCCGAC | |
| | GGCTGGGGGCCCTGTCTCCCCTCGCCGCATCCACTCTCCGGCC | |
| | GGCCGCCTGCCCGCCGCCTCCTCCGTGCGCCCGCCAGCCTCG | |
| | CCCGCGCCGTCACC (SEQ ID NO: 294) | |
| SP0381 | GGGCCCCACAGCAGCTGGGGGCATTTATGGGCCTTCCTATAA | 524 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCGAGGTACTATAA | |
| | ATACCCTTAGAGGTATTTTATCTTGGCAGCTAGGTCTGCCAGA | |
| | CAGATAAACGAGCTATCGGGATCTTGCAGCTGTCAGGGGAGG | |
| | GGAGGCGGGGGCTGATGTCAGGAGGGATACAAATAGTGCCG | |
| | ACGGCTGGGGGCCCTGTCTCCCCTCGCCGCATCCACTCTCCGG | |
| | CCGGCCGCCTGCCCGCCGCCTCCTCCGTGCGCCCGCCAGCCTC | |
| | GCCCGCGCCGTCACC (SEQ ID NO: 295) | |
| SP0382 | GGGCCCCACAGCAGCTGGGGGCATTTATGGGCCTTCCTATAA | 524 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTTAAACGAGCTATT | |
| | AGTTATGAGGTCCGTAGATTGAATAAACGAGCTATTAGTTAT | |
| | GAGGTCCGTAGATTGAACGGGATCTTGCAGCTGTCAGGGGAG | |
| | GGGAGGCGGGGGCTGATGTCAGGAGGGATACAAATAGTGCC | |
| | GACGGCTGGGGGCCCTGTCTCCCCTCGCCGCATCCACTCTCCG | |
| | GCCGGCCGCCTGCCCGCCGCCTCCTCCGTGCGCCCGCCAGCC | |
| | TCGCCCGCGCCGTCACC (SEQ ID NO: 296) | |
| SKM_14 | TTCTCCTCTATAAATACCCGCTCTGGTATTTGGGGTTGGCAGC | 240 |
| | TGTTGCTGCCAGGGAGATGGTTGGGTTGACGGGATCTTGCAG | |
| | CTGTCAGGGGAGGGGAGGCGGGGGCTGATGTCAGGAGGGAT | |
| | ACAAATAGTGCCGACGGCTGGGGGCCCTGTCTCCCCTCGCCG | |
| | CATCCACTCTCCGGCCGGCCGCCTGCCCGCCGCCTCCTCCGTG | |
| | CGCCCGCCAGCCTCGCCCGCGCCGTCACC (SEQ ID NO: 297) | |
| SKM 18 | ATAAATACCCGCTCTGGTATTTGGGGTTCTCCTCTATAAATAC | 242 |
| | CCGCTCTGGTATTTGGGGTTGGCAGCTGTTGCGGGATCTTGCA | |
| | GCTGTCAGGGGAGGGGAGGCGGGGGCTGATGTCAGGAGGGA | |
| | TACAAATAGTGCCGACGGCTGGGGGCCCTGTCTCCCCTCGCC | |
| | GCATCCACTCTCCGGCCGGCCGCCTGCCCGCCGCCTCCTCCGT | |
| | GCGCCCGCCAGCCTCGCCCGCGCCGTCACC (SEQ ID NO: 55) | |
| SKM 20 | ATTTTTAAAGACTGAGGAATTAGGCACCTGTCATTTTTGCCAG | 232 |
| | CTGGTGTAGATGTTAAAAATTACTGTCACTCTTCCGCCTGCTA | |
| | CTTTATTTTGCACCTGCTGTTACTTGAGTTACAGGCATTTCAC | |
| | ACATGGTAATTTAATAAGGTTAGTTCCCATGACACACCGCCT | |
| | GCTGCCACGGCCGGCCGTATAAATAGAGGCGAGGAGCAGCT | |
| | GGGCTCTCTTGGCAGTCACC (SEQ ID NO: 56) | |
| SP0357 | TCTGAGGGAGACAGGGAGGCATGATCACTGCCAAATGCCCAC | 335 |
| | CAAGGACAAGGCACATCCCAGGGAGACAGACGCAGACCTGG | |
| | TGCCCTCTGGACACTGGCATTCCTGGAGCCCGGCAGACGCTC | |
| | CTTATACGGCCCGGCCTCGCTCACCTGGGCCGCGGCCAGGAG | |
| | CGCCTTCTTTGGGCAGCGCCGGGCCGGGGCCGCGCCGGGCCC | |
| | GACACCCAAATATGGCGACGGCCGGGGCCGCATTCCTGGGGG | |
| | CCGGGCGGCGCTCCCGCCCGCCTCGATAAAAGGCTCCGGGGC | |
| | CGGCGGCGGCCCACGAGCTACCCGGAGGAGCGGGAGGCCAC | |
| | C (SEQ ID NO: 298) | |
| SP0437 | CACCGCGGTGGCGGCCGTCCGCCCTCGGATAGCTCGTTTAGA | 340 |
| | CACCCAAATATGGCGACGGTAAACGAGCTATTGGGAGTTATT | |
| | TTTAGAGCGTAAACGAGCTATTAGTTGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTCGGCCGGGGCCGCATTCCTGGGG | |
| | GCCGGGCGGTGCTCCCGCCCGCCTCGATAAAAGGCTCCGGGG | |
| | CCGGCGGCGGCCCACGAGCTACCCGGAGGAGCGGGAGGCGG | |
| | CCACC (SEQ ID NO: 299) | |
| SP0438 | CACCGCGGTGGCGGCCGTCCGCCCTCGGATAGCTCGTTTAGA | 365 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCGGCCGGGGCCGCATTCCTGGG | |
| | GGCCGGGCGGTGCTCCCGCCCGCCTCGATAAAAGGCTCCGGG | |
| | GCCGGCGGCGGCCCACGAGCTACCCGGAGGAGCGGGAGGCG | |
| | CCAAGCTCTAGAACTAGTGGATCCCGCCACC (SEQ ID NO: | |
| | 300) | |
| SP0439 | CACCGCGGTGGCGGCCGTCCGCCCTCGGCACCATCCTCACGA | 365 |
| | CACCCAAATATGGCGACGGTAAACGAGCTATTGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCGGCCGGGGCCGCATTCCTGGG | |
| | GGCCGGGCGGTGCTCCCGCCCGCCTCGATAAAAGGCTCCGGG | |
| | GCCGGCGGCGGCCCACGAGCTACCCGGAGGAGCGGGAGGCG | |
| | CCAAGCTCTAGAACTAGTGGATCCCGCCACC (SEQ ID NO: | |
| | 301) | |
| SP0440 | GGGCCCCACAGCAGCTGGGGGCATTTATGGGCCTTCCTATAA | 585 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTACACCCAAATATG | |
| | GCGACGGGTGAGGAATGGTGGGGAGTTATTTTTAGAGCGGTG | |
| | AGGAAGGTGGGCAGGCAGCAGGTGTTGGCGCTCTAAAAATA | |
| | ACTCCCGGGAGTTATTTTTAGAGCGGAGGAATGGTGGACACC | |
| | CAAATATGGCGACGGTTCCTCACCCGTCGCCATATTTGGGTGT | |
| | CCGCCCTCGGCCGGGGCCGCATTCCTGGGGGCCGGGCGGTGC | |
| | TCCCGCCCGCCTCGATAAAAGGCTCCGGGGCCGGCGGCGGCC | |
| | CACGAGCTACCCGGAGGAGCGGGAGGCGCCGCCACC (SEQ ID | |
| | NO: 302) | |
| SP0441 | GGGCCCCACAGCAGCTGGGGGCATTTATGGGCCTTCCTATAA | 546 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTACACCCAAATATG | |
| | GCGACGGGTGAGGAATGGTGGGGAGTTATTTTTAGAGCGGTG | |
| | AGGAAGGTGGGCAGGCAGCAGGTGTTGGCGCTCTAAAAATA | |
| | ACTCCCGGGAGTTATTTTTAGAGCGCCCGTCGCCATATTTGGG | |
| | TGTCCGCCCTCGGCCGGGGCCGCATTCCTGGGGGCCGGGCGG | |
| | TGCTCCCGCCCGCCTCGATAAAAGGCTCCGGGGCCGGCGGCG | |
| | GCCCACGAGCTACCCGGAGGAGCGGGAGGCGCCGCCACC | |
| | (SEQ ID NO: 303) | |
| SP0442 | GGGCCCCACAGCAGCTGGGGGCATTTATGGGCCTTCCTATAA | 585 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTACACCCAAATATG | |
| | GCGACGGGTGAGGAATGGTGGGGAGTTATTTTTAGAGCGGTG | |
| | AGGAAGGTGGGCAGGCAGCAGGTGTTGGCGCTCTAAAAATA | |
| | ACTCCCGGGAGTTATTTTTAGAGCGAGCTCTATAAATACCCG | |
| | CTCTGGTATTTGGGGTTTTGAACCCGTCGCCATATTTGGGTGT | |
| | CCGCCCTCGGCCGGGGCCGCATTCCTGGGGGCCGGGCGGTGC | |
| | TCCCGCCCGCCTCGATAAAAGGCTCCGGGGCCGGCGGCGGCC | |
| | CACGAGCTACCCGGAGGAGCGGGAGGCGCCGCCACC (SEQ ID | |
| | NO: 304) | |
| SP0443 | GGCCGTCCGCCCTCGGCACCATCCTCACGACACCCAAATATG | 328 |
| | GCGACGGGTGAGGAATGGTGGGGAGCTATTTTTAGAGCGTAA | |
| | ACGAGCTATTAGTTGCAGCAGGTGTTGGCGCTCTAAAAATAG | |
| | CTCCCGGGAGCTATTTTTAGAGCGGAGGAATGGTGGACACCC | |
| | AAATATGGCGACGGTTCCTCACCCGTCGCCATATTTGGGTGTC | |
| | CGCCCTCGGCCGGGGCCGCATTCCTGGGGGCCGGGCGGTGCT | |
| | CCCGCCCGCCTCGATAAAAGGCTCCGGGGCCGGCGGCGGCCC | |
| | ACGAGCTACCCGGAGGAGCGGGAGGCGGCCACC (SEQ ID | |
| | NO: 305) | |
| SP0444 | GGCCGTCCGCCCTCGGCACCATCCTCACGACACCCAAATATG | 328 |
| | GCGACGGGTGAGGAATGGTGGGGAGCTATTTTTAGAGCGTAA | |
| | ACGAGCTATTAGTTGCAGCAGGTGTTGGCGCTCTAAAAATAG | |
| | CTCCCGGGAGCTATTTTTAGAGCGAGCTCTATAAATACCCGCT | |
| | CTGGTATTTGGGGTTTTGAACCCGTCGCCATATTTGGGTGTCC | |
| | GCCCTCGGCCGGGGCCGCATTCCTGGGGGCCGGGCGGTGCTC | |
| | CCGCCCGCCTCGATAAAAGGCTCCGGGGCCGGCGGCGGCCCA | |
| | CGAGCTACCCGGAGGAGCGGGAGGCGGCCACC (SEQ ID NO: | |
| | 306) | |
| SP0445 | ACACCCAAATATGGCGACGGGTGAGGAATGGTGGGGAGTTA | 436 |
| | TTTTTAGAGCGGTGAGGAAGGTGGGCAGGCAGCAGGTGTTGG | |
| | CGCTCTAAAAATAACTCCCGGGAGTTATTTTTAGAGCGAGCT | |
| | CTATAAATACCCGCTCTGGTATTTGGGGTTTTGAACCCGTCGC | |
| | CATATTTGGGTGTCCGCCCTATAAATACCCGCTCTGGTATTTG | |
| | GGGTTCTCCTCTATAAATACCCGCTCTGGTATTTGGGGTTGGC | |
| | AGCTGTTGCGGGATCTTGCAGCTGTCAGGGGAGGGGAGGCGG | |
| | GGGCTGATGTCAGGAGGGATACAAATAGTGCCGACGGCTGG | |
| | GGGCCCTGTCTCCCCTCGCCGCATCCACTCTCCGGCCGGCCGC | |
| | CTGCCCGCCGCCTCCTCCGTGCGCCCGCCAGCCTCGCCCGCGC | |
| | CGTCACCGCCACC (SEQ ID NO: 307) | |
| SP0447 | GGCCGTCCGCCCTCGGGACACCCAAATATGGCGACGGGGGA | 291 |
| | GTTATTTTTAGAGCGGGCAGGCAGCAGGTGTTGGCGCTCTAA | |
| | AAATAACTCCCGGGAGTTATTTTTAGAGCGGAGGAATGGTGG | |
| | ACACCCAAATATGGCGACGGTTCCTCACCCGTCGCCATATTT | |
| | GGGTGTCCGCCCTCGGCCGGGGCCGCATTCCTGGGGGCCGGG | |
| | CGGTGCTCCCGCCCGCCTCGATAAAAGGCTCCGGGGCCGGCG | |
| | GCGGCCCACGAGCTACCCGGAGGAGCGGGAGGCGGCCACC | |
| | (SEQ ID NO: 308) | |
| SP0453 | GGGCCCCACAGCAGCTGGGGGCATTTATGGGCCTTCCTATAA | 761 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTATAAATACCCGCTCTGGTATTTGGGGTTCTCCTCTAT | |
| | AAATACCCGCTCTGGTATTTGGGGTTGGCAGCTGTTGCGGGA | |
| | TCTTGCAGCTGTCAGGGGAGGGGAGGCGGGGGCTGATGTCAG | |
| | GAGGGATACAAATAGTGCCGACGGCTGGGGGCCCTGTCTCCC | |
| | CTCGCCGCATCCACTCTCCGGCCGGCCGCCTGCCCGCCGCCTC | |
| | CTCCGTGCGCCCGCCAGCCTCGCCCGCGCCGTCACCGCCACC | |
| | (SEQ ID NO: 309) | |
| SP0454 | GGGCCCCACAGCAGCTGGGGGCATTTATGGGCCTTCCTATAA | 720 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTACACCCAAATATG | |
| | GCGACGGGTGAGGAATGGTGGGGAGTTATTTTTAGAGCGGTG | |
| | AGGAAGGTGGGCAGGCAGCAGGTGTTGGCGCTCTAAAAATA | |
| | ACTCCCGGGAGTTATTTTTAGAGCGAGCTCTATAAATACCCG | |
| | CTCTGGTATTTGGGGTTTTGAACCCGTCGCCATATTTGGGTGT | |
| | CCGCCCTATAAATACCCGCTCTGGTATTTGGGGTTCTCCTCTA | |
| | TAAATACCCGCTCTGGTATTTGGGGTTGGCAGCTGTTGCGGG | |
| | ATCTTGCAGCTGTCAGGGGAGGGGAGGCGGGGGCTGATGTCA | |
| | GGAGGGATACAAATAGTGCCGACGGCTGGGGGCCCTGTCTCC | |
| | CCTCGCCGCATCCACTCTCCGGCCGGCCGCCTGCCCGCCGCCT | |
| | CCTCCGTGCGCCCGCCAGCCTCGCCCGCGCCGTCACCGCCAC | |
| | C (SEQ ID NO: 310) | |
| SP0455 | CCACAGCAGCTGGGGGCATTTATGGGCCTTCCTATAAACTTCT | 551 |
| | GAGAGGGTAACTTTATCCTGCTTCTTTCAGCCAAGTATCCTCC | |
| | TCCAAAACCCGTGACTCACAGCACAGCCAGTGTGGGGGAGG | |
| | GGGTGGCTGCCTCCAATACGTGGCGCCCAGAGTCAGCTGTTC | |
| | TGGGGCCTTCTCTGGTTTCTCCAACTGAGTCCTGAGGTTTGGC | |
| | ACCGCGGTGGCGGCCGTCCGCCCTCGGCACCATCCTCACGAC | |
| | ACCCAAATATGGCGACGGGTGAGGAATGGTGGGGAGTTATTT | |
| | TTAGAGCGGTGAGGAAGGTGGGCAGGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTCGGCCGGGGCCGCATTCCTGGGG | |
| | GCCGGGCGGTGCTCCCGCCCGCCTCGATAAAAGGCTCCGGGG | |
| | CCGGCGGCGGCCCACGAGCTACCCGGAGGAGCGGGAGGCGG | |
| | CCACC (SEQ ID NO: 311) | |
| SP0456 | CCACAGCAGCTGGGGGCATTTATGGGCCTTCCTATAAACTTCT | 688 |
| | GAGAGGGTAACTTTATCCTGCTTCTTTCAGCCAAGTATCCTCC | |
| | TCCAAAACCCGTGACTCACAGCACAGCCAGTGTGGGGGAGG | |
| | GGGTGGCTGCCTCCAATACGTGGCGCCCAGAGTCAGCTGTTC | |
| | TGGGGCCTTCTCTGGTTTCTCCAACTGAGTCCTGAGGTTTGGC | |
| | ACCGCGGTGGCGGCCGTCCGCCCTCGGCACCATCCTCACGAC | |
| | ACCCAAATATGGCGACGGGTGAGGAATGGTGGGGAGTTATTT | |
| | TTAGAGCGGTGAGGAAGGTGGGCAGGCAGCAGGTGTTGGCG | |
| | CTCTAAAAATAACTCCCGGGAGTTATTTTTAGAGCGGAGGAA | |
| | TGGTGGACACCCAAATATGGCGACGGTTCCTCACCCGTCGCC | |
| | ATATTTGGGTGTCCGCCCTATAAATACCCGCTCTGGTATTTGG | |
| | GGTTCTCCTCTATAAATACCCGCTCTGGTATTTGGGGTTGGCA | |
| | GCTGTTGCGGGATCTTGCAGCTGTCAGGGGAGGGGAGGCGGG | |
| | GGCTGATGTCAGGAGGGATACAAATAGTGCCGACGGCTGGG | |
| | GGCCCTGTCTCCCCTCGCCGCATCCACTCTCCGGCCGGCCGCC | |
| | TGCCCGCCGCCTCCTCCGTGCGCCCGCCAGCCTCGCCCGCGCC | |
| | GTCACCGCCACC (SEQ ID NO: 312) | |
| SP0457 | CCCCACAGCAGCTGGGGGCATTTATGGGCCTTCCTATAAACT | 621 |
| | TCTGAGAGGGTAACTTTATCCTGCTTCTTTCAGCCAAGTATCC | |
| | TCCTCCAAGGCAGTGTATACTCTTCCATAAACGAGCTATTAGT | |
| | TATGAGGTCAAACCCGTGACTCACAGCACAGCCAGTGTGGGG | |
| | GAGGGGGTGGCTGCCTCCAATACGTGGCGCCCAGAGTCAGCT | |
| | GTTCTGGGGCCTTCTCTGGTTTCTCCAACTGAGTCCTGAGGTT | |
| | TGGTGACGGAATTCGGCCGAACGGGACACCGCGGTGGCGGC | |
| | CGTCCGCCCTCGGCACCATCCTCACGACACCCAAATATGGCG | |
| | ACGGGTGAGGAATGGTGGGGAGTTATTTTTAGAGCGGTGAGG | |
| | AAGGTGGGCAGGCAGCAGGTGTTGGCGCTCTAAAAATAACTC | |
| | CCGGGAGTTATTTTTAGAGCGGAGGAATGGTGGACACCCAAA | |
| | TATGGCGACGGTTCCTCACCCGTCGCCATATTTGGGTGTCCGC | |
| | CCTCGGCCGGGGCCGCATTCCTGGGGGCCGGGCGGTGCTCCC | |
| | GCCCGCCTCGATAAAAGGCTCCGGGGCCGGCGGCGGCCCACG | |
| | AGCTACCCGGAGGAGCGGGAGGCGGCCACC (SEQ ID NO: | |
| | 313) | |
| SP0458 | GGGCCCCACAGCAGCTGGGGGCATTTATGGGCCTTCCTATAA | 759 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTTTCTCCTCTATAAATACCCGCTCTGGTATTTGGGGTT | |
| | GGCAGCTGTTGCTGCCAGGGAGATGGTTGGGTTGACGGGATC | |
| | TTGCAGCTGTCAGGGGAGGGGAGGCGGGGGCTGATGTCAGG | |
| | AGGGATACAAATAGTGCCGACGGCTGGGGGCCCTGTCTCCCC | |
| | TCGCCGCATCCACTCTCCGGCCGGCCGCCTGCCCGCCGCCTCC | |
| | TCCGTGCGCCCGCCAGCCTCGCCCGCGCCGTCACCGCCACC | |
| | (SEQ ID NO: 314) | |
| SP0459 | GGGCCCCACAGCAGCTGGGGGCATTTATGGGCCTTCCTATAA | 837 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCATGTTCCCGGCGAAGGGCCAGCTGTCCCCCGCCAG | |
| | CTAGACTCAGCACTTAGTTTAGGAACCAGTGAGCAAGTCAGC | |
| | CCTTGGGGCAGCCCATACAAGGCCATGGGGCTGGGCAAGCTG | |
| | CACGCCTGGGTCCGGGGTGGGCACGGTGCCCGGGCAACGAG | |
| | CTGAAAGCTCATCTGCTCTCAGGGGCCCCTCCCTGGGGACAG | |
| | CCCCTCCTGGCTAGTCACACCCTGTAGGCTCCTCTATATAACC | |
| | CAGGGGCACAGGGGCTGCCCTCATTCTACCACCACCTCCACA | |
| | GCACAGACAGACACTCAGGAGCCAGCCAGCGCCACC (SEQ ID | |
| | NO: 315) | |
| SP0460 | CACCGCGGTGGCGGCCGTCCGCCCTCGGCACCATCCTCACGA | 298 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGCGGCC | |
| | GGGGCCGCATTCCTGGGGGCCGGGCGGTGCTCCCGCCCGCCT | |
| | CGATAAAAGGCTCCGGGGCCGGCGGCGGCCCACGAGCTACC | |
| | CGGAGGAGCGGGAGGCGCCAAGCTCTAGAACTAGTGGATCC | |
| | CGCCACC (SEQ ID NO: 316) | |
| SP0461 | CACCGCGGTGGCGGCCGTCCGCCCTCGGCACCATCCTCACGA | 365 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGAGGCA | |
| | GTGTATACTCTTCCATAAACGAGCTATTAGTTATGAGGTCCGT | |
| | AGATTGAAAAGGGTGACGGCGGCCGGGGCCGCATTCCTGGG | |
| | GGCCGGGCGGTGCTCCCGCCCGCCTCGATAAAAGGCTCCGGG | |
| | GCCGGCGGCGGCCCACGAGCTACCCGGAGGAGCGGGAGGCG | |
| | CCAAGCTCTAGAACTAGTGGATCCCGCCACC (SEQ ID NO: | |
| | 317) | |
| SP0462 | CTCTATAAATACCCGCTCTGGTATTTGGGGTTACACCCAAATA | 356 |
| | TGGCGACGGGTGAGGAATGGTGGGGAGTTATTTTTAGAGCGG | |
| | TGAGGAAGGTGGGCAGGCAGCAGGTGTTGGCGCTCTAAAAA | |
| | TAACTCCCGGGAGTTATTTTTAGAGCGGAGGAATGGTGGACA | |
| | CCCAAATATGGCGACGGTTCCTCACCCGTCGCCATATTTGGGT | |
| | GTCCGCCCTCGGCCGGGGCCGCATTCCTGGGGGCCGGGCGGT | |
| | GCTCCCGCCCGCCTCGATAAAAGGCTCCGGGGCCGGCGGCGG | |
| | CCCACGAGCTACCCGGAGGAGCGGGAGGCGCCAAGCTCTAG | |
| | AACTAGTGGATCCCGCCACC (SEQ ID NO: 318) | |
| SP0463 | GGGCCCCACAGCAGCTGGGGGCATTTATGGGCCTTCCTATAA | 772 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTCACCGCGGTGGCG | |
| | GCCGTCCGCCCTCGGCACCATCCTCACGACACCCAAATATGG | |
| | CGACGGGTGAGGAATGGTGGGGAGTTATTTTTAGAGCGGTGA | |
| | GGAAGGTGGGCAGGCAGCAGGTGTTGGCGCTCTAAAAATAA | |
| | CTCCCGGGAGTTATTTTTAGAGCGGAGGAATGGTGGACACCC | |
| | AAATATGGCGACGGTTCCTCACCCGTCGCCATATTTGGGTGTC | |
| | CGCCCTCCACTACGGGTCTAGGCTGCCCATGTAAGGAGGCAA | |
| | GGCCTGGGGACACCCGAGATGCCTGGTTATAATTAACCCAGA | |
| | CATGTGGCTGCCCCCCCCCCCCCAACACCTGCTGCCTCTAAAA | |
| | ATAACCCTGTCCCTGGTGGATCCCCTGCATGCGAAGATCTTCG | |
| | AACAAGGCTGTGGGGGACTGAGGGCAGGCTGTAACAGGCTT | |
| | GGGGGCCAGGGCTTATACGTGCCTGGGACTCCCAAAGTATTA | |
| | CTGTTCGCCACC (SEQ ID NO: 319) | |
| SP0464 | CACCGCGGTGGCGGCCGTCCGCCCTCGGCACCATCCTCACGA | 837 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTGGGCCCCACAGCAGCTGGGGGC | |
| | ATTTATGGGCCTTCCTATAAACTTCTGAGAGGGTAACTTTATC | |
| | CTGCTTCTTTCAGCCAAGTATCCTCCTCCAGCAGCTGGTCACA | |
| | AAGCTGGTTAATCTCCCAGAGTGCTCAGCTTAAAACCCGTGA | |
| | CTCACAGCACAGCCAGTGTGGGGGAGGGGGTGGCTGCCTCCA | |
| | ATACGTGGCGCCCAGAGTCAGCTGTTCTGGGGCCTTCTCTGGT | |
| | TTCTCCAACTGAGTCCTGAGGTTTGGGGCCTTGTCTTCCTTCC | |
| | TGGAGTCATGTTCCCGGCGAAGGGCCAGCTGTCCCCCGCCAG | |
| | CTAGACTCAGCACTTAGTTTAGGAACCAGTGAGCAAGTCAGC | |
| | CCTTGGGGCAGCCCATACAAGGCCATGGGGCTGGGCAAGCTG | |
| | CACGCCTGGGTCCGGGGTGGGCACGGTGCCCGGGCAACGAG | |
| | CTGAAAGCTCATCTGCTCTCAGGGGCCCCTCCCTGGGGACAG | |
| | CCCCTCCTGGCTAGTCACACCCTGTAGGCTCCTCTATATAACC | |
| | CAGGGGCACAGGGGCTGCCCTCATTCTACCACCACCTCCACA | |
| | GCACAGACAGACACTCAGGAGCCAGCCAGCGCCACC (SEQ ID | |
| | NO: 320) | |
| SP0465 | CACCGCGGTGGCGGCCGTCCGCCCTCGGCACCATCCTCACGA | 772 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTGGGCCCCACAGCAGCTGGGGGC | |
| | ATTTATGGGCCTTCCTATAAACTTCTGAGAGGGTAACTTTATC | |
| | CTGCTTCTTTCAGCCAAGTATCCTCCTCCAGCAGCTGGTCACA | |
| | AAGCTGGTTAATCTCCCAGAGTGCTCAGCTTAAAACCCGTGA | |
| | CTCACAGCACAGCCAGTGTGGGGGAGGGGGTGGCTGCCTCCA | |
| | ATACGTGGCGCCCAGAGTCAGCTGTTCTGGGGCCTTCTCTGGT | |
| | TTCTCCAACTGAGTCCTGAGGTTTGGGGCCTTGTCTTCCTTCC | |
| | TGGAGTCCACTACGGGTCTAGGCTGCCCATGTAAGGAGGCAA | |
| | GGCCTGGGGACACCCGAGATGCCTGGTTATAATTAACCCAGA | |
| | CATGTGGCTGCCCCCCCCCCCCCAACACCTGCTGCCTCTAAAA | |
| | ATAACCCTGTCCCTGGTGGATCCCCTGCATGCGAAGATCTTCG | |
| | AACAAGGCTGTGGGGGACTGAGGGCAGGCTGTAACAGGCTT | |
| | GGGGGCCAGGGCTTATACGTGCCTGGGACTCCCAAAGTATTA | |
| | CTGTTCGCCACC (SEQ ID NO: 321) | |
| SP0466 | CACCGCGGTGGCGGCCGTCCGCCCTCGGCACCATCCTCACGA | 764 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTCCACAGCAGCTGGGGGCATTTAT | |
| | GGGCCTTCCTATAAACTTCTGAGAGGGTAACTTTATCCTGCTT | |
| | CTTTCAGCCAAGTATCCTCCTCCAAAACCCGTGACTCACAGC | |
| | ACAGCCAGTGTGGGGGAGGGGGTGGCTGCCTCCAATACGTGG | |
| | CGCCCAGAGTCAGCTGTTCTGGGGCCTTCTCTGGTTTCTCCAA | |
| | CTGAGTCCTGAGGTTTGGCATGTTCCCGGCGAAGGGCCAGCT | |
| | GTCCCCCGCCAGCTAGACTCAGCACTTAGTTTAGGAACCAGT | |
| | GAGCAAGTCAGCCCTTGGGGCAGCCCATACAAGGCCATGGG | |
| | GCTGGGCAAGCTGCACGCCTGGGTCCGGGGTGGGCACGGTGC | |
| | CCGGGCAACGAGCTGAAAGCTCATCTGCTCTCAGGGGCCCCT | |
| | CCCTGGGGACAGCCCCTCCTGGCTAGTCACACCCTGTAGGCT | |
| | CCTCTATATAACCCAGGGGCACAGGGGCTGCCCTCATTCTAC | |
| | CACCACCTCCACAGCACAGACAGACACTCAGGAGCCAGCCA | |
| | GCGCCACC (SEQ ID NO: 322) | |
| SP0467 | GCCACTACGGGTCTAGGCTGCCCATGTAAGGAGGCAAGGCCT | 671 |
| | GGGGACACCCGAGATGCCTGGTTATAATTAACCCAGACATGT | |
| | GGCTGCCCCCCCCCCCCAACACCTGCTGCCTGAGCCTCACCCC | |
| | CACCCCGGTGCCTGGGTCTTAGGCTCTGTACACCATGGAGGA | |
| | GAAGCTCGCTCTAAAAATAACCCTGCACCGCGGTGGCGGCCG | |
| | TCCGCCCTCGGCACCATCCTCACGACACCCAAATATGGCGAC | |
| | GGGTGAGGAATGGTGGGGAGTTATTTTTAGAGCGGTGAGGAA | |
| | GGTGGGCAGGCAGCAGGTGTTGGCGCTCTAAAAATAACTCCC | |
| | GGGAGTTATTTTTAGAGCGGAGGAATGGTGGACACCCAAATA | |
| | TGGCGACGGTTCCTCACCCGTCGCCATATTTGGGTGTCCGCCC | |
| | TATAAATACCCGCTCTGGTATTTGGGGTTCTCCTCTATAAATA | |
| | CCCGCTCTGGTATTTGGGGTTGGCAGCTGTTGCGGGATCTTGC | |
| | AGCTGTCAGGGGAGGGGAGGCGGGGGCTGATGTCAGGAGGG | |
| | ATACAAATAGTGCCGACGGCTGGGGGCCCTGTCTCCCCTCGC | |
| | CGCATCCACTCTCCGGCCGGCCGCCTGCCCGCCGCCTCCTCCG | |
| | TGCGCCCGCCAGCCTCGCCCGCGCCGTCACCGCCACC (SEQ | |
| | ID NO: 323) | |
| SP0468 | CACCGCGGTGGCGGCCGTCCGCCCTCGGCACCATCCTCACGA | 671 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTGCCACTACGGGTCTAGGCTGCCC | |
| | ATGTAAGGAGGCAAGGCCTGGGGACACCCGAGATGCCTGGTT | |
| | ATAATTAACCCAGACATGTGGCTGCCCCCCCCCCCCAACACC | |
| | TGCTGCCTGAGCCTCACCCCCACCCCGGTGCCTGGGTCTTAGG | |
| | CTCTGTACACCATGGAGGAGAAGCTCGCTCTAAAAATAACCC | |
| | TGATAAATACCCGCTCTGGTATTTGGGGTTCTCCTCTATAAAT | |
| | ACCCGCTCTGGTATTTGGGGTTGGCAGCTGTTGCGGGATCTTG | |
| | CAGCTGTCAGGGGAGGGGAGGCGGGGGCTGATGTCAGGAGG | |
| | GATACAAATAGTGCCGACGGCTGGGGGCCCTGTCTCCCCTCG | |
| | CCGCATCCACTCTCCGGCCGGCCGCCTGCCCGCCGCCTCCTCC | |
| | GTGCGCCCGCCAGCCTCGCCCGCGCCGTCACCGCCACC (SEQ | |
| | ID NO: 324) | |
| SP0469 | CCACAGCAGCTGGGGGCATTTCTGAGAGGGTAACTTTATCCT | 506 |
| | GCTTCTTTCAGCCAAGTACTCACAGCACAGCCAGTGTGGGGG | |
| | AGGGGGTGGCTGCCTCCGTGGCGCCCAGAGTCAGCTGTTCTG | |
| | GGGCCTTCTCTGGTTTCTCCAACTGAGTCCTGAGGTTTGGCAC | |
| | CGCGGTGGCGGCCGTCCGCCCTCGGCACCATCCTCACGACAC | |
| | CCAAATATGGCGACGGGTGAGGAATGGTGGGGAGTTATTTTT | |
| | AGAGCGGTGAGGAAGGTGGGCAGGCAGCAGGTGTTGGCGCT | |
| | CTAAAAATAACTCCCGGGAGTTATTTTTAGAGCGGAGGAATG | |
| | GTGGACACCCAAATATGGCGACGGTTCCTCACCCGTCGCCAT | |
| | ATTTGGGTGTCCGCCCTCGGCCGGGGCCGCATTCCTGGGGGC | |
| | CGGGCGGTGCTCCCGCCCGCCTCGATAAAAGGCTCCGGGGCC | |
| | GGCGGCGGCCCACGAGCTACCCGGAGGAGCGGGAGGCGGCC | |
| | ACC (SEQ ID NO: 325) | |
| SP0470 | CACCGCGGTGGCGGCCGTCCGCCCTCGGCACCATCCTCACGA | 365 |
| | CACCCAAATATGGCGACGGGTGAGGAATGGTGGGGATAAAC | |
| | GAGCTATGCGGTGAGGAAGGTGGGCAGGCAGCAGGTGTTGG | |
| | CGCATAGCTCGTTTATCCCGGGATAAACGAGCTATGCGGAGG | |
| | AATGGTGGACACCCAAATATGGCGACGGTTCCTCACCCGTCG | |
| | CCATATTTGGGTGTCCGCCCTCGGCCGGGGCCGCATTCCTGG | |
| | GGGCCGGGCGGTGCTCCCGCCCGCCTCGATAAAAGGCTCCGG | |
| | GGCCGGCGGCGGCCCACGAGCTACCCGGAGGAGCGGGAGGC | |
| | GCCAAGCTCTAGAACTAGTGGATCCCGCCACC (SEQ ID NO: | |
| | 326) | |
| SP0471 | CACCGCGGTGGCGGCCGTCCGCCCTCGGCACCATCCTCACGA | 624 |
| | CACCCAAATATGGCGACGGGTGAGGAATGGTGGGGAGTTATT | |
| | TTTAGAGCGGTGAGGAAGGTGGGCAGGCAGCAGGTGTTGGC | |
| | GCTCTAAAAATAACTCCCGGGAGTTATTTTTAGAGCGGAGGA | |
| | ATGGTGGACACCCAAATATGGCGACGGTTCCTCACCCGTCGC | |
| | CATATTTGGGTGTCCGCCCTGGGCCCCACAGCAGCTGGGGGC | |
| | ATTTATGGGCCTTCCTATAAACTTCTGAGAGGGTAACTTTATC | |
| | CTGCTTCTTTCAGCCAAGTATCCTCCTCCAGCAGCTGGTCACA | |
| | AAGCTGGTTAATCTCCCAGAGTGCTCAGCTTAAAACCCGTGA | |
| | CTCACAGCACAGCCAGTGTGGGGGAGGGGGTGGCTGCCTCCA | |
| | ATACGTGGCGCCCAGAGTCAGCTGTTCTGGGGCCTTCTCTGGT | |
| | TTCTCCAACTGAGTCCTGAGGTTTGGGGCCTTGTCTTCCTTCC | |
| | TGGAGTCGGCCGGGGCCGCATTCCTGGGGGCCGGGCGGTGCT | |
| | CCCGCCCGCCTCGATAAAAGGCTCCGGGGCCGGCGGCGGCCC | |
| | ACGAGCTACCCGGAGGAGCGGGAGGCGGCCACC (SEQ ID | |
| | NO: 327) | |
| SP0473 | GGGCCCCACAGCAGCTGGGGGCATTTATGGGCCTTCCTATAA | 718 |
| | ACTTCTGAGAGGGTAACTTTATCCTGCTTCTTTCAGCCAAGTA | |
| | TCCTCCTCCAGCAGCTGGTCACAAAGCTGGTTAATCTCCCAG | |
| | AGTGCTCAGCTTAAAACCCGTGACTCACAGCACAGCCAGTGT | |
| | GGGGGAGGGGGTGGCTGCCTCCAATACGTGGCGCCCAGAGTC | |
| | AGCTGTTCTGGGGCCTTCTCTGGTTTCTCCAACTGAGTCCTGA | |
| | GGTTTGGGGCCTTGTCTTCCTTCCTGGAGTACACCCAAATATG | |
| | GCGACGGGTGAGGAATGGTGGGGAGTTATTTTTAGAGCGGTG | |
| | AGGAAGGTGGGCAGGCAGCAGGTGTTGGCGCTCTAAAAATA | |
| | ACTCCCGGGAGTTATTTTTAGAGCGAGCTCTATAAATACCCG | |
| | CTCTGGTATTTGGGGTTTTGAACCCGTCGCCATATTTGGGTGT | |
| | CCGCCCTTTCTCCTCTATAAATACCCGCTCTGGTATTTGGGGT | |
| | TGGCAGCTGTTGCTGCCAGGGAGATGGTTGGGTTGACGGGAT | |
| | CTTGCAGCTGTCAGGGGAGGGGAGGCGGGGGCTGATGTCAG | |
| | GAGGGATACAAATAGTGCCGACGGCTGGGGGCCCTGTCTCCC | |
| | CTCGCCGCATCCACTCTCCGGCCGGCCGCCTGCCCGCCGCCTC | |
| | CTCCGTGCGCCCGCCAGCCTCGCCCGCGCCGTCACCGCCACC | |
| | (SEQ ID NO: 328) | |
| SP0474 | CCACAGCAGCTGGGGGCATTTCTGAGAGGGTAACTTTATCCT | 465 |
| | GCTTCTTTCAGCCAAGTACTCACAGCACAGCCAGTGTGGGGG | |
| | AGGGGGTGGCTGCCTCCGTGGCGCCCAGAGTCAGCTGTTCTG | |
| | GGGCCTTCTCTGGTTTCTCCAACTGAGTCCTGAGGTTTGGACA | |
| | CCCAAATATGGCGACGGGTGAGGAATGGTGGGGAGTTATTTT | |
| | TAGAGCGGTGAGGAAGGTGGGCAGGCAGCAGGTGTTGGCGC | |
| | TCTAAAAATAACTCCCGGGAGTTATTTTTAGAGCGAGCTCTAT | |
| | AAATACCCGCTCTGGTATTTGGGGTTTTGAACCCGTCGCCATA | |
| | TTTGGGTGTCCGCCCTCGGCCGGGGCCGCATTCCTGGGGGCC | |
| | GGGCGGTGCTCCCGCCCGCCTCGATAAAAGGCTCCGGGGCCG | |
| | GCGGCGGCCCACGAGCTACCCGGAGGAGCGGGAGGCGGCCA | |
| | CC (SEQ ID NO: 329) | |

TABLE 8
Shortened Muscle-specific promoters active
in cardiac and skeletal muscle

| | | |
|---|---|---|
| SP0497 | TCTGAGGGAGACAGGGAGGCATGAT | 270 |
| (SEQ ID NO: 330) | CACTGCCAAATGCCCACCAAGGACA | |
| | AGGCACATCCCAGGGAGACAGACGC | |
| | AGACCTGGTGCCCTCTGGACACTGG | |
| | CATTCCTGGAGTTCTCCTCTATAAA | |
| | TACCCGCTCTGGTATTTGGGGTTGG | |
| | CAGCTGTTGCGGCCGGGGCCGCATT | |
| | CCTGGGGGCCGGGCGGTGCTCCCGC | |
| | CCGCCTCGATAAAAGGCTCCGGGGC | |
| | CGGCGGCGGCCCACGAGCTACCCGG | |
| | AGGAGCGGGAGGCGGCCACC | |
| SP0498 | TCTGAGGGAGACAGGGAGGCATGAT | 258 |
| (SEQ ID NO: 331) | CACTGCCAAATGCCCACCAAGGACA | |
| | AGGCACATCCCAGGGAGACAGACGC | |
| | AGACCTGGTGCCCTCTGGACACTGG | |
| | CATTCCTGGAGAGGGTCAGTGTCCT | |
| | GCCCCAACCCATGAGATGACAGACT | |
| | ATAATAGCCACAGGATTAACATAGC | |
| | AGGCATTGCACCGCCTGCTGCCACG | |
| | GCCGGCCGTATAAATAGAGGCGAGG | |
| | AGCAGCTGGGCTCTCTTGGCAGTCA | |
| | CCGCCACC | |
| SP0499 | TCTGAGGGAGACAGGGAGGCATGAT | 234 |
| (SEQ ID NO: 332) | CACTGCCAAATGCCCACCAAGGACA | |
| | AGGCACATCCCAGGGAGACAGACGC | |
| | AGACCTGGTGCCCTCTGGACACTGG | |
| | CATTCCTGGAGTTCTCCTCTATAAA | |
| | TACCCGCTCTGGTATTTGGGGTTGG | |
| | CAGCTGTTGCACCGCCTGCTGCCAC | |
| | GGCCGGCCGTATAAATAGAGGCGAG | |
| | GAGCAGCTGGGCTCTCTTGGCAGTC | |
| | ACCGCCACC | |
| SP0500 | CCAGCCCACCTGTCCCAATGCTGAC | 267 |
| (SEQ ID NO: 333) | TTAGTGCAAGGCGAGCCAGCAAGGA | |
| | GGGAGGACAGGTGGCAGTGGGGGGT | |
| | GAGGAGCATCTAAAAATAGCCCCAG | |
| | CCCACCTGTCCCAATGCTGACTTAG | |
| | TGCAAGGCGAGCCAGCAAGGAGGGA | |
| | GGACAGGTGGCAGTGGGGGGTGAGG | |
| | AGCATCTAAAAATAGCCCACCGCCT | |
| | GCTGCCACGGCCGGCCGTATAAATA | |
| | GAGGCGAGGAGCAGCTGGGCTCTCT | |
| | TGGCAGTCACCGCCACC | |
| SP0501 | CCAGCCCACCTGTCCCAATGCTGAC | 219 |
| (SEQ ID NO: 334) | TTAGTGCAAGGCGAGCCAGCAAGGA | |
| | GGGAGGACAGGTGGCAGTGGGGGGT | |
| | GAGGAGCATCTAAAAATAGCCTTCT | |
| | CCTCTATAAATACCCGCTCTGGTAT | |
| | TTGGGGTTGGCAGCTGTTGCACCGC | |
| | CTGCTGCCACGGCCGGCCGTATAAA | |
| | TAGAGGCGAGGAGCAGCTGGGCTCT | |
| | CTTGGCAGTCACCGCCACC | |
| SP0502 | CTAGACTAGCATGCTGCCCATGTAA | 251 |
| (SEQ ID NO: 335) | GGAGGCAAGGCCTGGGGACACCCGA | |
| | GATGCCTGGTTATAATTAACCCAGA | |
| | CATGTGGCTGCCCCCCCCCCCCCAA | |
| | CACCTGCTGCCTCTAAAAATAACCC | |
| | TGCTTCTCCTCTATAAATACCCGCT | |
| | CTGGTATTTGGGGTTGGCAGCTGTT | |
| | GCACCGCCTGCTGCCACGGCCGGCC | |
| | GTATAAATAGAGGCGAGGAGCAGCT | |
| | GGGCTCTCTTGGCAGTCACCGCCAC | |
| | C | |
| SP0503 | TCGTCCCCTGGCTGGCCCATGTAAT | 258 |
| (SEQ ID NO: 336) | CTGAGCCCAGCATTGTACATATCCT | |
| | GGGAACAGCTGACAATGCAGTGGTC | |
| | AGACAGCTGGTGGGGCCAGCTAGAG | |
| | CTGGCAGGGTTGGCTGGGAGGGGAG | |
| | TGTAGGCTGATTCTCCTCTATAAAT | |
| | ACCCGCTCTGGTATTTGGGGTTGGC | |
| | AGCTGTTGCACCGCCTGCTGCCACG | |
| | GCCGGCCGTATAAATAGAGGCGAGG | |
| | AGCAGCTGGGCTCTCTTGGCAGTCA | |
| | CCGCCACC | |
| SP0504 | TTGGCCCAGGTCACACTGGGGTGAG | 265 |
| (SEQ ID NO: 337) | GCTAGTGTTCCTGAGCCTTGACAAG | |
| | GAGACAGCTTGAAATAGACGAGTGT | |
| | CACATTTCTGAGCAGCTGTGTGGCG | |
| | ACAGCAGGAGGGGTAGGGAATAGAC | |
| | AGTATAAAAGAGAAAGCTTCTCCTC | |
| | TATAAATACCCGCTCTGGTATTTGG | |
| | GGTTGGCAGCTGTTGCACCGCCTGC | |
| | TGCCACGGCCGGCCGTATAAATAGA | |
| | GGCGAGGAGCAGCTGGGCTCTCTTG | |
| | GCAGTCACCGCCACC | |
| SP0505 | CCCTGCCATCTTGGGTTTCAGGGCA | 269 |
| (SEQ ID NO: 338) | GAGGAGTCTTGCTAATTTTGATGCC | |
| | TATTTTTGGACACTTCAGCTGCCAC | |
| | TGGCTCCTTATAAACGCATGACACC | |
| | CCATGCAAACACACTACCCCTCCCT | |
| | CCACTGCTGACAGGTGTGTGGTTCT | |
| | CCTCTATAAATACCCGCTCTGGTAT | |
| | TTGGGGTTGGCAGCTGTTGCACCGC | |
| | CTGCTGCCACGGCCGGCCGTATAAA | |
| | TAGAGGCGAGGAGCAGCTGGGCTCT | |
| | CTTGGCAGTCACCGCCACC | |
| SP0506 | AATTATTTTTAATAACACTTACTGG | 274 |
| (SEQ ID NO: 339) | TAAGAGAAAGGGGAGAAACCTTAGA | |
| | CAGGCACTTAGATGTGACTAAGGCA | |
| | GGTTTATCTCTGATTCCAAAGCACT | |
| | GGAGTGGAAGTCACACCGTGACTCA | |
| | GAGCATTGTGATGGGCCAGCTGTCC | |
| | ATTCTCCTCTATAAATACCCGCTCT | |
| | GGTATTTGGGGTTGGCAGCTGTTGC | |
| | ACCGCCTGCTGCCACGGCCGGCCGT | |
| | ATAAATAGAGGCGAGGAGCAGCTGG | |
| | GCTCTCTTGGCAGTCACCGCCACC | |
| SP0507 | CAGTGTTCCTATATTTATCCCACCA | 274 |
| (SEQ ID NO: 340) | TACAGAGCTTCCTTTGCCTCAGAAG | |
| | GACCAGCAGTTTCGCTAGCTTAACA | |
| | AAACCAGCCACTCAGGGTATTGGTT | |
| | TACAGTCAAGCAACTCTGGGAGAGG | |
| | GCAGCTGCTCTCAGACATCATACAG | |
| | CTTCTCCTCTATAAATACCCGCTCT | |
| | GGTATTTGGGGTTGGCAGCTGTTGC | |
| | ACCGCCTGCTGCCACGGCCGGCCGT | |
| | ATAAATAGAGGCGAGGAGCAGCTGG | |
| | GCTCTCTTGGCAGTCACCGCCACC | |
| SP0508 | CTTCTCAAGCCAAAGGAGCAAGAGT | 240 |
| (SEQ ID NO: 341) | TAAAAATAACAGGCTCACCCTGGCA | |
| | GCCACCTGTGCTGGCCAGCCCCACC | |
| | CCATCCCTCCCTCGGGGACAGCTGC | |
| | AGCTCCTCAGGCCCCGCCCGGGACA | |
| | TTTTGGGAACACTTTCTCCTCTTAC | |
| | TTCTCATCTTCAGGGCACCGCCTGC | |
| | TGCCACGGCCGGCCGTATAAATAGA | |
| | GGCGAGGAGCAGCTGGGCTCTCTTG | |
| | GCAGTCACCGCCACC | |
| SP0509 | AGGGTCAGTGTCCTGCCCCAACCCA | 275 |
| (SEQ ID NO: 342) | TGAGATGACAGACTATAATAGCCAC | |
| | AGGATTAACATAGCAGGCATTGATT | |
| | TTTAAAGACTGAGGAATTAGGCACC | |
| | TGTCATTTTTGCCAGCTGGTGTAGA | |
| | TGTTAAAAATTACTGTCACTCTTCC | |
| | GCCTGCTACTTTATTTTGCACCTGC | |
| | TGTTACTTGAGTTACAGGCATTTCA | |
| | CACCGCCTGCTGCCACGGCCGGCCG | |
| | TATAAATAGAGGCGAGGAGCAGCTG | |
| | GGCTCTCTTGGCAGTCACCGCCACC | |
| SP0510 | ATTTTTAAAGACTGAGGAATTAGGC | 251 |
| (SEQ ID NO: 343) | ACCTGTCATTTTTGCCAGCTGGTGT | |
| | AGATGTTAAAAATTACTGTCACTCT | |
| | TCCGCCTGCTACTTTATTTTGCACC | |
| | TGCTGTTACTTGAGTTACAGGCATT | |
| | TCATTCTCCTCTATAAATACCCGCT | |
| | CTGGTATTTGGGGTTGGCAGCTGTT | |
| | GCACCGCCTGCTGCCACGGCCGGCC | |
| | GTATAAATAGAGGCGAGGAGCAGCT | |
| | GGGCTCTCTTGGCAGTCACCGCCAC | |
| | C | |
| SP0511 | AGACTGGGGCAGGTGCAGGCTGGAT | 274 |
| (SEQ ID NO: 344) | TGGGTTTCCAGAGGCTATATATATA | |
| | AAGGCTGCCGGGAGCCCCAGGGCCG | |
| | CTCCCTGAGGGCACAACACTGTGGG | |
| | GGCCCAGCCAGGCCCACATTCCTTT | |
| | CCAGAGGCCAGCTCTCCATTTATAG | |
| | CCCCTGGGCAGAGCAGCTTCTCCTC | |
| | TATAAATACCCGCTCTGGTATTTGG | |
| | GGTTGGCAGCTGTTGGGGCTGGGCA | |
| | TAAAAGTCAGGGCAGAGCCATCTAT | |
| | TGCTTACATTTGCTTCTGGCCACC | |
| SP0512 | TCTGAGGGAGACAGGGAGGCATGAT | 218 |
| (SEQ ID NO: 345) | CACTGCCAAATGCCCACCAAGGACA | |
| | AGGCACATCCCAGGGAGACAGACGC | |
| | AGACCTGGTGCCCTCTGGACACTGG | |
| | CATTCCTGGAGTTCTCCTCTATAAA | |
| | TACCCGCTCTGGTATTTGGGGTTGG | |
| | CAGCTGTTGGGGCTGGGCATAAAAG | |
| | TCAGGGCAGAGCCATCTATTGCTTA | |
| | CATTTGCTTCTGGCCACC | |
| SP0513 | CCAGCCCACCTGTCCCAATGCTGAC | 275 |
| (SEQ ID NO: 346) | TTAGTGCAAGGCGAGCCAGCAAGGA | |
| | GGGAGGACAGGTGGCAGTGGGGGGT | |
| | GAGGAGCATCTAAAAATAGCCAGGG | |
| | TCAGTGTCCTGCCCCAACCCATGAG | |
| | ATGACAGACTATAATAGCCACAGGA | |
| | TTAACATAGCAGGCATTGTTCTCCT | |
| | CTATAAATACCCGCTCTGGTATTTG | |
| | GGGTTGGCAGCTGTTGGGGCTGGGC | |
| | ATAAAAGTCAGGGCAGAGCCATCTA | |
| | TTGCTTACATTTGCTTCTGGCCACC | |
| SP0514 | CCAGCCCACCTGTCCCAATGCTGAC | 251 |
| (SEQ ID NO: 347) | TTAGTGCAAGGCGAGCCAGCAAGGA | |
| | GGGAGGACAGGTGGCAGTGGGGGGT | |
| | GAGGAGCATCTAAAAATAGCCTTCT | |
| | CCTCTATAAATACCCGCTCTGGTAT | |
| | TTGGGGTTGGCAGCTGTTGTTCTCC | |
| | TCTATAAATACCCGCTCTGGTATTT | |
| | GGGGTTGGCAGCTGTTGGGGCTGGG | |
| | CATAAAAGTCAGGGCAGAGCCATCT | |
| | ATTGCTTACATTTGCTTCTGGCCAC | |
| | C | |
| SP0515 | CTAGACTAGCATGCTGCCCATGTAA | 235 |
| (SEQ ID NO: 348) | GGAGGCAAGGCCTGGGGACACCCGA | |
| | GATGCCTGGTTATAATTAACCCAGA | |
| | CATGTGGCTGCCCCCCCCCCCCCAA | |
| | CACCTGCTGCCTCTAAAAATAACCC | |
| | TGCTTCTCCTCTATAAATACCCGCT | |
| | CTGGTATTTGGGGTTGGCAGCTGTT | |
| | GGGGCTGGGCATAAAAGTCAGGGCA | |
| | GAGCCATCTATTGCTTACATTTGCT | |
| | TCTGGCCACC | |
| SP0516 | TCGTCCCCTGGCTGGCCCATGTAAT | 242 |
| (SEQ ID NO: 349) | CTGAGCCCAGCATTGTACATATCCT | |
| | GGGAACAGCTGACAATGCAGTGGTC | |
| | AGACAGCTGGTGGGGCCAGCTAGAG | |
| | CTGGCAGGGTTGGCTGGGAGGGGAG | |
| | TGTAGGCTGATTCTCCTCTATAAAT | |
| | ACCCGCTCTGGTATTTGGGGTTGGC | |
| | AGCTGTTGGGGCTGGGCATAAAAGT | |
| | CAGGGCAGAGCCATCTATTGCTTAC | |
| | ATTTGCTTCTGGCCACC | |
| SP0517 | TTGGCCCAGGTCACACTGGGGTGAG | 249 |
| (SEQ ID NO: 350) | GCTAGTGTTCCTGAGCCTTGACAAG | |
| | GAGACAGCTTGAAATAGACGAGTGT | |
| | CACATTTCTGAGCAGCTGTGTGGCG | |
| | ACAGCAGGAGGGGTAGGGAATAGAC | |
| | AGTATAAAAGAGAAAGCTTCTCCTC | |
| | TATAAATACCCGCTCTGGTATTTGG | |
| | GGTTGGCAGCTGTTGGGGCTGGGCA | |
| | TAAAAGTCAGGGCAGAGCCATCTAT | |
| | TGCTTACATTTGCTTCTGGCCACC | |
| SP0518 | CCCTGCCATCTTGGGTTTCAGGGCA | 253 |
| (SEQ ID NO: 351) | GAGGAGTCTTGCTAATTTTGATGCC | |
| | TATTTTTGGACACTTCAGCTGCCAC | |
| | TGGCTCCTTATAAACGCATGACACC | |
| | CCATGCAAACACACTACCCCTCCCT | |
| | CCACTGCTGACAGGTGTGTGGTTCT | |
| | CCTCTATAAATACCCGCTCTGGTAT | |
| | TTGGGGTTGGCAGCTGTTGGGGCTG | |
| | GGCATAAAAGTCAGGGCAGAGCCAT | |
| | CTATTGCTTACATTTGCTTCTGGCC | |
| | ACC | |
| SP0519 | CTTCTCAAGCCAAAGGAGCAAGAGT | 272 |
| (SEQ ID NO: 352) | TAAAAATAACAGGCTCACCCTGGCA | |
| | GCCACCTGTGCTGGCCAGCCCCACC | |
| | CCATCCCTCCCTCGGGGACAGCTGC | |
| | AGCTCCTCAGGCCCCGCCCGGGACA | |
| | TTTTGGGAACACTTTCTCCTCTTAC | |
| | TTCTCATCTTCAGGGTTCTCCTCTA | |
| | TAAATACCCGCTCTGGTATTTGGGG | |
| | TTGGCAGCTGTTGGGGCTGGGCATA | |
| | AAAGTCAGGGCAGAGCCATCTATTG | |
| | CTTACATTTGCTTCTGGCCACC | |
| SP0520 | AGGGTCAGTGTCCTGCCCCAACCCA | 272 |
| (SEQ ID NO: 353) | TGAGATGACAGACTATAATAGCCAC | |
| | AGGATTAACATAGCAGGCATTGATT | |
| | TTTAAAGACTGAGGAATTAGGCACC | |
| | TGTCATTTTTGCCAGCTGGTGTAGA | |
| | TGTTAAAAATTACTGTCACTCTTCC | |
| | GCCTGCTACTTTATTTTGCACCTGC | |
| | TGTTACTTGAGTTACAGGCATTTCA | |
| | GGGCTGGGCATAAAAGTCAGGGCAG | |
| | AGCCATCTATTGCTTACATTTGCTT | |
| | CTGGCCACC | |
| SP0521 | CTAGACTAGCATGCTGCCCATGTAA | 263 |
| (SEQ ID NO: 354) | GGAGGCAAGGCCTGGGGACACCCGA | |
| | GATGCCTGGTTATAATTAACCCAGA | |
| | CATGTGGCTGCCCCCCCCCCCCCAA | |
| | CACCTGCTGCCTCTAAAAATAACCC | |
| | TGCTTCTCCTCTATAAATACCCGCT | |
| | CTGGTATTTGGGGTTGGCAGCTGTT | |
| | GGTACTTATATAAGGGGGTGGGGGC | |
| | GCGTTCGTCCTCAGTCGCGATCGAA | |
| | CACTCGAGCCGAGCAGACGTGCCTA | |
| | CGGACCGGCCACC | |
| SP4169 | CTAGACTAGCATGCTGCCCATGTAA | 287 |
| (SEQ ID NO: 355) | GGAGGCAAGGCCTGGGGACACCCGA | |
| | GATGCCTGGTTATAATTAACCCAGA | |
| | CATGTGGCTGCCCCCCCCCCCCCAA | |
| | CACCTGCTGCCTCTAAAAATAACCC | |
| | TGCTTCTCCTCTATAAATACCCGCT | |
| | CTGGTATTTGGGGTTGGCAGCTGTT | |
| | GCGGCCGGGGCCGCATTCCTGGGGG | |
| | CCGGGCGGTGCTCCCGCCCGCCTCG | |
| | ATAAAAGGCTCCGGGGCCGGCGGCG | |
| | GCCCACGAGCTACCCGGAGGAGCGG | |
| | GAGGCGGCCACC | |
| SP0522 | GCTGCCCATGTAAGGAGGCAAGGCC | 237 |
| (SEQ ID NO: 356) | TGGGGACACCCGAGATGCCTGGTTA | |
| | TAATTAACCCAGACATGTGGCTGCC | |
| | CCCCCCCCCCAACACCTGCTGCCTC | |
| | TAAAAATAACCCTGTTCTCCTCTAT | |
| | AAATACCCGCTCTGGTATTTGGGGT | |
| | TGGCAGCTGTTGCACCGCCTGCTGC | |
| | CACGGCCGGCCGTATAAATAGAGGC | |
| | GAGGAGCAGCTGGGCTCTCTTGGCA | |
| | GTCACCGCCACC | |
| SP0523 | GCTGCCCATGTAAGGAGGCAAGGCC | 221 |
| (SEQ ID NO: 357) | TGGGGACACCCGAGATGCCTGGTTA | |
| | TAATTAACCCAGACATGTGGCTGCC | |
| | CCCCCCCCCCAACACCTGCTGCCTC | |
| | TAAAAATAACCCTGTTCTCCTCTAT | |
| | AAATACCCGCTCTGGTATTTGGGGT | |
| | TGGCAGCTGTTGGGGCTGGGCATAA | |
| | AAGTCAGGGCAGAGCCATCTATTGC | |
| | TTACATTTGCTTCTGGCCACC | |
| SP0524 | GCTGCCCATGTAAGGAGGCAAGGCC | 249 |
| (SEQ ID NO: 358) | TGGGGACACCCGAGATGCCTGGTTA | |
| | TAATTAACCCAGACATGTGGCTGCC | |
| | CCCCCCCCCCAACACCTGCTGCCTC | |
| | TAAAAATAACCCTGTTCTCCTCTAT | |
| | AAATACCCGCTCTGGTATTTGGGGT | |
| | TGGCAGCTGTTGGTACTTATATAAG | |
| | GGGGTGGGGGCGCGTTCGTCCTCAG | |
| | TCGCGATCGAACACTCGAGCCGAGC | |
| | AGACGTGCCTACGGACCGGCCACC | |

TABLE 2
CREs and other Elements found in the Promoters of Table 1

| | | | | | | 5′UTR |
|---|---|---|---|---|---|---|
| | | | | | Promoter | intron |
| Promoter | CRE | CRE | CRE | CRE | element | (SEQ ID NO) |
| (SEQ ID NO) | (SEQ ID NO) | (SEQ ID NO) | (SEQ ID NO) | (SEQ ID NO) | (SEQ ID NO) | and/or |
| SP0010 | | | | | CRE0010 | |
| (80) | | | | | (264) | |
| SP0020 | | | | CRE0020 | CRE0053.2 | |
| (81) | | | | (203) | SRL_mp | |
| | | | | | (271) | |
| SP0033 | | | | CRE0035 | CRE0053.2 | |
| (82) | | | | (208) | SRL_mp | |
| | | | | | (271) | |
| SP0038 | | | | CRE0031 | CRE0053.2 | |
| (83) | | | | (207) | SRL_mp | |
| | | | | | (271) | |
| SP0040 | | | | CRE0036 | CRE0053.2 | |
| (84) | | | | (209) | SRL_mp | |
| | | | | | (271) | |
| SP0042 | | | | CRE0036 | CRE0037 | |
| (85) | | | | (209) | (267) | |
| SP0051 | | | | CRE0020 | SKM_14 | |
| (86) | | | | (203) | (277) | |
| SP0057 | | | CRE0029 | CRE0071 | CRE0070 | |
| (87) | | | (206) | (216) | (42) | |
| SP0058 | | | | CRE0016 | CRE0005 | |
| (88) | | | | (201) | (262) | |
| SP0061 | | | | CRE0016 | SKM_18 | |
| (89) | | | | (201) | (55) | |
| SP0062 | | | | CRE0018 | SKM_18 | |
| (90) | | | | (202) | (55) | |
| SP0064 | | | | CRE0027 | SKM_18 | |
| (91) | | | | (205) | (55) | |
| SP0065 | | | | CRE0028 | SKM_18 | |
| (92) | | | | (40) | (55) | |
| SP0066 | | | | CRE0029 | SKM_18 | |
| (93) | | | | (206) | (55) | |
| SP0068 | | | | CRE0035 | SKM_18 | |
| (94) | | | | (208) | (55) | |
| SP0070 | | | | CRE0018 | SKM_20 | |
| (95) | | | | (202) | (56) | |
| SP0071 | | | | CRE0025 | SKM_20 | |
| (96) | | | | (204) | (56) | |
| SP0076 | | | | CRE0035 | SKM_20 | |
| (97) | | | | (208) | (56) | |
| SP0132 | | | | CRE0020 | SKM_18 | |
| (98) | | | | (203) | (55) | |
| SP0133 | | | | CRE0020 | SKM_20 | |
| (99) | | | | (203) | (56) | |
| SP0134 | | | CRE0020 | CRE0071 | CRE0070 | |
| (100) | | | (203) | (216) | (42) | |
| SP0136 | | | | CRE0020 | CRE0010 | |
| (101) | | | | (203) | (264) | |
| SP0146 | | | | CRE0050 | CRE0049 | HBB intron |
| (102) | | | | (211) | (270) | (283) |
| SP0147 | | | | CRE0020 | CRE0049 | HBB intron |
| (103) | | | | (203) | (270) | (283) |
| SP0148 | | | | CRE0020 | RSV (279) | |
| (104) | | | | (203) | | |
| SP0150 | | | | CRE0025 | RSV (279) | |
| (105) | | | | (204) | | |
| SP0153 | | | | CRE0035 | CRE0046 | |
| (106) | | | | (208) | (268) | |
| SP0155 | | CRE0035 | 48 bp (224) | 48 bp (224) | CRE0046 | |
| (107) | | (208) | | | (268) | |
| SP0156 | | | CRE0035 | CRE0020 | SKM_14 | |
| (108) | | | (208) | (203) | (277) | |
| SP0157 | | | | CRE0050 | CRE0053.2 | |
| (109) | | | | (211) | SRL_mp | |
| | | | | | (271) | |
| SP0158 | | | CRE0020 | CRE0036 | CRE0037 | |
| (110) | | | (203) | (209) | (267) | |
| SP0159 | | | CRE0035 | CRE0036 | CRE0037 | |
| (111) | | | (208) | (209) | (267) | |
| SP0160 | | | CRE0035 | CRE0031 | CRE0037 | |
| (112) | | | (208) | (207) | (267) | |
| SP0161 | | | CRE0020 | CRE0036 | CRE0009 | |
| (113) | | | (203) | (209) | (263) | |
| SP0162 | | | CRE0035 | CRE0036 | CRE0009 | |
| (114) | | | (208) | (209) | (263) | |
| SP0163 | | | CRE0035 | CRE0031 | CRE0009 | |
| (115) | | | (208) | (207) | (263) | |
| SP0164 | | | CRE0047 | CRE0020 | CRE0048 | |
| (116) | | | (210) | (203) | (269) | |
| SP0165 | | | | CRE0047 | CRE0048 | |
| (117) | | | | (210) | (269) | |
| SP0166 | | | | CRE0051 | RSV (279) | |
| (118) | | | | (212) | | |
| SP0169 | | | | | SKM_18 | |
| (119) | | | | | (55) | |
| SP0170 | | | | CRE0051 | SKM_18 | |
| (120) | | | | (212) | (55) | |
| SP0171 | | | | CRE0010 | SKM_18 | |
| (121) | | | | (264) | (55) | |
| SP0173 | | | CRE0010 | CRE0035 | SKM_18 | |
| (122) | | | (264) | (208) | (55) | |
| SP0228 | | CRE0020 | CRE0029 | CRE0071 | CRE0070 | |
| (123) | | (203) | (206) | (216) | (42) | |
| SP0229 | | CRE0020 | CRE0029 | CRE0071 | SKM_18 | |
| (124) | | (203) | (206) | (216) | (55) | |
| SP0230 | | CRE0020 | CRE0020 | CRE0071 | CRE0070 | |
| (125) | | (203) | (203) | (216) | (42) | |
| SP0231 | | | CRE0020 | CRE0071 | SKM_18 | |
| (126) | | | (203) | (216) | (55) | |
| SP0232 | | | CRE0035 | CRE0071 | SKM_18 | |
| (127) | | | (208) | (216) | (55) | |
| SP0257 | | | CRE0010 | CRE0035 | CRE0046 | |
| (128) | | | (264) | (208) | (268) | |
| SP0262 | | | CRE0010 | CRE0035 | CRE0054 | |
| (129) | | | (264) | (208) | (272) | |
| SP0264 | | | | CRE0035 | CRE0010 | |
| (130) | | | | (208) | (264) | |
| SP0265 | | | | CRE0010 | CRE0010_A | |
| (131) | | | | (264) | LDOA (265) | |
| SP0266 | | | CRE0010 | CRE0035 | CRE0010_A | |
| (132) | | | (264) | (208) | LDOA (265) | |
| SP0267 | | | CRE0033 | CRE0071 | CRE0070 | |
| (133) | | | (41) | (216) | (42) | |
| SP0268 | | | CRE0035 | CRE0010 | SKM_18 | |
| (134) | | | (208) | (264) | (55) | |
| SP0270 | | | CRE0035 | CRE0055 | DES_mp_v1 | |
| (135) | | | (208) | (273) | (280) | |
| SP0271 | | | | CRE0035 | CRE0056 | |
| (136) | | | | (208) | (274) | |
| SP0279 | | | CRE0020 | CRE0071 | CRE0070.2 | CMV-IE |
| (137) | | | (203) | (216) | (275) | (65) |
| SP0286 | | | | CRE0071 | CRE0070.2 | CMV-IE |
| (138) | | | | (216) | (275) | (65) |
| SP0305 | | | CRE0010 | CRE0035 | CRE0053.2 | |
| (139) | | | (264) | (208) | SRL_mp | |
| | | | | | (271) | |
| SP0306 | | | CRE0029 | CRE0035 | CRE0053.2 | |
| (140) | | | (206) | (208) | SRL_mp | |
| | | | | | (271) | |
| SP0307 | | | CRE0020 | CRE0035 | CRE0053.2 | |
| (141) | | | (203) | (208) | SRL_mp | |
| | | | | | (271) | |
| SP0309 | | | CRE0035 | CRE0035 | SKM_18 | |
| (142) | | | (208) | (208) | (55) | |
| SP0310 | | | | CRE0035 | SKM_18 | |
| (143) | | | | (208) | (55) | |
| SP0311 | | | CRE0035 | 48 bp (224) | CRE0053.2 | |
| (144) | | | (208) | | SRL_mp | |
| | | | | | (271) | |
| SP0312 | | | CRE0047 | CRE0035 | CRE0053.2 | |
| (145) | | | (210) | (208) | SRL_mp | |
| | | | | | (271) | |
| SP0313 | | | CRE0035 | CRE0059 | CRE0053.2 | |
| (146) | | | (208) | (213) | SRL_mp | |
| | | | | | (271) | |
| SP0314 | | CRE0035 | CRE0060 | CRE0060 | CRE0053.2 | |
| (147) | | (208) | (214) | (214) | SRL_mp | |
| | | | | | (271) | |
| SP0315 | | | | CRE0050 | CRE0053.2 | |
| (148) | | | | (211) | SRL_mp | |
| | | | | | (271) | |
| SP0316 | | | | CRE0050 | SKM_18 | |
| (149) | | | | (211) | (55) | |
| SP0320 | | | CRE0010 | CRE0035 | SKM_18 | CMV-IE |
| (150) | | | (264) | (208) | (55) | (65) |
| SP0322 | | | CRE0069 | CRE0051 | SKM_18 | |
| (151) | | | (215) | (212) | (55) | |
| SP0323 | | | CRE0069.2 | CRE0051 | SKM_18 | |
| (152) | | | (242) | (212) | (55) | |
| SP0324 | | | | CRE0069 | SKM_14 | |
| (153) | | | | (215) | (277) | |
| SP0325 | | | | CRE0069 | SKM_18 | |
| (154) | | | | (215) | (55) | |
| SP0326 | | | | CRE0071 | SKM_18 | |
| (155) | | | | (216) | (55) | |
| SP0327 | | | CRE0069 | CRE0071 | CRE0070 | |
| (156) | | | (215) | (216) | (42) | |
| SP0328 | | CRE0020 | CRE0069 | CRE0071 | CRE0070 | |
| (157) | | (203) | (215) | (216) | (42) | |
| SP0329 | | | | CRE0071.13 | CRE0070 | |
| (158) | | | | (243) | (42) | |
| SP0330 | | | | CRE0071.3 | CRE0070 | |
| (159) | | | | (43) | (42) | |
| SP0331 | | | | CRE0071.4 | CRE0070 | |
| (160) | | | | (236) | (42) | |
| SP0332 | | | CRE0035 | CRE0071 | CRE0070 | |
| (161) | | | (208) | (216) | (42) | |
| SP0333 | | | | CRE0035 | CRE0072 | |
| (162) | | | | (208) | (276) | |
| SP0334 | | | | CRE0035 | DES_mp_v1 | |
| (163) | | | | (208) | (280) | |
| SP0335 | | | CRE0035 | CRE0055 | CRE0034 | |
| (164) | | | (208) | (273) | (266) | |
| SP0336 | | | | CRE0055 | CRE0034 | |
| (165) | | | | (273) | (266) | |
| SP0337 | | | CRE0035 | CRE0055 | CRE0046 | |
| (166) | | | (208) | (273) | (268) | |
| SP0338 | | CRE0069 | CRE0035 | CRE0055 | DES_mp_v1 | |
| (167) | | (215) | (208) | (273) | (280) | |
| SP0339 | | CRE0035 | 48 bp (224) | CRE0055 | DES_mp_v1 | |
| (168) | | (208) | | (273) | (280) | |
| SP0340 | | | CRE0035 | CRE0046 | SKM_18 | |
| (169) | | | (208) | (268) | (55) | |
| SP0341 | | | CRE0035 | CRE0055 | CRE0010_A | |
| (170) | | | (208) | (273) | LDOA (265) | |
| SP0343 | | | | CRE0035 | SKM_18.2 | CMV-IE |
| (171) | | | | (208) | (278) | (65) |
| SP0345 | | | CRE0020 | CRE0071 | DES_mp_v1 | |
| (172) | | | (203) | (216) | (280) | |
| SP0346 | | | CRE0069 | CRE0071 | DES_mp_v1 | |
| (173) | | | (215) | (216) | (280) | |
| SP0347 | | | CRE0029 | CRE0050 | SKM_18 | |
| (174) | | | (206) | (211) | (55) | |
| SP0348 | | | CRE0029.2 | CRE0050 | SKM_18 | |
| (175) | | | (241) | (211) | (55) | |
| SP0349 | | CRE0029 | CRE0035 | CRE0071 | SKM_18 | |
| (176) | | (206) | (208) | (216) | (55) | |
| SP0350 | | CRE0020 | 72 bp (246) | CRE0071 | CRE0070 | |
| (177) | | (203) | | (216) | (42) | |
| SP0351 | | | | CRE0071.14 | CRE0070 | |
| (178) | | | | (244) | (42) | |
| SP0352 | | | | CRE0071.15 | CRE0070 | |
| (179) | | | | (245) | (42) | |
| SP0353 | | | | CRE0073 | CRE0046 | |
| (180) | | | | (218) | (268) | |
| SP0354 | | | | CRE0074 | CRE0046 | |
| (181) | | | | (219) | (268) | |
| SP0355 | | | | CRE0075 | CRE0046 | |
| (182) | | | | (220) | (268) | |
| SP0356 | | | | CRE0076 | CRE0046 | |
| (183) | | | | (221) | (268) | |
| SP0358 | | | | CRE0078 | CRE0046 | |
| (184) | | | | (222) | (268) | |
| SP0359 | | | | CRE0079 | CRE0046 | |
| (185) | | | | (223) | (268) | |
| SP0361 | | | | CRE0071.14 | SKM_18 | |
| (186) | | | | (244) | (55) | |
| SP0362 | | | CRE0069 | CRE0071.5 | DES_mp_v1 | |
| (187) | | | (215) | (217) | (280) | |
| SP0363 | | | CRE0029 | CRE0071.5 | DES_mp_v1 | |
| (188) | | | (206) | (217) | (280) | |
| SP0364 | | | CRE0029 | CRE0071 | CRE0046 | |
| (189) | | | (206) | (216) | (268) | |
| SP0365 | | | | CRE0071 | CRE0046 | |
| (190) | | | | (216) | (268) | |
| SP0366 | | | CRE0079 | CRE0071 | SKM_18 | |
| (191) | | | (223) | (216) | (55) | |
| SP0367 | | | | CRE0079 | CRE0034 | |
| (192) | | | | (223) | (266) | |
| SP0368 | | | CRE0079 | CRE0035 | SKM_18 | |
| (193) | | | (223) | (208) | (55) | |
| SP0369 | | | | CRE0071.6 | CRE0070 | |
| (194) | | | | (237) | (42) | |
| SP0370 | | | | CRE0071.7 | CRE0070 | |
| (195) | | | | (225) | (42) | |
| SP0371 | | | | CRE0071.8 | CRE0070 | |
| (196) | | | | (238) | (42) | |
| SP0372 | | | | CRE0071.9 | CRE0070 | |
| (197) | | | | (239) | (42) | |
| SP0373 | | | | CRE0071.10 | CRE0070 | |
| (198) | | | | (226) | (42) | |
| SP0374 | | | | CRE0071.11 | CRE0070 | |
| (199) | | | | (227) | (42) | |
| SP0375 | | | | CRE0071.12 | CRE0070 | |
| (200) | | | | (228) | (42) | |
| SP0376 | | CRE0035 | HTMB ev_4 | DES_MT_ | DES_mp_v1 | |
| (290) | | (208) | (282) | enhancer_48bp_ | (280) | |
| | | | | v2 (229) | | |
| SP0377 | | CRE0035 | HTMB ev_4 | DES_MT_ | DES_mp_v1 | |
| (291) | | (208) | (282) | enhancer_48bp_ | (280) | |
| | | | | 3 (230) | | |
| SP0378 | | | CRE0020 | DES_MT_ | DES_mp_v1 | |
| (292) | | | (203) | enhancer_72bp_ | (280) | |
| | | | | 2 (231) | | |
| SP0379 | | | CRE0020 | DES_MT_ | DES_mp_v1 | |
| (293) | | | (203) | enhancer_72bp_ | (280) | |
| | | | | v3 (232) | | |
| SP0380 | | | CRE0020 | DES_MT_ | DES_mp_v1 | |
| (294) | | | (203) | enhancer_72bp_ | (280) | |
| | | | | v4 (233) | | |
| SP0381 | | | CRE0020 | DES_MT_ | DES_mp_v1 | |
| (295) | | | (203) | enhancer_72bp_ | (280) | |
| | | | | v5 (234) | | |
| SP0382 | | | CRE0020 | DES_MT_ | DES_mp_v1 | |
| (296) | | | (203) | enhancer_72bp_ | (280) | |
| | | | | v6 (235) | | |
| SKM_14 | | | | | SKM_14 | |
| (297) | | | | | (277) | |
| SKM_18 | | | | | SKM_18 | |
| (55) | | | | | (55) | |
| SKM_20 | | | | | SKM_20 | |
| (56) | | | | | (56) | |
| SP0357 | | | | CRE0077 | CRE0046 | |
| (298) | | | | (240) | (268) | |
| SP0437 | | | | CRE0071.16 | CRE0070 | |
| (299) | | | | (249) | (42) | |
| SP0438 | | | | CRE0071.17 | CRE0070 | |
| (300) | | | | (250) | (42) | |
| SP0439 | | | | CRE0071.18 | CRE0070 | |
| (301) | | | | (251) | (42) | |
| SP0440 | | | CRE0020 | CRE0071.13 | CRE0070 | |
| (302) | | | (203) | (243) | (42) | |
| SP0441 | | | CRE0020 | CRE0071.19 | CRE0070 | |
| (303) | | | (203) | (252) | (42) | |
| SP0442 | | | CRE0020 | CRE0071.5 | CRE0070 | |
| (304) | | | (203) | (217) | (42) | |
| SP0443 | | | | CRE0071.20 | CRE0070 | |
| (305) | | | | (253) | (42) | |
| SP0444 | | | | CRE0071.21 | CRE0070 | |
| (306) | | | | (254) | (42) | |
| SP0445 | | | | CRE0071.5 | SKM_18 | |
| (307) | | | | (217) | (55) | |
| SP0447 | | | | CRE0071.22 | CRE0070 | |
| (308) | | | | (255) | (42) | |
| SP0453 | | | CRE0020 | CRE0071 | SKM_18 | |
| (309) | | | (203) | (216) | (55) | |
| SP0454 | | | CRE0020 | CRE0071.5 | SKM_18 | |
| (310) | | | (203) | (217) | (55) | |
| SP0455 | | CRE0093 | CRE0094 | CRE0071 | CRE0070 | |
| (311) | | (247) | (248) | (216) | (42) | |
| SP0456 | | CRE0093 | CRE0094 | CRE0071 | SKM_18 | |
| (312) | | (247) | (248) | (216) | (55) | |
| SP0457 | CRE0093 | CNTRL_001 | CRE0094 | CRE0071 | CRE0070 | |
| (313) | (247) | (259) | (248) | (216) | (42) | |
| SP0458 | | | CRE0020 | CRE0071 | SKM_14 | |
| (314) | | | (203) | (216) | (277) | |
| SP0459 | | | CRE0020 | CRE0071 | CRE0049 | |
| (315) | | | (203) | (216) | (270) | |
| SP0460 | | | | CRE0071.23 | CRE0070 | |
| (316) | | | | (256) | (42) | |
| SP0461 | | | CRE0071.23 | CNTRL_001 | CRE0070 | |
| (317) | | | (256) | (67 bp) (257) | (42) | |
| SP0462 | | | CRE0060 | CRE0071.13 | CRE0070 | |
| (318) | | | (214) | (243) | (42) | |
| SP0463 | | | CRE0020 | CRE0071 | CRE0099 | |
| (319) | | | (203) | (216) | (281) | |
| SP0464 | | | CRE0071 | CRE0020 | CRE0049 | |
| (320) | | | (216) | (203) | (270) | |
| SP0465 | | | CRE0071 | CRE0020 | CRE0099 | |
| (321) | | | (216) | (203) | (281) | |
| SP0466 | | CRE0071 | CRE0093 | CRE0094 | CRE0049 | |
| (322) | | (216) | (247) | (248) | (270) | |
| SP0467 | | | CRE0035 | CRE0071 | SKM_18 | |
| (323) | | | (208) | (216) | (55) | |
| SP0468 | | | CRE0071 | CRE0035 | SKM_18 | |
| (324) | | | (216) | (208) | (55) | |
| SP0469 | | CRE0093.2 | CRE0094.2 | CRE0071 | CRE0070 | |
| (325) | | (260) | (261) | (216) | (42) | |
| SP0470 | | | | CRE0071.24 | CRE0070 | |
| (326) | | | | (258) | (42) | |
| SP0471 | | | CRE0071 | CRE0020 | CRE0070 | |
| (327) | | | (216) | (203) | (42) | |
| SP0473 | | | CRE0020 | CRE0071.5 | SKM_14 | |
| (328) | | | (203) | (217) | (277) | |
| SP0474 | | CRE0093.2 | CRE0094.2 | CRE0071.5 | CRE0070 | |
| (329) | | (260) | (261) | (217) | (42) | |

TABLE 9
Schematic representation of the shortened muscle-specific promoters active in cardiac
and skeletal muscle of Table 8 according to embodiments of this invention with
the cis-regulatory elements and minimal or proximal promoters indicated

| | | | | Promoter |
|---|---|---|---|---|
| | CRE | CRE | CRE | element |
| SP0497 | | CRE0077 | DES_MT_enhancer_48 bp | CRE0070 |
| SP0498 | | CRE0077 | CRE0075 | CRE0053 |
| SP0499 | | CRE0077 | DES_MT_enhancer_48 bp | CRE0053 |
| SP0500 | | CRE0083 | CRE0083 | CRE0053 |
| SP0501 | | CRE0083 | DES_MT_enhancer_48 bp | CRE0053 |
| SP0502 | | CRE0050 | DES_MT_enhancer_48 bp | CRE0053 |
| SP0503 | | CRE0143 | DES_MT_enhancer_48 bp | CRE0053 |
| SP0504 | | CRE0127 | DES_MT_enhancer_48 bp | CRE0053 |
| SP0505 | | CRE0137 | DES_MT_enhancer_48 bp | CRE0053 |
| SP0506 | | CRE0119 | DES_MT_enhancer_48 bp | CRE0053 |
| SP0507 | | CRE0139 | DES_MT_enhancer_48 bp | CRE0053 |
| SP0508 | | | CRE0138 | CRE0053 |
| SP0509 | | CRE0075 | Ch2EnhMYL1_3_v1 | CRE0053 |
| SP0510 | | Ch2EnhMYL1_3_v1 | DES_MT_enhancer_48 bp | CRE0053 |
| SP0511 | | CRE0069 | DES_MT_enhancer_48 bp | BG mp |
| SP0512 | | CRE0077 | DES_MT_enhancer_48 bp | BG mp |
| SP0513 | CRE0083 | CRE0075 | DES_MT_enhancer_48 bp | BG mp |
| SP0514 | CRE0083 | DES_MT_enhancer_48 bp | DES_MT_enhancer_48 bp | BG mp |
| SP0515 | | CRE0050 | DES_MT_enhancer_48 bp | BG mp |
| SP0516 | | CRE0143 | DES_MT_enhancer_48 bp | BG mp |
| SP0517 | | CRE0127 | DES_MT_enhancer_48 bp | BG mp |
| SP0518 | | CRE0137 | DES_MT_enhancer_48 bp | BG mp |
| SP0519 | | CRE0138 | DES_MT_enhancer_48 bp | BG mp |
| SP0520 | | CRE0075 | Ch2EnhMYL1_3_v1 | BG mp |
| SP0521 | | CRE0050 | DES_MT_enhancer_48 bp | SCP1 |
| SP4169 | | CRE0050 | DES_MT_enhancer_48 bp | CRE0070 |
| SP0522 | | CRE0145 | DES_MT_enhancer_48 bp | CRE0053 |
| SP0523 | | CRE0145 | DES_MT_enhancer_48 bp | BG mp |
| SP0524 | | CRE0145 | DES_MT_enhancer_48 bp | SCP1 |

TABLE 3
Cis-regulatory elements comprised in the promoters of Tables 1 and 2

| Name | SEQUENCE |
|---|---|
| CRE0016 (SEQ | CCTTGCCTGACTATTGGCAGGCGGACCTGGTGGTCAGACCTCAGTGATC |
| ID NO: 201) | CTCAGGGACCAGTGAATATTTCAGGCTGGGGCTGAGCATCACCTGCTCC |
| | CTTGGCCCCACTTATAGGGCAAAGGGGAGTCTACCAGCCTACTCACTGA |
| | TGACAAACTGGAAAAGTTTGTCCTGTCTCTGCTCTGGCCCCACCTCGCC |
| | CTCTCCCCTACTTGGAAGTTCCTTTCCTGAACCACTGACTGCCAAAGCTT |
| | GAGGGATTAAATAAATCATCTGGCCCAA |
| CRE0018 (SEQ | CTGTGTGTTTCTGTGGCTGAGTCAGATGGAGGAGTCCTCATGTTTCACT |
| ID NO: 202) | GCTTAGCAGTTTTTGTCCTTCCTAGTACCCGTTCCCAGCCCACAAGATG |
| | CAGAAAGAGCTGTTGCTAGCGTGAGTTATTTTTGTCAGCTGAGTCACCA |
| | CGCCAGAAAGCAAGAAATGACCCGCTTTATGTCTGCTCTGAGGAGCTG |
| | GAACC |
| CRE0020 (SEQ | GGGCCCCACAGCAGCTGGGGGCATTTATGGGCCTTCCTATAAACTTCTG |
| ID NO: 203) | AGAGGGTAACTTTATCCTGCTTCTTTCAGCCAAGTATCCTCCTCCAGCA |
| | GCTGGTCACAAAGCTGGTTAATCTCCCAGAGTGCTCAGCTTAAAACCCG |
| | TGACTCACAGCACAGCCAGTGTGGGGGAGGGGGTGGCTGCCTCCAATA |
| | CGTGGCGCCCAGAGTCAGCTGTTCTGGGGCCTTCTCTGGTTTCTCCAAC |
| | TGAGTCCTGAGGTTTGGGGCCTTGTCTTCCTTCCTGGAGT |
| CRE0025 (SEQ | GCGCCCTGATGAATATGCATCGCGGCGCGCCCGCCCCCGGCTCCTCCTT |
| ID NO: 204) | TCGGTTTCCTTCCCGCCGCCAGGCGGAAGCGAAGAGCCGCGCTTCCCGC |
| | GCGCCCAGGCCGGCCGTGGTAGGGTGGGGCGGGGCGGGCCGCGAGCC |
| | GGAGAAAGAGAAAGC |
| CRE0027 (SEQ | TACATCATTTACCTAGAAAAGAGGACAGCTGTCCTTTCCCAAAGCTCCG |
| ID NO: 205) | GTGACCCTGCCCCGCCCAGTGTGACTAGCCCAGGTTGGTGATTCTGATC |
| | TGTTGCCAAACCAAACTGGCTCCCCGGGGAGCCATTTGGTAATGTTCCC |
| | TGGAGTCATTTCCTTGCGAAGCATTCCTTTTCGGTGAGAGGACATTTTTT |
| | TCATCCCTGATAAACAACCACAGCCTGCGCCAG |
| CRE0028 (SEQ | TAAGTGTGATGCACAGTGCTTGCATTTTCTTGATACGTTAGTCATATGA |
| ID NO: 40) | GAGCTGACAAAGAAGGAAAAAGAGCAGCGATGTGGTGCAATATTAAC |
| | AGGCAGCTGTCCCCTGGCTTCCCGATACGTGGGATGACTCGCATTGCTG |
| | AGCGGTGTGGTCACTGCCAAAGGAATGACCCTCTCACATTTCTTCCTGA |
| | TTCGCATACGCCGCGGC |
| CRE0029 (SEQ | CTCTGTCTCCTCAGGTGCCTGGCTCCCAGTCCCCAGAACGCCTCTCCTG |
| ID NO: 206) | TACCTTGCTTCCTAGCTGGGCCTTTCCTTCTCCTCTATAAATACCAGCTC |
| | TGGTATTTCGCCTTGGCAGCTGTTGCTGCTAGGGAGACGGCTGGCTTGA |
| | CATGCATCTCCTGACAAAACACAAACCCGTGGTGTGAGTGGGTGTGGG |
| | CGGTGTGAGTAGGGGGATGAATCAGAGAGGGGGC |
| CRE0031 (SEQ | TAAGTCCGGGCAGGGTCCTGTCCATAAAAGGCTTTTCCCGGGCCGGCTC |
| ID NO: 207) | CCCGCCGGCAGCGTGCCCCGCCCCGGCCCGCTCCATCTCCAAAGCATGC |
| | AGAGAATGTCTCGGCAGCCCCGGTAGACTGCTCCAACTTGGTGTCTTTC |
| | CCCAAATATGGAGCCTGTGTGGAGTCACTGGGGGAGCCGGGGGTGGGG |
| | AGCGGAGCCGGCTTCCTCTAG |
| CRE0033 (SEQ | CCCTTCAGATTAAAAATAACTGAGGTAAGGGCCTGGGTAGGGGAGGTG |
| ID NO: 41) | GTGTGAGACGCTCCTGTCTCTCCTCTATCTGCCCATCGGCCCTTTGGGG |
| | AGGAGGAATGTGCCCAAGGACTAAAAAAAGGCCATGGAGCCAGAGGG |
| | GCGAGGGCAACAGACCTTTCATGGGCAAACCTTGGGGCCCTGCTG |
| CRE0035 (SEQ | GCCACTACGGGTCTAGGCTGCCCATGTAAGGAGGCAAGGCCTGGGGAC |
| ID NO: 208) | ACCCGAGATGCCTGGTTATAATTAACCCAGACATGTGGCTGCCCCCCCC |
| | CCCCAACACCTGCTGCCTGAGCCTCACCCCCACCCCGGTGCCTGGGTCT |
| | TAGGCTCTGTACACCATGGAGGAGAAGCTCGCTCTAAAAATAACCCTG |
| CRE0036 (SEQ | CTGAGATTTTCCTAGCATTTTGTGTTTCATGACTAAATATGGTTTGTGTT |
| ID NO: 209) | TCAAGACCAATGAGCTGGGAACTGTACTGTTCTTTCCCCTCCCATCAAC |
| | TCATTTTTGGCACAAGACGCACTCTAGTCAGTTGGAGCAAATCCCCTGA |
| | CCCGGGTGCAGTTCCAAAAGCAGACACTCGAGCGTGTTTTACCTAATTA |
| | GGAAATGCTTTGCTCCAAACCGAACTGCTCATTCAGGTTAGAGAGGAG |
| CRE0047 (SEQ | CCCACCCATGCCTCCTCAGGTACCCCCTGCCCCCCACAGCTCCTCTCCT |
| ID NO: 210) | GTGCCTTGTTTCCCAGCCATGCGTTCTCCTCTATAAATACCCGCTCTGGT |
| | ATTTGGGGTTGGCAGCTGTTGCTGCCAGGGAGATGGTTGGGTTGACATG |
| | CGGCTCCTGACAAAACACAAACCCCTGGTGTGTGTGGGCGTGGGTGGT |
| | GTGAGTAGGGGGATGAATCAGGGAGGGGGCGGGGG |
| CRE0050 (SEQ | CTAGACTAGCATGCTGCCCATGTAAGGAGGCAAGGCCTGGGGACACCC |
| ID NO: 211) | GAGATGCCTGGTTATAATTAACCCAGACATGTGGCTGCCCCCCCCCCCC |
| | CAACACCTGCTGCCTCTAAAAATAACCCTGC |
| CRE0051 (SEQ | CACCGCGGTGGCGGCCGTCCGCCCTCGGCACCATCCTCACGACACCCA |
| ID NO: 212) | AATATGGCGACGGGTGAGGAATGGTGGGGAGTTATTTTTAGAGCGGTG |
| | AGGAAGGTGGGCAGGCAGCAGGTGTTGGCGCTCTAAAAATAACTCCCG |
| | GGAGTTATTTTTAGAGCGGAGGAATGGTGGACACCCAAATATGGCGAC |
| | GGTTCCTCACCCGTCGCCATATTTGGGTGTCCGCCCTCGGCCGGGGCC |
| CRE0059 (SEQ | CCCCTGCCCCCCACAGCTCCTCTCCTGTGCCTTGTTTCCCAGCCATGCGT |
| ID NO: 213) | TCTCCTCTATAAATACCCGCTCTGGTATTTGGGGTTGGCAGCTGTTGCTG |
| | CCAGGGAGATGGTTGGGTTGACATG |
| CRE0060 (SEQ | CTCTATAAATACCCGCTCTGGTATTTGGGGTT |
| ID NO: 214) | |
| CRE0069 (SEQ | AGACTGGGGCAGGTGCAGGCTGGATTGGGTTTCCAGAGGCTATATATA |
| ID NO: 215) | TAAAGGCTGCCGGGAGCCCCAGGGCCGCTCCCTGAGGGCACAACACTG |
| | TGGGGGCCCAGCCAGGCCCACATTCCTTTCCAGAGGCCAGCTCTCCATT |
| | TATAGCCCCTGGGCAGAGCAGC |
| CRE0071 (SEQ | CACCGCGGTGGCGGCCGTCCGCCCTCGGCACCATCCTCACGACACCCA |
| ID NO: 216) | AATATGGCGACGGGTGAGGAATGGTGGGGAGTTATTTTTAGAGCGGTG |
| | AGGAAGGTGGGCAGGCAGCAGGTGTTGGCGCTCTAAAAATAACTCCCG |
| | GGAGTTATTTTTAGAGCGGAGGAATGGTGGACACCCAAATATGGCGAC |
| | GGTTCCTCACCCGTCGCCATATTTGGGTGTCCGCCCT |
| CRE0071.5 | ACACCCAAATATGGCGACGGGTGAGGAATGGTGGGGAGTTATTTTTAG |
| (SEQ ID NO: | AGCGGTGAGGAAGGTGGGCAGGCAGCAGGTGTTGGCGCTCTAAAAATA |
| 217) | ACTCCCGGGAGTTATTTTTAGAGCGAGCTCTATAAATACCCGCTCTGGT |
| | ATTTGGGGTTTTGAACCCGTCGCCATATTTGGGTGTCCGCCCT |
| CRE0073 (SEQ | TCCCTAACCTCCTGCTTGCGAGGCCTCTCTCTGGCCTCTGAGAGGGTCA |
| ID NO: 218) | GTGTCCTGCCCCAACCCATGAGATGACAGACTATAATAGCCACAGGAT |
| | TAACATAGCAGGCATTGTCTTTCTCTGACTATAGGGTGGGTATTATGTG |
| | TTCATCAACCATCCTAAAAATACCCGGTAAACAGGTGCAGCCCCTGTGG |
| | CTCCAGTCCCCTGGGATCTGTTGGCTTCTGGCTGGAGATGAAGATTAGG |
| | GCAGAGGAGAGGTGAATTAGTCTCACTGAGTTCCAGGCATGAGACTCG |
| | GGTGTCCTTTGGAACCTGGGAAATCTAGATTCCAGGAAACCCATCTGGA |
| | GGG |
| CRE0074 (SEQ | CCATCCTAAAAATACCCGGTAAACAGGTGCAGCCCCTGTGGCTCCAGT |
| ID NO: 219) | CCCCTGGGATCTGTTGGCTTCTGGCTGGAGATGAAGATTAGGGCAGAG |
| | GAGAGGTGAATTAGTCTCACTGAGTTCCAGGCATGAGACTCGGGTGTC |
| | CTTTGGAA |
| CRE0075 (SEQ | AGGGTCAGTGTCCTGCCCCAACCCATGAGATGACAGACTATAATAGCC |
| ID NO: 220) | ACAGGATTAACATAGCAGGCATTG |
| CRE0076 (SEQ | CTGAGGGGTGTCAGAGCACAGGCTGAGGCCTCTTGCCTGACGTGGGAC |
| ID NO: 221) | CCCTTGGTCTGGCATTTGTCAGTGAGGCAGGCTGGGGGCAGGCCCCGG |
| | AGCTTGGCAGGAGGTGTAAACCGGCCTTGGAAGGTAGGGCCCCACAAT |
| | GGGGACAGTTGGATCTCTGAGGGAGACAGGGAGGCATGATCACTGCCA |
| | AATGCCCACCAAGGACAAGGCACATCCCAGGGAGACAGACGCAGACC |
| | TGGTGCCCTCTGGACACTGGCATTCCTGGAGGCTGATGATGGACAGATG |
| | GGCCTGGAGGTGGCTCTTCGCCAGCTGGTGTTTCCTTTGGACTTCCTCA |
| | GTGTCTTTGGAGAAGCAGAGCCCTAAGAATAAGCAGCTGCCCATAAAA |
| | TCTAATACCAGCCAAGCATCTCAGGAATTCATGGATTGTCTCCAT |
| CRE0078 (SEQ | TTCTGAGTCCTCTAAGGTCCCTCACTCCCAACTCAGCCCCATGTCCTGTC |
| ID NO: 222) | AATTCCCACTCAGTGTCTGATCTCCTTCTCCTCACCTTTCCCATCTCCCG |
| | TTTGACCCAAGCTTCCTGAGCTCTCCTCCCATTCCCCTTTTTGGAGTCCT |
| | CCTCCTCTCCCAGAACCCAGTAATAAGTGGGCTCCTCCCTGGCCTGGAC |
| | CCCCGTGGTAACCCTATAAGGCGAGGCAGCTGCTGTCTGAGGCAGGGA |
| | GGGGCTGGTGTGGGAGGCTAAGGGCAGCTGCTAAGTTTAGGGTGGCTC |
| | CTTCTCTCTTCTTAGAGACAACAGGTGGCTGGGGCCTCAGTGCCCAGAA |
| | AAGAAAATGTCTTAGAGGTATCGGCATGGGCCTGGAGGAGGGGGGACA |
| | GGGCAGGGGGAGGCATCTTCCTCAGGACATCGGGTCCTAGAGG |
| CRE0079 (SEQ | CCTCCCTGGCCTGGACCCCCGTGGTAACCCTATAAGGCGAGGCAGCTG |
| ID NO: 223) | CTGTCTGAGGCAGGGAGGGGCTGGTGTGGGAGGCTAAGGGCAGCTGCT |
| | AAGTTTAGGGTG |
| 48 bp (SEQ ID | TTCTCCTCTATAAATACCCGCTCTGGTATTTGGGGTTGGCAGCTGTTG |
| NO: 224) | |
| CRE0071.7 | CGACACCCAAATATGGCGACGGGTGAGGAATGGTGGGGAGTTATTTTT |
| (SEQ ID NO: | AGAGCGGTGAGGAAGGTGGGCAGGCAGCAGGTGTTGGCGCTCTAAAA |
| 225) | ATAACTCCCGGGAGTTATTTTTAGAGCGGAGCGACACCCAAATATGGC |
| | GACGGGTGAGGAATGGTGGGGAGTTATTTTTAGAGCGGTGAGGAAGGT |
| | GGGCAGGCAGCAGGTGTTGGCGCTCTAAAAATAACTCCCGGGAGTTAT |
| | TTTTAGAGCGGAGCGACACCCAAATATGGCGACGGGTGAGGAATGGTG |
| | GGGAGTTATTTTTAGAGCGGTGAGGAAGGTGGGCAGGCAGCAGGTGTT |
| | GGCGCTCTAAAAATAACTCCCGGGAGTTATTTTTAGAGCGGAG |
| CRE0071.10 | TAAGGCGAGGCAGCTGCTGTCTGAGGCAGGACACCCAAATATGGCGAC |
| (SEQ ID NO: | GGGTGAGGAATGGTGGGGAGTTATTTTTAGAGCGGTGAGGAAGGTGGG |
| 226) | CAGGCAGCAGGTGTTGGCGCTCTAAAAATAACTCCCGGGAGTTATTTTT |
| | AGAGCGCTCTAAGGTCCCTCACTCCCAACTCAGCCCCATGTCCTGTCAA |
| | TTCACCCGTCGCCATATTTGGGTGTCCGCCCT |
| CRE0071.11 | CTCTAAGGTCCCTCACTCCCAACTCAGCCCCATGTCCTGTCAATTCGAC |
| (SEQ ID NO: | ACCCAAATATGGCGACGGGTGAGGAATGGTGGGGAGTTATTTTTAGAG |
| 227) | CGGTGAGGAAGGTGGGCAGGCAGCAGGTGTTGGCGCTCTAAAAATAAC |
| | TCCCGGGAGTTATTTTTAGAGCGTAAGGCGAGGCAGCTGCTGTCTGAGG |
| | CAGACCCGTCGCCATATTTGGGTGTCCGCCCT |
| CRE0071.12 | TAAGGCGAGGCAGCTGCTGTCTGAGGCAGAGGCTAAGGGCAGCTGCTA |
| (SEQ ID NO: | AGTTTAGGGTCTCTAAGGTCCCTCACTCCCAACTCAGCCCCATGTCCTG |
| 228) | TCAATTCCGACACCCAAATATGGCGACGGGTGAGGAATGGTGGGGAGT |
| | TATTTTTAGAGCAGGCAGCAGGTGTTGGCGCTCTAAAAATAACTCCCGG |
| | GAGTTATTTTTAGAGCGACCCGTCGCCATATTTGGGTGTCCGCCCT |
| DES_MT_ | TACTAAAAATAGAACGACTATTTTTAGGCTTTTCTGGCAGCTGGCC |
| Enhancer_48 bp_v2 | |
| (SEQ ID NO: | |
| 229) | |
| DES_MT_ | CGAGGTACTATAAATACCCTTAGAGGTATTTTATCTTGGCAGCTAGGT |
| Enhancer 48 bp_v3 | |
| (SEQ ID NO: | |
| 230) | |
| DES_MT_ | TACTAAAAATAGAACGACTATTTTTAGGCTTTTCTGGCAGCTGGCCCTG |
| Enhancer_72 bp_v2 | CCAGACAGAGTTCCTCAGTAA |
| (SEQ ID NO: | |
| 231) | |
| DES_MT_ | CGAGGTACTATAAATACCCTTAGAGGTATTTTATCTTGGCAGCTAGGTC |
| nEnhacer_72 bp_v3 | TGCCAGACAGAGTTCCTCAGTAA |
| (SEQ ID NO: | |
| 232) | |
| DES_MT_ | TACTAAAAATAGAACGACTATTTTTAGGCTTTTCTGGCAGCTGGCCCTG |
| Enhancer_72 bp_v4 | CCAGACAGATAAACGAGCTAT |
| (SEQ ID NO: | |
| 233) | |
| DES_MT_ | CGAGGTACTATAAATACCCTTAGAGGTATTTTATCTTGGCAGCTAGGTC |
| Enhancer_ | TGCCAGACAGATAAACGAGCTAT |
| 72 bp_v5 | |
| (SEQ ID NO: | |
| 234) | |
| DES_MT_ | TTAAACGAGCTATTAGTTATGAGGTCCGTAGATTGAATAAACGAGCTAT |
| Enhancer_ | TAGTTATGAGGTCCGTAGATTGAA |
| 72 bp_v6 | |
| (SEQ ID NO: | |
| 235) | |
| CRE0071.3 | CACCGCGGTGGCGGCCGTCCGCCCTCGGCACCATCCTCACGACACCCA |
| (SEQ ID NO: | AATATGGCGACGGGTGAGGAATGGTGGGGAGTTATTTTTAGAGCGTAA |
| 43) | ACGAGCTATTAGTTGCAGCAGGTGTTGGCGCTCTAAAAATAACTCCCGG |
| | GAGTTATTTTTAGAGCGGAGGAATGGTGGACACCCAAATATGGCGACG |
| | GTTCCTCACCCGTCGCCATATTTGGGTGTCCGCCCT |
| CRE0071.4 | CACCGCGGTGGCGGCCGTCCGCCCTCGGCACCATCCTCACGACACCCA |
| (SEQ ID NO: | AATATGGCGACGGGTGAGGAATGGTGGGGAGTTATTTTTAGAGCGGTG |
| 236) | AGGAAGGTGGGCAGGCAGCAGGTGTTGGCGCTCTAAAAATAACTCCCG |
| | GGAGTTATTTTTAGAGCGAGGTAAACGAGCTATTAGTTATGAGGTCCGT |
| | AGATTGAACCCGTCGCCATATTTGGGTGTCCGCCCT |
| CRE0071.6 | CGACACCCAAATATGGCGACGGGTGAGGAATGGTGGGGAGTTATTTTT |
| (SEQ ID NO: | AGAGCGGTGAGGAAGGTGGGCAGGCAGCAGGTGTTGGCGCTCTAAAA |
| 237) | ATAACTCCCGGGAGTTATTTTTAGAGCGGAGCGACACCCAAATATGGC |
| | GACGGGTGAGGAATGGTGGGGAGTTATTTTTAGAGCGGTGAGGAAGGT |
| | GGGCAGGCAGCAGGTGTTGGCGCTCTAAAAATAACTCCCGGGAGTTAT |
| | TTTTAGAGCGGAG |
| CRE0071.8 | TAAGGCGAGGCAGCTGCTGTCTGAGGCAGGACACCCAAATATGGCGAC |
| (SEQ ID NO: | GGGTGAGGAATGGTGGGGAGTTATTTTTAGAGCGGTGAGGAAGGTGGG |
| 238) | CAGGCAGCAGGTGTTGGCGCTCTAAAAATAACTCCCGGGAGTTATTTTT |
| | AGAGCGGAGGAATGGTGGACACCCAAATATGGCGACGGTTCCTCACCC |
| | GTCGCCATATTTGGGTGTCCGCCCT |
| CRE0071.9 | AGGCTAAGGGCAGCTGCTAAGTTTAGGGTGACACCCAAATATGGCGAC |
| (SEQ ID NO: | GGGTGAGGAATGGTGGGGAGTTATTTTTAGAGCGGTGAGGAAGGTGGG |
| 239) | CAGGCAGCAGGTGTTGGCGCTCTAAAAATAACTCCCGGGAGTTATTTTT |
| | AGAGCGGAGGAATGGTGGACACCCAAATATGGCGACGGTTCCTCACCC |
| | GTCGCCATATTTGGGTGTCCGCCCT |
| CRE0077 | TCTGAGGGAGACAGGGAGGCATGATCACTGCCAAATGCCCACCAAGGA |
| (SEQ ID NO: | CAAGGCACATCCCAGGGAGACAGACGCAGACCTGGTGCCCTCTGGACA |
| 240) | CTGGCATTCCTGGAG |
| CRE0029.2 | CTCTGTCTCCTCAGGTGCCTGGCTGCTTCCTAGCTGGGCCTTTCCTTCTC |
| (SEQ ID NO: | CTCTATAAATACCAGCTCTGGTATTTCGCCTTGGCAGCTGTTGCTGCTA |
| 241) | GGGAGACGGCTGGCTTGACATGCATCTCCTGACAAAACACAAACCCGT |
| | GGTGTGAGTGGGTGTGGGCGGTGTGAGTAGGGGGATGAATCAGAGAGG |
| | GGGC |
| CRE0069.2 | AGACTGGGGCAGGTGCAGGCTGGATTGGGTTTCCAGAGGCTATATATA |
| (SEQ ID NO: | TAAAGGCTGCCGGGAGCCCACATTCCTTTCCAGAGGCCAGCTCTCCATT |
| 242) | TATAGCCCCTGGGCAGAGCAGC |
| CRE0071.13 | ACACCCAAATATGGCGACGGGTGAGGAATGGTGGGGAGTTATTTTTAG |
| (SEQ ID NO: | AGCGGTGAGGAAGGTGGGCAGGCAGCAGGTGTTGGCGCTCTAAAAATA |
| 243) | ACTCCCGGGAGTTATTTTTAGAGCGGAGGAATGGTGGACACCCAAATA |
| | TGGCGACGGTTCCTCACCCGTCGCCATATTTGGGTGTCCGCCCT |
| CRE0071.14 | CACCGCGGTGGCGGCCGTCCGCCCTCGGCACCATCCTCACGACACCCA |
| (SEQ ID NO: | AATATGGCGACGGGTGAGGAATGGTGGGGAGTTATTTTTAGAGCGGTG |
| 244) | AGGAAGGTGGGCAGGCAGCAGGTGTTGGCGCTCTAAAAATAACTCCCG |
| | GGAGTTATTTTTAGAGCGAGCTCTATAAATACCCGCTCTGGTATTTGGG |
| | GTTTTGAACCCGTCGCCATATTTGGGTGTCCGCCCT |
| CRE0071.15 | CACCGCGGTGGCGGCCGTCCGCCCTCGGCACCATCCTCACGACACCCA |
| (SEQ ID NO: | AATATGGCGACGGGTGAGGAATGGTGGGGAGCTATTTTTAGAGCGGTG |
| 245) | AGGAAGGTGGGCAGGCAGCAGGTGTTGGCGCTCTAAAAATAGCTCCCG |
| | GGAGCTATTTTTAGAGCGGAGGAATGGTGGACACCCAAATATGGCGAC |
| | GGTTCCTCACCCGTCGCCATATTTGGGTGTCCGCCCT |
| 72 bp (SEQ ID | TTCTCCTCTATAAATACCCGCTCTGGTATTTGGGGTTGGCAGCTGTTGCT |
| NO: 246) | GCCAGGGAGATGGTTGGGTTGA |
| CRE0093 (SEQ | CCACAGCAGCTGGGGGCATTTATGGGCCTTCCTATAAACTTCTGAGAGG |
| ID NO: 247) | GTAACTTTATCCTGCTTCTTTCAGCCAAGTATCCTCCTCCA |
| CRE0094 (SEQ | AAACCCGTGACTCACAGCACAGCCAGTGTGGGGGAGGGGGTGGCTGCC |
| ID NO: 248) | TCCAATACGTGGCGCCCAGAGTCAGCTGTTCTGGGGCCTTCTCTGGTTT |
| | CTCCAACTGAGTCCTGAGGTTTGG |
| CRE0071.16 | CACCGCGGTGGCGGCCGTCCGCCCTCGGATAGCTCGTTTAGACACCCA |
| (SEQ ID NO: | AATATGGCGACGGTAAACGAGCTATTGGGAGTTATTTTTAGAGCGTAA |
| 249) | ACGAGCTATTAGTTGCAGCAGGTGTTGGCGCTCTAAAAATAACTCCCGG |
| | GAGTTATTTTTAGAGCGGAGGAATGGTGGACACCCAAATATGGCGACG |
| | GTTCCTCACCCGTCGCCATATTTGGGTGTCCGCCCT |
| CRE0071.17 | CACCGCGGTGGCGGCCGTCCGCCCTCGGATAGCTCGTTTAGACACCCA |
| (SEQ ID NO: | AATATGGCGACGGGTGAGGAATGGTGGGGAGTTATTTTTAGAGCGGTG |
| 250) | AGGAAGGTGGGCAGGCAGCAGGTGTTGGCGCTCTAAAAATAACTCCCG |
| | GGAGTTATTTTTAGAGCGGAGGAATGGTGGACACCCAAATATGGCGAC |
| | GGTTCCTCACCCGTCGCCATATTTGGGTGTCCGCCCT |
| CRE0071.18 | CACCGCGGTGGCGGCCGTCCGCCCTCGGCACCATCCTCACGACACCCA |
| (SEQ ID NO: | AATATGGCGACGGTAAACGAGCTATTGGGAGTTATTTTTAGAGCGGTG |
| 251) | AGGAAGGTGGGCAGGCAGCAGGTGTTGGCGCTCTAAAAATAACTCCCG |
| | GGAGTTATTTTTAGAGCGGAGGAATGGTGGACACCCAAATATGGCGAC |
| | GGTTCCTCACCCGTCGCCATATTTGGGTGTCCGCCCT |
| CRE0071.19 | ACACCCAAATATGGCGACGGGTGAGGAATGGTGGGGAGTTATTTTTAG |
| (SEQ ID NO: | AGCGGTGAGGAAGGTGGGCAGGCAGCAGGTGTTGGCGCTCTAAAAATA |
| 252) | ACTCCCGGGAGTTATTTTTAGAGCGCCCGTCGCCATATTTGGGTGTCCG |
| | CCCT |
| CRE0071.20 | GGCCGTCCGCCCTCGGCACCATCCTCACGACACCCAAATATGGCGACG |
| (SEQ ID NO: | GGTGAGGAATGGTGGGGAGCTATTTTTAGAGCGTAAACGAGCTATTAG |
| 253) | TTGCAGCAGGTGTTGGCGCTCTAAAAATAGCTCCCGGGAGCTATTTTTA |
| | GAGCGGAGGAATGGTGGACACCCAAATATGGCGACGGTTCCTCACCCG |
| | TCGCCATATTTGGGTGTCCGCCCT |
| CRE0071.21 | GGCCGTCCGCCCTCGGCACCATCCTCACGACACCCAAATATGGCGACG |
| (SEQ ID NO: | GGTGAGGAATGGTGGGGAGCTATTTTTAGAGCGTAAACGAGCTATTAG |
| 254) | TTGCAGCAGGTGTTGGCGCTCTAAAAATAGCTCCCGGGAGCTATTTTTA |
| | GAGCGAGCTCTATAAATACCCGCTCTGGTATTTGGGGTTTTGAACCCGT |
| | CGCCATATTTGGGTGTCCGCCCT |
| CRE0071.22 | GGCCGTCCGCCCTCGGGACACCCAAATATGGCGACGGGGGAGTTATTT |
| (SEQ ID NO: | TTAGAGCGGGCAGGCAGCAGGTGTTGGCGCTCTAAAAATAACTCCCGG |
| 255) | GAGTTATTTTTAGAGCGGAGGAATGGTGGACACCCAAATATGGCGACG |
| | GTTCCTCACCCGTCGCCATATTTGGGTGTCCGCCCT |
| CRE0071.23 | CACCGCGGTGGCGGCCGTCCGCCCTCGGCACCATCCTCACGACACCCA |
| (SEQ ID NO: | AATATGGCGACGGGTGAGGAATGGTGGGGAGTTATTTTTAGAGCGGTG |
| 256) | AGGAAGGTGGGCAGGCAGCAGGTGTTGGCGCTCTAAAAATAACTCCCG |
| | GGAGTTATTTTTAGAGCG |
| CNTRL 001 | AGGCAGTGTATACTCTTCCATAAACGAGCTATTAGTTATGAGGTCCGTA |
| (67 bp) | GATTGAAAAGGGTGACGG |
| (SEQ ID NO: | |
| 257) | |
| CRE0071.24 | CACCGCGGTGGCGGCCGTCCGCCCTCGGCACCATCCTCACGACACCCA |
| (SEQ ID NO: | AATATGGCGACGGGTGAGGAATGGTGGGGATAAACGAGCTATGCGGTG |
| 258) | AGGAAGGTGGGCAGGCAGCAGGTGTTGGCGCATAGCTCGTTTATCCCG |
| | GGATAAACGAGCTATGCGGAGGAATGGTGGACACCCAAATATGGCGAC |
| | GGTTCCTCACCCGTCGCCATATTTGGGTGTCCGCCCT |
| CNTRL_001 | AGGCAGTGTATACTCTTCCATAAACGAGCTATTAGTTATGAGGTC |
| (SEQ ID NO: | |
| 259) | |
| CRE0093.2 | CCACAGCAGCTGGGGGCATTTCTGAGAGGGTAACTTTATCCTGCTTCTT |
| (SEQ ID NO: | TCAGCCAAGTA |
| 260) | |
| CRE0094.2 | CTCACAGCACAGCCAGTGTGGGGGAGGGGGTGGCTGCCTCCGTGGCGC |
| (SEQ ID NO: | CCAGAGTCAGCTGTTCTGGGGCCTTCTCTGGTTTCTCCAACTGAGTCCT |
| 261) | GAGGTTTGG |

TABLE 10
CREs from promoters of Table 8 and 9.

| | | |
|---|---|---|
| CRE0119 | AATTATTTTTAATAACACTTACTGGTAAGAGAAAGGGGAGAAA | 151 |
| (SEQ ID NO: | CCTTAGACAGGCACTTAGATGTGACTAAGGCAGGTTTATCTCTG | |
| 388) | ATTCCAAAGCACTGGAGTGGAAGTCACACCGTGACTCAGAGCA | |
| | TTGTGATGGGCCAGCTGTCCA | |
| CRE0127 | TTGGCCCAGGTCACACTGGGGTGAGGCTAGTGTTCCTGAGCCTT | 142 |
| (SEQ ID NO: | GACAAGGAGACAGCTTGAAATAGACGAGTGTCACATTTCTGAG | |
| 389) | CAGCTGTGTGGCGACAGCAGGAGGGGTAGGGAATAGACAGTAT | |
| | AAAAGAGAAAGC | |
| CRE0137 | CCCTGCCATCTTGGGTTTCAGGGCAGAGGAGTCTTGCTAATTTT | 146 |
| (SEQ ID NO: | GATGCCTATTTTTGGACACTTCAGCTGCCACTGGCTCCTTATAA | |
| 390) | ACGCATGACACCCCATGCAAACACACTACCCCTCCCTCCACTGC | |
| | TGACAGGTGTGTGG | |
| CRE0138 | CTTCTCAAGCCAAAGGAGCAAGAGTTAAAAATAACAGGCTCAC | 165 |
| (SEQ ID NO: | CCTGGCAGCCACCTGTGCTGGCCAGCCCCACCCCATCCCTCCCT | |
| 391) | CGGGGACAGCTGCAGCTCCTCAGGCCCCGCCCGGGACATTTTG | |
| | GGAACACTTTCTCCTCTTACTTCTCATCTTCAGGG | |
| CRE0139 | CAGTGTTCCTATATTTATCCCACCATACAGAGCTTCCTTTGCCTC | 151 |
| (SEQ ID NO: | AGAAGGACCAGCAGTTTCGCTAGCTTAACAAAACCAGCCACTC | |
| 392) | AGGGTATTGGTTTACAGTCAAGCAACTCTGGGAGAGGGCAGCT | |
| | GCTCTCAGACATCATACAGC | |
| CRE0143 | TCGTCCCCTGGCTGGCCCATGTAATCTGAGCCCAGCATTGTACA | 135 |
| (SEQ ID NO: | TATCCTGGGAACAGCTGACAATGCAGTGGTCAGACAGCTGGTG | |
| 393) | GGGCCAGCTAGAGCTGGCAGGGTTGGCTGGGAGGGGAGTGTAG | |
| | GCTGA | |
| CRE0145 | GCTGCCCATGTAAGGAGGCAAGGCCTGGGGACACCCGAGATGC | 114 |
| (SEQ ID NO: | CTGGTTATAATTAACCCAGACATGTGGCTGCCCCCCCCCCCCAA | |
| 394) | CACCTGCTGCCTCTAAAAATAACCCTG | |
| CRE0077 | TCTGAGGGAGACAGGGAGGCATGATCACTGCCAAATGCCCACC | 111 |
| (SEQ ID NO: | AAGGACAAGGCACATCCCAGGGAGACAGACGCAGACCTGGTG | |
| 395) | CCCTCTGGACACTGGCATTCCTGGAG | |
| DES_MT_ | TTCTCCTCTATAAATACCCGCTCTGGTATTTGGGGTTGGCAGCT | 48 |
| enhancer_48 bp | GTTG | |
| (SEQ ID NO: | | |
| 396) | | |
| CRE0075 | AGGGTCAGTGTCCTGCCCCAACCCATGAGATGACAGACTATAA | 72 |
| (SEQ ID NO: | TAGCCACAGGATTAACATAGCAGGCATTG | |
| 397) | | |
| CRE0083 | CCAGCCCACCTGTCCCAATGCTGACTTAGTGCAAGGCGAGCCA | 96 |
| (SEQ ID NO: | GCAAGGAGGGAGGACAGGTGGCAGTGGGGGGTGAGGAGCATC | |
| 398) | TAAAAATAGCC | |
| Ch2EnhMYL | ATTTTTAAAGACTGAGGAATTAGGCACCTGTCATTTTTGCCAGC | |
| 1_3_v1 | TGGTGTAGATGTTAAAAATTACTGTCACTCTTCCGCCTGCTACT | |
| (SEQ ID NO: | TTATTTTGCACCTGCTGTTACTTGAGTTACAGGCATTTCA | |
| 399) | | |
| CRE0050 | CTAGACTAGCATGCTGCCCATGTAAGGAGGCAAGGCCTGGGGA | 128 |
| (SEQ ID | CACCCGAGATGCCTGGTTATAATTAACCCAGACATGTGGCTGCC | |
| NO: 400) | CCCCCCCCCCCAACACCTGCTGCCTCTAAAAATAACCCTGC | |
| CRE069 | AGACTGGGGCAGGTGCAGGCTGGATTGGGTTTCCAGAGGCTAT | 167 |
| (SEQ ID NO: | ATATATAAAGGCTGCCGGGAGCCCCAGGGCCGCTCCCTGAGGG | |
| 401) | CACAACACTGTGGGGGCCCAGCCAGGCCCACATTCCTTTCCAG | |
| | AGGCCAGCTCTCCATTTATAGCCCCTGGGCAGAGCAGC | |

TABLE 4
Minimal/Proximal Promoters comprised in the promoters of Table 1, 2 and 3

| Name | SEQUENCE |
|---|---|
| CRE0005 (SEQ | ACTCGGGGGCCAGGCACTGGCGCTGACGCAGGCTAGCAGGGCGCCACT |
| ID NO: 262) | GGCTGGTCCCCACCCACCTCGGTGGGTTGGGGGATGGGCGCACCAGCC |
| | CCTCCTGGGTGAGCCCTAGCCTGGGGCTTCCTATTTCGGGAGCCGGGGG |
| | CGTGGGCCACGTCTCCTCATGTGATGCGAGGGCTATTTAAAGCGGCAGC |
| | CCGGGCAGGGAGCCGCCGTCGGAGCCCTTGCACGCCTGCTCTCTTGTAG |
| | CT |
| CRE0009 (SEQ | CTGAGTCCTTTTGCATACATTTTTCAAATGATAACTCACTCTACCCACCC |
| ID NO: 263) | CCCTTCCCTACCCCCAAGGCGATTTATTGAAAAAACCACCTTATATGGT |
| | AATATTGCTAACACACCGTCAGCTGGCCTTTTTAGGGACTTTGTTTAAA |
| | GAAGATCCGCCTCTGGGGTTTTATATTGCTCTGGTATTCATGCCAAAGA |
| | CACACCAG |
| CRE0010_ITG | GTTTCTTAGCAGCTGCTGCTGTGTCCAAGGCTTGGAATTGCTGTGGTGA |
| B1BP2 (SEQ | ATCTAAAACTGTCTCAGTAGTGGTGAGCTGACCTCACCCAAGTTCAAAG |
| ID NO: 264) | CCCTACTCTGCCTGATCCTTTTTTCCTGAGCCTCAGAGCTAAAATGCCCC |
| | CGAGCTCTTTCCTATTGGCTGGAAAGACGAATTGAAGTTCCCTTGCCCA |
| | TGTTAGGAGGTGTACGCCTCCTGAACTAAAGATAGAAACAGCTGGCCC |
| | TTCCAGGCAGCTAAAAGCCTCCAGACTAAGAGGTGTTCCCCATTCGG |
| CRE0010_ | GCCGCGAAGACCGGAAGCTGGGGCGGCCCCGGGCCGCGCGCGCTGGG |
| ALDOA | CCTGGGAGGCGAAACTCAGCTTCCTTCGTTTCCGACTTTTCCATCCGCG |
| (SEQ ID | TCCTCCACTTCCCCGTTCCGCCCTCCCCCATTGCCAACATTCTGGCTGAG |
| NO: 265) | TCACGGCGCCCCAGAGCGCGCCAGGCTGGGGGAAAGGAGCAGAAGGG |
| | AGGGCCCTAGCGACCCGCGGGATGTGGTCCGAGTCACGTCCGAGGGGG |
| | GTGGGGAGGGATCGTGTTCTCGGCGCCCGCCCCTTCCTAGCGCGGCCTC |
| | TGGGCTGCGCCTCTCGGGGGCGGCCCGTAGCCCAGTCCGTCGCCTGCCA |
| | TTGGACGCCGCCCGCTCCTCGTAAAGGAAAAAGCTCGGCGGAGGGCGG |
| | AGTGGTGCCTTTAAAAGGCCGGGCGCCGCCTTCCGCCTGCCCGCCTCCT |
| | GCGCCGCCCCTTCCGAGGCTAAATCGGCTGCGTTCCTCTCGGAACGCGC |
| | CGCAGAAGGGGTCCTGGTGACGAGTCCCGCGTTCTCTCC |
| CRE0034 (SEQ | CCATGTTCCCGGCGAAGGGCCAGCTGTCCCCCGCCAGCTAGACTCAGC |
| ID NO: 266) | ACTTAGTTTAGGAACCAGTGAGCAAGTCAGCCCTTGGGGCAGCCCATA |
| | CAAGGCCATGGGGCTGGGCAAGCTGCACGCCTGGGTCCGGGGTGGGCA |
| | CGGTGCCCGGGCAACGAGCTGAAAGCTCATCTACTCTCAGGGGCCCCT |
| | CCCTGGGGACAGCCCCTCCTGGCTAGTCACACCCTGTAGGCTCCTCTAT |
| | ATAACCCAGGGGCACAGGGGCTGCCCCCGGGTCACCACCACCTCCACA |
| | GCACAGACAGACACTCAGGAGCCAGC |
| CRE0037 (SEQ | AGGTCCCTATATGGTTGTGTTAGAGTGAACGGCCAGCTTCAGCCCGTCT |
| ID NO: 267) | TTGCTCCTTGTTTGGGAAGCGAGTGGGAGGGGATCAGAGCAAGGGGCT |
| | ATATAACCCTTCAGCGTTCAGCCTCCCGGGACACCACCCACCCAGAGTG |
| | GAGAAGCCCAGCCAGTCGCTGTCA |
| CRE0046 (SEQ | CCCGGCAGACGCTCCTTATACGGCCCGGCCTCGCTCACCTGGGCCGCGG |
| ID NO: 268) | CCAGGAGCGCCTTCTTTGGGCAGCGCCGGGCCGGGGCCGCGCCGGGCC |
| | CGACACCCAAATATGGCGACGGCCGGGGCCGCATTCCTGGGGGCCGGG |
| | CGGCGCTCCCGCCCGCCTCGATAAAAGGCTCCGGGGCCGGCGGCGGCC |
| | CACGAGCTACCCGGAGGAGCGGGAG |
| CRE0048 (SEQ | GACTCAGGGGCGCAGGCCTCTTGCGGGGGAGCTGGCCTCCCCGCCCCC |
| ID NO: 269) | ACGGCCACGGGCCGCCCTTTCCTGGCAGGACAGCGGGATCTTGCAGCT |
| | GTCAGGGGAGGGGAGGCGGGGGCTGATGTCAGGAGGGATACAAATAG |
| | TGCCGACGGCTGGGGGCCCTGTCTCCCCTCGCCGCATCCACTCTCCGGC |
| | CGGCCGCCTGCCCGCCGCCTCCTCCGTGCGCCCGCCAGCCTCGCCCGCG |
| | CCGTCACC |
| CRE0049 (SEQ | CATGTTCCCGGCGAAGGGCCAGCTGTCCCCCGCCAGCTAGACTCAGCA |
| ID NO: 270) | CTTAGTTTAGGAACCAGTGAGCAAGTCAGCCCTTGGGGCAGCCCATAC |
| | AAGGCCATGGGGCTGGGCAAGCTGCACGCCTGGGTCCGGGGTGGGCAC |
| | GGTGCCCGGGCAACGAGCTGAAAGCTCATCTGCTCTCAGGGGCCCCTC |
| | CCTGGGGACAGCCCCTCCTGGCTAGTCACACCCTGTAGGCTCCTCTATA |
| | TAACCCAGGGGCACAGGGGCTGCCCTCATTCTACCACCACCTCCACAG |
| | CACAGACAGACACTCAGGAGCCAGCCAGC |
| CRE0053.2 | CCACCGCCTGCTGCCACGGCCGGCCGTATAAATAGAGGCGAGGAGCAG |
| SRL_mp (SEQ | CTGGGCTCTCTTGGCAGTCACC |
| ID NO: 271) | |
| CRE0054 (SEQ | CCAGCTGCCTGCCCCCTGCCTGGCACAGCCCGTACCTGGCCGCACGCTC |
| ID NO: 272) | CCTCACAGGTGAAGCTCGAAAACTCCGTCCCCGTAAGGAGCCCCGCTG |
| | CCCCCCGAGGCCTCCTCCCTCACGCCTCGCTGCGCTCCCGGCTCCCGCA |
| | CGGCCCTGGGAGAGGCCCCCACCGCTTCGTCCTTAACGGGCCCGGCGG |
| | TGCCGGGGGATTATTTCGGCCCCGGCCCCGGGGGGGCCCGGCAGACGC |
| | TCCTTATACGGCCCGGCCTCGCTCACCTGGGCCGCGGCCAGGAGCGCCT |
| | TCTTTGGGCAGCGCCGGGCCGGGGCCGCGCCGGGCCCGACACCCAAAT |
| | ATGGCGACGGCCGGGGCCGCATTCCTGGGGGCCGGGCGGCGCTCCCGC |
| | CCGCCTCGATAAAAGGCTCCGGGGCCGGCGGCGGCCCACGAGCTACCC |
| | GGAGGAGCGGGAGGCG |
| CRE0055 (SEQ | TCAAAGCCCTACTCTGCCTGATCCTTTTTTCCTGAGCCTCAGAGCTAAA |
| ID NO: 273) | ATGCCCCCGAGCTCTTTCCTATTGGCTGGAAAGACGAATTGAAGTTCCC |
| | TTGCCCATGTTAGGAGGTGTACGCCTCCTGAACTAAAGATAGAAACAG |
| | CTGGCCCTTCCAGGCAGCTAAAAGCCTCCAGACTAAGAGGTGTTCCCC |
| | ATTCGG |
| CRE0056 (SEQ | TCAAAGCCCTACTCTGCCTGATCCTTTTTTCCTGAGCCTCAGAGCTAAA |
| ID NO: 274) | ATGCCCCCGAGCTCTTTCCTATTGGCTGGAAAGACGAATTGAAGTTCCC |
| | TTGCCCATGTTAGGAGGTGTACGCCTCCTGAACTAAAGATAGAAACAG |
| | CTGGCCCTTCCAGGCAGCTAAAAGCCTCCAGACTAAGAGGTGTTCCCC |
| | ATTCGGCAGCCAGACTCCTTGAAATACCCTTTCAGTAATCATTCAACCA |
| | ACGCTTCC |
| CRE0070 (SEQ | CGGCCGGGGCCGCATTCCTGGGGGCCGGGCGGTGCTCCCGCCCGCCTC |
| ID NO: 42) | GATAAAAGGCTCCGGGGCCGGCGGCGGCCCACGAGCTACCCGGAGGA |
| | GCGGGAGGCG |
| CRE0070.2 | CGGCCGGGGCCGCATTCCTGGGGGCCGGGCGGTGCTCCCGCCCGCCTC |
| (SEQ ID NO: | GATAAAAGGCTCCGGGGCCGGCGGCGGCCCAC |
| 275) | |
| CRE0072 (SEQ | GTTTCTTAGCAGCTGCTGCTGTGTCCAAGGCTTGGAATTGCTGTGGTGA |
| ID NO: 276) | ATCTAAAACTGTCTCAGTAGTGGTGAGCTGACCTCACCCAAGTTCAAAG |
| | CCCTACTCTGCCTGATCCTTTTTTCCTGAGCCTCAGAGCTAAAATGCCCC |
| | CGAGCTCTTTCCTATTGGCTGGAAAGACGAATTGAAGTTCCCTTGCCCA |
| | TGTTAGGAGGTGTACGCCTCCTGAACTAAAGATAGAAACAGCTGGCCC |
| | TTCCAGGCAGCTAAAAGCCTCCAGACTAAGAGGTGTTCCCCATTCGGCA |
| | GCCAGACTCCTTGAAATACCCTTTCAGTAATCATTCAACCAACGCTTCC |
| SKM_14 (SEQ | TTCTCCTCTATAAATACCCGCTCTGGTATTTGGGGTTGGCAGCTGTTGCT |
| ID NO: 277) | GCCAGGGAGATGGTTGGGTTGACGGGATCTTGCAGCTGTCAGGGGAGG |
| | GGAGGCGGGGGCTGATGTCAGGAGGGATACAAATAGTGCCGACGGCTG |
| | GGGGCCCTGTCTCCCCTCGCCGCATCCACTCTCCGGCCGGCCGCCTGCC |
| | CGCCGCCTCCTCCGTGCGCCCGCCAGCCTCGCCCGCGCCGTCACC |
| SKM_18.2 | ATAAATACCCGCTCTGGTATTTGGGGTTCTCCTCTATAAATACCCGCTCT |
| (SEQ ID NO: | GGTATTTGGGGTTGGCAGCTGTTGCGGGATCTTGCAGCTGTCAGGGGAG |
| 278) | GGGAGGCGGGGGCTGATGTCAGGAGGGATACAAATAGTGCCGACGGCT |
| | GGGGGCCCTGTCTCCCCTCGC |
| SKM_18 (SEQ | ATAAATACCCGCTCTGGTATTTGGGGTTCTCCTCTATAAATACCCGCTCT |
| ID NO: 55) | GGTATTTGGGGTTGGCAGCTGTTGCGGGATCTTGCAGCTGTCAGGGGAG |
| | GGGAGGCGGGGGCTGATGTCAGGAGGGATACAAATAGTGCCGACGGCT |
| | GGGGGCCCTGTCTCCCCTCGCCGCATCCACTCTCCGGCCGGCCGCCTGC |
| | CCGCCGCCTCCTCCGTGCGCCCGCCAGCCTCGCCCGCGCCGTCACC |
| SKM_20 (SEQ | ATTTTTAAAGACTGAGGAATTAGGCACCTGTCATTTTTGCCAGCTGGTG |
| ID NO: 56) | TAGATGTTAAAAATTACTGTCACTCTTCCGCCTGCTACTTTATTTTGCAC |
| | CTGCTGTTACTTGAGTTACAGGCATTTCACACATGGTAATTTAATAAGG |
| | TTAGTTCCCATGACACACCGCCTGCTGCCACGGCCGGCCGTATAAATAG |
| | AGGCGAGGAGCAGCTGGGCTCTCTTGGCAGTCACC |
| RSV promoter | CAATTCTCATGTTTGACAGCTTATCATCGCAGATCCGTATGGTGCACTC |
| (SEQ ID NO: | TCAGTACAATCTGCTCTGATGCCGCATAGTTAAGCCAGTATCTGCTCCC |
| 279) | TGCTTGTGTGTTGGAGGTCGCTGAGTAGTGCGCGAGCAAAATTTAAGCT |
| | ACAACAAGGCAAGGCTTGACCGACAATTGCATGAAGAATCTGCTTAGG |
| | GTTAGGCGTTTTGCGCTGCTTCGCGATGTACGGGCCAGATATACGCGTA |
| | TCTGAGGGGACTAGGGTGTGTTTAGGCGAAAAGCGGGGCTTCGGTTGT |
| | ACGCGGTTAGGAGTCCCCTCAGGATATAGTAGTTTCGCTTTTGCATAGG |
| | GAGGGGGAAATGTAGTCTTATGCAATACTCTTGTAGTCTTGCAACATGG |
| | TAACGATGAGTTAGCAACATGCCTTACAAGGAGAGAAAAAGCACCGTG |
| | CATGCCGATTGGTGGAAGTAAGGTGGTACGATCGTGCCTTATTAGGAA |
| | GGCAACAGACGGGTCTGACATGGATTGGACGAACCACTGAATTCCGCA |
| | TTGCAGAGATATTGTATTTAAGTGCCTAGCTCGATACAATAAACGCCAT |
| | TTGACCATTCACCACATTGGTGTGCACCTCCAAGCTG |
| DES_mp_v1 | CGGGATCTTGCAGCTGTCAGGGGAGGGGAGGCGGGGGCTGATGTCAGG |
| SEQ ID NO: | AGGGATACAAATAGTGCCGACGGCTGGGGGCCCTGTCTCCCCTCGCCG |
| 280) | CATCCACTCTCCGGCCGGCCGCCTGCCCGCCGCCTCCTCCGTGCGCCCG |
| | CCAGCCTCGCCCGCGCCGTCACC |
| CRE0099 (SEQ | CCACTACGGGTCTAGGCTGCCCATGTAAGGAGGCAAGGCCTGGGGACA |
| ID NO: 281) | CCCGAGATGCCTGGTTATAATTAACCCAGACATGTGGCTGCCCCCCCCC |
| | CCCCAACACCTGCTGCCTCTAAAAATAACCCTGTCCCTGGTGGATCCCC |
| | TGCATGCGAAGATCTTCGAACAAGGCTGTGGGGGACTGAGGGCAGGCT |
| | GTAACAGGCTTGGGGGCCAGGGCTTATACGTGCCTGGGACTCCCAAAG |
| | TATTACTGTTC |
| HTMB ev_4 | ATAAATACCCGCTCTGGTATTTGGGG |
| (SEQ ID NO: | |
| 282) | |

TABLE 11
Promoter elements from shortened
synthetic promoters of Table 8

| | | |
|---|---|---|
| BG_mp | GGGCTGGGCATAAAAGTCAGGGCAG | 53 |
| (SEQ ID NO: | AAGCCATCTATTGCTTACTTTGCTT | |
| 402) | CTG | |
| SCP1 | GTACTTATATAAGGGGGTGGGGGCG | 81 |
| (SEQ ID NO: | CGTTCGTCCTCAGTCGCGATCGAAC | |
| 403) | ACTCGAGCCGAGCAGACGTGCCTAC | |
| | GGACCG | |
| CRE0070 | CGGCCGGGGCCGCATTCCTGGGGGC | 105 |
| (SEQ ID NO: | CCGGGCGGTGCTCCCGCCGCCTCGA | |
| 404) | TAAAAGGCTCCGGGGCCGGCGGCGG | |
| | CCCACGAGCTACCCGGAGGAGCGGG | |
| | AGGCG | |
| CRE0053 | CACCGCCTGCTGCCACGGCCGGCCG | 69 |
| (SEQ ID NO: | TATAAATAGAGGCGAGGAGCAGCTG | |
| 405) | GGCTCTCTTGGCAGTCACC | |

TABLE 5
Other elements (e.g. introns/UTR)

| Name | SEQUENCE |
|---|---|
| HBB | TACTAGCAGCTACAATCCAG |
| (SEQ ID | CTACCATTCTGCTTTTATTT |
| NO: 283) | TATGGTTGGGATAAGGCTGG |
| | ATTATTCTGAGTCCAAGCTA |
| | GGCCCTTTTGCTAATCATGT |
| | TCATACCTCTTATCTTCCTC |
| | CCACAGCTCCTGGGCAACGT |
| | GCTGGTCTGTGTGCTGGCCC |
| | ATCACTTTGGCAAAGAATT |
| CMV-IE | TCAGATCGCCTGGAGACGCC |
| 5′UTR and | ATCCACGCTGTTTTGACCTC |
| intron | CATAGAAGACACCGGGACCG |
| (SEQ ID | ATCCAGCCTCCGCGGCCGGG |
| NO: 65) | AACGGTGCATTGGAACGCGG |
| | ATTCCCCGTGCCAAGAGTGA |
| | CGTAAGTACCGCCTATAGAC |
| | TCTATAGGCACACCCCTTTG |
| | GCTCTTATGCATGAACGGTG |
| | GAGGGCAGTGTAGTCTGAGC |
| | AGTACTCGTTGCTGCCGCGC |
| | GCGCCACCAGACATAATAGC |
| | TGACAGACTAACAGACTGTT |
| | CCTTTCCATGGGTCTTTTCT |
| | GCAG |

TABLE 12
CRMs from promoters of Table 8

| | | |
|---|---|---|
| CRM from | TCTGAGGGAGACAGGGAGGCATGATCACTGCCAAATGCCCAC | 159 |
| SP0497, SP0499 | CAAGGACAAGGCACATCCCAGGGAGACAGACGCAGACCTGG | |
| and SP0512 | TGCCCTCTGGACACTGGCATTCCTGGAGTTCTCCTCTATAAAT | |
| (SEQ ID NO: 359) | ACCCGCTCTGGTATTTGGGGTTGGCAGCTGTTG | |
| CRM_SP0498 | TCTGAGGGAGACAGGGAGGCATGATCACTGCCAAATGCCCAC | 183 |
| (SEQ ID NO: 360) | CAAGGACAAGGCACATCCCAGGGAGACAGACGCAGACCTGG | |
| | TGCCCTCTGGACACTGGCATTCCTGGAGAGGGTCAGTGTCCTG | |
| | CCCCAACCCATGAGATGACAGACTATAATAGCCACAGGATTA | |
| | ACATAGCAGGCATTG | |
| CRM_SP0500 | CCAGCCCACCTGTCCCAATGCTGACTTAGTGCAAGGCGAGCC | 192 |
| (SEQ ID NO: 362) | AGCAAGGAGGGAGGACAGGTGGCAGTGGGGGGTGAGGAGCA | |
| | TCTAAAAATAGCCCCAGCCCACCTGTCCCAATGCTGACTTAGT | |
| | GCAAGGCGAGCCAGCAAGGAGGGAGGACAGGTGGCAGTGGG | |
| | GGGTGAGGAGCATCTAAAAATAGCC | |
| CRM_SP0501 | CCAGCCCACCTGTCCCAATGCTGACTTAGTGCAAGGCGAGCC | 144 |
| (SEQ ID NO: 363) | AGCAAGGAGGGAGGACAGGTGGCAGTGGGGGGTGAGGAGCA | |
| | TCTAAAAATAGCCTTCTCCTCTATAAATACCCGCTCTGGTATT | |
| | TGGGGTTGGCAGCTGTTG | |
| CRM from | CTAGACTAGCATGCTGCCCATGTAAGGAGGCAAGGCCTGGGG | 176 |
| SP0502, SP0515, | ACACCCGAGATGCCTGGTTATAATTAACCCAGACATGTGGCT | |
| SP0521 and | GCCCCCCCCCCCCCAACACCTGCTGCCTCTAAAAATAACCCTG | |
| SP4169 | CTTCTCCTCTATAAATACCCGCTCTGGTATTTGGGGTTGGCAG | |
| (SEQ ID NO: 364) | CTGTTG | |
| CRM from SP0503 | TCGTCCCCTGGCTGGCCCATGTAATCTGAGCCCAGCATTGTAC | 183 |
| and SP0516 | ATATCCTGGGAACAGCTGACAATGCAGTGGTCAGACAGCTGG | |
| (SEQ ID NO: 365) | TGGGGCCAGCTAGAGCTGGCAGGGTTGGCTGGGAGGGGAGTG | |
| | TAGGCTGATTCTCCTCTATAAATACCCGCTCTGGTATTTGGGG | |
| | TTGGCAGCTGTTG | |
| CRM from SP0504 | TTGGCCCAGGTCACACTGGGGTGAGGCTAGTGTTCCTGAGCCT | 190 |
| and SP0517 | TGACAAGGAGACAGCTTGAAATAGACGAGTGTCACATTTCTG | |
| (SEQ ID NO: 366) | AGCAGCTGTGTGGCGACAGCAGGAGGGGTAGGGAATAGACA | |
| | GTATAAAAGAGAAAGCTTCTCCTCTATAAATACCCGCTCTGGT | |
| | ATTTGGGGTTGGCAGCTGTTG | |
| CRM | CCCTGCCATCTTGGGTTTCAGGGCAGAGGAGTCTTGCTAATTT | 194 |
| from SP0505 and | TGATGCCTATTTTTGGACACTTCAGCTGCCACTGGCTCCTTAT | |
| SP0518 | AAACGCATGACACCCCATGCAAACACACTACCCCTCCCTCCA | |
| (SEQ ID NO: 367) | CTGCTGACAGGTGTGTGGTTCTCCTCTATAAATACCCGCTCTG | |
| | GTATTTGGGGTTGGCAGCTGTTG | |
| CRM_SP0506 | AATTATTTTTAATAACACTTACTGGTAAGAGAAAGGGGAGAA | 199 |
| (SEQ ID NO: 368) | ACCTTAGACAGGCACTTAGATGTGACTAAGGCAGGTTTATCTC | |
| | TGATTCCAAAGCACTGGAGTGGAAGTCACACCGTGACTCAGA | |
| | GCATTGTGATGGGCCAGCTGTCCATTCTCCTCTATAAATACCC | |
| | GCTCTGGTATTTGGGGTTGGCAGCTGTTG | |
| CRM_SP0507 | CAGTGTTCCTATATTTATCCCACCATACAGAGCTTCCTTTGCCT | 199 |
| (SEQ ID NO: 369) | CAGAAGGACCAGCAGTTTCGCTAGCTTAACAAAACCAGCCAC | |
| | TCAGGGTATTGGTTTACAGTCAAGCAACTCTGGGAGAGGGCA | |
| | GCTGCTCTCAGACATCATACAGCTTCTCCTCTATAAATACCCG | |
| | CTCTGGTATTTGGGGTTGGCAGCTGTTG | |
| CRM_SP0508 | CTTCTCAAGCCAAAGGAGCAAGAGTTAAAAATAACAGGCTCA | 165 |
| (SEQ ID NO: 370) | CCCTGGCAGCCACCTGTGCTGGCCAGCCCCACCCCATCCCTCC | |
| | CTCGGGGACAGCTGCAGCTCCTCAGGCCCCGCCCGGGACATT | |
| | TTGGGAACACTTTCTCCTCTTACTTCTCATCTTCAGGG | |
| CRM from | AGGGTCAGTGTCCTGCCCCAACCCATGAGATGACAGACTATA | 200 |
| SP0509 and | ATAGCCACAGGATTAACATAGCAGGCATTGATTTTTAAAGAC | |
| SP0520 | TGAGGAATTAGGCACCTGTCATTTTTGCCAGCTGGTGTAGATG | |
| (SEQ ID NO: 371) | TTAAAAATTACTGTCACTCTTCCGCCTGCTACTTTATTTTGCAC | |
| | CTGCTGTTACTTGAGTTACAGGCATTTCA | |
| CRM_SP0510 | ATTTTTAAAGACTGAGGAATTAGGCACCTGTCATTTTTGCCAG | 176 |
| (SEQ ID NO: 372) | CTGGTGTAGATGTTAAAAATTACTGTCACTCTTCCGCCTGCTA | |
| | CTTTATTTTGCACCTGCTGTTACTTGAGTTACAGGCATTTCATT | |
| | CTCCTCTATAAATACCCGCTCTGGTATTTGGGGTTGGCAGCTG | |
| | TTG | |
| CRM_SP0511 | AGACTGGGGCAGGTGCAGGCTGGATTGGGTTTCCAGAGGCTA | 215 |
| (SEQ ID NO: 373) | TATATATAAAGGCTGCCGGGAGCCCCAGGGCCGCTCCCTGAG | |
| | GGCACAACACTGTGGGGGCCCAGCCAGGCCCACATTCCTTTC | |
| | CAGAGGCCAGCTCTCCATTTATAGCCCCTGGGCAGAGCAGCTT | |
| | CTCCTCTATAAATACCCGCTCTGGTATTTGGGGTTGGCAGCTG | |
| | TTG | |
| CRM_SP0513 | CCAGCCCACCTGTCCCAATGCTGACTTAGTGCAAGGCGAGCC | 216 |
| (SEQ ID NO: 375) | AGCAAGGAGGGAGGACAGGTGGCAGTGGGGGGTGAGGAGCA | |
| | TCTAAAAATAGCCAGGGTCAGTGTCCTGCCCCAACCCATGAG | |
| | ATGACAGACTATAATAGCCACAGGATTAACATAGCAGGCATT | |
| | GTTCTCCTCTATAAATACCCGCTCTGGTATTTGGGGTTGGCAG | |
| | CTGTTG | |
| CRM_SP0514 | CCAGCCCACCTGTCCCAATGCTGACTTAGTGCAAGGCGAGCC | 192 |
| (SEQ ID NO: 376) | AGCAAGGAGGGAGGACAGGTGGCAGTGGGGGGTGAGGAGCA | |
| | TCTAAAAATAGCCTTCTCCTCTATAAATACCCGCTCTGGTATT | |
| | TGGGGTTGGCAGCTGTTGTTCTCCTCTATAAATACCCGCTCTG | |
| | GTATTTGGGGTTGGCAGCTGTTG | |
| CRM_SP0519 | CTTCTCAAGCCAAAGGAGCAAGAGTTAAAAATAACAGGCTCA | 213 |
| (SEQ ID NO: 381) | CCCTGGCAGCCACCTGTGCTGGCCAGCCCCACCCCATCCCTCC | |
| | CTCGGGGACAGCTGCAGCTCCTCAGGCCCCGCCCGGGACATT | |
| | TTGGGAACACTTTCTCCTCTTACTTCTCATCTTCAGGGTTCTCC | |
| | TCTATAAATACCCGCTCTGGTATTTGGGGTTGGCAGCTGTTG | |
| CRM | GCTGCCCATGTAAGGAGGCAAGGCCTGGGGACACCCGAGATG | 162 |
| from SP0522, | CCTGGTTATAATTAACCCAGACATGTGGCTGCCCCCCCCCCCC | |
| SP0523 and | AACACCTGCTGCCTCTAAAAATAACCCTGTTCTCCTCTATAAA | |
| SP0524 | TACCCGCTCTGGTATTTGGGGTTGGCAGCTGTTG | |
| (SEQ ID NO: 385) | | |

### Functional Variants of Muscle-Specific Promoters

In some embodiments, a functional variant of a muscle-specific promoter can be viewed as a promoter element which, when substituted in place of a reference promoter element in a promoter, substantially retains its activity. For example, a functional variant of muscle-specific promoter which comprises a functional variant of a given promoter disclosed herein preferably retains at least 350%, or at least 40%, or at least 45%, or at least 50%, or at least 55%, or at least 60%, or at least 70% or at least 80% of its activity, more preferably at least 90% of its activity, more preferably at least 95% of the activity of the unchanged promoter, and yet more preferably 100% of the activity (as compared to the unchanged promoter sequence comprising the unmodified promoter element). Suitable assays for assessing muscle-specific promoter activity are known in the art.

In some embodiments, a functional variant or a functional fragment of a muscle-specific promoter disclosed herein has at least about 75% sequence identity to, or at least about 80% sequence identity to, at least about 90% sequence identity to, at least about 95% sequence identity to, at least about 98% sequence identity to the original unmodified sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence.

### Compositions Containing the FKRP Nucleic Acid and Vectors

A further aspect of the invention relates to a cell comprising the synthetic nucleic acid of the invention and/or vector comprising the synthetic nucleic acid of the invention (e.g., an isolated cell, a transformed cell, a recombinant cell, etc.). Thus, various embodiments of the invention are directed to recombinant host cells containing a vector (e.g., expression cassette) comprising the synthetic nucleic acid of the invention. Such a cell can be isolated and/or present in an animal, e.g., a transgenic animal. Transformation of cells is described further below.

Another aspect of the invention relates to a transgenic animal comprising the synthetic nucleic acid, vector, and/or transformed cell of the invention. A transgenic animal may include, but is not limited to, a farm animal (e.g., pig, goat, sheep, cow, horse, rabbit and the like), rodents (such as mice, rats and guinea pigs), and domestic pets (for example, cats and dogs). In some embodiments, the transgenic animal is not a human.

A transgenic animal may be produced by introducing into a single cell embryo the synthetic nucleic acid of the invention encoding FKRP in a manner such that the synthetic nucleic acid is stably integrated into the DNA of germ line cells of the mature animal, and is inherited in normal Mendelian fashion. The transgenic animal of this invention would have a phenotype of producing FKRP in body fluids and/or tissues. In some embodiments, the FKRP may be removed from these fluids and/or tissues and processed, for example for therapeutic use. (See, e.g., Clark et al. “Expression of human anti-hemophilic factor IX in the milk of transgenic sheep” Bio/Technology 7:487-492 (1989); Van Cott et al. “Haemophilic factors produced by transgenic livestock: abundance can enable alternative therapies worldwide” Haemophilia 10(4):70-77 (2004), the entire contents of which are incorporated by reference herein).

DNA molecules can be introduced into cells and embryos by a variety of means including but not limited to microinjection, calcium phosphate mediated precipitation, liposome fusion, or retroviral infection of totipotent or pluripotent stem cells. The transformed cells can then be introduced into embryos and incorporated therein to form transgenic animals. Methods of making transgenic animals are described, for example, in Transgenic Animal Generation and Use by L. M. Houdebine, Harwood Academic Press, 1997. Transgenic animals also can be generated using methods of nuclear transfer or cloning using embryonic or adult cell lines as described for example in Campbell et al., Nature 380:64-66 (1996) and Wilmut et al., Nature 385:810-813 (1997). Further a technique utilizing cytoplasmic injection of DNA can be used as described in U.S. Pat. No. 5,523,222.

FKRP-producing transgenic animals can be obtained by introducing a chimeric construct comprising the synthetic nucleic acid of the invention. Methods for obtaining transgenic animals are well-known. See, for example, Hogan et al., Manipulating the Mouse Embryo, (Cold Spring Harbor Press 1986); Krimpenfort et al., Bio Technology 9:88 (1991); Palmiter et al., Cell 41:343 (1985), Kraemer et al., Genetic Manipulation of the Early Mammalian Embryo, (Cold Spring Harbor Laboratory Press 1985); Hammer et al., Nature 315:680 (1985); Wagner et al., U.S. Pat. No. 5,175,385; Krimpenfort et al., U.S. Pat. No. 5,175,384, Janne et al., Ann. Med. 24:273 (1992), Brem et al., Chim. Oggi. 11:21 (1993), Clark et al., U.S. Pat. No. 5,476,995, all incorporated by reference herein in their entireties.

The synthetic nucleic acid encoding FKRP, or vector and/or cell comprising said synthetic polynucleotide can be included in a pharmaceutical composition. Containers of such pharmaceutical compositions are encompassed in the invention. Some embodiments are directed to a kit which includes said synthetic nucleic acid, or vector and/or cell comprising said synthetic nucleic acid of the invention and/or reagents and/or instructions for using the kit, e.g., to carry out the methods of this invention.

A further aspect of the invention relates to the use of the synthetic nucleic acid encoding FKRP, or vector, expression cassette, and/or cell comprising one or more synthetic nucleic acid encoding FKRP. Thus, one aspect relates to a method of producing a FKRP polypeptide in a cell or in a subject, comprising delivering to the cell or the subject the synthetic nucleic acid, vector, and/or transformed cell of the invention, thereby producing the FKRP polypeptide in said cell or said subject. The synthetic nucleic acid, vector, and/or transformed cell are delivered under conditions whereby expression of the synthetic nucleic acid encoding FKRP occurs to produce a FKRP polypeptide. Such conditions are well known in the art.

In some embodiments, the pharmaceutical composition comprises recombinant AAV vector in a buffer (e.g., excipient) of about pH 7.0 to about pH 8.0. In some embodiments, the pH of the buffer is from about 7.0 to about 7.5. In preferred embodiment, the pH of the buffer is less than 7.5. In several embodiments, the buffer is phosphate buffer saline (PBS). In certain embodiments, the buffer or, excipient comprises ions selected from the group consisting of sodium, potassium, phosphate, chloride, calcium, magnesium, sulfate, citrate and any combination thereof. The pharmaceutical composition further comprises polyol, sugar or, similar. In some embodiment, the pharmaceutical composition comprises glycerol or, propylene glycol, or, polyethylene glycol, or, sorbitol, or, mannitol. In several embodiments, the sorbitol concentration ranges from about 1% (w/v) to about 10% (w/v). In some embodiments, the sorbitol concentration ranges from about 2% (w/v) to about 8% (w/v). In preferred embodiments, the sorbitol concentration ranges from about 3% (w/v) to about 6% (w/v). In certain embodiments, the sorbitol concentration is 1% (w/v), 2% (w/v), 3% (w/v), 4% (w/v), 5% (w/v), 6% (w/v), 7% (w/v), 8% (w/v), 9% (w/v), or, 10% (w/v). The pharmaceutical composition further comprises a non-ionic surfactant. In some embodiments, the non-ionic surfactant is selected from the group consisting of polyoxyethylene-polyoxypropylene block copolymers, alkylglucosides, alkyl phenol ethoxylates, polysorbates, polyoxyethylene alkyl phenyl ethers, and any combinations thereof. In some embodiments, the non-ionic surfactant is poloxamer 188or, Ecosurf SA-15. In certain embodiments, poloxamer 188, or Ecosurf SA-15 concentration is 0.0005% (w/v), 0.0008% (w/v), 0.0009% (w/v), 0.001% (w/v), 0.002% (w/v), 0.0025% (w/v), 0.003% (w/v), 0.0035% (w/v), 0.004% (w/v), 0.0045% (w/v), 0.005% (w/v), 0.006% (w/v), 0.007% (w/v), 0.008% (w/v), 0.009% (w/v), or, 0.01% (w/v).

The pharmaceutical composition comprises at least 1×10¹⁰ vg/ml recombinant AAV vector as disclosed in the present invention. In some embodiments the pharmaceutical composition comprises about 1×10¹¹ vg/ml to about 1×10¹⁴ vg/ml recombinant AAV vector. In some embodiments, the pharmaceutical composition comprises about 1×10¹² vg/ml to about 8×10³ vg/ml recombinant AAV vector. In several embodiments, the pharmaceutical composition comprises about 1e¹³ vg/ml to about 6e¹³ vg/ml recombinant AAV9sc vector comprising nucleic acid encoding FKRP polypeptide as disclosed in the present invention, wherein the nucleic acid is operatively linked with a promoter selected from the group consisting of MCK promoter, dMCK promoter, tMCK promoter, enh358MCK promoter, CK6 promoter and Syn100 promoter, any promoter listed in Table 1-4 or 8-12, and derivatives thereof.

In some embodiments, a subject having limb girdle disease or, disorder or, in need thereof is administered with the rAAV of the present invention, wherein the rAAV is administered at a dose ranging from about 5e¹² vg/kg to about 6e¹³ vg/kg. In some embodiments, rAAV is administered at 5e² vg/kg, 9e² vg/kg, 1e¹³ vg/kg, 2e¹³ vg/kg, 3e¹³ vg/kg, 4e¹³ vg/kg, 5e¹³ vg/kg, or, 6e¹³ vg/kg. In some embodiments, the total dose of rAAV administered is 2e¹⁴ vg, 3e¹⁴ vg, 5e¹⁴ vg, 6e¹⁴ vg, 7e¹⁴ vg, 8e¹⁴ vg, 9e¹⁴ vg, 1e¹⁵ vg, 2e⁵ vg, or 3e¹⁵ vg.

In some embodiments, the rAAV of the present invention is administered at increasing doses over time. For example, the rAAV can be delivered in a first dose at 1e13 vg/kg, and then at 3e13 vg/kg in a second dose. In one embodiment, the subject is administered at least 2 doses at 1e13 vg/kg, and at least 1 dose (e.g., at least 2, 3, 4 doses or more) at 3e13 vg/kg. In one embodiment, the doses are administered in intervals, e.g., at least 45 days apart.

Exemplary Formulation Pharmaceutical Compositions:

In various aspects of the present invention, the pharmaceutical composition comprises recombinant AAV vector comprising rAAV-FKRP (e.g. AAV9sc.Syn100.coHuFKRP), in 30 mM Phosphate pH 7.4, 200 mM NaCl, 5 mM KCl, 1% (w/v) mannitol, 0.0005% (w/v) IGEPAL CA 720 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In one aspect of the present invention, the pharmaceutical composition comprises recombinant AAV vector comprising rAAV-FKRP (e.g. AAV9sc.Syn100.coHuFKRP), in 20 mM Phosphate pH 7.4, 300 mM NaCl, 3 mM KCl, 3% (w/v) mannitol, 0.001% (w/v) Brij S20 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In several aspects of the present invention, the pharmaceutical composition comprises recombinant AAV vector comprising rAAV-FKRP (e.g. AAV9sc.Syn100.coHuFKRP), in 20 mM Phosphate pH 7.4, 300 mM NaCl, 3 mM KCl, 3% (w/v) sorbitol, 0.001% (w/v) Ecosurf SA-15 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In various aspects of the present invention, the pharmaceutical composition comprises recombinant AAV vector comprising rAAV-FKRP (e.g. AAV9sc.Syn100.coHuFKRP), in 10 mM Phosphate pH 7.4, 350 mM NaCl, 2.7 mM KCl, 5% (w/v) sorbitol, 0.001% (w/v) poloxamer 188 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

Several aspects of the present invention provided herein, the pharmaceutical composition comprises recombinant AAV vector comprising rAAV-FKRP (e.g. AAV9sc.Syn100.coHuFKRP), in 15 mM Phosphate pH 7.4, 375 mM NaCl, 3.5 mM KCl, 5% (w/v) sorbitol, 0.0005% (w/v) Tergitol NP-10 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

In one of the aspects of the present invention, the pharmaceutical composition comprises recombinant AAV vector comprising rAAV-FKRP (e.g. AAV9sc.Syn100.coHuFKRP), in 15 mM Phosphate pH 7.4, 375 mM NaCl, 3.5 mM KCl, 3% (w/v) glycerol, 0.0005% (w/v) Tween 80 to a fill volume of 5 ml. In some embodiments, the fill volume is 1 ml, 2 ml, 3 ml, 4 ml, 5 ml, 6 ml, 7 ml, 8 ml, 9 ml, or, 10 ml.

### Treatment and Therapeutics

Aspects of the invention relate to the use of the synthetic nucleic acid encoding FKRP, and the vectors and compositions comprising the synthetic nucleic acid, to increase the amount of functional FKRP in a cell (e.g., muscle cell) or in cells and tissues of a subject (e.g., muscle cells such as skeletal and/or cardiac muscle) in need thereof. In one aspect of the invention, synthetic nucleic acid encoding FKRP, the vectors and compositions comprising the synthetic nucleic acid can be delivered to a cell (e.g. a muscle cell such as skeletal and/or cardiac muscle) under conditions appropriate for expression of the FKRP, to thereby increase the amount of functional FKRP in the cell. In some embodiments, increasing the functional FKRP in the cell will also increase the glycosylation of α-dystroglycan in the cell. In one embodiment the cell is in vitro. In some embodiments, the cell is in vivo.

### Modulating FKRP Levels in a Cell Ex Vivo

The nucleic acids, vector, and virions as described herein can be used to modulate levels of functional FKRP in a cell. The method includes the step of administering to the cell a composition including a synthetic nucleic acid encoding FKRP described herein interposed between two AAV ITRs, as described herein. The cell can be from any animal into which a nucleic acid of the invention can be administered. Mammalian cells (e.g., humans, dogs, cats, pigs, sheep, mice, rats, rabbits, cattle, goats, etc.) from a subject with FKRP anomaly are typical target cells for use in the invention. In some embodiments, the cell is a skeletal muscle or heart muscle cell.

In another aspect, disclosed herein is a method of administering a nucleic acid encoding FKRP to a cell, comprising contacting the cell with a rAAV vector and/or rAAV genome as disclosed herein, under conditions for the nucleic acid to be introduced into the cell and expressed to produce FKRP. In some embodiments, the cell is a cultured cell. In some embodiments, the cell is a cell in vivo. In some embodiments, the cell is a mammalian cell (e.g. human). In some embodiments, the cell is a muscle cell (e.g., skeletal or cardiac muscle).

Another aspect of the invention relates to ex vivo delivery of cells transduced with rAAV vector disclosed herein (e.g., expressing the encoded FKRP protein). Such ex vivo gene delivery may be used to transplant cells originally obtained from a subject transduced with a rAAV vector as disclosed herein back into the subject. In a further embodiment, ex vivo stem cell (e.g., mesenchymal stem cell) therapy may be used to transplant cells transduced with a rAAV vector as disclosed herein cells back into the subject. A suitable ex vivo protocol may include several steps. For example, a segment of target tissue (e.g., muscle) may be harvested from the subject, and the rAAV vector described herein used to transduce the FKRP-encoding nucleic acid into the cells of the tissue. These genetically modified cells may then be transplanted back into the subject. Several approaches may be used for the reintroduction of cells into the subject, including intravenous injection, intraperitoneal injection, subcutaneous injection, or in situ injection into target tissue (muscle tissue). Microencapsulation of modified ex vivo cells transduced or infected with an rAAV vector as described herein is another technique that may be used with the invention. Autologous and allogeneic cell transplantation may be used according to the invention.

Such methods described herein can be used to treat a subject in need thereof (e.g., a subject having a FKRP anomaly). In one embodiment, the method comprises administering to the subject cells expressing FKRP produced by the above-discussed methods, in a pharmaceutically acceptable carrier and in a therapeutically effective amount. In some embodiments, the subject is a human.

### Increasing FKRP Levels and Activity in a Subject

The nucleic acids, vectors, and virions as described herein can be used to modulate levels of functional FKRP in a subject. The method includes administering to the subject a composition comprising the rAAV vector, comprising the rAAV genome as described herein, comprising the synthetic nucleic acid encoding FKRP interposed between two AAV ITRs, where the hFKRP is operatively linked to muscle-specific promoter. In one embodiment the subject is in need of such modulation.

As the term is used herein, “subject in need thereof” refers to the immediate or expected condition of the subject. Such a subject may have a diagnosed dystroglycanopathy disorder (e.g., a resulting from a FKRP anomaly such as LGMD2I) or be at risk of developing such a disorder. The subject can be any animal, e.g., mammals (e.g., human beings, dogs, cats, pigs, sheep, mice, rats, rabbits, cattle, goats, etc.). The methods and compositions of the invention are particularly applicable to FKRP-deficient human subjects that would benefit from an increase in the glycosylation of α-dystroglycan in one or more of their muscles (e.g., skeletal muscle and/or cardiac muscle). In one embodiment, the subject has an FKRP anomaly (e.g., a deficiency of FKRP). An FKRP anomaly is a condition that results in reduced levels of functional FKRP in muscle tissue of the subject as compared to the levels of functional FKRP in the same tissue of a normal subject. This may result in a deficiency in glycosylation α-dystroglycan. Such a condition may result from a direct mutation in the FKRP gene of the subject, or may result in an indirect disruption of the expression and/or processing of endogenous FKRP. Mutations in the FKRP gene have been found to contribute to various diseases/disorders such as limb-girdle muscular dystrophy 2I. Disorders known to benefit from an increase in the level of functional FKRP include, without limitation, limb-girdle muscular dystrophy 2I, congenital muscular dystrophy, Walker-Warburg syndrome, and muscle-eye-brain disease. A subject in need thereof, may have or be at risk for developing one or a combination of such conditions or disorders. A subject having, or at risk for developing, another condition that results in a dystroglycanopathy disorder that may improve from an increase in the levels of functional FKRP in their muscle tissue may also constitute a subject “in need thereof”. A subject may be determined as at risk for developing a condition by various means known in the art, e.g., genetic analysis, familial history, and/or preconditions associated with a predisposition for the disease or disorder.

In some embodiments, the subject is an adult. In some embodiments, the subject is a juvenile. In some embodiments, the subject is an infant. In some embodiments the subject manifests one or more symptoms of the disorder. In some embodiments the subject fails to manifest one or more symptoms of the disorder. In some embodiments, the subject demonstrates significant disease pathology prior to administration. In some embodiments, the subject demonstrates no significant disease pathology prior to administration.

Furthermore, the nucleic acids, vectors, and virions described herein may be administered to animals including human beings in any suitable formulation by any suitable method. For example, in any embodiment of the methods and compositions as disclosed herein, an rAAV vector, or rAAV genome as disclosed herein can be directly introduced into a subject, for delivery to skeletal muscle and heart muscle of the subject. Administration may be by any means that results in expression of the FKRP transgene in the target tissue (muscle). In some embodiments, administration is systemic (e.g., intravenous infusion). Various systemic routes of administration are known to the skilled practitioner and provided herein. The appropriate systemic route will depend upon the vector and the subject. In some embodiments, the administration is localized (e.g., directly to the muscle target).

In any embodiment of the methods and compositions as disclosed herein, the method is directed to treating the disorder (e.g., a dystroglycanopathy disorder and/or LGMD2I or another disorder that results from a deficiency of functional FKRP protein) in a subject, wherein a therapeutically effective amount rAAV vector and/or rAAV genome as disclosed herein is administered to a patient suffering from the disorder. Following administration, the exogenous FKRP nucleic acid is expressed in the target cells (muscle) of the subject, thereby increasing functional FKRP protein levels in the muscle tissue. Such an increase is detectable either directly (e.g., biopsy) or indirectly (e.g., functionally). In one embodiment, the increased functional FKRP protein levels compensates for a functional FKRP-deficiency that contributes to the disorder. In some embodiments, the effectiveness of a therapeutic compound disclosed herein to treat the disorder (e.g., LGMD2I) can be determined, without limitation, by observing an improvement in an individual based upon one or more clinical symptoms, and/or physiological indicators associated with the disorder. In some embodiments, an improvement in the symptoms associated with the disorder (e.g., LGMD2I) can be indicated by a reduced need for a concurrent therapy.

In some embodiments, the functional glycosylation of α-dystroglycan is substantially increased in skeletal muscle and or cardiac muscle of the subject. Such an increase may be detected by direct (e.g. biopsy) or indirect means (e.g., functionally). In some embodiments, the subject that receives the treatment exhibits a significant or substantial (sustained, statistically significant amount) reduction in serum creatine kinase compared to their serum creatine kinase levels prior to receiving the treatment. In some embodiments, the subject that receives the treatment exhibits a significant or substantial reduction in collagen deposition in the recipient skeletal muscle compared to their collagen deposition prior to receiving the treatment. In some embodiments, the treatment results in a significant increase in in vitro muscle force of the subject's recipient muscle tissue (e.g., soleus, diaphragm, and/or EDL). In some embodiments, the treatment results in the subject having the ability to perform physical tasks better or for a longer period of time, such as to run significantly further (e.g., in a treadmill test).

In some embodiments of the methods and compositions as disclosed herein, administration of a rAAV FKRP construct described herein (e.g., an AAV vector of any serotype as described in Table 6, including AAV9), the AAV vector or AAV genome, as disclosed herein is capable of reducing one or more of the following in the recipient subject (e.g., having a dystroglycanopathy described herein) serum creatine kinase levels, collagen deposition levels, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to before the administration or to a subject not receiving the same treatment. In some embodiments of the methods and compositions as disclosed herein, administration of a rAAV FKRP construct described herein (e.g., an AAV vector of any serotype as described in Table 6, including AAV9) as disclosed herein is capable of reducing one or more of the following in the recipient subject (e.g., having a dystroglycanopathy described herein) serum creatine kinase levels, collagen deposition levels, pain and/or lethargy e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to prior to the administration or to a subject not receiving the same treatment.

In some embodiments of the methods and compositions as disclosed herein, administration of a rAAV FKRP construct described herein (e.g., an AAV vector of any serotype as described in Table 6, including AAV9), the AAV vector or AAV genome, as disclosed herein is capable of reducing the adverse effects associated with the dystroglycanopathy disorder by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity of the adverse effects associated with the dystroglycanopathy disorder are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, the adverse effects associated with the dystroglycanopathy disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to prior to the administration or to a subject not receiving the same treatment. Such adverse effects include, without limitation, limited muscle strength, limited muscle mobility, muscle cramps, heart problems, vision problems, breathing difficulties, difficulty swallowing, weakness in muscles of the face, difficulty standing up, difficulty climbing stairs, difficulty running, difficulty jumping.

In some embodiments of the methods and compositions as disclosed herein, administration of a rAAV FKRP construct described herein (e.g., an AAV vector of any serotype as described in Table 6, including AAV9), the AAV vector or AAV genome, as disclosed herein is capable of increasing expression of functional FKRP and/or increasing functional glycosylation of α-dystroglycan in the skeletal muscle and/or cardiac muscle of the subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% as compared to prior to the administration or to a subject not receiving the same treatment.

In another embodiment, the expression of functional FKRP and/or the level of functional glycosylation of α-dystroglycan in the skeletal muscle and/or cardiac muscle of the subject is increased by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to prior to the administration or to a subject not receiving the same treatment.

In some embodiments of the methods and compositions as disclosed herein, administration of a rAAV FKRP construct described herein (e.g., an AAV vector of any serotype as described in Table 6, including AAV9), the AAV vector or AAV genome, as disclosed herein is capable of increasing the ability of the recipient subject to perform a given physical task (e.g., walk or run) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, or by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to prior to the administration or to a subject not receiving the same treatment.

In some embodiments of the methods and compositions as disclosed herein, administration of a rAAV FKRP construct described herein (e.g., an AAV vector of any serotype as described in Table 6, including AAV9), the AAV vector or AAV genome, as disclosed herein is capable of increasing the ability of the recipient subject to perform a given physical task (e.g., walk or run) by about 100%, 120%, 130%, 140%, 150%, 160%, 170%, 180%, 190%, 200%, 250%, 300%, 350%, 400%, 450%, 500%, etc. as compared to prior to the administration or to a subject not receiving the same treatment. Put another way, the ability to perform a given physical task is increased 2×, 3×, 4×, 5×, 6×, 7×, 8×, 10×, or more as compared to prior to the administration or to a subject not receiving the same treatment.

“Tidal volume” is the lung volume representing the normal volume of air displaced between normal inhalation and exhalation when extra effort is not applied. In a healthy, young human adult, tidal volume is approximately 500 mL per inspiration or 7 mL/kg of body mass. Tidal volume is compromised in subjects with dystroglycanopathy disorders (e.g., LGMD2I). In some embodiments of the invention, administration of a therapeutically effective amount of rAAV FKRP construct to a subject with a dystroglycanopathy disorder such as LGMD2I significantly improves the tidal volume of the subject.

In some embodiments of the methods and compositions as disclosed herein, administration of a rAAV FKRP construct described herein (e.g., an AAV vector of any serotype as described in Table 6, including AAV9), the AAV vector or AAV genome, as disclosed herein is capable of increasing the in vitro muscle force (e.g., soleus, diaphragm and/or EDL muscle), and/or tidal volume, (e.g., as analyzed as described herein), by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, or by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to prior to the administration or to a muscle in the subject not receiving the same treatment, or to the muscle of a subject not receiving the same treatment.

### Immunosuppression

In any embodiment of the methods and compositions as disclosed herein, a subject being administered a rAAV vector or rAAV genome comprising the FKRP transgene as disclosed herein is administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors 31 and 02, TNF and others that are publicly known).

Immunosuppressive agents and methods for suppressing the immune system in a subject is described in, e.g., U.S. Pat. Nos. 10,028,993; 9,592,247; 8,809,282; 9,186,420; and 10,098,905.

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO 2010057626, U.S. Pat. Nos. 9,707,279, 8,323,908, US 20190345533, US 20190262434, and, WO 2020016318, each of which are incorporated in their entirety by reference.

### Steroids

In one embodiment, the subject is further administered a steroid with an AAV or any therapeutic described herein. In one embodiment, the steroid is prednisone. In one embodiment, the steroid is a corticosteroid. Exemplary corticosteroids include (1) hydrocortisone/cortisone; (2) prednisolone/prednisone/methylprednisolone; (3) betamethasone/dexamethasone; and (4) triamcinolone. In one embodiment, the steroid is selected from alclometasone, alclometasone dipropionate, amcinonide, augmented betamethasone, augmented betamethasone dipropionate, beclomethasone, beclomethasone dipropionate, betamethasone, betamethasone benzoate, betamethasone dipropionate, betamethasone sodium phosphate, betamethasone valerate, budesonide, clobetasol, clobetasol propionate, clocortolone, clocortolone pivalate, cortisone, desonide, desoximetasone, dexamethasone, dexamethasone acetate, dexamethasone sodium phosphate, diflorasone, diflorasone acetonide, diflorasone diacetate, flucinolone, fludroxycortide, flunisolide, fluocinolone acetonide, fluocinonide, flurandrenolide, fluticasone, fluticasone propionate, halcinonide, halobetasol, halobetasol propionate, hydrocortisone, hydrocortisone acetate, hydrocortisone butyrate, hydrocortisone sodium phosphate, hydrocortisone valerate, methylprednisolone, methylprednisolone acetate, methylprednisolone sodium succinate, mometasone, mometasone furoate, prednicarbate, prednisolone, prednisolone acetate, prednisolone sodium phosphate, prednisolone tebutate, prednisone, triamcinolone, triamcinolone acetonide, triamcinolone diacetate, tiamcinolone hexacetonide, ulobetasol, a combination of two or more of these steroids, or commercial products of these steroids.

In one embodiment, the steroid is administered orally. Steroids of the invention may be administered through any route encompassed by systemic or local administration as defined. For example, steroids of the invention may be applied locally to the skin, applied locally to the eye, ingested orally, inhaled directly into the lungs, injected into a vein or muscle, or injected directly into inflamed joints. Steroids that may be administered by an oral route include, but are not limited to the following steroids: betamethasone, budesonide, cortisone, dexamethasone, hydrocortisone, methylprednisolone, prednisolone, prednisone, triamcinolone, a combination of two or more of these steroids, and commercial products of these steroids. Steroids that may be administered by a parenteral route, such as parenteral injection, include, but are not limited to the following steroids: betamethasone, cortisone, dexamethasone, hydrocortisone, methylprednisolone, prednisolone, triamcinolone, a combination of two or more of these steroids, and commercial products of these steroids. Steroids that may be administered by inhalation include, but are not limited to the following steroids: beclomethasone, budesonide, flunisolide, fluticasone, mometasone, triamcinolone, a combination of two or more of these steroids, and commercial products of these steroids. Steroids that may be administered by a topical route include, but are not limited to the following steroids: alclometasone, amcinonide, augmented betamethasone, betamethasone, clobetasol, clocortolone, desonide, desoximetasone, dexamethasone, diflorasone, flucinolone, fluocinonide, flurandrenolide, fluticasone, halcinonide, halobetasol, hydrocortisone, methylprednisolone, mometasone, prednicarbate, triamcinolone, a combination of two or more of these steroids, and commercial products of these steroids. One of skill in the art would understand that a particular steroid may be applied by more than one route, e.g. a steroid utilized in a topical formulation may be adapted for intravenous or oral administration.

In one embodiment, the steroid is administered at substantially the same time of the AAV or therapeutic described herein. In one embodiment, the steroid is administered at least 8 hours, 16 hours, 24 hours, 32 hours, 40 hours for more following administration of the AAV or therapeutic described herein. In one embodiment, the steroid is administered at least 8 hours, 16 hours, 24 hours, 32 hours, 40 hours for more prior administration of the AAV or therapeutic described herein. In one embodiment, the steroid, e.g., prednisone, is administered at a dose of 1 mg/kg body mass daily up to a total dose of 60 mg for 4 weeks followed by a ˜0.08 mg/kg taper to the nearest 1 mg, e.g., 5 mg if taking 60 mg, each week for at least 12 weeks.

One of ordinary skill in the art would understand that steroids have various medical uses, including but not limited to: (1) anti-inflammatory uses, e.g. betamethasone, budesonide, cortisone, dexamethasone, hydrocortisone, methylprednisolone, prednisolone, prednisone, and triamcinolone; (2) antiemetic uses, e.g. dexamethasone, hydrocortisone, and prednisone; (3) diagnostic uses, e.g. dexamethasone, as used to detect Cushing's syndrome; and (4) immunosuppressant uses, e.g. betamethasone, cortisone, dexamethasone, hydrocortisone, methylprednicolone, prednisolone, prednisone, and triamcinolone. Moreover, one of ordinary skill in the art would understand that corticosteroid drugs can be used as ingredients contained in eye products (to treat various eye conditions), inhalers (to treat asthma or bronchial disease), nasal drops and sprays (to treat various nasal conditions), and topical products such as ointments and creams (to treat various skin conditions).

One of ordinary skill in the art would understand that potencies may vary among steroids. For example, as associated with systemic administration, betamethasone and dexamethasone exhibit high overall potencies and high anti-inflammatory potencies; methylprednisolone, triamcinolone, prednisolone, and prednisone exhibit medium overall potencies and medium anti-inflammatory potencies; and hydrocortisone and cortisone exhibit low overall potencies and anti-inflammatory potencies.

### Ribitol

In one embodiment, the rAAV or therapeutic described herein is administered concurrently with ribitol. Ribitol is a crystalline pentose alcohol formed by the reduction of ribose. Ribitol enhances the flux of D-glucose to the pentose phosphate pathway in Saccharomyces cerevisiae for the production of D-ribose and ribitol. Ribitol has previously been shown to effect glycosylation of α-dystroglycan in a dystrophic mouse model; this effect is further described in, e.g., Cataldi, M P, et al. Molecular Therapy: Methods and Clinical Dev., Volume 17, June 2020, which is incorporated herein by reference.

Ribitol is commercially available, e.g., via Selleck Chem (Houston, TX), and has a chemical structure of

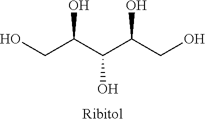

In one embodiment, the ribitol is administered at substantially the same time of the AAV or therapeutic described herein. In one embodiment, the ribitol is administered at least 8 hours, 16 hours, 24 hours, 48 hours, 72 hours, 124 hours or more following administration of the AAV or therapeutic described herein. In one embodiment, the ribitol is administered at least 8 hours, 16 hours, 24 hours, 48 hours, 72 hours, 124 hours or more prior administration of the AAV or therapeutic described herein.

In one embodiment, the ribitol is administered at least 1 time. In one embodiment, the ribitol is administered at least 2 times, e.g., at least 1, 2, 3, 4, 5, 6, 7, 8, 9, 10, 11, 12, 13, 14, 15 times or more. In one embodiment, ribitol is administered at least once daily, at least once weekly, at least once monthly, at least once yearly. An exemplary ribitol regimen, when co-administered with the rAAV comprising FKRP therapeutic as described herein, comprises about 6 grams to about 12 grams Ribitol administered orally once or, twice daily. In some embodiments, in the co-administration regimen when administered together, before, or after the rAAV comprising FKRP therapeutic as described in this invention, the ribitol is administered orally about 1 gram, about 2 grams, about 3 grams, about 4 grams, about 5 grams, about 6 grams, about 7 grams, about 8 grams, about 9 grams, about 10 grams, about 11 grams, or, about 12 grams. In some embodiments, in the co-administration regimen when administered together, before, or after the rAAV comprising FKRP therapeutic as described in this invention, the ribitol is administered orally more than about 12 grams. In one embodiment, Ribitol is co-administered twice daily. In one embodiment, Ribitol is co-administered three times daily. In one embodiment, Ribitol is co-administered more than three times daily. In some embodiments, Ribitol is co-administered orally, or, intranasally, or, via intravenous route, or, via subcutaneous route, or, via intramuscular rote, or, via intrathecal route, or, via sublingual and buccal route, or, via rectal route, or, via nasal route, or, via inhalation route, or, via nebulization route, or, via cutaneous route, or, via transdermal route. In a preferred embodiment, Ribitol is co-administered orally when administered together, before, or after the rAAV comprising FKRP therapeutic as described in this invention.

### Dosage

Dosages of the a rAAV vector or rAAV genome as disclosed herein to be administered to a subject will depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 0¹¹, 10¹², 10¹³, 10¹⁴, 10¹⁵ transducing units, optionally about 10⁸ to about 10¹³ transducing units. In some embodiments of the invention the dosage is from about 1E13 vg/kg to about 6E13 vg/kg. In some embodiments, the dosage is from about 1E13 vg/kg to about 3E13 vg/kg. In some embodiments, the dosage is from about 3E13 vg/kg to about 6E13 vg/kg. In some embodiments, the dosage is about 1E13 vg/kg, 1.5E13 vg/kg, 2E13 vg/kg, 2.5E13 vg/kg, 3E13 vg/kg, 3.5E13 vg/kg, 4E13 vg/kg, 4.5E13 vg/kg, 5E13 vg/kg, 5.5E13 vg/kg, or 6E13 vg/kg. In some embodiments, the dosage is from about 1E14 vg/kg to about 6E14 vg/kg. In one embodiment, the dosage is 3E14 vg/kg.

### Administration Routes and Regimens

Routes of administration include, without limitation, oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to skeletal muscle, cardiac muscle, diaphragm muscle) or other parenteral route depending on the desired route of administration and the tissue that is being targeted.

In some embodiments of the methods and compositions as disclosed herein, localized administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator extemus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, stemocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

In some embodiments of the methods and compositions as disclosed herein, localized administration to cardiac muscle includes administration to the left atrium, right atrium, left ventricle, right ventricle and/or septum. The virus vector and/or capsid can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

In some embodiments of the methods and compositions as disclosed herein, administration to a diaphragm muscle can be by any suitable method including intravenous administration, intra-arterial administration, and/or intra-peritoneal administration, and direct muscular injection.

In some embodiments of the methods and compositions as disclosed herein, the rAAV vectors and/or rAAV genome as disclosed herein are administered to the skeletal muscle, diaphragm, costal, and/or cardiac muscle cells of a subject. For example, a conventional syringe and needle can be used to inject a rAAV virion suspension into a subject locally or systemically. Parenteral administration of a the rAAV vectors and/or rAAV genome, by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain agents for a pharmaceutical formulation, such as suspending, stabilizing and/or dispersing agents. Alternatively, the rAAV vectors and/or rAAV genome as disclosed herein can be in powder form (e.g., lyophilized) for constitution with a suitable vehicle, for example, sterile pyrogen-free water, before use.

In some embodiments, a single administration is employed. In some embodiments, more than one administration (e.g., two, three, four, five, six, seven, eight, nine, 10, etc., or more administrations) may be employed to achieve the desired level of gene expression over a period of various intervals, e.g., hourly, daily, weekly, monthly, yearly, etc. Dosing can be single dosage or cumulative (serial dosing), and can be readily determined by the skilled practitioner. For instance, treatment of a disease or disorder may comprise a one-time administration of an effective dose of a pharmaceutical composition virus vector disclosed herein. Alternatively, treatment of a disease or disorder may comprise multiple administrations of an effective dose of a virus vector carried out over a range of time periods, such as, e.g., once daily, twice daily, trice daily, once every few days, or once weekly. In some embodiments, the administration of a rAAV vector or rAAV genome as disclosed herein to a subject is every day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The timing of administration can vary from individual to individual, depending upon such factors as the age of the individual and/or the severity of an individual's symptoms. For example, an effective dose of a virus vector disclosed herein can be administered to an individual once every six months for an indefinite period of time, or until the individual no longer requires therapy. The skilled practitioner will recognize that the condition of the individual can be monitored throughout the course of treatment and that the effective amount of a virus vector disclosed herein that is administered can be adjusted accordingly.

In some embodiments, administration of rAAV vector or rAAV genome as disclosed herein to a subject results in production of a FKRP protein with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

Efficacy of administration can be assessed various assays known in the art, e.g., a North Star Assessment for Limb Girdle Muscular Dystrophies (NSAD) (e.g., as described in Jacobs M B, et al. Ann Neurol. 2021 May; 89(5):967-978. doi: 10.1002/ana.26044. Epub 2021 Feb. 26.); Clinical Global Impression (CGI) for disease improvement, severity, and therapeutic efficacy; 10-meter walk test (10 MWT) (e.g., as described in McDonald C M, et al. Muscle Nerve. 2013 September; 48(3):357-68. doi: 10.1002/mus.23905. Epub 2013 Jul. 17.); 100-meter walk test (100 MWT) (e.g., as described in Mendel, et al. JAMA Neurol. 2020; 77(9):1122-1131. doi: 10.1001/jamaneurol.2020.1484); 4-stair climb (4SC);-Timed-Up and -Go (TUG); Performance Upper Limb (PUL) (e.g., as described in Gandolla M, et al. PLoS One. 2020 Sep. 28; 15(9):e⁰²³⁹⁰⁶⁴. doi: 10.1371/journal.pone.0239064); and/or patient reported outcome measures (e.g., individualized quality of life, fatigue, sleepiness, depression scores).

In one embodiment, a subject receiving the rAAV comprising FKRP therapeutic described herein displays NSAD score that is at least or about 1.73 points from baseline. In one embodiment, a subject receiving the therapeutic described herein displays NSAD score that is at least or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 or more points from baseline. As used herein, “baseline” refers to NSAD score of the subject prior to administration of the therapeutic. One skilled in the art will understand how to assess the NSAD score, for example, as described in Jacobs M B, et al. Assessing Dysferlinopathy Patients Over Three Years With a New Motor Scale. Ann Neurol. 2021 May; 89(5):967-978, which is incorporated herein by reference.

In one embodiment, a subject receiving the rAAV comprising FKRP therapeutic described herein displays 10 MWT score that is at least or about 31% (e.g., 2.3 seconds) baseline. In one embodiment, a subject receiving the therapeutic described herein displays 10 MWT score that is at least or about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more from baseline. As used herein, “baseline” refers to 10 MWT score of the subject prior to administration of the therapeutic. One skilled in the art will understand how to assess the 10 MWT score, for example, as described in McDonald C M, et al. The 6-minute walk test and other clinical endpoints in duchenne muscular dystrophy: reliability, concurrent validity, and minimal clinically important differences from a multicenter study. Muscle Nerve. 2013 September; 48(3):357-68., which is incorporated herein by reference.

In one embodiment, a subject receiving the rAAV comprising FKRP therapeutic described herein displays 100 MWT score that is at least or about 6 seconds baseline. In one embodiment, a subject receiving the therapeutic described herein displays 100 MWT score that is at least or about 0.5 sec, 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, 60 secor more from baseline. As used herein, “baseline” refers to 100 MWT score of the subject prior to administration of the therapeutic. One skilled in the art will understand how to assess the 100 MWT score, for example, as described in Mendell J R, et al. Assessment of Systemic Delivery of rAAVrh74.MHCK7.micro-dystrophin in Children With Duchenne Muscular Dystrophy: A Nonrandomized Controlled Trial. JAMA Neurol. 2020; 77(9):1122-1131., which is incorporated herein by reference.

In one embodiment, a subject receiving the rAAV comprising FKRP therapeutic described herein displays 4SC score that is at least or about 30% baseline. In one embodiment, a subject receiving the therapeutic described herein displays 4SC score that is at least or about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more from baseline. As used herein, “baseline” refers to 4SC score of the subject prior to administration of the therapeutic. One skilled in the art will understand how to assess the 4SC score.

In one embodiment, a subject receiving the rAAV comprising FKRP therapeutic described herein displays TUG score that is at least or about 30% baseline. In one embodiment, a subject receiving the therapeutic described herein displays TUG score that is at least or about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 8, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99% or more from baseline. As used herein, “baseline” refers to TUG score of the subject prior to administration of the therapeutic. One skilled in the art will understand how to assess the TUG score.

In one embodiment, a subject receiving the rAAV comprising FKRP therapeutic described herein displays PUL score that is at least or about 4 points from baseline. In one embodiment, a subject receiving the therapeutic described herein displays PUL score that is at least or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8.0 or more points from baseline. As used herein, “baseline” refers to PUL score of the subject prior to administration of the therapeutic. One skilled in the art will understand how to assess the PUL score, for example, as described in Gandolla M, et al. Test-retest reliability of the Performance of Upper Limb (PUL) module for muscular dystrophy patients. PLoS One. 2020 Sep. 28; 15(9):e02390, which is incorporated herein by reference.

Viral Shedding Assay:

A viral shedding assay will be developed for the product scAAV9-Syn100-coFKRP, or, any derivative thereof e.g., wherein, a synthetic muscle promoter selected from any of Tables 1-4 or, from 8-12, replaces the Syn100 promoter. Shedding assays are typically performed to collect information about the likelihood of transmission to the untreated individuals. In a public presentation posted on Jul. 6, 2020 by Pfizer on AAV gene therapy treating Duchenne muscular dystrophy, the viral shedding phenomenon was demonstrated as possibility of seroconversion of a family member (e.g., sibling who did not receive the treatment) of the person receiving the treatment. Seroconversion indicates the change of not having antibodies to having antibodies, to the therapeutic product, e.g., to AAV serotype used in the treatment. After the virus administered as part of the gene therapy, it exits the body for a short time through bodily fluids e.g., through saliva and if a person not receiving the treatment came in contact of the fluid within that shedding period, there might be a possibility the untreated person might develop antibodies to the virus that would preclude them from getting a gene therapy in future if needed one.

The presence of the shed product is often tested in the clinical samples of a subject e.g., from feces, urine, nasal swabs, saliva. The analytical assay will measure the shedding in the clinical sample by detecting the nucleic acid encoding the therapeutic product or, by the presence of infectious viral particles. Viral shedding assay results will help to determine if the therapeutic product is shed, if the shed product is infectious, whether the amount of infectivity in the clinical samples is comparable to that needed to initiate infection in a third party, whether the clinical sample containing the shed product represents the natural route for transmission. The details of viral shedding assays including its objective, assay design, analysis is discussed in Design and Analysis of shedding studies for virus or, bacteria based gene therapy and oncolytic products, US Department of Health and Human Services, Food and Drug Administration, Center for Biologics Evaluation and Research, August 2015, which is incorporated by reference in its entirety.

Potency Assay Development

An in vitro potency assay is developed by the inventors for the therapeutic product scAAV9-syn100-coFKRP, or its derivative thereof to support comparability studies and different lots or, batches of therapeutic product preparations, and/or, to compare the response of a test article to a designated reference, and/or, to reflect complex biological activity of FKRP (e.g., glycosylation of α-DG, laminin binding). The assay is contemplated to be developed in several cells e.g., human aortic vascular smooth muscle cell lines (HA-VSMC, or, HASMC cells), LGMD2I patient derived FKRP deficient paravertebral skeletal muscle cell, iPSC stem cell line to be differentiated into cardiac or, skeletal muscle cell line, FKRP knock down or, knock out cell line.

Assays described herein can, e.g., quantify vector viral genome copies within muscle biopsy tissues, mRNA expression and transgene protein expression (protein expression measured by western blot, and/or, immunohistochemistry) in muscle biopsy tissues, characterize downstream effects of transgene expression in muscle biopsy tissues (e.g., glycosylation of α-DG, laminin binding in biopsy tissues).

Assays described herein can, e.g., further measure the degree of target activity in open muscle biopsies, e.g., whether there is sufficient target activity above baseline. The biomarkers (e.g., de novo muscle biomarkers) of interest and key output muscle assays to determine the target activity within the (scAAV9-syn100-coFKRP) treated muscle biopsies include: evidence oftransduction in the muscle (e.g., high number of AAV9-Syn100-FKRP vector genome copies within the muscle tissues), evidence of mRNA expression of the hFKRP (human FKRP) transgene in the muscle, above baseline mRNA levels, evidence of increased healthy FKRP enzyme levels in the muscle (e.g., via immunofluorescence, western blot, ELISA, etc.) above baseline, evidence of increased downstream activity directly related to increased FKRP enzyme levels (e.g., increased terminal glycosylation of the α-DG subunits; increased laminin binding) above baseline. The baseline refers to the level before treatment. In some instances, the baseline refers to the level obtained aster mock treatment that did not receive the therapeutic product of interest e.g scAAV9-sun100-coFKRP.

### Formulations

In some embodiments, the rAAV vectors and/or rAAV genome as disclosed herein can be formulated in a solvent, emulsion or other diluent in an amount sufficient to dissolve an rAAV vector disclosed herein. In other aspects of this embodiment, the rAAV vectors and/or rAAV genome as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

To facilitate delivery of a rAAV vector and/or rAAV genome as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

In addition to the formulations described previously, a rAAV vector and/or rAAV genome as disclosed herein can also be formulated as a depot preparation. Such long acting formulations may be administered by implantation (for example subcutaneously or intramuscularly) or by IM injection. Thus, for example, a rAAV vector and/or rAAV genome as disclosed herein may be formulated with suitable polymeric or hydrophobic materials (for example as an emulsion in an acceptable oil) or ion exchange resins, or as sparingly soluble derivatives.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1). The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

All aspects of the compositions and methods of the technology disclosed herein can be defined in any one or more of the following numbered paragraphs:
1. A recombinant adenovirus associated (AAV) vector comprising in its genome in the 5′ to 3′ direction:
   a) a 5′ AAV inverted terminal repeat (ITR);
   b) a muscle specific promoter;
   c) an intron sequence;
   d) a nucleic acid encoding human fukutin-related protein (FKRP) which has a nucleotide sequence shown in SEQ ID NO: 2, and is operatively linked to the muscle specific promoter;
   e) a polyA signal sequence operatively linked to the nucleic acid encoding FKRP;
   f) a 3′ AAV ITR.
2. The recombinant AAV vector of paragraph 1, wherein the 5′ITR is ITR2m.
3. The recombinant AAV vector of any one of paragraphs 1-2, wherein the 3′ITR is ITR2.
4. The recombinant AAV vector of any one of paragraphs 1-3, wherein the muscle-specific promoter is Syn100 (SEQ ID NO: 3).
5. The recombinant AAV vector of any one of paragraphs 1-4, wherein the intron sequence is VH4-Ig-Intron 3 (SEQ ID NO: 4) or a derivative thereof.
6. The recombinant AAV vector of any one of paragraphs 1-5, wherein the polyA signal sequence is SEQ ID NO: 5.
7. The recombinant AAV vector of any one of paragraphs 1-6, wherein the muscle specific promoter, intron sequence, nucleic acid encoding FKRP, and polyA signal sequence are comprised within SEQ ID NO: 1.
8. The recombinant AAV vector of any one of paragraphs 1-7, wherein the serotype is AAV9.
9. A pharmaceutical composition comprising the recombinant AAV vector of any one of paragraphs 1-8.
10. A method to treat a subject with a dystroglycanopathy disorder comprising systemically administering a therapeutically effective amount of the recombinant AAV vector of any one of paragraphs 1-8, and/or the pharmaceutical composition of paragraph 9, to the subject, to thereby increase expression of functional FKRP in muscle tissue of the subject.
11. The method of paragraph 10, wherein the dystroglycanopathy disorder is limb-girdle muscular dystrophy 2I.
12. The method of any one of paragraphs 10-11, wherein a single dose is administered to the subject.
13. The method of any one of paragraphs 10-12, wherein administration is by intravenous infusion.
14. The method of any one of paragraphs 10-13, wherein the dose administered is from about 1E13 vg/kg to about 6E13 vg/kg (e.g. about 3E13 vg/kg).
15. The method of any one of paragraphs 10-14, wherein one or more of the following occur in the subject following administration:
   a) functional glycosylation of α-DG is substantially increased in skeletal muscle and/or cardiac muscle of the subject;
   b) serum creatine kinase levels of the subject are substantially reduced;
   c) collagen deposition in skeletal muscle of the subject is substantially reduced;
   d) in vitro muscle force analysis of the subject's muscle tissue (e.g., soleus, diaphragm and/or EDL) is significantly increased; and/or
   e) the subject can run significantly further in a treadmill test.
16. The method of any one of paragraphs 10-15, wherein the subject is an adult.
17. A synthetic nucleic acid encoding human fukutin-related protein (FKRP), wherein:
   a) the nucleic acid has reduced CpG site content relative to the CpG site content of SEQ ID NO: 6;
   b) the GC content is reduced by greater than 10% relative to the GC content of SEQ ID NO:6; and/or
   c) the nucleic acid has at least 80% identity to SEQ ID NO: 2.
18. The nucleic acid of paragraph 17, wherein the coding sequence has at least 50% reduced CpG site content relative to the CpG site content of SEQ ID NO: 6.
19. The nucleic acid of any one of paragraphs 17-18, wherein the coding sequence has at least 75%, 80%, 85%, 90%, 95% reduced CpG site content relative to the CpG site content of SEQ ID NO: 6.
20. The nucleic acid of any one of paragraphs 17-19, wherein the coding sequence has 0% CpG site content.
21. The synthetic nucleic acid of any one of paragraph 17, wherein the GC content is reduced by greater than 15% relative to the GC content of SEQ ID NO:6.
22. The synthetic nucleic acid of any one of paragraph 17, wherein the nucleic acid has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 2.
23. The synthetic nucleic acid of paragraph 17, wherein the nucleic acid has a sequence shown in SEQ ID NO: 2.
24. The synthetic nucleic acid of any one of paragraphs 17-23 that is operably linked to a promoter.
25. The synthetic nucleic acid of paragraph 24, wherein the promoter is a muscle-specific promoter.
26. The synthetic nucleic acid of any one of paragraphs 24-25, wherein the promoter is a synthetic promoter.
27. The synthetic nucleic acid of any one of paragraph 24-26, wherein the promoter is Syn100.
28. The synthetic nucleic acid of any one of paragraphs 23-26, wherein the promoter is selected from promoters listed in Tables 1-4.
29. The synthetic nucleic acid of any one of paragraphs 24-25, wherein the promoter is a creatine kinase (CK) promoter, a chicken R-actin promoter (CB).
30. The synthetic nucleic acid of any one of paragraphs 17-29, further comprising an enhancer sequence.
31. The synthetic nucleic acid of paragraph 30, wherein the enhancer sequence comprises a CMV enhancer, a muscle creatine kinase enhancer, and/or a myosin light chain enhancer.
32. A nucleic acid comprising:
   a) 5′ and 3′ AAV inverted terminal repeats (ITR);
   b) a coding sequence encoding human fukutin-related protein (FKRP) operatively linked to a muscle-specific promoter located between the 5′ITR and 3′ITR, wherein the coding sequence has:
   i) reduced CpG site content relative to the CpG site content of SEQ ID NO: 6;
   ii) reduced GC content greater than 10% relative to the GC content of SEQ ID NO:6; and/or
   iii) at least 80% identity to SEQ ID NO: 2.
33. The nucleic acid of paragraph 32, further comprising an intron sequence located between the muscle-specific promoter and the coding sequence.
34. The nucleic acid of paragraph 33, wherein the intron sequence is VH4-Ig-Intron 3 (SEQ ID NO: 4) or a derivative thereof.
35. The nucleic acid of any one of paragraphs 32-34, further comprising at least one polyA signal sequence located downstream of the coding sequence.
36. The nucleic acid of paragraph 35, wherein the polyA signal sequence is SEQ ID NO: 5.
37. The nucleic acid of any one of paragraph 32-36, wherein the 5′ITR is ITR2m.
38. The nucleic acid of any one of paragraphs 32-37, wherein the 3′ITR is ITR2.
39. The nucleic acid of any one of paragraphs 32-38, wherein the GC content of the coding sequence is reduced by greater than 15% relative to the GC content of SEQ ID NO:6.
40. The nucleic acid of any one of paragraphs 32-40, wherein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 2.
41. The nucleic acid of any one of paragraphs 32-40, wherein the coding sequence has at least 50% reduced CpG site content relative to the CpG site content of SEQ ID NO: 6.
42. The nucleic acid of any one of paragraphs 32-41, wherein the coding sequence has at least 75%, 80%, 85%, 90%, 95% reduced CpG site content relative to the CpG site content of SEQ ID NO: 6.
43. The nucleic acid of any one of paragraphs 32-42, wherein the coding sequence has 0% CpG site content.
44. The nucleic acid sequence of any one of paragraphs 32-43, wherein the coding sequence is SEQ ID NO: 2.
45. A vector comprising the synthetic nucleic acid of any one of paragraphs 17 to 44.
46. The vector of paragraph 45, wherein the vector is a viral vector.
47. The vector of paragraph 46, wherein the vector is a recombinant adeno-associated virus (AAV) vector.
48. The vector of paragraph 47, wherein the AAV vector is any serotype listed in Table 6.
49. The vector of paragraph 47 or paragraph 48, wherein the AAV vector is an AAV9 vector.
50. A recombinant adenovirus associated (AAV) vector comprising in its genome:
   a) a 5′ AAV inverted terminal repeat (ITR) and a 3′ AAV ITR;
   b) located between the 5′ITR and 3′ITR, a nucleic acid encoding human fukutin-related protein (FKRP) which has:
   i) reduced CpG site content relative to the CpG site content of SEQ ID NO: 6;
   ii) reduced GC content greater than 10% relative to the GC content of SEQ ID NO:6; and/or
   iii) at least 80% identity to SEQ ID NO: 2,
and is operatively linked to a muscle-specific promoter.
51. The recombinant AAV vector of paragraph 50, wherein the AAV genome comprises, in the 5′ to 3′ direction:
   a. the 5′ITR,
   b. the muscle-specific promoter,
   c. an intron sequence,
   d. the nucleic acid encoding FKRP; and,
   e. the 3′ITR.
52. The recombinant AAV vector of any of paragraphs 50-51, wherein the muscle-specific promoter is selected from the group consisting of MCK promoter, dMCK promoter, tMCK promoter, enh358MCK promoter, CK6 promoter and Syn100 promoter, any promoter listed in Table 1-4 or 8-12, and derivatives thereof.
53. The recombinant AAV vector of any of paragraphs 50-52, wherein the nucleic acid encoding FKRP has reduced CpG site content relative to the CpG site content of SEQ ID NO: 6.
54. The recombinant AAV vector of any of paragraphs 50-53, wherein the nucleic acid encoding FKRP has at least 50% reduced CpG site content relative to the CpG site content of SEQ ID NO: 6.
55. The recombinant AAV vector of any of paragraphs 50-53, wherein the nucleic acid encoding FKRP has at least 75%, 80%, 85%, 90%, 95% reduced CpG site content relative to the CpG site content of SEQ ID NO: 6.
56. The recombinant AAV vector of any of paragraphs 50-55, wherein the nucleic acid encoding FKRP has 0% CpG site content.
57. The recombinant AAV vector of any of paragraphs 50-56, wherein the nucleic acid encoding FKRP has reduced GC content greater than 10% relative to the GC content of SEQ ID NO:6.
58. The recombinant AAV vector of any of paragraphs 50-57, wherein the nucleic acid encoding FKRP has at least 80% identity to SEQ ID NO: 2.
59. The recombinant AAV vector of any one of paragraphs 50-58, wherein the nucleic acid encoding FKRP has a sequence shown in SEQ ID NO: 2.
60. The recombinant AAV vector of any one of paragraphs 50-59, further comprising at least one polyA signal sequence located 3′ of the nucleic acid encoding the FKRP polypeptide and 5′ of the 3′ITR sequence.
61. The recombinant AAV vector of paragraph 60, wherein the polyA signal sequence is SEQ ID NO: 5.
62. The recombinant AAV vector of any one of paragraphs 50-61, wherein the ITR comprises an insertion, deletion or substitution.
63. The recombinant AAV vector of any one of paragraphs 50-62, wherein one or more CpG site sites in the ITR are removed.
64. The recombinant AAV vector of any one of paragraphs 50-63, wherein the 5′ITR is ITR2m.
65. The recombinant AAV vector of any one of paragraphs 50-64, wherein the 3′ITR is ITR2.
66. The recombinant AAV vector of any one of paragraphs 50-65, wherein the intron sequence is VH4-Ig-Intron 3 (SEQ ID NO: 4) or a derivative thereof.
67. The recombinant AAV vector of any one of paragraphs 50-66, wherein the recombinant AAV vector is a chimeric AAV vector, haploid AAV vector, a hybrid AAV vector or polyploid AAV vector.
68. The recombinant AAV vector of any of paragraphs 50-66, wherein the recombinant AAV vector is any AAV serotype listed in Table 6.
69. The recombinant AAV vector of paragraph 68 wherein the serotype is AAV9.
70. The recombinant AAV vector of any one of paragraphs 50-69, wherein the recombinant AAV vector comprises a capsid protein selected from Table 7 or any AAV serotype in the group consisting of those listed in Table 6, and combinations thereof.
71. A pharmaceutical composition comprising the recombinant AAV vector of any one of paragraphs 50-70 in a pharmaceutically acceptable carrier.
72. A transformed cell comprising the nucleic acid of any one of paragraphs 17-44 and/or the vector of any one of paragraphs 45 to 70.
73. A transgenic animal comprising the nucleic acid of any one of paragraphs 17-44, the vector of any one of paragraphs 45 to 70, and/or the transformed cell of paragraph 72.
74. A method of increasing glycosylation of α-dystroglycan (α-DG) in a subject in need thereof, comprising: administering to said subject a therapeutically effective amount of the nucleic acid of any one of paragraphs 17-44, the vector of any one of paragraphs 45 to 70, the pharmaceutical composition of paragraph 71, and/or the transformed cell of paragraph 72, wherein the synthetic nucleic acid is expressed in said subject, thereby producing human FKRP and increasing glycosylation of α-DG.
75. The method of paragraph 74, wherein the subject has or is at risk for developing a dystroglycanopathy disorder.
76. A method of treating or a dystroglycanopathy disorder in a subject, comprising administering to the subject a therapeutically effective amount of the nucleic acid of any one of paragraphs 17 to 44, the vector of any one of paragraphs 45-70, the pharmaceutical composition of paragraph 71, and/or the transformed cell of paragraph 72, wherein the synthetic nucleic acid is expressed in said subject, thereby treating the dystroglycanopathy disorder in the subject.
77. The method of any one of paragraphs 75 or 76, wherein the dystroglycanopathy disorder is associated with a FKRP anomaly.
78. The method of any one of paragraphs 75-77, wherein the dystroglycanopathy disorder comprises a mutation in the nucleic acid encoding FKRP and/or a deficiency in glycosylation of α-dystroglycan (α-DG).
79. The method of any one of paragraphs 75-78, wherein the dystroglycanopathy disorder is limb-girdle muscular dystrophy 2I, congenital muscular dystrophy (CMD1C), Walker-Warburg syndrome, muscle-eye-brain disease, or any combination thereof.
80. A method to treat a subject with a dystroglycanopathy disorder comprising administering a therapeutically effective amount of any of the recombinant AAV vector, the rAAV genome, the nucleic acid sequence, and/or the pharmaceutical compositions, of any one of the previous paragraphs to the subject, to thereby increase expression of functional FKRP in muscle tissue of the subject.
81. The method of any one of paragraphs 74-80, wherein a single dose is administered to the subject.
82. The method of any one of paragraphs 74-81, wherein administration is systemic.
83. The method of any one of paragraph 82, wherein administration is by intravenous infusion.
84. The method of any one of paragraphs 74-83, wherein functional glycosylation of α-DG is substantially increased in skeletal muscle and/or cardiac muscle of the subject following administration.
85. The method of any one of paragraphs 74-84, wherein serum creatine kinase levels of the subject are substantially reduced following administration.
86. The method of any one of paragraphs 74-85, wherein collagen deposition in skeletal muscle of the subject is substantially reduced following administration.
87. The method of any one of paragraphs 74-86, wherein the subject is an adult.
88. The method of any one of paragraphs 74-86, wherein the subject is a juvenile.
89. The method of any one of paragraphs 74-86, wherein the subject is an infant.
90. The method of any one of paragraphs 74-89, wherein the subject demonstrates significant disease pathology prior to administration.
91. The method of any one of paragraphs 74-89, wherein the subject demonstrates no significant disease pathology prior to administration.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The following non-limiting examples are provided for illustrative purposes only in order to facilitate a more complete understanding of representative embodiments now contemplated. These examples are intended to be a mere subset of all possible contexts in which the AAV virions and rAAV vectors may be utilized. Thus, these examples should not be construed to limit any of the embodiments described in the present specification, including those pertaining to AAV virions and rAAV vectors and/or methods and uses thereof. Ultimately, the AAV virions and vectors may be utilized in virtually any context where gene delivery is desired.

### EXAMPLES

The following non-limiting examples are provided for illustrative purposes only in order to facilitate a more complete understanding of representative embodiments now contemplated. These examples are intended to be a mere subset of all possible contexts in which the AAV virions and rAAV vectors may be utilized. Thus, these examples should not be construed to limit any of the embodiments described in the present specification, including those pertaining to AAV virions and rAAV vectors and/or methods and uses thereof. Ultimately, the AAV virions and vectors may be utilized in virtually any context where gene delivery is desired.

### Example 1: Vector Constructs Used for the Treatment of LGMD

An AAV gene therapy product candidate containing FKRP, for the treatment of LGMD2I was developed. LGMD2I is classified as a dystroglycanopathy and is a rare muscular dystrophy caused by mutations in the FKRP gene that codes for the fukutin-related protein, a golgi-bound transferase implicated in glycosylation, the cellular modification of the structure and activity, of α-Dystroglycan, or α-DG. Currently, there are no FDA-approved therapies for the treatment of LGMD2I. The experiments described herein indicate that the AAV gene therapy products described herein administered to human patients will provide patients with significant therapeutic results to produce significantly improved outcomes.

The AAV9 vector encoding the therapeutic FKRP delivered in the experiments described herein is shown in FIG. 13.

The nucleic acid sequence of the entire FKRP transgene cassette is also provided (FIG. 13, SEQ ID NO: 1). The cassette includes a Syn100 promoter (SEQ ID NO: 3), which is a synthetic muscle specific promoter (Qiao et al., Molecular Therapy Vol. 22 no. 11, p. 1890-1899 (2014)). The cassette further contains the VH4-Ig intron 3 (SEQ ID NO: 4), and also a poly A sequence (SEQ ID NO: 5), with spacer sequences between the promoter and the intron (actagta), the intron and the coding sequences (ccgcgggccacc (SEQ ID NO: 11)), and the coding sequences and the polyA sequences (gtcgac). The cassette is flanked by two ITRs, ITR2m and ITR2 (FIG. 13).

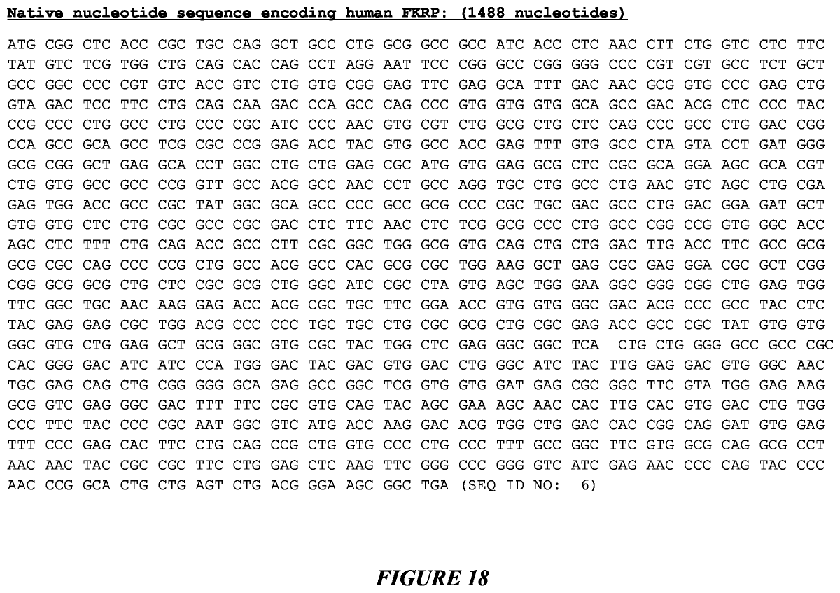

The nucleic acid sequence encoding the FKRP protein is also shown (SEQ ID NO: 2). This sequence has been codon optimized and further has 0% CpGs. In addition, the total GC content of the FKRP coding sequences is %, with is a 15% reduction in GC content from the native nucleotide sequence encoding human FKRP (FIG. 18, SEQ ID NO: 6).

### Example 2: Administration to Mouse Model of LGMD2I

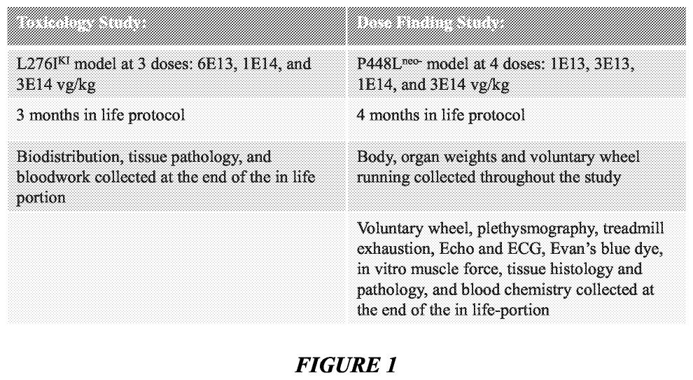

Delivery of the AAV9 FKRP vector results in expression of FKRP protein primarily in muscle tissue through the action of the Syn-100 promoter that is incorporated in the vector. The following experiments were performed in the mouse model system to determine whether delivery of AAV9-FKRP can ameliorates muscle pathology in LGMD2I patients and therefore serve as an effective therapy. FIG. 1 outlines the dose finding and toxicology studies performed.

Two mouse models were used in these studies. The homozygous knock-in mouse model (L276I^{KI}) mouse model harboring the human mutation leucine 276 to isoleucine (L276I) in the mouse alleles mimics the classic late onset phenotype of LGMD2I in both skeletal and cardiac muscles (Qiao et al. Mol Ther. 2014 November; 22(11): 1890-1899). This was used initially for toxicology and biodistribution studies. The mouse model for LGMD2I containing a homozygous missense mutation (c.1343C>T, p.Pro448Leu) in the FKRP gene (FKRP^{P448L} mutant) (Chan et al. (2010) Hum. Mol. Genet. 19, 3995-1006; Blaeser et al. (2013) Hum. Genet. 132, 923-934)) was used to demonstrate construct efficacy and dose-finding functionality.

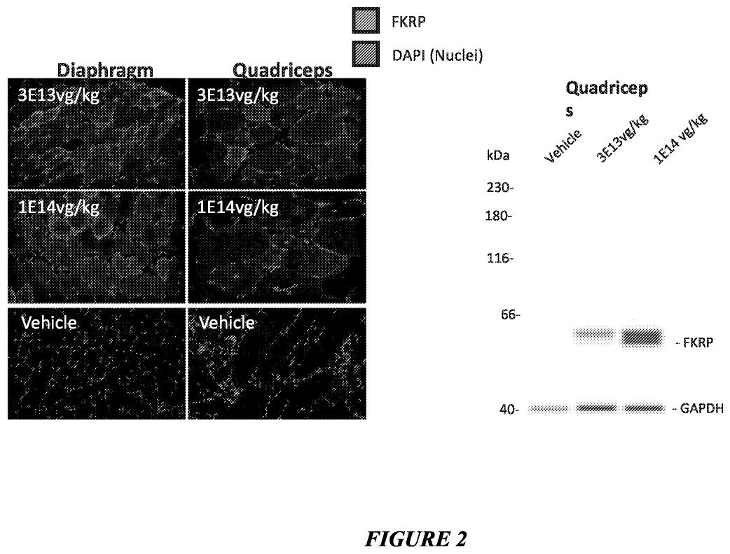

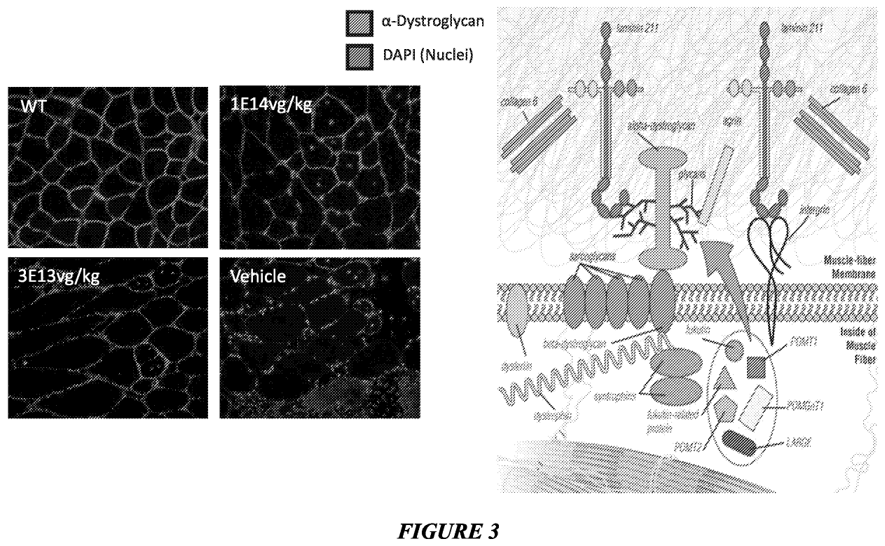

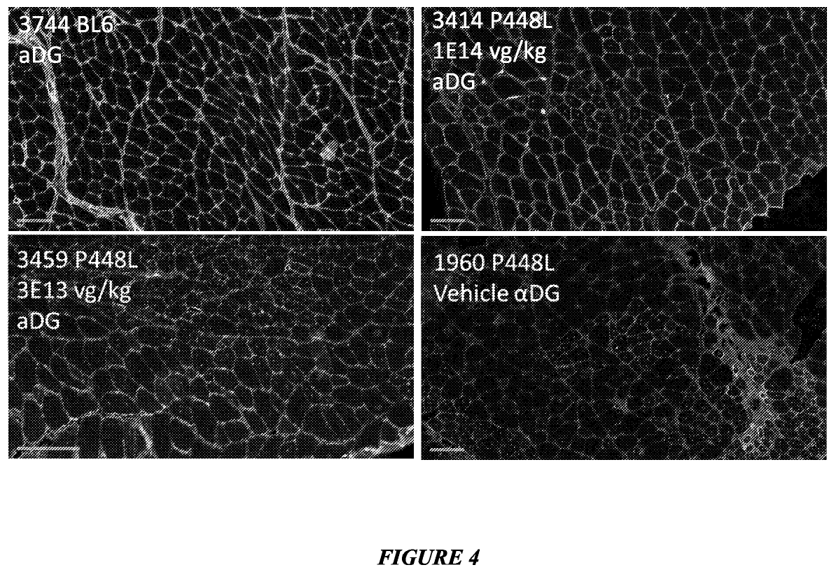

FKRP protein expression was observed in various muscle tissue in the mouse model mice which had received various doses (3E13 vg/kg, 1E14 vg/kg) of the AAV9-FKRP vector, compared to mice which had received empty vehicle. FIG. 2 shows photos of representative levels of expression in diaphragm and quadriceps of recipient mice, compared to mice which had received empty vehicle. Therapeutic FKRP protein expression correlated with increased functional glycosylation of α-dystroglycan expression in skeletal muscle. FIG. 3 shows photos of representative α-dystroglycan expression in normal BL6 mice (upper left photo) serving as a positive control, P448L mice that had received 1E14 vg/kg (upper right) and 3E13 vg/kg (lower left) AAV9-FKRP, and P448L mice that had received empty vehicle (lower right) serving as a negative control. Immunofluorescent staining from quadriceps sections showed a clear response to AAV9-FKRP, and the sham-injected P448L mice showed decreased expression of α-DG compared to untreated BL6 mice (FIG. 4). Compared to untreated P448 mice, mice treated with AAV9-FKRP at all dose levels showed increased expression of α-DG, suggesting effective FKRP expression and subsequent glycosylation of α-DG.

Increased collagen content is a reflection of ongoing fibrosis resulting from dystrophic pathology. Both picrosirius red staining and quantitative analysis of quadriceps muscle was performed in the P448L mice given various amounts of AAV9-FKRP or vehicle. Representative results of cross-sections of quadriceps muscle are shown in photos in FIG. 5 (left). Without treatment, P448L mice (center top photo) demonstrate large collagen deposition and irregular muscle fiber shape. These features progressively return to normal at different doses of AAV9-FKRP. The results are quantitated in the graphical representation shown in FIG. 5 (right). A reduction in dystrophic pathology and reduced fibrosis were observed. Sham-injected P448L mice showed increased collagen content compared to normal BL6 mice. Mice treated with AAV9-FKRP at all dose levels had reduced expression of collagen (reduced percent collagen) which suggests that there was less contraction-induced damage and deposition of fibrotic material.

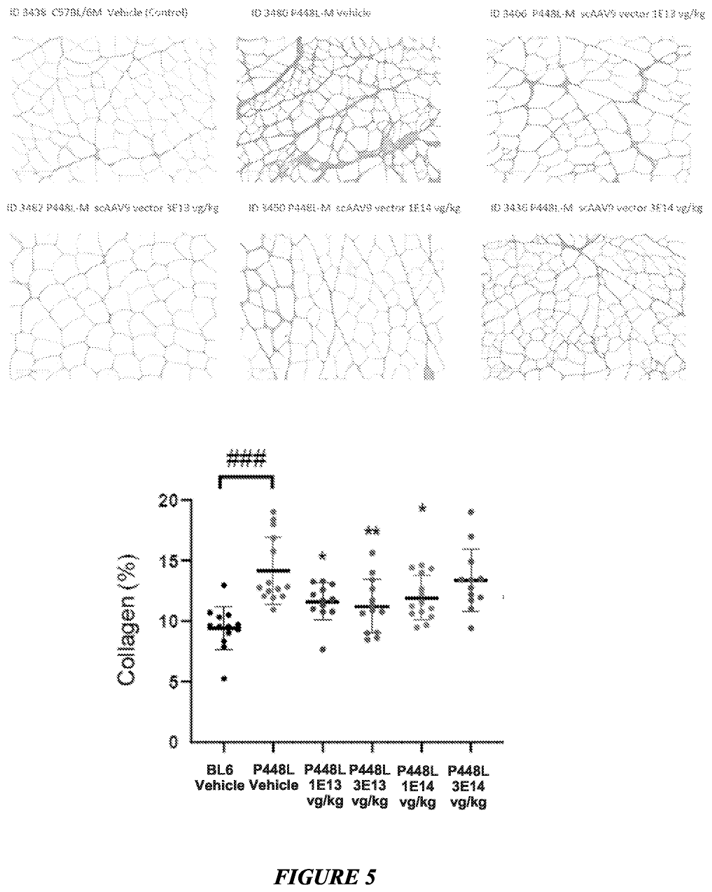

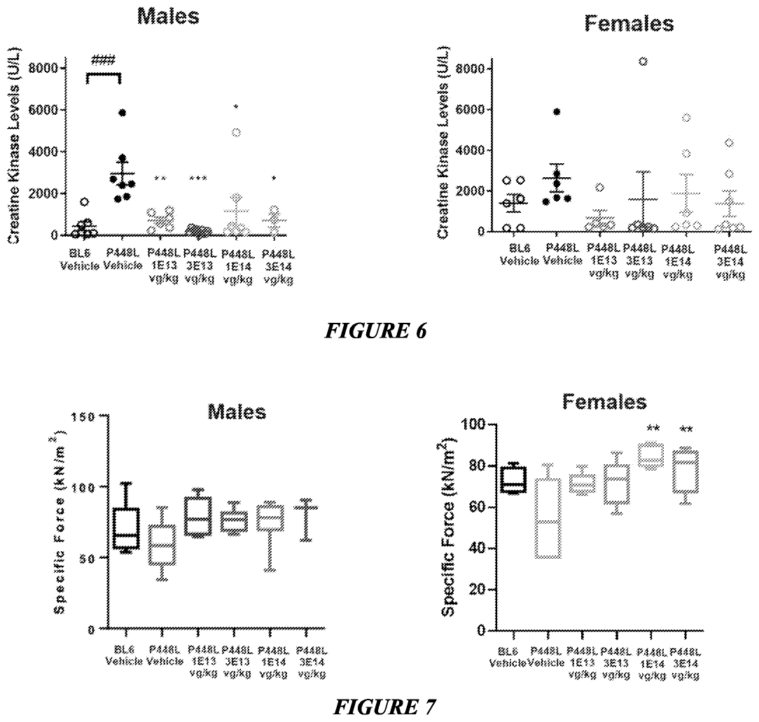

Creatine kinase is released when skeletal muscle is damaged. The levels of creative kinase are a readout of ongoing LGMD disease pathology. Serum creatine kinase levels were analyzed in the P448L mice. Results are presented in FIG. 6. The BL6 mice that had received empty vehicle are an indication of normal creatine kinase levels. The P448L mice that had received empty vehicle are an indication of the disease state levels of creatine kinase. Surprisingly, all P448L mice that had received any dose of AAV9-FKRP had reduced serum creatine kinase levels to the point of normalization.

Functional measures of muscle strength, endurance and physical activity were made in the recipient mice in order to examine whether recovery of those measures to baseline could be obtained. As shown in FIGS. 7-9, P448L mice administered only vehicle showed significantly lower diaphragm and soleus muscle force as compared to BL6 vehicle mice. Treatment with AAV9-FKRP at all dose levels resulted in significantly higher muscle force than in BL6 vehicle mice in diaphragm and soleus muscles. Similar trends were also observed in the EDL muscle.

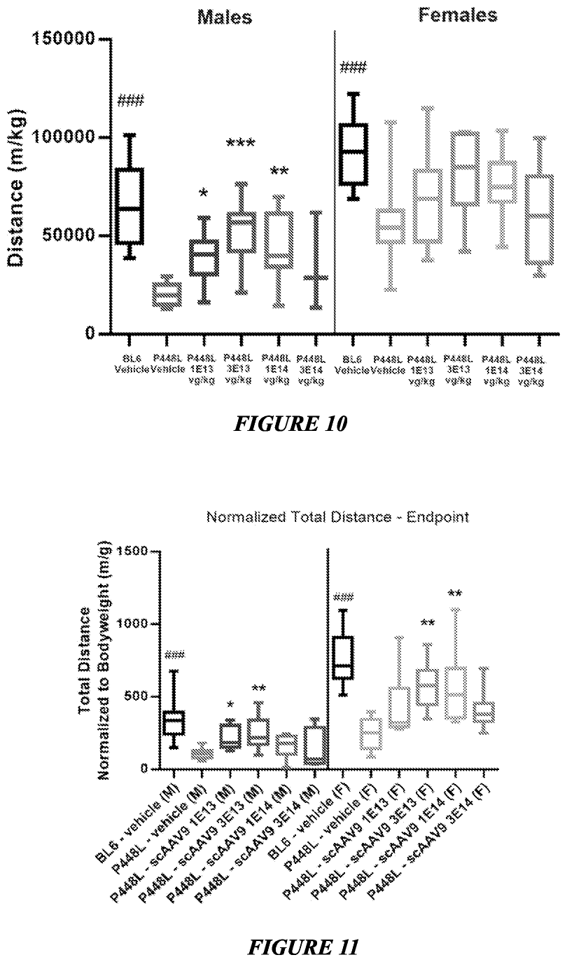

As shown in FIG. 11, in an endpoint voluntary wheel study with both male and female mice, the P448L vehicle mice ran a significantly shorter distance compared to the BL6 vehicle mice, both on an absolute basis and when the data was normalized to body weight. In addition, mice treated with AAV9-FKRP ran a greater distance compared to the P448L vehicle mice. Similar results were obtained from mice in a treadmill exhaustion state (FIG. 10), although statistical significance was not observed in female mice treated with AAV9-FKRP when the data was normalized to body weight. While the reason for the difference in responses between the sexes in the mice remains unclear, these results indicate that the response to AAV9-FKRP in P448L mice is meaningful, improving endurance and physical activity.

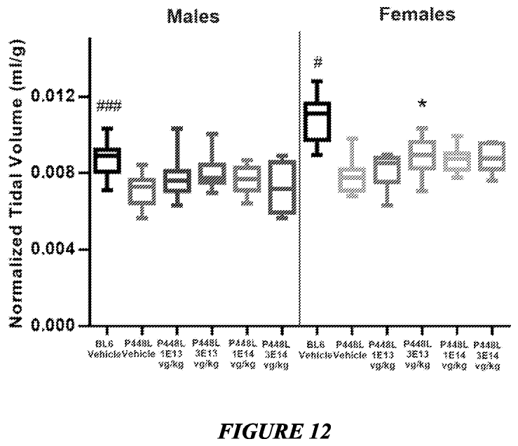

As shown in FIG. 12, plethysmography studies performed revealed a dose dependent improvement in breathing, indicated by increased normalized tidal volume. This was more readily observe in females than in males.

Discussion

The results from these preclinical dose-finding and toxicology studies indicate that AAV9-FKRP will have therapeutic benefits following systemic delivery at analogous doses for human patients, including restoration of skeletal muscle contractile function, body-wide expression of functional glycosylation of alpha-dystroglycan in skeletal muscles, reduction in the progressive loss of contractile tissue (muscle) with reduced appearance of non-contractile tissue (fibrosis and fat), and improvement in functional measures such as physical ability and endurance.

### Example 3—Clinical Trials

The pilot/pivotal clinical study is multi-center, double-blind, randomized, placebo-controlled Phase 1/2 clinical trial of AAV9-FKRP in human patients with rare, autosomal recessive mutations in the gene encoding fukutin-related proteins (genotypically confirmed LGMD21/R9). This trial will be carried out in two parts: Study Part 1 will be a pilot study to evaluate safety, target activity, and preliminary efficacy to help identify the recommended Phase 2 dose (RP2D) of gene therapy; and Study Part 2 will be a pivotal study to confirm safety and efficacy of the gene therapy at the R2PD.

The clinical study will enroll human subjects who are homozygous for the L2761/R9 (c.826C>A) mutation (Pilot Study, Part 1) and subsequently who are either homozygous or heterozygous for the L2761/R9 (c.826C>A) mutation (Pivotal Study, Part 2), to assess single IV infusion doses of 1E13 vg/kg, and 3E13 vg/kg. The pilot trial will have two dose escalating cohorts with 4 patients in cohort 1 (low dose, 1E13 vg/kg), and 6 patients in cohort 2 (high dose, 3E13 vg/kg).

The pivotal study is estimated to enroll 51 subjects, dosed at the R2PD. Within both the pilot and pivotal studies, subjects who will be randomized to placebo, will be offered gene therapy, if they remain eligible at the end of their respective parts of the study.

When receiving therapy, subjects will receive immune suppression medications. Steroid prophylaxis will begin 24 hours+/−8 hours prior to vector dosing of Day 1; oral prednisone will be given at a dose of 1 mg/kg body mass daily up to a total dose of 60 mg for 4 weeks followed by a ˜0.08 mg/kg, or 5 mg if taking 60 mg, taper (to the nearest 1 mg) each week for 12 weeks. A diary for steroid compliance will be kept by the subject and monitored by the study team during on-site, home and phone visits.

Pre-specified co-primary endpoints will include safety and efficacy. Efficacy will be evaluated by primary and secondary function endpoints, which will include but not limited to: a North Star Assessment for Limb Girdle Muscular Dystrophies (NSAD); Clinical Global Impression (CGI) for disease improvement, severity, and therapeutic efficacy; 10-meter walk test (10 MWT); 100-meter walk test (100 MWT); 4-stair climb (4SC);-Timed-Up and -Go (TUG); Performance Upper Limb (PUL); and/or patient reported outcome measures (e.g., individualized quality of life, fatigue, sleepiness, depression scores).

Additionally, physiologic assessments for cardiac and respiratory function will be evaluated to examine the progression of heart and pulmonary disease. MRI assessment of lower extremities will be performed to assess acute on chronic muscle injury and disease progression (e.g. muscle edema, fatty replacement, and wasting). Muscle, diaphragm and heart tissue of recipient subjects will be targeted and analyzed for FKRP expression, α-dystroglycan content, glycosylated α-dystroglycan content, collagen content by muscle biopsy analysis. Serum creatine kinase levels and other proteomic and metabolomic biomarkers will also be analyzed.

For all these endpoints, the change from baseline will be measured at different time points, e.g., at baseline, 16 weeks, 24 weeks, 40 weeks, and 52 weeks. These endpoints are exploratory, and thus a statistically significant difference from baseline would be noted. In addition, a statistically significant reduction of serum CK levels (e.g., towards normal) may be a surrogate of reduced muscle injury, i.e., indicating the efficacy of the therapeutic product described herein. Change from baseline in L VEF diastolic and systolic volume and cardiac output will be measured at one or, more of said timepoints. In addition, B-cell and T-cell immunological responses (total/circulating and neutralizing anti-adeno-associated virus serotype 9 (AAV9) antibody titers); and/or, T-cell reactivity to AAV and FKRP) from baseline to up to 12 months; and/or AAV9 vector shedding will be analysed.

Furthermore, exploratory endpoints will be measured that includes analysis of one or, more of the following; Immunophenotyping of B cells and T cells; will be observed and changes from baseline at 24, 40, and 52 weeks in pulmonary function as measured by forced vital capacity (FVC), will be observed and changes from baseline at 24, 40, and 52 weeks in forced expiratory volume in the first second (FEV 1), will be observed and changes from baseline at 24, 40, and 52 weeks in maximum inspiratory pressure (MIP), will be observed and changes from baseline at 24, 40, and 52 weeks in maximum expiratory pressure (MEP), will be observed and changes from baseline at 8, 24, and 52 weeks in cardiac structure and function as measured by ejection fraction (EF), will be observed and changes from baseline to 52 weeks in left ventricular end systolic volume index (LVESVI), will be observed and changes from baseline at 8, 24 and 52 weeks in myocardial peak circumferential strain by echocardiogram, will be observed and changes from baseline at 52 weeks in lower extremity muscle quality and quantity as measured by MRI T2w(STIR), fat suppressed edema signal, centrally scored that are observed and changes from baseline and 52 weeks in lower extremity muscle in active treatment group compared to control group as measured by MRI 3-point-Dixon fat fraction sequences.

It is expected that one or, more of the primary/secondary endpoints, e.g., creatine kinase level, will be improved over time for the treated patients compared to placebo. For example, it is expected that for the treated patients, creatine kinase level will be reduced over time surprisingly coming to about the normal level.

With the gene therapy as described in the invention, it is expected that clinical meaningful changes will be observed for the endpoints discussed herein. For example, in the NSAD, an observable and clinical meaningful difference of about 1.73 points from baseline will be observed¹. In the 10 MWT, an observable and clinically meaningful change of about 31% from baseline (e.g., 2.3 seconds) will be observed². In the 100 MWT, an observable and clinically meaningful change of about 6 seconds from baseline will be observed³. In the 4SC, an observable and clinically meaningful difference of about 30% from baseline (e.g., 2.1 seconds) will be observed. In the TUG, similar to what is seen in DM-1 patients, an observable and clinically meaningful change of about 30% from baseline will be observed. In the PUL, seen in LGMD patients, an observable and clinically meaningful change of about 4 points from baseline will be observed⁴.

### REFERENCES FOR EXAMPLE 3

1) Jacobs M B, James M, Lowes L P, Alfaio L N, Eagle M, Muni Lofra R, Moore U, Feng J, Rufibach L E, Rose K, Duong T, Bello L, Pedrosa-Hernández I, Hoisten S. Sakamoto C, Canal A, Sanchez-Aguilera Práxedes N, Thiele S, Siener C, Vandevelde B, DeWolf B, Maron E, Guglieri M, Hogrel J Y, Blamire A M, Carlier P G, Spuler S, Day J W, Jones K J, Bharucha-Goebel D X, Salort-Campana E, Pestronk A, Walter M C, Paradas C, Stojkovic T, Mori-Yoshimura M, Bravver E, Diaz-Manera J, Pegoraro E, Mendell J R; Jain COS Consortium, Mahew A G, Straub V. Assessing Dysferlinopathy Patients Over Three Years With a New Motor Scale. Ann Neurol. 2021 May; 89(5):967-978. doi: 10.1002/ana.26044. Epub 2021 Feb. 26. PMID: 33576057.
2) McDonald C M, Henricson E K, Abresch R T, Florence J, Eagle M, Gappmaier E, Glanzman A M; PTC124GD-007-DMD Study Group, Spiegel R, Barth J, Elfring G, Reba A, Peltz S W. The 6-minute walk test and other clinical endpoints in duchenne muscular dystrophy: reliability, concurrent validity, and minimal clinically imnportait differences from a multicenter study. Muscle Nerve. 2013 September; 48(3):357-68. doi: 10.1002/mus23905. Epub 2013 Jul. 17. PMID: 23674289; PMCID: PMC3826053.
3) Mendell J R, Sahenk Z, Lehman K, et al. Assessment of Systemic Delivery of rAAVrh74.MHCK7.micro-dystrophin in Children With Duchenne Muscular Dystrophy: A Nonrandomized Controlled Trial. JAMA Neurol. 2020; 77(9):1122-1131. doi:10.1001/jamaneurol.2020.1484
4) Gandolla M, Antonietti A, Longatelli V, Biffi E, Diella E, Delle Fave M, Rossini M, Molteni F, D'Angelo G, Bocciolone M, Pedrocchi A. Test-retest reliability of the Performance of Upper Limb (PUL) module for muscular dystrophy patients. PLoS One. 2020 Sep. 28; 15(9):e0239064. doi: 10.1371/journal.pone.0239064. PMID: 32986757 PMCID: PMC7521751.

### Example 4—Production of Viral Vectors Comprising Nucleic Acid Encoding FKRP Polypeptide Operatively Linked to a Muscle-Specific Promoter Using HEK293 Cells

Derivation of suspension HEK293 cells from an adherent HEK293 Qualified Master Cell Bank. The derivation of the suspension cell line from the parental HEK293 Master Cell Bank (MCB), is performed in a Class 10,000 clean room facility. The derivation of the suspension cell line is carried out in a two phase process that involved first weaning the cells off of media containing bovine serum and then adapting the cells to serum free suspension media compatible with HEK293 cells. The suspension cell line is created as follows. First, a vial of qualified Master Cell Bank (MCB) is thawed and placed into culture in DMEM media containing 10% fetal bovine serum (FBS) and cultured for several days to allow the cells to recover from the freeze/thaw cycle. The MCB cells are cultured and passaged over a 4 week period while the amount of FBS in the tissue culture media is gradually reduced from 10% to 2.5%. The cells are then transferred from DMEM 2.5% FBS into serum free suspension media and grown in shaker flasks. The cells are then cultured in the serum-free media for another 3 weeks while their growth rate and viability is monitored. The adapted cells are then expanded and frozen down. A number of vials from this cell bank are subsequently thawed and used during process development studies to create a scalable manufacturing process using shaker flasks and wave bioreactor systems to generate rAAV vectors. Suspension HEK293 cells are grown in serum-free suspension media that supports both growth and high transfection efficiency in shaker flasks and wave bioreactor bags. Multitron Shaker Incubators (ATR) are used for maintenance of the cells and generation of rAAV vectors at specific rpm shaking speeds (based on cell culture volumes), 80% humidity, and 5% CO₂.

Transfection of suspension HEK293 cells. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The plasmid DNA has a sequence comprising a heterologous nucleic acid sequence encoding a FKRP protein operatively linked to muscle-specific promoter with other required regulatory sequences (SEQ ID NO: 1). In addition, AAV rep and AAV cap genes and adenovirus helper genes (e.g., encoded on one or more additional plasmids) are also added. The cocktail is inverted to mix prior to being incubated at room temperature. The transfection cocktail is then pipetted into the flasks and placed back in the shaker/incubator. All optimization studies are carried out at 30 mL culture volumes followed by validation at larger culture volumes. Cells are harvested 48 hours post-transfection.

Production of rAAV using wave bioreactor systems. Wave bags are seeded 2 days prior to transfection. Two days post-seeding the wave bag, cell culture counts are taken and the cell culture is then expanded/diluted before transfection. The wave bioreactor cell culture is then transfected. 48 hours post-transfection, wave bioreactor cell culture are cultured under conditions that induce expression of the rep and cap proteins. Such conditions for rep expression require administering NKH 477 at a concentration of from 1 μM to 100 μM, e.g. 8 μM in the wave bioreactor cell culture. Such conditions for cap expression require culturing the cells under hypoxic conditions, i.e. 5% oxygen. Cell culture is harvested from the wave bioreactor bag at least 48 hours post-induction.

Analyzing transfection efficiency using Flow Cytometry. Approximately 24 hours post-induction, 1 mL of cell culture is removed from each flask or wave bioreactor bag as well as an uninduced control. Samples are analyzed using a Dako Cyan flow cytometer to confirm that the plasmid DNA.

Harvesting suspension cells from shaker flasks and wave bioreactor bags. 48 hours post-induction, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 min. At this point, the pellet could either be stored in NLT-60° C. or continued through purification.

Titering rAAV from cell lysate using qPCR 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl₂, 5 mM MgCl₂, 50 mM Tris-HCl pH 8.0) followed by sonication to lyse the cells efficiently. 300 ul is then removed and placed into a 1.5 mL microfuge tube. 140 units of DNase I is then added to each sample and incubated at 37° C. for 1 hour. To determine the effectiveness of the DNase digestion, 4-5 ug of plasmid DNA is spiked into a non-transfected cell lysate with and without the addition of DNase. 50 ul of EDTA/Sarkosyl solution (6.3% sarkosyl, 62.5 mM EDTA pH 8.0) is then added to each tube and incubated at 70° C. for 20 minutes. 50 ul of Proteinase K (10 mg/mL) is then added and incubated at 55° C. for at least 2 hours. Samples are then boiled for 15 minutes to inactivate the Proteinase K. An aliquot is removed from each sample to be analyzed by qPCR. Two qPCR reactions are carried out in order to effectively determine how much rAAV vector is generated per cell.

Purification of rAAV from crude lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art could use mechanical methods such as microfluidization or chemical methods such as detergents, etc., followed by a clarification step using depth filtration or Tangential Flow Filtration (TFF).

AAV vector purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc).

Titering rAAV using dot blot. 100 ul of DNase buffer (140 units DNase, 5 mM CaCl₂, 5 mM MgCl₂, 50 mM Tris-HCl pH 8.0) is added to each well of a 96-well microtiter plate. 1-3 ul or serial dilutions of virus is added to each well and incubated at 37° C. for 30 min. The samples are then supplemented with 15 ul Sarkosyl/EDTA solution (6.3% sarkosyl, 62.5 mM EDTA pH 8.0) and placed at 70° C. for 20 min. Next, 15 ul of Proteinase K (10 mg/mL) is added and incubated at 50° C. for at least 2 hours. 125 ul of NaOH buffer (80 mM NaOH, 4 mM EDTA pH 8.0) is added to each well. A series of transgene specific standards are created through a dilution series. NaOH buffer is then added and incubated. Nylon membrane is incubated at RT in 0.4 M Tris-HCl, pH 7.5 and then set up on dot blot apparatus. After a 10-15 minute incubation in NaOH buffer, the samples and standards are loaded into the dot blot apparatus onto the GeneScreen PlusR hybridization transfer membrane (PerkinElmer). The sample is then applied to the membrane using a vacuum. The nylon membrane is soaked in 0.4 M Tris-HCl, pH 7.5 and then cross linked using UV strata linker 1800 (Stratagene) at 600 ujouls×100. The membrane is then pre-hybridized in CHURCH buffer (1% BSA, 7% SDS, 1 mM EDTA, 0.5 M Na₃PO₄, pH 7.5). After pre-hybridization, the membrane is hybridized overnight with a ³²P-CTP labeled transgene probe (Roche Random Prime DNA labeling kit). The following day, the membrane is washed with low stringency SSC buffer (1×SSC, 0.1% SDS) and high stringency (0.1×SSC, 0.1% SDS). It is then exposed on a phosphorimager screen and analyzed for densitometry using a STORM840 scanner (GE).

Analyzing rAAV vector purity using silver stain method. Samples from purified vector are loaded onto NuPage 10% Bis-Tris gels (Invitrogen) and run using 1× NuPage running buffer. Typically, 1×10¹⁰ particles are loaded per well. The gels are treated with SilverXpress Silver staining kit #LC6100 (Invitrogen).

Analysis of self-complementary genomes using alkaline gel electrophoresis and southern blot. Briefly, purified self-complementary rAAV is added to 200 ul, of DNase I buffer (140 units DNase, 5 mM CaCl₂, 5 mM MgCl₂, 50 mM Tris-HCl pH 8.0) and incubated at 37° C. for 60 minutes, followed by inactivation of the DNase by adding 30 ul, of EDTA Sarkosyl/EDTA solution (6.3% sarkosyl, 62.5 mM EDTA pH 8.0) and placed at 70° C. for 20 min. 20 ul of Proteinase K (10 mg/mL) is then added to the sample and incubated for a minimum of 2 hours at 50° C. Phenol/Chloroform is added in a 1:1 ratio, followed by ethanol precipitation of the viral vector DNA. The pelleted DNA is then resuspended in alkaline buffer (50 mM NaOH, 1 mM EDTA) for denaturation, loaded onto a 1% alkaline agarose gel, and run at 25V overnight. The gel is then equilibrated in alkaline transfer buffer (0.4 M NaOH, 1 M NaCl) and a southern blot is performed via an overnight transfer of the vector DNA to a GeneScreen PlusR hybridization transfer membrane (PerkinElmer). The membrane is then neutralized using 0.5 M Tris pH 7.5 with 1 M NaCl, and is hybridized overnight with a ³²P-CTP labeled transgene probe. After washing the membrane as previously described, the membrane is exposed to a phosphorimager screen and analyzed using a STORM840 scanner.

Transduction Assays. HeLaRC-32 cells (Chadeuf et al., J Gene Med. 2:260 (2000)) are plated at 2×10⁵ cells/well of a 24 well plate and incubated at 37° C. overnight. The cells are observed for 90-100% confluence. 50 mL of DMEM with 2% FBS, 1% Pen/Strep is pre-warmed, and adenovirus (d1309) is added at a MOI of 10. The d1309 containing media is aliquoted in 900 ul fractions and used to dilute the rAAV in a series of ten-fold dilutions. The rAAV is then plated at 400 μl and allowed to incubate for 48 hours at 37° C.

Concentration Assays. The starting vector stock is sampled and loaded onto a vivaspin column and centrifuged at 470×g (Sorvall H1000B) in 10 minute intervals. Once the desired volume/concentration had been achieved, both sides of the membrane are rinsed with the retentate, which is then harvested. Samples of the pre-concentrated and concentrated rAAV are taken to determine physical titers and transducing units.

Transmission electron microscopy (TEM) of negatively stained rAAV particles. Electron microscopy allows a direct visualization of the viral particles. Purified dialyzed rAAV vectors are placed on a 400-mesh glow-discharged carbon grid by inversion of the grid on a 20 ul drop of virus. The grid is then washed 2 times by inversion on a 20 ul drop of ddH₂O followed by inversion of the grid onto a 20 ul drop of 2% uranyl acetate for 30 seconds. The grids are blotted dry by gently touching Whatman paper to the edges of the grids. Each vector is visualized using a Zeiss EM 910 electron microscope.

### Example 5—Production of Viral Vectors Comprising Nucleic Acid Encoding FKRP Polypeptide Operatively Linked to a Muscle-Specific Promoter Using Pro10 Cells

Nucleic acid constructs comprising the FKRP expression cassette (SEQ ID NO: 1) (e.g., in the plasmid of FIG. 13,) are used to manufacture viral vectors in a stable cell line for AAV production, Pro10 cells. These stable Pro10 cells for AAV production, e.g., as described in U.S. Pat. No. 9,441,206, are ideal for scalable production of AAV vectors. The cell line is contacted with FKRP nucleic acid construct (e.g. the plasmid shown in FIG. 13) via transfection to receive the nucleic acid. The presence of the nucleic acid constructs is confirmed via PCR-based assays using primers specific for the plasmid.

Transfection. Stable Pro10 cells are transfected with FKRP nucleic acid constructs and are also transfected with a packaging plasmid encoding Rep and serotype-specific Cap: alternatively, AAV-Rep/Cap is provided as self-annealed circular nucleic acids, and/or the Ad-Helper plasmid (XX680: encoding adenoviral helper sequences) is provided on one or more plasmids, or as self-annealed circular nucleic acids.

On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The cocktail is inverted to mix prior to being incubated at room temperature. The transfection cocktail is pipetted into the flasks and placed back in the shaker/incubator. All optimization studies are carried out at 30 mL culture volumes followed by validation at larger culture volumes. Cells are harvested 48 hours post-transfection.

Production of rAAV Using Wave Bioreactor Systems. Wave bags are seeded 2 days prior to transfection. Two days post-seeding the wave bag, cell culture counts are taken and the cell culture is then expanded/diluted before so transfection. The wave bioreactor cell culture is then transfected. Cell culture are harvested from the wave bio-reactor bag at least 48 hours post-transfection.

Titer: AAV titers are calculated after DNase digestion using qPCR against a standard curve (AAV ITR specific) and primers specific to the factor IX nucleic acid construct.

Harvesting Suspension Cells from Shaker Flasks and 60 Wave Bioreactor Bags. 48 hours post-transfection, cell cultures are collected into 500 mL polypropylene conical tubes (Corning) either by pouring from shaker flasks or pumping from wave bioreactor bags. The cell cultures are then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 mM. At this point, the pellet could either be stored in NLT-60° C. or continued through purification.

Titering rAAV from Cell Lysate Using qPCR 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl2, 5 mM MgCl2, 50 mM Tris-HCl pH 8.0) followed by sonication to lyse the cells efficiently. 300 uL is then removed and placed into a 1.5 mL microfuge tube. 140 units of DNase I is then added to each sample and incubated at 37° C. for 1 hour. To determine the effectiveness of the DNase digestion, 4-5 mg of the factor IX nucleic acid construct is spiked into a non-transfected cell lysate with and without the addition of DNase. 50 μL of EDTA/Sarkosyl solution (6.3% sarkosyl, 62.5 mM EDTA pH 8.0) is added to each tube and incubated at 70° C. for 20 minutes. 50 μL of Proteinase K (10 mg/mL) is then added and incubated at 55° C. for at least 2 hours. Samples are boiled for 15 minutes to inactivate the Proteinase K. An aliquot is removed from each sample to be analyzed by qPCR. Two qPCR reactions are carried out in order to effectively determine how much rAAV vector is generated per cell. One qPCR reaction is set up using a set of primers 2s designed to bind to a homologous sequence on the backbones of plasmids XX680, pXR2 and factor IX nucleic acid constructs. The second qPCR reaction is set up using a set of primers to bind and amplify a region within the factor IX mini gene. qPCR is conducted using Sybr green reagents and Light cycler 480 from 30 Roche. Samples are denatured at 95° C. for 10 minutes followed by 45 cycles (90° C. for 10 sec, 62° C. for 10 sec and 72° C. for 10 sec) and melting curve (1 cycle 99° C. for 30 sec, 65° C. for 1 minute continuous).

Purification of rAAV from Crude Lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art can use as mechanical methods such as microfluidization or chemical methods such as detergents, etc., followed by a clarification step using depth filtration or Tangential Flow Filtration (TFF).

AAV Vector Purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc), which are incorporated herein by reference in their entireties.

### Example 6—In Vitro Testing

The strength of the synthetic muscle-specific promoters or skeletal muscle-specific promoters according to certain embodiments of this invention are tested by operably linking them to the reporter gene luciferase. The expression cassette comprising of the muscle-specific promoter or skeletal muscle-specific promoter to be tested and the luciferase gene is inserted into a suitable plasmid which is then transfected into a cell in order to test the expression from the promoters in these cells.

Materials and Methods

DNA preparations are transfected into H9C2 (a rat BDIX heart myoblast cell line, available from ATCC) to assess transcriptional activity. H9C2 cell line was used as previous experiments have shown it to be a good predictor of skeletal and cardiac muscle activity in vivo.

H9C2 Cell Culture and Transfection

H9C2 are a rat BDIX heart myoblast cell line. They have cardiac muscle properties, e.g. myotubes formed at confluency respond to acetylcholine.

Cell Maintenance

H9C2 cells are cultured in DMEM (High Glucose, D6546, Sigma) with 1% FBS (Heat inactivated-Gibco 10270-106, lot number 42G2076K), 1% Glutamax (35050-038, Gibco), 1% Penicillin-streptomycin solution (15140-122, Gibco), in T-75 flasks. Cells are passaged at a sub confluent stage (70-80%) to avoid risk of the cells becoming confluent and fusing to form myotubes.

For passaging during cell maintenance, culture media is removed, cells are washed twice with 5 ml DPBS without CaCl₂, without MgCl₂ (14190-094, Gibco). The cells are detached from the flask by incubating with 1 ml Trypsin EDTA (25200-056, Gibco) for approximately 5 minutes. Then, 4 ml of culture medium is added to the flask and the mixture is gently pipetted up and down to help detach the cells from the flask surface. Cells are pelleted at 100 g for 3 minutes. Supernatant is disposed and cells are resuspended in 3 ml of culture medium. Cells are counted on the Countess automated cell counter, seeded at 1:3 to 1:10 i.e. seeding 1-3×10,000 cells/cm² and incubated at 37° C. 5% CO₂.

Cell Transfection and Differentiation

H9C2 cells are collected from two T-75 flasks of approximately 70-80% confluency, by washing with DPBS, detaching from the flask using 1 ml Trypsin EDTA, washing off the flask's surface with 4 ml of culture medium and pelleting at 100 g for 3 minutes, as described above. Cells are resuspended in 45 ml culture medium and seed at a density of 40,000 cells/well in a 48 well flat bottom plate (300 μl/well) (353230, Corning). Cells in 48-well plates are incubated at 37° C. 5% CO₂.

Twenty-four hours later, the culture medium on the cells is replaced with 300 μl antibiotic-free culture medium (i.e DMEM (High Glucose, D6546, Sigma) with 1% FBS (Heat inactivated—Gibco 10270-106, lot number 42G2076K), 1% Glutamax (35050-038, Gibco)). 300 ng of DNA per well is transfected with viafect (E4981, Promega) in a total complex volume of 30 μl per well. Plates are gently mixed following transfection and incubated at 37° C. 5% CO₂.

Twenty-four hours later, culture medium is removed from transfected cells and replaced with 300 μl differentiation media consisting of DMEM (High Glucose, D6546, Sigma), 1% Glutamax (35050-038, Gibco), 1% FBS (Heat inactivated—Gibco 10270-106, lot number 42G2076K), 1% Penicillin/streptomycin solution (15140-122, Gibco) and 0.1% Retinoid Acid (Sigma-R2625). Plates are incubated at 37° C. 5% CO₂ for 7 days to induce differentiation. After differentiation, cell morphology is observed to confirm differentiation into myotubes.

Cells are then washed with 500 μl DPBS, and lysed with 100 μl Luciferase Cell Culture Lysis 5×Reagent (E1531, Promega) diluted to 1×using Milli-Q water. Cell lysis reagent is pipetted up and down ten times and plates are then vortexed on a medium power for 30 minutes to promote cell lysis. Plates are sealed and stored at −80° C. prior to completing a luciferase assay. The data collected from luciferase assays following transfections in H9C2 cells is based on three technical replicates of at one biological replicate.

Measurement of Luciferase Activity
Luciferase activity is measured using LARII (Dual Luciferase Reporter 1000 assay system, Promega, E1980)
24 h after transfection, the media is removed from the cell
The cells are washed once in 300 μl of DPBS
Cells are lysed using 100 μl of passive lysis buffer and incubated with rocking for 15 minutes.
The cell debris is pelleted by centrifugation of the plate at max speed in a benchtop centrifuge for 1 min
10 μl sample is transferred into white 96-well plate and luminescence measured by injection of 50 μl of LARII substrate on a BMG Labtech FLUOstar Omega plate reader.

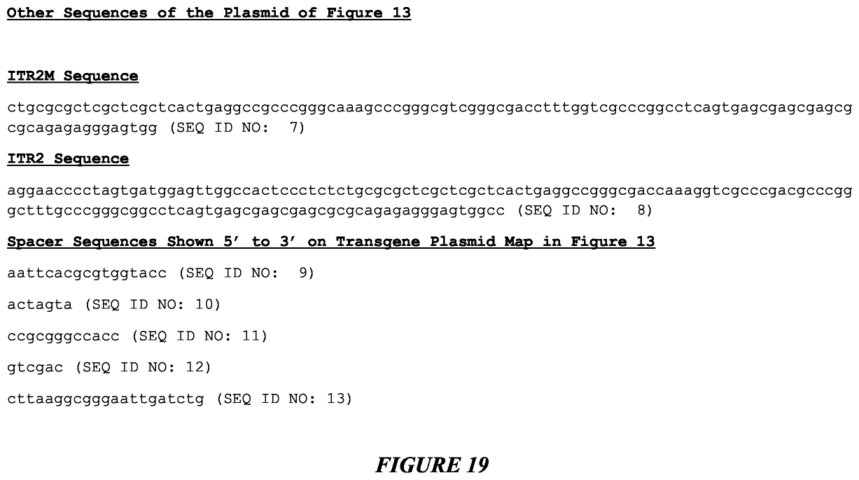

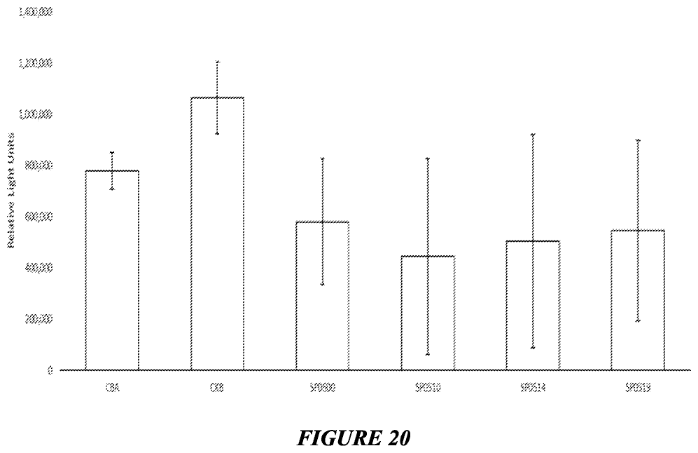

Results generated from these cell cultures are shown in FIG. 20. This figure shows that synthetic promoters SP0500, SP0510, SP0514 and SP0519 show good activity in the muscle cell line H9C2. Other similar promoters described herein are expected to have the same or better performance.

### REFERENCES FOR EXAMPLE 6

Llanga, T. et al. (2017) ‘Structure-Based Designed Nano-Dysferlin Significantly Improves Dysferlinopathy in BLA/J Mice’, Molecular Therapy. Elsevier Ltd., 25(9), pp. 2150-2162. doi: 10.1016/j.ymthe.2017.05.013.

### Example 7—In Vitro Testing of Short Skeletal Muscle-Specific Promoters

Materials and Methods

DNA preparations were transfected into H9C2 (a rat BDIX heart myoblast cell line, available from ATCC) to assess transcriptional activity. H9C2 cell line was used as previous experiments have shown it to be a good predictor of skeletal and cardiac muscle activity in vivo.

H9C2 Cell Culture and Transfection

H9C2 are a rat BDIX heart myoblast cell line. They have cardiac muscle properties, e.g. myotubes formed at confluency respond to acetylcholine.

Cell Maintenance

H9C2 cells were cultured in DMEM (High Glucose, D6546, Sigma) with 1% FBS (Heat inactivated—Gibco 10270-106, lot number 42G2076K), 1% Glutamax (35050-038, Gibco), 1% Penicillin-streptomycin solution (15140-122, Gibco), in T-75 flasks. Cells were passaged at a sub confluent stage (70-80%) to avoid risk of the cells becoming confluent and fusing to form myotubes.

For passaging during cell maintenance, culture media was removed, cells were washed twice with 5 ml DPBS without CaCl₂, without MgCl₂ (14190-094, Gibco). The cells were detached from the flask by incubating with 1 ml Trypsin EDTA (25200-056, Gibco) for approximately 5 minutes. Then, 4 ml of culture medium was added to the flask and the mixture was gently pipetted up and down to help detach the cells from the flask surface. Cells were pelleted at 100 g for 3 minutes. Supernatant was disposed and cells were resuspended in 3 ml of culture medium. Cells were counted on the Countess automated cell counter, seeded at 1:3 to 1:10 i.e. seeding 1-3×10,000 cells/cm² and incubated at 37° C. 5% CO₂.

Cell Transfection and Differentiation

H9C2 cells were collected from two T-75 flasks of approximately 70-80% confluency, by washing with DPBS, detaching from the flask using 1 ml Trypsin EDTA, washing off the flask's surface with 4 ml of culture medium and pelleting at 100 g for 3 minutes, as described above. Cells were resuspended in 45 ml culture medium and seed at a density of 40,000 cells/well in a 48 well flat bottom plate (300 μl/well) (353230, Corning). Cells in 48-well plates were incubated at 37° C. 5% CO₂.

Twenty-four hours later, the culture medium on the cells was replaced with 300 μl antibiotic-free culture medium (i.e DMEM (High Glucose, D6546, Sigma) with 1% FBS (Heat inactivated—Gibco 10270-106, lot number 42G2076K), 1% Glutamax (35050-038, Gibco)). 300 ng of DNA per well was transfected with viafect (E4981, Promega) in a total complex volume of 30 μl per well. Plates were gently mixed following transfection and incubated at 37° C. 5% CO₂.

Twenty-four hours later, culture medium was removed from transfected cells and replaced with 300 μl differentiation media consisting of DMEM (High Glucose, D6546, Sigma), 1% Glutamax (35050-038, Gibco), 1% FBS (Heat inactivated—Gibco 10270-106, lot number 42G2076K), 1% Penicillin/streptomycin solution (15140-122, Gibco) and 0.1% Retinoid Acid (Sigma-R2625). Plates were incubated at 37° C. 5% CO₂ for 7 days to induce differentiation. After differentiation, cell morphology was observed to confirm differentiation into myotubes.

Cells were then washed with 500 μl DPBS, and lysed with 100 μl Luciferase Cell Culture Lysis 5× Reagent (E1531, Promega) diluted to 1× using Milli-Q water. Cell lysis reagent was pipetted up and down ten times and plates were then vortexed on a medium power for 30 minutes to promote cell lysis. Plates were sealed and stored at −80° C. prior to completing a luciferase assay. The data collected from luciferase assays following transfections in H9C2 cells is based on three biological replicates each of which is an average of three technical replicates.

Measurement of Luciferase Activity
Luciferase activity was measured using LARII (Dual Luciferase Reporter 1000 assay system, Promega, E1980)
24 h after transfection, the media was removed from the cells
The cells were washed once in 300 μl of DPBS.
Cells were lysed using 100 μl of passive lysis buffer and incubated with rocking for 15 minutes.
The cell debris was pelleted by centrifugation of the plate at max speed in a benchtop centrifuge for 1 min
10 μl sample was transferred into white 96-well plate and luminescence measured by injection of 50 μl of LARII substrate on a BMG Labtech FLUOstar Omega plate reader

Results

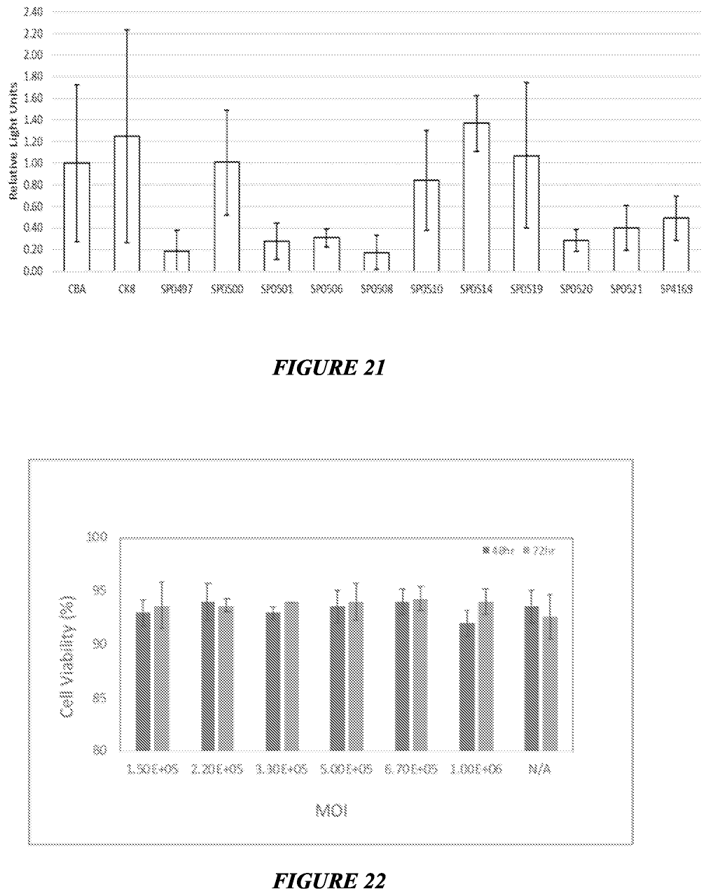

Results generated from these cell cultures are shown in FIG. 21. FIG. 21 shows that synthetic promoters SP0497, SP0500, SP0501, SP0506, SP0508, SP0510, SP0514, SP0519, SP0520, SP0521 and SP4169 have good activity in the muscle cell line H9C2. Promoters SP0498, SP0499, SP0502, SP0503, SP0504, SP0505, SP0507, SP0509, SP0511, SP0512, SP0513, SP0515, SP0516, SP0517, SP0518, SP0522, SP0523 and SP0524 were also tested experimentally in the H9C2 cell line but showed lower activity (data not shown). Experiments were performed in triplicate.

### Example 8—Preparation for Determination of FKRP Activity Using HA-VSMC

Human Aortic Smooth Muscle cells (HA-VSMC or, HASMC cells) were purchased from American Type Culture Collection (ATCC)

Reagent Preparation

25 mL FBS and vascular smooth muscle cell supplement kit were thawed. The thawed FBS and smooth muscle cell supplement kit (see table 2 for components) were added to the graduated filter of a sterile, 0.22 μm PES filter system. The graduated filter was filled to 500 mL with F-12K medium and then filter sterilized.

TABLE 14
Vascular smooth Muscle Cell Growth Kit Components.

| | | |
|---|---|---|
| | | Final |
| Component | Volume | concentration |

| | | | | |
|---|---|---|---|---|
| rh FGF-basic | 0.5 | mL | 5 | ng/mL |
| rh Insulin | 0.5 | mL | 5 | μg/mL |
| Ascorbic acid | 0.5 | mL | 50 | μg/mL |
| L-glutamine | 0.5 | mL | 5 | ng/mL |
| rh EGF | 0.5 | mL | 5 | ng/mL |

| | | | |
|---|---|---|---|
| Fetal Bovine | 25 | mL | 5% |
| Serum | | | |

Procedure for Splitting and Counting HASMC Cells

Cell Preparation included the following steps:
1. Growth medium was brought to room temperature (about 30 minutes), growth medium was aspirated from the HA-VSMC flasks, flasks were washed twice with PBS, trypsin was then added (1.5 ml trypsin for 75 cm2 flask) to detach the cells from the growth surface.
2. Once the cells were detached in about at room temperature for ≤5 minutes, 9.5 ml growth medium added to neutralize the trypsin and rinsing the surface of the flask, the cell suspension was then centrifuged at 1200 rpm for 5 minutes to pellet the cells, cell pellet was then resuspended in 1 ml growth medium and cell viability was checked with Trypan blue testing using Countess II Automated Cell Counters.
3. 10,000 cells were plated per well of a 96 well plate with the final volume of 150 ul/well.
4. The assay plate was incubated for 22±2 hours in a 37° C./5% CO2 incubator

Procedure Vector Preparation and Transduction

The amount of AAV9-syn-coFKRP stock was determined that was needed. The reference standard stock from storage at ≤−80° C. was removed and thawed at room temperature. The thawed stock was equilibrated to room temperature.

Low serum media (5%) was prepared, vascular smooth muscle cell supplement kit was added to the graduated filter of a sterile, 0.22m PES filter system. The graduated filter was filled to 500 mL with F-12K medium.

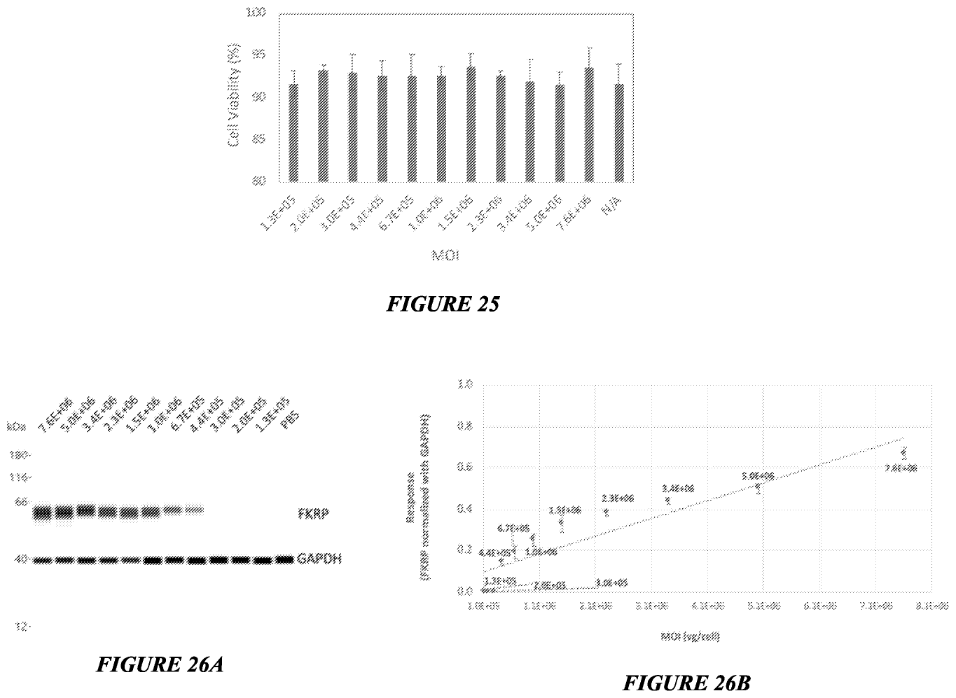

Vector solution was prepared using adjusted titer, 4.67E+12 as Table 15, results shown at FIGS. 22, 23 and 24; and Table 16, results shown at FIGS. 25 and 26.

Note for the Table: all these need to add dilution buffer to the same volume as the highest dose cohort.

TABLE 15
rAAV(scAAV9-syn100-coFKRP) dilutions
for each well in 96-well plate.

| | | | Volume | |
|---|---|---|---|---|
| | scAAV9-syn- | Volume of | Assay | |
| | coFKRP | scAAV9-syn- | Medium | Final Volume |
| Point | MOI | coFKRP (μL) | (μL) | per-well (uL) |
| 1 | 1.0E+06 | 13.4 | 136.6 | 150 |
| 2 | 6.7E+05 | 8.9 | 141.1 | 150 |
| 3 | 5.0E+05 | 6.6 | 143.4 | 150 |
| 4 | 3.3E+05 | 4.4 | 145.6 | 150 |
| 5 | 2.2E+05 | 2.93 | 147.03 | 150 |
| 6 | 1.50E+05 | 1.95 | 148.05 | 150 |

TABLE 16
rAAV(scAAV9-syn100-coFKRP) dilutions
for each well in 96-well plate.

| | | | Volume | |
|---|---|---|---|---|
| | scAAV9-syn- | Volume of | Assay | |
| | coFKRP | scAAV9-syn- | Medium | Final Volume |
| Point | MOI | coFKRP (μL) | (μL) | per-well (uL) |
| 1 | 7.6E+06 | 102 | 48 | 150 |
| 2 | 5.0E+06 | 68 | 82 | 150 |
| 3 | 3.4E+06 | 45.2 | 104.8 | 150 |
| 4 | 2.3E+06 | 30.2 | 119.8 | 150 |
| 5 | 1.5E+06 | 20.1 | 129.9 | 150 |
| 6 | 1.0E+06 | 13.4 | 136.6 | 150 |
| 7 | 6.7E+05 | 8.9 | 141.1 | 150 |
| 8 | 4.4E+05 | 5.9 | 144.1 | 150 |
| 9 | 3.0E+05 | 3.9 | 146.1 | 150 |
| 10 | 2.0E+05 | 2.6 | 147.4 | 150 |
| 11 | 1.3E+05 | 1.73 | 148.27 | 150 |

It is noted that the Research Grade vector titer was over-estimated (˜10-fold), the original titer, 4.67E+13 has no dose response detected.

HASMC Cell Lysate for Determination of FKRP Activity

Preparation of Harvested Cell Lysate

After 48-hour and 72-hour post transduction, 150 μL/well low serum media was removed and replaced it with 150 μL/well PBS. Removal of media and addition of PBS were repeated another three times. After the final wash, all of the liquid was removed, being careful not to disturb the cell layer. 50 μL RIPA buffer with protease inhibitor was added to each well and was incubated at room temperature for 5 minutes. The plate was sealed and froze at ≤−80° C. When ready to test the samples, the samples were thawed. Carefully cell lysates were moved to a ddPCR Plate and centrifuged at 4.7 k RPM for 20 minutes at 2-8° C. The lysates were kept on ice.

FKRP Assay for Cell Lysate

The reagents were prepared included in the EZ Standard Pack (Protein Simple) by completing the following:
1. 40 μL of Deionized (DI) Water was added to the Dithiothreitol (DTT) to make a 400 mM concentration solution.
2. 20 μL of 10× Sample Buffer and 20 μL of 400 mM DTT Solution were added to the Fluorescent 5× Master Mix to make a 1× Solution.
3. 20 μL of DI Water was added to the Biotinylated Ladder. Reagents were vortexed to mix and maintain on ice until use.
4. The lysates were prepared by combining 1.5 μL of the Fluorescent Master Mix and 6 μL of the lysate. These were then combined in a clean set of tubes and the samples were vortexed to mix.
5. The samples were denatured by placing in a heat block set at 95° C. for five minutes.
6. The samples were vortexed again. Then, briefly centrifuged to collect the sample at the bottom of the tube and retained on ice until ready to use.
7. The anti-FKRP (PA5-65349, Invitrogen; 1:250) and anti-GAPDH (NB100-56875, Novus Bio; 1:5000) primary antibodies were prepared by diluting with Antibody Diluent 2.
8. Substrate was prepared by mixing 100 μL of Luminol-S and 100 μL of Peroxide.
9. According to the provided plate map, 5 μL of the Biotin Ladder, 5 μL of the prepared samples, 20 L of the Antibody Diluent 2, 15 μL of each of the primary and secondary antibodies, 15 μL of the Luminol-peroxide substrate were pipetted into the 384 well plate.
10. 15 μL of the Stacking Matrix first, then 30 μL of DI water, and lastly, 15 μL of the Separation Matrix were pipetted.
11. The plate was centrifuged for five minutes at 2500 rpm (˜1000×g) at room temperature.
12. The desired assay template the Protein Simple® Compass Western software was opened.
13. Then the run was started following the remaining instructions to ready the machine for the assay run.
14. The run was saved when it was complete.

Run Analysis-After the run was completed, the data was analyzed through the Protein Simple® Compass for Simple Western program. Fluorescent Sizing Standards.

Results

Cell Viability Assay

Extra set of transduced cells in same condition and MOI were prepared for cell viability assay. Cells were gently removed from the culture plates with trypsin. Aliquots of cell suspension being tested for viability were centrifuged for 5 min at 1000×g. The pellets were re-suspended in 200 uL of PBS. 10 uL of cell suspension was mixed with 10 uL of trypan blue and incubated 2 min at room temperature. Then 10 ul of the suspension was placed in a disposable slide and cells were counted using Countess II Automated Cell Counters (ThermoFisher Scientific) within 3 min from the end of the incubation.

FIG. 22 demonstrates that cell survival/cell viability was unaffected by transduction at 48-hour and 72-hour incubation regardless of the MOI.

FKRP Activity in Cell Lysate

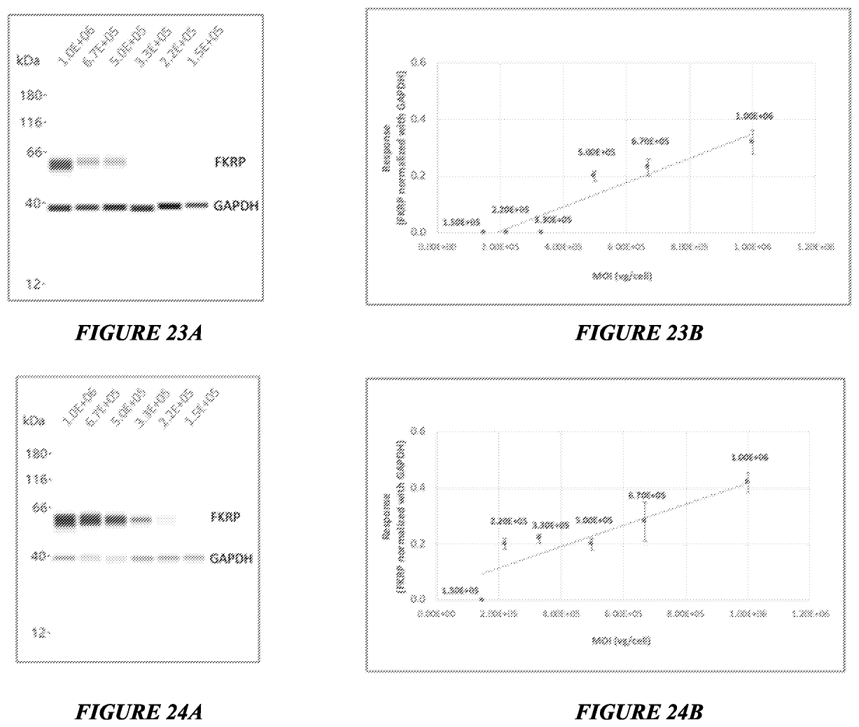

FKRP activity in the cell lysate was increased with increased in 48-hour and 72 hour and MOI as indicated in FIGS. 23A, 23B, 24A, 24B, 26A, and 26B.

For the cell lysate, FKRP activity increased with MOI when normalized to protein (FIGS. 23A, 23B, 24A, 24B and 26A) and reduced per vector genome when MOI increased (FIGS. 23A, 23B, 24A, 24B and 26B).

According to FIGS. 24A and 24B, the data from 72-hour post transduction showed better dose response then 48-hour post transduction (FIGS. 23A and 23B), therefore more MOI levels were next performed to further check the cells response to (scAAV9-syn-coFKRP) in higher MOI. Vector preparation and dilution between 1.3E+05 to 7.6E+06 according to Table 16.

FIG. 25 demonstrated that cell survival/cell viability was unaffected by transduction at 72-hour incubation regardless of the MOI.

Discussion

An in vitro potency assay is developed for a therapeutic rAAV comprising FKRP (scAAV9-syn-coFKRP). The assay is reproducible and linear over the range of 4.4E5 to 7.5E6 vector genomes per assay and is useful for assessing the relative potency of multiple independently synthesized vector lots. The assay is therefore suitable for release testing and for assessing stability of vector lots over time.

The results support the following conclusions: (1) The greatest production of FKRP in lysate occurs at 72 hours; (2) The normalized FKRP shows activity over the range of 4.4E5 to 7.5E6 MOI; and (3) Cell survival is unaffected by transduction with the vector.

These results indicate the validation and use of this assay for determining the activity of drug product, scAAV9-syn-coFKRP, for clinical dosing as a replacement to the current in vivo assay. In vitro potency assays developed by the inventors can also work as a bridging assay if there is a change in vectors, or, change in promoter, or, change in the transgene (e.g., codon optimization of the transgene) or, change in any component of the rAAV comprising expression cassette, and will help validate the potency of the altered therapeutic product as compared to the parent one or, to a reference. This can also suitably perform as a bridging assay if the rAAV is manufactured from a plasmid template or, from a close ended linear duplexed DNA (celDNA) template and thus can validate the potency of the therapeutic product obtained from each format.

### Example 9—Preparation for Determination of FKRP Activity Using LGMD2I Patient-Derived Cell Line

The LGMD2I patient-derived cell line is α-dystroglycan deficient and expresses decreased levels of FKRP.

Reagent Preparation

Thaw 25 mL FBS and muscle cell supplement kit. Add the thawed FBS and smooth muscle cell supplement kit (see table 2 for components) to the graduated filter of a sterile, 0.22 μm PES filter system. Fill the graduated filter to 500 mL with F-12K medium. Sterile filter and label the bottle with the reagent name, reagent lot number, expiration date, initials, date and storage condition. The growth medium expires in 1 month.

TABLE 17
Muscle Cell Growth Kit Components.

| | | |
|---|---|---|
| | | Final |
| Component | Volume | concentration |

| | | | | |
|---|---|---|---|---|
| rh FGF-basic | 0.5 | mL | 5 | ng/mL |
| rh Insulin | 0.5 | mL | 5 | μg/mL |
| Ascorbic acid | 0.5 | mL | 50 | μg/mL |
| L-glutamine | 0.5 | mL | 5 | ng/mL |
| rh EGF | 0.5 | mL | 5 | ng/mL |

| | | | |
|---|---|---|---|
| Fetal Bovine | 25 | mL | 5% |
| Serum | | | |

Procedure for Splitting and Counting LGMD2I Patient-Derived Cells

Cell Preparation includes the following steps:
1. Bring growth medium to room temperature (about 30 minutes).
2. Determine which flasks of LGMD2I patient-derived cells will be harvested.
3. Aspirate growth medium from the flasks of LGMD2I patient-derived cells.
4. Rinse cells in each 75 cm2 flask with 10 mL of PBS twice.
5. Add 1.5 mL of trypsin to each 75 cm2 flask. Rock the flask to evenly distribute the trypsin.
6. Incubate the cells at room temperature for ≤5 minutes. Tap the side of the flask to release the cells.
7. Add 9.5 mL of growth medium to neutralize the trypsin, rinsing the growth surface of the flask during addition.
8. Transfer the cell suspension to a sterile 15 mL centrifuge tube. If harvesting multiple flasks of cells, pool the cell suspension in a 50 mL centrifuge tube.
9. Centrifuge the cell suspension for 5 minutes at 1200 rpm at room temperature to pellet the cells.
10. Aspirate the supernatant and resuspend the cell pellet in 1 mL of growth medium using a p1000 pipette. Pipette up and down 5-20 times to break up clumps of cells.
11. Pipet 10 ul of the cell suspension to a 1.5 mL Eppendorf and add 10 ul of Trypan blue and incubate for 2 min at room temperature.
12. Then pipet 10 ul of the suspension and place in a disposable slide and count the cell viability number using Countess II Automated Cell Counters within 3 min from the end of the incubation
13. Plate 10000 cell per well into 96-well plate with final volume of 150 ul/well.
14. Incubate the assay plate for 22±2 hours in a 37° C./5% CO2 incubator.

Procedure Vector Preparation and Transduction

Determine the amount of AAV9-syn-coFKRP stock that is needed. Remove the reference standard stock from storage at ≤−80° C. and thaw at room temperature. Equilibrate the thawed stock to room temperature.

Prepare low serum media (5%), thaw muscle cell supplement kit and add the smooth muscle cell supplement kit to the graduated filter of a sterile, 0.22 m PES filter system. Fill the graduated filter to 500 mL with F-12K medium.

Prepare vector solution using adjusted titer, 4.67E+12 as Table 18 and Table 19.

Note for the Table: all these need to add dilution buffer to the same volume as the highest dose cohort.

TABLE 18
rAAV dilutions for each well in 96-well plate.

| | | | Volume | |
|---|---|---|---|---|
| | scAAV9-syn- | Volume of | Assay | |
| | coFKRP | scAAV9-syn- | Medium | Final Volume |
| Point | MOI | coFKRP (μL) | (μL) | per-well (uL) |
| 1 | 1.0E+06 | 13.4 | 136.6 | 150 |
| 2 | 6.7E+05 | 8.9 | 141.1 | 150 |
| 3 | 5.0E+05 | 6.6 | 143.4 | 150 |
| 4 | 3.3E+05 | 4.4 | 145.6 | 150 |
| 5 | 2.2E+05 | 2.93 | 147.03 | 150 |
| 6 | 1.50E+05 | 1.95 | 148.05 | 150 |

TABLE 19
rAAV dilutions for each well in 96-well plate.

| | | | Volume | |
|---|---|---|---|---|
| | scAAV9-syn- | Volume of | Assay | |
| | coFKRP | scAAV9-syn- | Medium | Final Volume |
| Point | MOI | COFKRP (μL) | (μL) | per-well (uL) |
| 1 | 7.6E+06 | 102 | 48 | 150 |
| 2 | 5.0E+06 | 68 | 82 | 150 |
| 3 | 3.4E+06 | 45.2 | 104.8 | 150 |
| 4 | 2.3E+06 | 30.2 | 119.8 | 150 |
| 5 | 1.5E+06 | 20.1 | 129.9 | 150 |
| 6 | 1.0E+06 | 13.4 | 136.6 | 150 |
| 7 | 6.7E+05 | 8.9 | 141.1 | 150 |
| 8 | 4.4E+05 | 5.9 | 144.1 | 150 |
| 9 | 3.0E+05 | 3.9 | 146.1 | 150 |
| 10 | 2.0E+05 | 2.6 | 147.4 | 150 |
| 11 | 1.3E+05 | 1.73 | 148.27 | 150 |

It is noted that the Research Grade vector titer was over-estimated (˜10-fold), the original titer, 4.67E+13 has no dose response detected.

LGMD2I Patient-Derived Cell Lysate for Determination of FKRP Activity

Preparation of Harvested Cell Lysate

After 48-hour and 72-hour post transduction, remove 150 μL/well low serum media and replace it with 150 μL/well PBS. Repeat removal of media and addition of PBS another three times. After the final wash, remove all of the liquid, being careful not to disturb the cell layer. Add 50 μL RIPA buffer with protease inhibitor to each well. Incubate at room temperature for 5 minutes. Seal the plate and freeze at ≤−80° C. Seal the plate and freeze at ≤−80° C. When ready to test the samples, thaw the samples. Carefully move lysates to a ddPCR Plate and centrifuge at 4.7 k RPM for 20 minutes at 2-8° C. Keep the lysates on ice.

FKRP Assay for Cell Lysate

Prepare the reagents included in the EZ Standard Pack (Protein Simple) by completing the following:
15. Add 40 μL of Deionized (DI) Water to the Dithiothreitol (DTT) to make a 400 mM concentration solution.
16. Add 20 μL of 10× Sample Buffer and 20 μL of 400 mM DTT Solution to the Fluorescent 5× Master Mix to make a 1× Solution.
17. Add 20 μL of DI Water to the Biotinylated Ladder. Vortex reagents to mix and maintain on ice until use.
18. Prepare the lysates by combining 1.5 μL of the Fluorescent Master Mix and 6 μL of the lysate. Combine in a clean set of tubes and vortex the samples to mix.
19. Denature the samples by placing in a heat block set at 95° C. for five minutes.
20. Vortex the samples again. Then, briefly centrifuge to collect the sample at the bottom of the tube and retain on ice until ready to use.
21. Prepare the anti-FKRP (PA5-65349, Invitrogen; 1:250) and anti-GAPDH (NB100-56875, Novus Bio; 1:5000) primary antibodies by diluted with Antibody Diluent 2.
22. Prepare Substrate by mixing 100 μL of Luminol-S and 100 μL of Peroxide.
23. According to the provided plate map, pipette 5 μL of the Biotin Ladder, 5 μL of the prepared samples, 20 μL of the Antibody Diluent 2, 15 μL of each of the primary and secondary antibodies, 15 μL of the Luminol-peroxide substrate into the 384 well plate.
24. Pipette 15 μL of the Stacking Matrix first, then 30 μL of DI water, and lastly, 15 μL of the Separation Matrix.
25. Centrifuge the plate for five minutes at 2500 rpm (˜1000×g) at room temperature.
26. Open the desired assay template the Protein Simple® Compass Western software.
27. Press start and follow the remaining instructions to ready the machine for the assay run.
28. Save the run when it is complete.

Run Analysis-After the run is completed, the data can be analyzed through the Protein Simple® Compass for Simple Western program. Fluorescent Sizing Standards.

Results

Cell Viability Assay

Extra set of transduced cells in same condition and MOI are prepared for cell viability assay. Cells are gently removed from the culture plates with trypsin. Aliquots of cell suspension being tested for viability were centrifuged for 5 min at 1000×g. The pellets were re-suspended in 200 uL of PBS. Mix 10 uL of cell suspension with 10 uL of trypan blue and incubated 2 min at room temperature. Then 10 ul of the suspension was placed in a disposable slide and cells were counted using Countess II Automated Cell Counters (ThermoFisher Scientific) within 3 min from the end of the incubation.

Cell survival/cell viability is unaffected by transduction at 48-hour and 72-hour incubation regardless of the MOI.

FKRP Activity in Cell Lysate

FKRP activity in the cell lysate is increased with increased in 48-hour and 72 hour and MOI. For the cell lysate, FKRP activity is increased with MOI when normalized to protein and reduced per vector genome when MOI increases.

Data from 72-hour post transduction shows better dose response then 48-hour post transduction, therefore the MOI levels are next performed to further check the cells response to AAV9-syn-coFKRP in higher MOI. Vector preparation and dilution between 1.3E+05 to 7.6E+06 according to Table 19.

Cell survival/cell viability was unaffected by transduction at 72-hour incubation regardless of the MOI.

Discussion

An in vitro potency assay is developed for a therapeutic AAV9-syn-coFKRP in LGMD2I patient-derived cells. The assay is reproducible and linear over the range of 4.4E5 to 7.5E6 vector genomes per assay and is useful for assessing the relative potency of multiple independently synthesized vector lots. The assay is therefore suitable for release testing and for assessing stability of vector lots overtime.

The results support the following conclusions: (1) The greatest production of FKRP in lysate occurs at 72 hours; (2) The normalized FKRP shows activity over the range of 4.4E5 to 7.5E6 MOI; and (3) Cell survival is unaffected by transduction with the vector.

These results indicate the validation and use of this assay for determining the activity of drug product, scAAV9-syn-coFKRP, for clinical dosing as a replacement to the current in vivo assay.

It is expected that in vitro potency assay as described herein in will be effective for iPSC stem cell line to be differentiated into cardiac or, skeletal muscle cell line, or, FKRP knock down cell line or, FKRP knock out cell line. The assay will be used in any one or more of the cell lines as described in Example 8 and example 9 and will serve as a platform for determining the activity of the therapeutic product scAAV9-syn100-coFKRP, and for clinical dosing as a replacement to the current in vivo assay. This assay can work as a bridging assay if there is a change in vectors, or change in promoter, or change in the transgene (e.g., codon optimization of the transgene), or change in any component of the rAAV comprising expression cassette, and will validate the potency of the altered therapeutic product as compared to the parent one or, to a reference.

### Example 9—Close Ended Linear DNA Sequence for the LGMD2i Construct

(SEQ ID NO: 406)

| | |
|---|---|
| 1 | cagctggcgt aatagcgaag aggcccgcac |
| | cgatcgccct tcccaacagt tgcgcagcct |
| 61 | gaatggcgaa tggcgattcc gttgcaatgg |
| | ctggcggtaa tattgttctg gatattacca |
| 121 | gcaaggccga tagtttgagt tcttctactc |
| | aggcaagtga tcttattact aatcaaagaa |
| 181 | gtattgcgac aacggttaat ttgcgtgatg |
| | gacagactct tttactcggt ggcctcactg |
| 241 | attataaaaa cacttctcag gattctggcg |
| | taccgttcct gtctaaaatc cctttaatcg |
| 301 | gcctcctgtt tagctcccgc tctgattcta |
| | acgaggaaag cacgttatac gtgctcgtca |
| 361 | aagcaaccat agtacgcgcc ctgtagcggc |
| | gcattaagcg cggcgggtgt ggtggttacg |
| 421 | cgcagcgtga ccgctacact tcccagcgcc |
| | ctagcgcccg ctcctttcgc tttcttccct |
| 481 | tcctttctcg ccacgttcgc cggctttccc |
| | cgtcaagctc taaatcgggg gctcccttta |
| 541 | gggttccgat ttagtgcttt acggcacctc |
| | gaccccaaaa aacttgatta gggtgatggt |
| 601 | tcacgtagtg ggccatcgcc ctgatagacg |
| | gtttttcgcc ctttgacgtt ggagtccacg |
| 661 | ttctttaata gtggactctt gttccaaact |
| | ggaacaacac tcaaccctat ctcggtctat |
| 721 | tcttttgatt tataagggat tttgccgatt |
| | tcggcctatt gcttaaaaaa tgagctgatt |
| 781 | taacaaaaat ttaacgcgaa ttttaacaaa |
| | atattaacgc ttacaattta aatatttgct |
| 841 | tatacaatct tcctgttttt ggggcttttc |
| | tgattatcaa ccggggtaca tatgattgac |
| 901 | atgctagttt tacgattacc gttcatcgtc |
| | tagagctagc atatggatcc atcgatttag |
| 961 | ggataacagg gtaattatca gcacacaatt |
| | gcccattata cgcgcgtata atggactatt |
| 1021 | gtgtgctgat atctgtacac ttaagggcta |
| | gatcttagct tacgtcacta gagggtccac |
| 1081 | gtttagtttt taagatccat tgatctccta |
| | aacgctgcaa gattcgcaac ctggtatact |
| 1141 | tagcctaggc gctaggtcct agtgcagcgg |
| | gacttttttt ctaaagtcgt tgagaggagg |
| 1201 | agtcgtcaga ccagatagct ttgatgtcct |
| | gatcggaagg atcgttggcc cccctgcagg |
| 1261 | cagctgttaa ttaaccgatt cattaatgca |
| | gcagctgcgc gctcgctcgc tcactgaggc |
| 1321 | cgcccgggca aagcccgggc gtcgggcgac |
| | ctttggtcgc ccggcctcag tgagcgagcg |
| 1381 | agcgcgcaga gagggagtgg cgcgccacgc |
| | gtcggccgtc cgccctcggc accattcctc |
| 1441 | acgacaccga aatatggcga cgggtgagga |
| | atggtgggga gttattttta gagcggtgag |
| 1501 | gaatggtggg caggcagcag gtgttggggg |
| | agttattttt agagcgggga gttattttta |
| 1561 | gagcggtgag gaatggtgga caccgaaata |
| | tggcgacggg tgaggaatgg tgccgtcgcc |
| 1621 | atatttgggt gtcccgtccg ccctcggccg |
| | gggccgcatt cctgggggcc gggcggtgct |
| 1681 | cccgcccgcc tcgataaaag gctccggggc |
| | cggcggcggc ccacgagcta cccggaggag |
| 1741 | cgggaggcgt ctctgccagc ggtccgacgc |
| | gcagtcagca ccaggtaggt gggcaccgcg |
| 1801 | ccgtgccgtg ccactagtat ctaggtgagt |
| | atctcaggga tccagacatg gggatatggg |
| 1861 | aggtgcctct gatcccaggg ctcactgtgg |
| | gtctctctgt tcacagagac cgcgggccac |
| 1921 | catgagactg acaagatgtc aagctgccct |
| | ggctgctgcc atcacactga atctgctggt |
| 1981 | gctgttctat gtgtcctggc tgcagcatca |
| | gcccagaaac tccagagcca gaggacccag |
| 2041 | aagggcctct gctgctggac ctagagtgac |
| | agtgcttgtc agagagtttg aggcctttga |
| 2101 | caatgctgtg cctgagctgg tggacagctt |
| | cctgcagcaa gatcctgctc agcctgtggt |
| 2161 | ggtggctgct gatacactgc cttatcctcc |
| | actggctctg cctagaatcc ccaatgttag |
| 2221 | actggccctc ctgcagcctg ctctggatag |
| | acctgctgct gcttccagac ctgagacata |
| 2281 | tgtggccact gagtttgtgg ccctggtgcc |
| | tgatggtgcc agagctgaag ctcctggcct |
| 2341 | gctggaaaga atggttgagg ccctgagagc |
| | tggatctgcc agactggttg ctgctcctgt |
| 2401 | ggctacagcc aatcctgcca gatgtctggc |
| | cctgaatgtg tccctgagag aatggacagc |
| 2461 | cagatatggt gctgccccag ctgctcctag |
| | atgtgatgct cttgatgggg atgctgtggt |
| 2521 | cctgctgaga gccagggatc tcttcaatct |
| | gtctgcccca ctggccagac ctgtgggcac |
| 2581 | atctctgttt ctgcagacag ctctgagagg |
| | ctgggctgtg cagctgctgg atctcacctt |
| 2641 | tgctgctgca agacagcctc ctctggccac |
| | agctcatgcc agatggaagg ctgagagaga |
| 2701 | gggcagagct agaagggctg ctctgctcag |
| | agcactgggc atcagactgg tgtcttggga |
| 2761 | aggtggcaga cttgagtggt ttggctgcaa |
| | caaagaaacc accagatgct ttggcacagt |
| 2821 | tgtgggagac acccctgcct acctgtatga |
| | ggaaagatgg accccacctt gctgtctgag |
| 2881 | agccctgagg gaaacagcta gatatgttgt |
| | tggagtgctt gaggctgctg gtgtcagata |
| 2941 | ctggctggaa ggtggaagtc tgctgggagc |
| | tgctaggcat ggggacatca tcccttggga |
| 3001 | ctatgatgtg gacctgggca tctacctgga |
| | agatgtgggc aattgtgaac agctgagagg |
| 3061 | ggctgaagct ggctctgttg tggatgagag |
| | aggctttgtc tgggagaaag ctgttgaggg |
| 3121 | agacttcttc agagtgcagt actctgagag |
| | caaccacctc catgtggatc tgtggccatt |
| 3181 | ctaccccaga aatggggtca tgacaaagga |
| | cacctggctg gaccacagac aggatgtgga |
| 3241 | attccctgag cactttctgc agcctctggt |
| | gccactgcct tttgctggat ttgtggctca |
| 3301 | ggcccctaac aactacagaa gattcctgga |
| | actgaagttt ggccctgggg tcatagagaa |
| 3361 | ccctcagtac cctaatcctg cactgctgag |
| | cctgactgga tctggctgat gagtcgacag |
| 3421 | gcctaataaa gagctcagat gcatcgatca |
| | gagtgtgttg gttttttgtg tggtttaaac |
| 3481 | gcggccgcag gaacccctag tgatggagtt |
| | ggccactccc tctctgcgcg ctcgctcgct |
| 3541 | cactgaggcc gggcgaccaa aggtcgcccg |
| | acgcccgggc tttgcccggg cggcctcagt |
| 3601 | gagcgagcga gcgcgcagag agggagtggc |
| | caactggcgt aatagcgaag aggttaatta |
| 3661 | aggcgcccta ggccgaccct tagactctgt |
| | actcagttct ataaacgagc cattggatac |
| 3721 | gagatccgta gattgataag ggacacggaa |
| | tatccccgga cgcaatagac accggtggac |
| 3781 | agcttggtat cctgagcaca gtcgcgcgtc |
| | cgaatctagc tctactttag aggccccgga |
| 3841 | ttctgatggt cgtagaccgc agaaccgatt |
| | ggggggatga gatctactag ttatcagcac |
| 3901 | acaattgccc attatacgcg cgtataatgg |
| | actattgtgt gctgatatag ggataacagg |
| 3961 | gtaattctag agctagcata tcgatccatc |
| | gatttattct cttgtttgct ccagactctc |
| 4021 | aggcaatgac ctgatagcct ttgtagagac |
| | ctctcaaaaa tagctaccct ctccggcatg |
| 4081 | aatttatcag ctagaacggt tgaatatcat |
| | attgatggtg atttgactgt ctccggcctt |
| 4141 | tctcacccgt ttgaatcttt acctacacat |
| | tactcaggca ttgcatttaa aatatatgag |
| 4201 | ggttctaaaa atttttatcc ttgcgttgaa |
| | ataaaggctt ctcccgcaaa agtattacag |
| 4261 | ggtcataatg tttttggtac aaccgattta |
| | gctttatgct ctgaggcttt attgcttaat |
| 4321 | tttgctaatt ctttgccttg cctgtatgat |
| | ttattggatg ttggaatcgc ctgatgcggt |
| 4381 | attttctcct tacgcatctg tgcggtattt |
| | cacaccgcat atggtgcact ctcagtacaa |
| 4441 | tctgctctga tgccgcatag ttaagccagc |
| | cccgacaccc gccaacaccc gctgacgcgc |
| 4501 | cctgacgggc ttgtctgctc ccggcatccg |
| | cttacagaca agctgtgacc gtctccggga |
| 4561 | gctgcatgtg tcagaggttt tcaccgtcat |
| | caccgaaacg cgcgagacga aagggcctcg |
| 4621 | tgatacgcct atttttatag gttaatgtca |
| | tgataataat ggtttcttag acgtcagggt |
| 4681 | accgggcccc ccctcgaggt cgacggtatc |
| | gataagcttg atatcgaatt cctcggggaa |
| 4741 | atgtgcgcgg aacccctatt tctttatttt |
| | tctaaataca ttcaaatatg tatccgctca |
| 4801 | tgagacaata accctgataa atgcttcaat |
| | aatattgaaa aaggaagagt atgagccata |
| 4861 | ttcaacggga aacgtcgagg ccgcgattaa |
| | attccaacat ggatgctgat ttatatgggt |
| 4921 | ataaatgggc tcgcgataat gtcgggcaat |
| | caggtgcgac aatctatcgc ttgtatggga |
| 4981 | agcccgatgc gccagagtt tttctgaaac |
| | atggcaaagg tagcgttgcc aatgatgtta |
| 5041 | cagatgagat ggtcagacta aactggctga |
| | cggaatttat gcctcttccg accatcaagc |
| 5101 | attttatccg tactcctgat gatgcatggt |
| | tactcaccac tgcgatcccc ggaaaaacag |
| 5161 | cattccaggt attagaagaa tatcctgatt |
| | caggtgaaaa tattgttgat gcgctggcag |
| 5221 | tgttcctgcg ccggttgcat tcgattcctg |
| | tttgtaattg tccttttaac agcgatcgcg |
| 5281 | tatttcgtct cgctcaggcg caatcacgaa |
| | tgaataacgg tttggttgat gcgagtgatt |
| 5341 | ttgatgacga gcgtaatggc tggcctgttg |
| | aacaagtctg gaaagaaatg cataaacttt |
| 5401 | tgccattctc accggattca gtcgtcactc |
| | atggtgattt ctcacttgat aaccttattt |
| 5461 | ttgacgaggg gaaattaata ggttgtattg |
| | atgttggacg agtcggaatc gcagaccgat |
| 5521 | accaggatct tcccatccta tggaactgcc |
| | tcggtgagtt ttctccttca ttacagaaac |
| 5581 | ggctttttca aaaatatggt attgataatc |
| | ctgatatgaa taaattgcag tttcatttga |
| 5641 | tgctcgatga gtttttctaa gcgtataatg |
| | gtctagagct agcatatgga tccatcgatt |
| 5701 | ccattatacg cctgtcagac caagtttact |
| | catatatact ttagattgat ttaaaacttc |
| 5761 | atttttaatt taaaaggatc taggtgaaga |
| | tcctttttga taatctcatg accaaaatcc |
| 5821 | cttaacgtga gttttcgttc cactgagcgt |
| | cagaccccgt agaaaagatc aaaggatctt |
| 5881 | cttgagatcc tttttttctg cgcgtaatct |
| | gctgcttgca aacaaaaaaa ccaccgctac |
| 5941 | cagcggtggt ttgtttgccg gatcaagagc |
| | taccaactct ttttccgaag gtaactggct |
| 6001 | tcagcagagc gcagatacca aatactgttc |
| | ttctagtgta gccgtagtta ggccaccact |
| 6061 | tcaagaactc tgtagcaccg cctacatacc |
| | tcgctctgct aatcctgtta ccagtggctg |
| 6121 | ctgccagtgg cgataagtcg tgtcttaccg |
| | ggttggactc aagacgatag ttaccggata |
| 6181 | aggcgcagcg gtcgggctga acggggggtt |
| | cgtgcacaca gcccagcttg gagcgaacga |
| 6241 | cctacaccga actgagatac ctacagcgtg |
| | agctatgaga aagcgccacg cttcccgaag |
| 6301 | ggagaaaggc ggacaggtat ccggtaagcg |
| | gcagggtcgg aacaggagag cgcacgaggg |
| 6361 | agcttccagg gggaaacgcc tggtatcttt |
| | atagtcctgt cgggtttcgc cacctctgac |
| 6421 | ttgagcgtcg atttttgtga tgctcgtcag |
| | gggggcggag cctatggaaa aacgccagca |
| 6481 | acgcggcctt tttacggttc ctggcctttt |
| | tttcctgcag cccgggggat ccaagttcta |
| 6541 | gagcccgcca ccgcggtgga gctcgctcac |
| | atgttctttc ctgcgttatc ccctgattct |
| 6601 | gtggataacc gtattaccgc ctttgagtga |
| | gctgataccg ctcgccgcag ccgaacgacc |
| 6661 | gagcgcagcg agtcagtgag cgaggaagcg |
| | gaagagcgcc caatacgcaa accgcctctc |
| 6721 | cccgcgcgtt ggccgattca ttaatg |

SEQ ID NO: 406 is a close ended linear DNA sequence for the LGMD2i construct, including the backbone sequence. A nucleic acid sequence as set forth in SEQ ID NO: 406 is used to manufacture rAAV that lacks bacterial sequence.

Base pairs 1922-3412 of SEQ ID NO: 406 (SEQ ID NO: 407) is the sequence of a CpG depleted-FKRP coding sequence. The FKRP expression from FKRP coding sequence as set forth in SEQ ID NO:407, or, SEQ ID NO:2 can be driven by different muscle promoters including synthetic and synthetic short ones e.g., promoters and/or, cis regulatory elements selected from the tables 1-4, or, 8-12, or, any combination thereof. The FKRP expression from FKRP coding sequence as set forth in SEQ ID NO:407, or, SEQ ID NO:2 can be driven by different muscle promoters e.g., Syn100.

(SEQ ID NO: 407)

| | |
|---|---|
| | atgagactg acaagatgtc aagctgccct |
| | ggctgctgcc atcacactga atctgctggt |
| 1981 | gctgttctat gtgtcctggc tgcagcatca |
| | gcccagaaac tccagagcca gaggacccag |
| 2041 | aagggcctct gctgctggac ctagagtgac |
| | agtgcttgtc agagagtttg aggcctttga |
| 2101 | caatgctgtg cctgagctgg tggacagctt |
| | cctgcagcaa gatcctgctc agcctgtggt |
| 2161 | ggtggctgct gatacactgc cttatcctcc |
| | actggctctg cctagaatcc ccaatgttag |
| 2221 | actggccctc ctgcagcctg ctctggatag |
| | acctgctgct gcttccagac ctgagacata |
| 2281 | tgtggccact gagtttgtgg ccctggtgcc |
| | tgatggtgcc agagctgaag ctcctggcct |
| 2341 | gctggaaaga atggttgagg ccctgagagc |
| | tggatctgcc agactggttg ctgctcctgt |
| 2401 | ggctacagcc aatcctgcca gatgtctggc |
| | cctgaatgtg tccctgagag aatggacagc |
| 2461 | cagatatggt gctgccccag ctgctcctag |
| | atgtgatgct cttgatgggg atgctgtggt |
| 2521 | cctgctgaga gccagggatc tgttcaatct |
| | gtctgcccca ctggccagac ctgtgggcac |
| 2581 | atctctgttt ctgcagacag ctctgagagg |
| | ctgggctgtg cagctgctgg atctcacctt |
| 2641 | tgctgctgca agacagcctc ctctggccac |
| | agctcatgcc agatggaagg ctgagagaga |
| 2701 | gggcagagct agaagggctg ctctgctcag |
| | agcactgggc atcagactgg tgtcttggga |
| 2761 | aggtggcaga cttgagtggt ttggctgcaa |
| | caaagaaacc accagatgct ttggcacagt |
| 2821 | tgtgggagac acccctgcct acctgtatga |
| | ggaaagatgg accccacctt gctgtctgag |
| 2881 | agccctgagg gaaacagcta gatatgttgt |
| | tggagtgctt gaggctgctg gtgtcagata |
| 2941 | ctggctggaa ggtggaagtc tgctgggagc |
| | tgctaggcat ggggacatca tcccttggga |
| 3001 | ctatgatgtg gacctgggca tctacctgga |
| | agatgtgggc aattgtgaac agctgagagg |
| 3061 | ggctgaagct ggctctgttg tggatgagag |
| | aggctttgtc tgggagaaag ctgttgaggg |
| 3121 | agacttcttc agagtgcagt actctgagag |
| | caaccacctc catgtggatc tgtggccatt |
| 3181 | ctaccccaga aatggggtca tgacaaagga |
| | cacctggctg gaccacagac aggatgtgga |
| 3241 | attccctgag cactttctgc agcctctggt |
| | gccactgcct tttgctggat ttgtggctca |
| 3301 | ggcccctaac aactacagaa gattcctgga |
| | actgaagttt ggccctgggg tcatagagaa |
| 3361 | ccctcagtac cctaatcctg cactgctgag |
| | cctgactgga tctggctgat ga |

Base pairs 1295-3633 of SEQ ID NO: 406 (SEQ ID NO: 408) is the sequence of an rAAV comprising left ITR (LITRm-self complementary) to right ITR sequence is. In some embodiments, the rAAV comprising SEQ Id NO: 408 comprises Syn100 promoter as set forth in SEQ ID NO:3, wherein the Syn100 promoter of SEQ ID NO: 408 is replaced by any of the synthetic muscle promoters and/or, cis regulatory elements selected from the tables 1-4, or, 8-12, or, any fragment thereof, or, any combination thereof.

(SEQ ID NO: 408)

| | |
|---|---|
| | cctggc tgcagcatca |
| | gcccagaaac tccagagcca gaggacccag |
| 2041 | aagggcctct gctgctggac ctagagtgac |
| | agtgcttgtc agagagtttg aggcctttga |
| 2101 | caatgctgtg cctgagctgg tggacagctt |
| | cctgcagcaa gatcctgctc agcctgtggt |
| 2161 | ggtggctgct gatacactgc cttatcctcc |
| | actggctctg cctagaatcc ccaatgttag |
| 2221 | actggccctc ctgcagcctg ctctggatag |
| | acctgctgct gcttccagac ctgagacata |
| 2281 | tgtggccact gagtttgtgg ccctggtgcc |
| | tgatggtgcc agagctgaag ctcctggcct |
| 2341 | gctggaaaga atggttgagg ccctgagagc |
| | tggatctgcc agactggttg ctgctcctgt |
| 2401 | ggctacagcc aatcctgcca gatgtctggc |
| | cctgaatgtg tccctgagag aatggacagc |
| 2461 | cagatatggt gctgccccag ctgctcctag |
| | atgtgatgct cttgatgggg atgctgtggt |
| 2521 | cctgctgaga gccagggatc tgttcaatct |
| | gtctgcccca ctggccagac ctgtgggcac |
| 2581 | atctctgttt ctgcagacag ctctgagagg |
| | ctgggctgtg cagctgctgg atctcacctt |
| 2641 | tgctgctgca agacagcctc ctctggccac |
| | agctcatgcc agatggaagg ctgagagaga |
| 2701 | gggcagagct agaagggctg ctctgctcag |
| | agcactgggc atcagactgg tgtcttggga |
| 2761 | aggtggcaga cttgagtggt ttggctgcaa |
| | caaagaaacc accagatgct ttggcacagt |
| 2821 | tgtgggagac acccctgcct acctgtatga |
| | ggaaagatgg accccacctt gctgtctgag |
| 2881 | agccctgagg gaaacagcta gatatgttgt |
| | tggagtgctt gaggctgctg gtgtcagata |
| 2941 | ctggctggaa ggtggaagtc tgctgggagc |
| | tgctaggcat ggggacatca tcccttggga |
| 3001 | ctatgatgtg gacctgggca tctacctgga |
| | agatgtgggc aattgtgaac agctgagagg |
| 3061 | ggctgaagct ggctctgttg tggatgagag |
| | aggctttgtc tgggagaaag ctgttgaggg |
| 3121 | agacttcttc agagtgcagt actctgagag |
| | caaccacctc catgtggatc tgtggccatt |
| 3181 | ctaccccaga aatggggtca tgacaaagga |
| | cacctggctg gaccacagac aggatgtgga |
| 3241 | attccctgag cactttctgc agcctctggt |
| | gccactgcct tttgctggat ttgtggctca |
| 3301 | ggcccctaac aactacagaa gattcctgga |
| | actgaagttt ggccctgggg tcatagagaa |
| 3361 | ccctcagtac cctaatcctg cactgctgag |
| | cctgactgga tctggctgat gagtcgacag |
| 3421 | gcctaataaa gagctcagat gcatcgatca |
| | gagtgtgttg gttttttgtg tggtttaaac |
| 3481 | gcggccgcag gaacccctag tgatggagtt |
| | ggccactccc tctctgcgcg ctcgctcgct |
| 3541 | cactgaggcc gggcgaccaa aggtcgcccg |
| | acgcccgggc tttgcccggg cggcctcagt |
| 3601 | gagcgagcga gcgcgcagag agggagtggc |
| | caa |

1. A recombinant adenovirus associated (AAV) vector comprising in its genome in the 5′ to 3′ direction:
a) a 5′ AAV inverted terminal repeat (ITR);
b) a muscle specific promoter;
c) an intron sequence;
d) a nucleic acid encoding human fukutin-related protein (FKRP) which has a nucleotide sequence shown in SEQ ID NO: 2, or, SEQ ID NO: 407, and is operatively linked to the muscle specific promoter;
e) a polyA signal sequence operatively linked to the nucleic acid encoding FKRP;
f) a 3′ AAV ITR.

2. The recombinant AAV vector of claim 1, wherein the 5′ITR is ITR2m.

3. The recombinant AAV vector of any one of claims 1-2, wherein the 3′ITR is ITR2.

4. The recombinant AAV vector of any one of claims 1-3, wherein the muscle-specific promoter is Syn100 (SEQ ID NO: 3).

5. The recombinant AAV vector of any one of claims 1-4, wherein the intron sequence is VH4-Ig-Intron 3 (SEQ ID NO: 4) or a derivative thereof.

6. The recombinant AAV vector of any one of claims 1-5, wherein the polyA signal sequence is SEQ ID NO: 5.

7. The recombinant AAV vector of any one of claims 1-6, wherein the muscle specific promoter, intron sequence, nucleic acid encoding FKRP, and polyA signal sequence are comprised within SEQ ID NO: 1.

8. The recombinant AAV vector of any one of claims 1-7, wherein the serotype is AAV9.

9. A pharmaceutical composition comprising the recombinant AAV vector of any one of claims 1-8.

10. A method to treat a subject with a dystroglycanopathy disorder comprising systemically administering a therapeutically effective amount of the recombinant AAV vector of any one of claims 1-8, and/or the pharmaceutical composition of claim 9, to the subject, to thereby increase expression of functional FKRP in muscle tissue of the subject.

11. The method of claim 10, wherein the dystroglycanopathy disorder is limb-girdle muscular dystrophy 2I.

12. The method of claims 10-11, wherein a single dose is administered to the subject.

13. The method of claims 10-12, wherein administration is by intravenous infusion.

14. The method of any one of claims 10-13, wherein the dose administered is from about 1E13 vg/kg to about 6E13 vg/kg (e.g. about 3E13 vg/kg).

15. The method of claims 10-14, wherein one or more of the following occur in the subject following administration:
a) functional glycosylation of α-DG is substantially increased in skeletal muscle and/or cardiac muscle of the subject;
b) serum creatine kinase levels of the subject are substantially reduced;
c) collagen deposition in skeletal muscle of the subject is substantially reduced;
d) in vitro muscle force analysis of the subject's muscle tissue (e.g., soleus, diaphragm and/or EDL) is significantly increased;
e) tidal volume of the subject is substantially increased; and/or
f) the subject can run significantly further in a treadmill test.

16. The method of claims 10-15, wherein the subject is an adult.

17. A synthetic nucleic acid encoding human fukutin-related protein (FKRP), wherein:
a) the nucleic acid has reduced CpG site content relative to the CpG site content of SEQ ID NO: 6;
b) the GC content is reduced by greater than 10% relative to the GC content of SEQ ID NO:6; and/or
c) the nucleic acid has at least 80% identity to SEQ ID NO: 2.

18. The nucleic acid of claim 17, wherein the coding sequence has at least 50% reduced CpG site content relative to the CpG site content of SEQ ID NO: 6.

19. The nucleic acid of claims 17-18, wherein the coding sequence has at least 75%, 80%, 85%, 90%, 95% reduced CpG site content relative to the CpG site content of SEQ ID NO: 6.

20. The nucleic acid of claims 17-19, wherein the coding sequence has 0% CpG site content.

21. The synthetic nucleic acid of claim 17, wherein the GC content is reduced by greater than 15% relative to the GC content of SEQ ID NO:6.

22. The synthetic nucleic acid of claim 17, wherein the nucleic acid has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 2.

23. The synthetic nucleic acid of claim 17, wherein the nucleic acid has a sequence shown in SEQ ID NO: 2, or, SEQ ID NO: 407.

24. The synthetic nucleic acid of claims 17-23 that is operably linked to a promoter.

25. The synthetic nucleic acid of claim 24, wherein the promoter is a muscle-specific promoter.

26. The synthetic nucleic acid of any one of claims 24-25, wherein the promoter is a synthetic promoter.

27. The synthetic nucleic acid of any one of claim 24-26, wherein the promoter is Syn100.

28. The synthetic nucleic acid of any one of claims 23-26, wherein the promoter is selected from promoters listed in Tables 1-4, or, from Tables 8-12.

29. The synthetic nucleic acid of any one of claims 24-25, wherein the promoter is a creatine kinase (CK) promoter, a chicken R-actin promoter (CB).

30. The synthetic nucleic acid of any one of claims 17-29, further comprising an enhancer sequence.

31. The synthetic nucleic acid of claim 30, wherein the enhancer sequence comprises a CMV enhancer, a muscle creatine kinase enhancer, and/or a myosin light chain enhancer.

32. A nucleic acid comprising:
a) 5′ and 3′ AAV inverted terminal repeats (ITR);
b) a coding sequence encoding human fukutin-related protein (FKRP) operatively linked to a muscle-specific promoter located between the 5′ITR and 3′ITR, wherein the coding sequence has:
i) reduced CpG site content relative to the CpG site content of SEQ ID NO: 6;
ii) reduced GC content greater than 10% relative to the GC content of SEQ ID NO:6; and/or
iii) at least 80% identity to SEQ ID NO: 2.

33. The nucleic acid of claim 32, further comprising an intron sequence located between the muscle-specific promoter and the coding sequence.

34. The nucleic acid of claim 33, wherein the intron sequence is VH4-Ig-Intron 3 (SEQ ID NO: 4) or a derivative thereof.

35. The nucleic acid of claims 32-34, further comprising at least one polyA signal sequence located downstream of the coding sequence.

36. The nucleic acid of claim 35, wherein the polyA signal sequence is SEQ ID NO: 5.

37. The nucleic acid of claim 32-36, wherein the 5′ITR is ITR2m.

38. The nucleic acid of claims 32-37, wherein the 3′ITR is ITR2.

39. The nucleic acid of claims 32-38, wherein the GC content of the coding sequence is reduced by greater than 15% relative to the GC content of SEQ ID NO:6.

40. The nucleic acid of claims 32-40, wherein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 2.

41. The nucleic acid of claims 32-40, wherein the coding sequence has at least 50% reduced CpG site content relative to the CpG site content of SEQ ID NO: 6.

42. The nucleic acid of claims 32-41, wherein the coding sequence has at least 75%, 80%, 85%, 90%, 95% reduced CpG site content relative to the CpG site content of SEQ ID NO: 6.

43. The nucleic acid of claims 32-42, wherein the coding sequence has 0% CpG site content.

44. The nucleic acid sequence of claims 32-43, wherein the coding sequence is SEQ ID NO: 2.

45. A vector comprising the synthetic nucleic acid of any one of claims 17 to 44.

46. The vector of claim 45, wherein the vector is a viral vector.

47. The vector of claim 46, wherein the vector is a recombinant adeno-associated virus (AAV) vector.

48. The vector of claim 47, wherein the AAV vector is from any serotype listed in Table 6.

49. The vector of claim 47 or claim 48, wherein the AAV vector is an AAV9 vector.

50. A recombinant adenovirus associated (AAV) vector comprising in its genome:
a) a 5′ AAV inverted terminal repeat (ITR) and a 3′ AAV ITR;
b) located between the 5′ITR and 3′ITR, a nucleic acid encoding human fukutin-related protein (FKRP) which has:
i) reduced CpG site content relative to the CpG site content of SEQ ID NO: 6;
ii) reduced GC content greater than 10% relative to the GC content of SEQ ID NO:6; and/or
iii) at least 80% identity to SEQ ID NO: 2,
and is operatively linked to a muscle-specific promoter.

51. The recombinant AAV vector of claim 50, wherein the AAV genome comprises, in the 5′ to 3′ direction:
a) the 5′ITR,
b) the muscle-specific promoter,
c) an intron sequence,
d) the nucleic acid encoding FKRP; and,
e) the 3′ITR.

52. The recombinant AAV vector of any of claims 50-51, wherein the muscle-specific promoter is selected from the group consisting of MCK promoter, dMCK promoter, tMCK promoter, enh358MCK promoter, CK6 promoter and Syn100 promoter, any promoter listed in Table 1-4 or 8-12, and derivatives thereof.

53. The recombinant AAV vector of any of claims 50-52, wherein the nucleic acid encoding FKRP has reduced CpG site content relative to the CpG site content of SEQ ID NO: 6.

54. The recombinant AAV vector of any of claims 50-53, wherein the nucleic acid encoding FKRP has at least 50% reduced CpG site content relative to the CpG site content of SEQ ID NO: 6.

55. The recombinant AAV vector of any of claims 50-53, wherein the nucleic acid encoding FKRP has at least 75%, 80%, 85%, 90%, 95% reduced CpG site content relative to the CpG site content of SEQ ID NO: 6.

56. The recombinant AAV vector of any of claims 50-55, wherein the nucleic acid encoding FKRP has 0% CpG site content.

57. The recombinant AAV vector of any of claims 50-56, wherein the nucleic acid encoding FKRP has reduced GC content greater than 10% relative to the GC content of SEQ ID NO:6.

58. The recombinant AAV vector of any of claims 50-57, wherein the nucleic acid encoding FKRP has at least 80% identity to SEQ ID NO: 2.

59. The recombinant AAV vector of claims 50-58, wherein the nucleic acid encoding FKRP has a sequence shown in SEQ ID NO: 2.

60. The recombinant AAV vector of any of claims 50-59, further comprising at least one polyA signal sequence located 3′ of the nucleic acid encoding the FKRP polypeptide and 5′ of the 3′ITR sequence.

61. The recombinant AAV vector of claim 60, wherein the polyA signal sequence is SEQ ID NO: 5.

62. The recombinant AAV vector of any of claims 50-61, wherein the ITR comprises an insertion, deletion or substitution.

63. The recombinant AAV vector of claims 50-62, wherein one or more CpG site sites in the ITR are removed.

64. The recombinant AAV vector of any one of claims 50-63, wherein the 5′ITR is ITR2m.

65. The recombinant AAV vector of any one of claims 50-64, wherein the 3′ITR is ITR2.

66. The recombinant AAV vector of any one of claims 50-65, wherein the intron sequence is VH4-Ig-Intron 3 (SEQ ID NO: 4) or a derivative thereof.

67. The recombinant AAV vector of any of claims 50-66, wherein the recombinant AAV vector is a chimeric AAV vector, haploid AAV vector, a hybrid AAV vector or polyploid AAV vector.

68. The recombinant AAV vector of any of claims 50-66, wherein the recombinant AAV vector is any AAV serotype listed in Table 6.

69. The recombinant AAV vector of claim 68 wherein the serotype is AAV9.

70. The recombinant AAV vector of any of claims 50-69, wherein the recombinant AAV vector comprises a capsid protein selected from Table 7 or any AAV serotype in the group consisting of those listed in Table 6, and combinations thereof.

71. A pharmaceutical composition comprising the recombinant AAV vector of any one of claims 50-70 in a pharmaceutically acceptable carrier.

72. A transformed cell comprising the nucleic acid of any one of claims 17-44 and/or the vector of any one of claims 45 to 70.

73. A transgenic animal comprising the nucleic acid of any one of claims 17-44, the vector of any one of claims 45 to 70, and/or the transformed cell of claim 72.

74. A method of increasing glycosylation of α-dystroglycan (α-DG) in a subject in need thereof, comprising: administering to said subject a therapeutically effective amount of the nucleic acid of any one of claims 17-44, the vector of any one of claims 45 to 70, the pharmaceutical composition of claim 71, and/or the transformed cell of claim 72, wherein the synthetic nucleic acid is expressed in said subject, thereby producing human FKRP and increasing glycosylation of α-DG.

75. The method of claim 74, wherein the subject has or is at risk for developing a dystroglycanopathy disorder.

76. A method of treating or a dystroglycanopathy disorder in a subject, comprising administering to the subject a therapeutically effective amount of the nucleic acid of any one of claims 17 to 44, the vector of any one of claims 45-70, the pharmaceutical composition of claim 71, and/or the transformed cell of claim 72, wherein the synthetic nucleic acid is expressed in said subject, thereby treating the dystroglycanopathy disorder in the subject.

77. The method of claim 75 or 76, wherein the dystroglycanopathy disorder is associated with a FKRP anomaly.

78. The method of claims 75-77, wherein the dystroglycanopathy disorder comprises a mutation in the nucleic acid encoding FKRP and/or a deficiency in glycosylation of α-dystroglycan (α-DG).

79. The method of claims 75-78, wherein the dystroglycanopathy disorder is limb-girdle muscular dystrophy 2I, congenital muscular dystrophy (CMD1C), Walker-Warburg syndrome, muscle-eye-brain disease, or any combination thereof.

80. A method to treat a subject with a dystroglycanopathy disorder comprising administering a therapeutically effective amount of any of the recombinant AAV vector, the rAAV genome, the nucleic acid sequence, and/or the pharmaceutical compositions, of any one of the previous claims to the subject, to thereby increase expression of functional FKRP in muscle tissue of the subject.

81. The method of claims 74-80, wherein a single dose is administered to the subject.

82. The method of claims 74-81, wherein administration is systemic.

83. The method of claim 82, wherein administration is by intravenous infusion.

84. The method of claims 74-83, wherein functional glycosylation of α-DG is substantially increased in skeletal muscle and/or cardiac muscle of the subject following administration.

85. The method of claims 74-84, wherein serum creatine kinase levels of the subject are substantially reduced following administration.

86. The method of claims 74-85, wherein collagen deposition in skeletal muscle of the subject is substantially reduced following administration.

87. The method of claims 74-86, wherein the subject is an adult.

88. The method of claims 74-86, wherein the subject is a juvenile.

89. The method of claims 74-86, wherein the subject is an infant.

90. The method of claims 74-89, wherein the subject demonstrates significant disease pathology prior to administration.

91. The method of claims 74-89, wherein the subject demonstrates no significant disease pathology prior to administration.


SEQUENCE LISTING (411 sequences)

SEQ ID NO: 1 - DNA, 2349 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtggaattcacgcgtggtacccggccgtccgccctcggcaccattcctcacgacaccgaaatatggcgacgggtgaggaatggtggggagttatttttagagcggtgaggaatggtgggcaggcagcaggtgttgggggagttatttttagagcggggagttatttttagagcggtgaggaatggtggacaccgaaatatggcgacgggtgaggaatggtgccgtcgccatatttgggtgtcccgtccgccctcggccggggccgcattcctgggggccgggcggtgctcccgcccgcctcgataaaaggctccggggccggcggcggcccacgagctacccggaggagcgggaggcgtctctgccagcggtccgacgcgcagtcagcaccaggtaggtgggcaccgcgccgtgccgtgccactagtatctaggtgagtatctcagggatccagacatggggatatgggaggtgcctctgatcccagggctcactgtgggtctctctgttcacagagaccgcgggccaccatgagactgacaagatgtcaagctgccctggctgctgccatcacactgaatctgctggtgctgttctatgtgtcctggctgcagcatcagcccagaaactctagagccagaggacccagaagggcctctgctgctggacctagagtgacagtgcttgtcagagagtttgaggcctttgacaatgctgtgcctgagctggtggacagcttcctgcagcaagatcctgctcagcctgtggtggtggctgctgatacactgccttatcctccactggctctgcctagaatccccaatgttagactggccctcctgcagcctgctctggatagacctgctgctgcttccagacctgagacatatgtggccactgagtttgtggccctggtgcctgatggtgccagagctgaagctcctggcctgctggaaagaatggttgaggccctgagagctggatctgccagactggttgctgctcctgtggctacagccaatcctgccagatgtctggccctgaatgtgtccctgagagaatggacagccagatatggtgctgccccagctgctcctagatgtgatgctcttgatggggatgctgtggtcctgctgagagccagggatctgttcaatctgtctgccccactggccagacctgtgggcacatctctgtttctgcagacagctctgagaggctgggctgtgcagctgctggatctcacctttgctgctgcaagacagcctcctctggccacagctcatgccagatggaaggctgagagagagggcagagctagaagggctgctctgctcagagcactgggcatcagactggtgtcttgggaaggtggcagacttgagtggtttggctgcaacaaagaaaccaccagatgctttggcacagttgtgggagacacccctgcctacctgtatgaggaaagatggaccccaccttgctgtctgagagccctgagggaaacagctagatatgttgttggagtgcttgaggctgctggtgtcagatactggctggaaggtggaagtctgctgggagctgctaggcatggggacatcatcccttgggactatgatgtggacctgggcatctacctggaagatgtgggcaattgtgaacagctgagaggggctgaagctggctctgttgtggatgagagaggctttgtctgggagaaagctgttgagggagacttcttcagagtgcagtactctgagagcaaccacctccatgtggatctgtggccattctaccccagaaatggggtcatgacaaaggacacctggctggaccacagacaggatgtggaattccctgagcactttctgcagcctctggtgccactgccttttgctggatttgtggctcaggcccctaacaactacagaagattcctggaactgaagtttggccctggggtcatagagaaccctcagtaccctaatcctgcactgctgagcctgactggatctggctgatgagtcgacaggcctaataaagagctcagatgcatcgatcagagtgtgttggttttttgtgtgcttaaggcgggaattgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccc

SEQ ID NO: 2 - DNA, 1491 bp, Artificial Sequence
  Description of Artificial Sequence: Synthetic polynucleotide
atgagactgacaagatgtcaagctgccctggctgctgccatcacactgaatctgctggtgctgttctatgtgtcctggctgcagcatcagcccagaaactctagagccagaggacccagaagggcctctgctgctggacctagagtgacagtgcttgtcagagagtttgaggcctttgacaatgctgtgcctgagctggtggacagcttcctgcagcaagatcctgctcagcctgtggtggtggctgctgatacactgccttatcctccactggctctgcctagaatccccaatgttagactggccctcctgcagcctgctctggatagacctgctgctgcttccagacctgagacatatgtggccactgagtttgtggccctggtgcctgatggtgccagagctgaagctcctggcctgctggaaagaatggttgaggccctgagagctggatctgccagactggttgctgctcctgtggctacagccaatcctgccagatgtctggccctgaatgtgtccctgagagaatggacagccagatatggtgctgccccagctgctcctagatgtgatgctcttgatggggatgctgtggtcctgctgagagccagggatctgttcaatctgtctgccccactggccagacctgtgggcacatctctgtttctgcagacagctctgagaggctgggctgtgcagctgctggatctcacctttgctgctgcaagacagcctcctctggccacagctcatgccagatggaaggctgagagagagggcagagctagaagggctgctctgctcagagcactgggcatcagactggtgtcttgggaaggtggcagacttgagtggtttggctgcaacaaagaaaccaccagatgctttggcacagttgtgggagacacccctgcctacctgtatgaggaaagatggaccccaccttgctgtctgagagccctgagggaaacagctagatatgttgttggagtgcttgaggctgctggtgtcagatactggctggaaggtggaagtctgctgggagctgctaggcatggggacatcatcccttgggactatgatgtggacctgggcatctacctggaagatgtgggcaattgtgaacagctgagaggggctgaagctggctctgttgtggatgagagaggctttgtctgggagaaagctgttgagggagacttcttcagagtgcagtactctgagagcaaccacctccatgtggatctgtggccattctaccccagaaatggggtcatgacaaaggacacctggctggaccacagacaggatgtggaattccctgagcactttctgcagcctctggtgccactgccttttgctggatttgtggctcaggcccctaacaactacagaagattcctggaactgaagtttggccctggggtcatagagaaccctcagtaccctaatcctgcactgctgagcctgactggatctggctgatga

SEQ ID NO: 3 - DNA, 400 bp, Artificial Sequence
  Description of Artificial Sequence: Synthetic polynucleotide
cggccgtccgccctcggcaccattcctcacgacaccgaaatatggcgacgggtgaggaatggtggggagttatttttagagcggtgaggaatggtgggcaggcagcaggtgttgggggagttatttttagagcggggagttatttttagagcggtgaggaatggtggacaccgaaatatggcgacgggtgaggaatggtgccgtcgccatatttgggtgtcccgtccgccctcggccggggccgcattcctgggggccgggcggtgctcccgcccgcctcgataaaaggctccggggccggcggcggcccacgagctacccggaggagcgggaggcgtctctgccagcggtccgacgcgcagtcagcaccaggtaggtgggcaccgcgccgtgccgtgcc

SEQ ID NO: 4 - DNA, 90 bp, Artificial Sequence
  Description of Artificial Sequence: Synthetic oligonucleotide
tctaggtgagtatctcagggatccagacatggggatatgggaggtgcctctgatcccagggctcactgtgggtctctctgttcacagaga

SEQ ID NO: 5 - DNA, 54 bp, Artificial Sequence
  Description of Artificial Sequence: Synthetic oligonucleotide
aggcctaataaagagctcagatgcatcgatcagagtgtgttggttttttgtgtg

SEQ ID NO: 6 - DNA, 1488 bp, Homo sapiens
atgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctga

SEQ ID NO: 7 - DNA, 106 bp, Artificial Sequence
  Description of Artificial Sequence: Synthetic polynucleotide
ctgcgcgctcgctcgctcactgaggccgcccgggcaaagcccgggcgtcgggcgacctttggtcgcccggcctcagtgagcgagcgagcgcgcagagagggagtgg

SEQ ID NO: 8 - DNA, 143 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 9 - DNA, 17 bp, Artificial Sequence
  Description of Artificial Sequence: Synthetic oligonucleotide
aattcacgcgtggtacc

SEQ ID NO: 10 - DNA, 7 bp, Artificial Sequence
  Description of Artificial Sequence: Synthetic oligonucleotide
actagta

SEQ ID NO: 11 - DNA, 12 bp, Artificial Sequence
  Description of Artificial Sequence: Synthetic oligonucleotide
ccgcgggccacc

SEQ ID NO: 12 - DNA, 6 bp, Artificial Sequence
  Description of Artificial Sequence: Synthetic oligonucleotide
gtcgac

SEQ ID NO: 13 - DNA, 21 bp, Artificial Sequence
  Description of Artificial Sequence: Synthetic oligonucleotide
cttaaggcgggaattgatctg

SEQ ID NO: 14-35 - skipped (22 entries)

SEQ ID NO: 36 - DNA, 167 bp, Artificial Sequence
  Description of Artificial Sequence: Synthetic polynucleotide
aggaacccctagtgatggagttggccactccctctctgcgcgctcgctcgctcactgaggcaatttgataaaaatcgtcaaattataaacaggctttgcctgtttagcctcagtgagcgagcgagcgcgcagagagggagtggccaactccatcactaggggttcct

SEQ ID NO: 37 - DNA, 143 bp, Artificial Sequence
  Description of Artificial Sequence: Synthetic polynucleotide
aggaacccctagtgatggagttggccactccctctctgcgcgctcgctcgctcactgagggataaaaatccaggctttgcctgcctcagtgagcgagcgagcgcgcagagagggagtggccaactccatcactaggggttcct

SEQ ID NO: 38 - DNA, 143 bp, Artificial Sequence; identical to SEQ ID NO: 37
  Description of Artificial Sequence: Synthetic polynucleotide

SEQ ID NO: 39 - DNA, 208 bp, Artificial Sequence
  Description of Artificial Sequence: Synthetic polynucleotide
aggaacccctagtgatggagttggccactccctctctgggattgggattgcgcgctcgctcgcgggattgggattgggattgggattgggattgggattgataaaaatcaatcccaatcccaatcccaatcccaatcccaatcccgcgagcgagcgcgcaatcccaatcccagagagggagtggccaactccatcactaggggttcct

SEQ ID NO: 40 - DNA, 211 bp, Artificial Sequence
  Description of Artificial Sequence: Synthetic polynucleotide
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggc

SEQ ID NO: 41 - DNA, 189 bp, Artificial Sequence
  Description of Artificial Sequence: Synthetic polynucleotide
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 42 - DNA, 105 bp, Artificial Sequence
  Description of Artificial Sequence: Synthetic polynucleotide
cggccggggccgcattcctgggggccgggcggtgctcccgcccgcctcgataaaaggctccggggccggcggcggcccacgagctacccggaggagcgggaggcg

SEQ ID NO: 43 - DNA, 229 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccct

SEQ ID NO: 44-54 - skipped (11 entries)

SEQ ID NO: 55 - DNA, 242 bp, Artificial Sequence
  Description of Artificial Sequence: Synthetic polynucleotide
ataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 56 - DNA, 232 bp, Artificial Sequence
  Description of Artificial Sequence: Synthetic polynucleotide
atttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcacc

SEQ ID NO: 57-64 - skipped (8 entries)

SEQ ID NO: 65 - DNA, 284 bp, Artificial Sequence
  Description of Artificial Sequence: Synthetic polynucleotide
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 66-79 - skipped (14 entries)

SEQ ID NO: 80 - DNA, 298 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccacc

SEQ ID NO: 81 - DNA, 354 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtccaccgcctgctgccacggccggccgtataaatagaggcgaggagcagctgggctctcttggcagtcacc

SEQ ID NO: 82 - DNA, 270 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 83 - DNA, 286 bp, Artificial Sequence
  Description of Artificial Sequence: Synthetic polynucleotide
taagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagccaccgcctgctgccacggccggccgtataaatagaggcgaggagcagctgggctctcttggcagtcacc

SEQ ID NO: 84 - DNA, 315 bp, Artificial Sequence
  Description of Artificial Sequence: Synthetic polynucleotide
ctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagccaccgcctgctgccacggccggccgtataaatagaggcgaggagcagctgggctctcttggcagtcacc

SEQ ID NO: 85 - DNA, 421 bp, Artificial Sequence
  Description of Artificial Sequence: Synthetic polynucleotide
ctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 86 - DNA, 524 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 87 - DNA, 601 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 88 - DNA, 531 bp, Artificial Sequence
  Description of Artificial Sequence: Synthetic polynucleotide
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaaactcgggggccaggcactggcgctgacgcaggctagcagggcgccactggctggtccccacccacctcggtgggttgggggatgggcgcaccagcccctcctgggtgagccctagcctggggcttcctatttcgggagccgggggcgtgggccacgtctcctcatgtgatgcgagggctatttaaagcggcagcccgggcagggagccgccgtcggagcccttgcacgcctgctctcttgtagctgcggccgccacc

SEQ ID NO: 89 - DNA, 528 bp, Artificial Sequence
  Description of Artificial Sequence: Synthetic polynucleotide
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaaataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 90 - DNA, 454 bp, Artificial Sequence
  Description of Artificial Sequence: Synthetic polynucleotide
ctgtgtgtttctgtggctgagtcagatggaggagtcctcatgtttcactgcttagcagtttttgtccttcctagtacccgttcccagcccacaagatgcagaaagagctgttgctagcgtgagttatttttgtcagctgagtcaccacgccagaaagcaagaaatgacccgctttatgtctgctctgaggagctggaaccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 91 - DNA, 484 bp, Artificial Sequence
  Description of Artificial Sequence: Synthetic polynucleotide
tacatcatttacctagaaaagaggacagctgtcctttcccaaagctccggtgaccctgccccgcccagtgtgactagcccaggttggtgattctgatctgttgccaaaccaaactggctccccggggagccatttggtaatgttccctggagtcatttccttgcgaagcattccttttcggtgagaggacatttttttcatccctgataaacaaccacagcctgcgccagataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 92 - DNA, 465 bp, Artificial Sequence
  Description of Artificial Sequence: Synthetic polynucleotide
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 93 - DNA, 484 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 94 - DNA, 448 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 95 - DNA, 444 bp, Artificial Sequence
  Description of Artificial Sequence: Synthetic polynucleotide
ctgtgtgtttctgtggctgagtcagatggaggagtcctcatgtttcactgcttagcagtttttgtccttcctagtacccgttcccagcccacaagatgcagaaagagctgttgctagcgtgagttatttttgtcagctgagtcaccacgccagaaagcaagaaatgacccgctttatgtctgctctgaggagctggaaccatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 96 - DNA, 404 bp, Artificial Sequence
  Description of Artificial Sequence: Synthetic polynucleotide
gcgccctgatgaatatgcatcgcggcgcgcccgcccccggctcctcctttcggtttccttcccgccgccaggcggaagcgaagagccgcgcttcccgcgcgcccaggccggccgtggtagggtggggcggggcgggccgcgagccggagaaagagaaagcatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 97 - DNA, 438 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 98 - DNA, 538 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 99 - DNA, 528 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 100 - DNA, 655 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 101 - DNA, 588 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggcggccgccacc

SEQ ID NO: 102 - DNA, 660 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtagggactgtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgcctctagaacc

SEQ ID NO: 103 - DNA, 806 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtagggactgtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 104 - DNA, 938 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccacc

SEQ ID NO: 105 - DNA, 814 bp, Artificial Sequence
  Description of Artificial Sequence: Synthetic polynucleotide
gcgccctgatgaatatgcatcgcggcgcgcccgcccccggctcctcctttcggtttccttcccgccgccaggcggaagcgaagagccgcgcttcccgcgcgcccaggccggccgtggtagggtggggcggggcgggccgcgagccggagaaagagaaagccaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccacc

SEQ ID NO: 106 - DNA, 418 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 107 - DNA, 508 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgttctcctctataaatacccgctctggtatttggggttggcagctgttgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggag

SEQ ID NO: 108 - DNA, 718 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 109 - DNA, 202 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcatgcccaccgcctgctgccacggccggccgtataaatagaggcgaggagcagctgggctctcttggcagtcacc

SEQ ID NO: 110 - DNA, 705 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 111 - DNA, 615 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 112 - DNA, 586 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 113 - DNA, 740 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccaggccacc

SEQ ID NO: 114 - DNA, 650 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccaggccacc

SEQ ID NO: 115 - DNA, 621 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccaggccacc

SEQ ID NO: 116 - DNA, 764 bp, Artificial Sequence
  Description of Artificial Sequence: Synthetic polynucleotide
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 117 - DNA, 480 bp, Artificial Sequence
  Description of Artificial Sequence: Synthetic polynucleotide
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 118 - DNA, 894 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggcccaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccacc

SEQ ID NO: 119 - DNA, 248 bp, Artificial Sequence
  Description of Artificial Sequence: Synthetic polynucleotide
ataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 120 - DNA, 482 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 121 - DNA, 534 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 122 - DNA, 728 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 123 - DNA, 885 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 124 - DNA, 1003 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 125 - DNA, 953 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtatcaagcttggtacgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 126 - DNA, 773 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 127 - DNA, 683 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 128 - DNA, 710 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 129 - DNA, 943 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccagctgcctgccccctgcctggcacagcccgtacctggccgcacgctccctcacaggtgaagctcgaaaactccgtccccgtaaggagccccgctgccccccgaggcctcctccctcacgcctcgctgcgctcccggctcccgcacggccctgggagaggcccccaccgcttcgtccttaacgggcccggcggtgccgggggattatttcggccccggccccgggggggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggcggccacc

SEQ ID NO: 130 - DNA, 724 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 131 - DNA, 822 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 132 - DNA, 1016 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 133 - DNA, 560 bp, Artificial Sequence
  Description of Artificial Sequence: Synthetic polynucleotide
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 134 - DNA, 728 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 135 - DNA, 562 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 136 - DNA, 451 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttccgccacc

SEQ ID NO: 137 - DNA, 883 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccactcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 138 - DNA, 616 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccactcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagtcaccgtccttgacacggccacc

SEQ ID NO: 139 - DNA, 562 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 140 - DNA, 500 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 141 - DNA, 554 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 142 - DNA, 636 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 143 - DNA, 442 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 144 - DNA, 318 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgccaccgcctgctgccacggccggccgtataaatagaggcgaggagcagctgggctctcttggcagtcaccgccacc

SEQ ID NO: 145 - DNA, 501 bp, Artificial Sequence
  Description of Artificial Sequence: Synthetic polynucleotide
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 146 - DNA, 395 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccctgccccccacagctcctctcctgtgccttgtttcccagccatgcgttctcctctataaatacccgctctggtatttggggttggcagctgttgctgccagggagatggttgggttgacatgccaccgcctgctgccacggccggccgtataaatagaggcgaggagcagctgggctctcttggcagtcaccgccacc

SEQ ID NO: 147 - DNA, 334 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctctataaatacccgctctggtatttggggttctctataaatacccgctctggtatttggggttccaccgcctgctgccacggccggccgtataaatagaggcgaggagcagctgggctctcttggcagtcaccgccacc

SEQ ID NO: 148 - DNA, 204 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcccaccgcctgctgccacggccggccgtataaatagaggcgaggagcagctgggctctcttggcagtcaccgccacc

SEQ ID NO: 149 - DNA, 376 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 150 - DNA, 944 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 151 - DNA, 661 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 152 - DNA, 613 bp, Artificial Sequence
  Description of Artificial Sequence: Synthetic polynucleotide
agactggggcaggtgcaggctggattgggtttccagaggctatatatataaaggctgccgggagcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 153 - DNA, 407 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 154 - DNA, 409 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 155 - DNA, 483 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 156 - DNA, 538 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 157 - DNA, 822 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtagactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 158 - DNA, 324 bp, Artificial Sequence
  Description of Artificial Sequence: Synthetic polynucleotide
acacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 159 - DNA, 365 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 160 - DNA, 365 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggtaaacgagctattagttatgaggtccgtagattgaacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 161 - DNA, 565 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 162 - DNA, 543 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttccgccacc

SEQ ID NO: 163 - DNA, 362 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 164 - DNA, 715 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 165 - DNA, 521 bp, Artificial Sequence
  Description of Artificial Sequence: Synthetic polynucleotide
tcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 166 - DNA, 618 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 167 - DNA, 729 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 168 - DNA, 610 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 169 - DNA, 654 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggagataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 170 - DNA, 924 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 171 - DNA, 652 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 172 - DNA, 693 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 173 - DNA, 576 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 174 - DNA, 606 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcctagactagcatgctgcccatgtaaggaggcaaggcctggggacacccgagatgcctggttataattaacccagacatgtggctgcccccccccccccaacacctgctgcctctaaaaataaccctgcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 175 - DNA, 575 bp, Artificial Sequence
  Description of Artificial Sequence: Synthetic polynucleotide
ctctgtctcctcaggtgcctggctgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcctagactagcatgctgcccatgtaaggaggcaaggcctggggacacccgagatgcctggttataattaacccagacatgtggctgcccccccccccccaacacctgctgcctctaaaaataaccctgcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 176 - DNA, 907 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 177 - DNA, 727 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 178 - DNA, 365 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 179 - DNA, 365 bp, Artificial Sequence
  Description of Artificial Sequence: Synthetic polynucleotide
caccgcggtggcggccgtccgccctcggcaccatcctcacgacacccaaatatggcgacgggtgaggaatggtggggagctatttttagagcggtgaggaaggtgggcaggcagcaggtgttggcgctctaaaaatagctcccgggagctatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 180 - DNA, 568 bp, Artificial Sequence
  Description of Artificial Sequence: Synthetic polynucleotide
tccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacctgggaaatctagattccaggaaacccatctggagggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 181 - DNA, 376 bp, Artificial Sequence
  Description of Artificial Sequence: Synthetic polynucleotide
ccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 182 - DNA, 296 bp, Artificial Sequence
  Description of Artificial Sequence: Synthetic polynucleotide
agggtcagtgtcctgccccaacccatgagatgacagactataatagccacaggattaacatagcaggcattgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 183 - DNA, 654 bp, Artificial Sequence
  Description of Artificial Sequence: Synthetic polynucleotide
ctgaggggtgtcagagcacaggctgaggcctcttgcctgacgtgggaccccttggtctggcatttgtcagtgaggcaggctgggggcaggccccggagcttggcaggaggtgtaaaccggccttggaaggtagggccccacaatggggacagttggatctctgagggagacagggaggcatgatcactgccaaatgcccaccaaggacaaggcacatcccagggagacagacgcagacctggtgccctctggacactggcattcctggaggctgatgatggacagatgggcctggaggtggctcttcgccagctggtgtttcctttggacttcctcagtgtctttggagaagcagagccctaagaataagcagctgcccataaaatctaataccagccaagcatctcaggaattcatggattgtctccatcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 184 - DNA, 659 bp, Artificial Sequence
  Description of Artificial Sequence: Synthetic polynucleotide
ttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 185 - DNA, 332 bp, Artificial Sequence
  Description of Artificial Sequence: Synthetic polynucleotide
cctccctggcctggacccccgtggtaaccctataaggcgaggcagctgctgtctgaggcagggaggggctggtgtgggaggctaagggcagctgctaagtttagggtgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 186 - DNA, 483 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 187 - DNA, 535 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 188 - DNA, 598 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 189 - DNA, 683 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 190 - DNA, 453 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 191 - DNA, 591 bp, Artificial Sequence
  Description of Artificial Sequence: Synthetic polynucleotide
cctccctggcctggacccccgtggtaaccctataaggcgaggcagctgctgtctgaggcagggaggggctggtgtgggaggctaagggcagctgctaagtttagggtgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 192 - DNA, 429 bp, Artificial Sequence
  Description of Artificial Sequence: Synthetic polynucleotide
cctccctggcctggacccccgtggtaaccctataaggcgaggcagctgctgtctgaggcagggaggggctggtgtgggaggctaagggcagctgctaagtttagggtgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 193 - DNA, 550 bp, Artificial Sequence
  Description of Artificial Sequence: Synthetic polynucleotide
cctccctggcctggacccccgtggtaaccctataaggcgaggcagctgctgtctgaggcagggaggggctggtgtgggaggctaagggcagctgctaagtttagggtggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 194 - DNA, 388 bp, Artificial Sequence
  Description of Artificial Sequence: Synthetic polynucleotide
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 195 - DNA, 514 bp, Artificial Sequence
  Description of Artificial Sequence: Synthetic polynucleotide
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 196 - DNA, 354 bp, Artificial Sequence
  Description of Artificial Sequence: Synthetic polynucleotide
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 197 - DNA, 354 bp, Artificial Sequence
  Description of Artificial Sequence: Synthetic polynucleotide
aggctaagggcagctgctaagtttagggtgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 198 - DNA, 362 bp, Artificial Sequence
  Description of Artificial Sequence: Synthetic polynucleotide
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcgctctaaggtccctcactcccaactcagccccatgtcctgtcaattcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 199 - DNA, 362 bp, Artificial Sequence
  Description of Artificial Sequence: Synthetic polynucleotide
ctctaaggtccctcactcccaactcagccccatgtcctgtcaattcgacacccaaatatggcgacgggtgaggaatggtggggagttatttttagagcggtgaggaaggtgggcaggcagcaggtgttggcgctctaaaaataactcccgggagttatttttagagcgtaaggcgaggcagctgctgtctgaggcagacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 200 - DNA, 376 bp, Artificial Sequence
  Description of Artificial Sequence: Synthetic polynucleotide
taaggcgaggcagctgctgtctgaggcagaggctaagggcagctgctaagtttagggtctctaaggtccctcactcccaactcagccccatgtcctgtcaattccgacacccaaatatggcgacgggtgaggaatggtggggagttatttttagagcaggcagcaggtgttggcgctctaaaaataactcccgggagttatttttagagcgacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 201 - DNA, 274 bp, Artificial Sequence
  Description of Artificial Sequence: Synthetic polynucleotide
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaa

SEQ ID NO: 202 - DNA, 200 bp, Artificial Sequence
  Description of Artificial Sequence: Synthetic polynucleotide
ctgtgtgtttctgtggctgagtcagatggaggagtcctcatgtttcactgcttagcagtttttgtccttcctagtacccgttcccagcccacaagatgcagaaagagctgttgctagcgtgagttatttttgtcagctgagtcaccacgccagaaagcaagaaatgacccgctttatgtctgctctgaggagctggaacc

SEQ ID NO: 203 - DNA, 284 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagt

SEQ ID NO: 204 - DNA, 160 bp, Artificial Sequence
  Description of Artificial Sequence: Synthetic polynucleotide
gcgccctgatgaatatgcatcgcggcgcgcccgcccccggctcctcctttcggtttccttcccgccgccaggcggaagcgaagagccgcgcttcccgcgcgcccaggccggccgtggtagggtggggcggggcgggccgcgagccggagaaagagaaagc

SEQ ID NO: 205 - DNA, 230 bp, Artificial Sequence
  Description of Artificial Sequence: Synthetic polynucleotide
tacatcatttacctagaaaagaggacagctgtcctttcccaaagctccggtgaccctgccccgcccagtgtgactagcccaggttggtgattctgatctgttgccaaaccaaactggctccccggggagccatttggtaatgttccctggagtcatttccttgcgaagcattccttttcggtgagaggacatttttttcatccctgataaacaaccacagcctgcgccag

SEQ ID NO: 206 - DNA, 230 bp, Artificial Sequence
  Description of Artificial Sequence: Synthetic polynucleotide
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggc

SEQ ID NO: 207 - DNA, 216 bp, Artificial Sequence
  Description of Artificial Sequence: Synthetic polynucleotide
taagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctag

SEQ ID NO: 208 - DNA, 194 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 209 - DNA, 245 bp, Artificial Sequence
  Description of Artificial Sequence: Synthetic polynucleotide
ctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggag

SEQ ID NO: 210 - DNA, 231 bp, Artificial Sequence
  Description of Artificial Sequence: Synthetic polynucleotide
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggg

SEQ ID NO: 211 - DNA, 128 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgc

SEQ ID NO: 212 - DNA, 240 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggcc

SEQ ID NO: 213 - DNA, 125 bp, Artificial Sequence
  Description of Artificial Sequence: Synthetic polynucleotide
cccctgccccccacagctcctctcctgtgccttgtttcccagccatgcgttctcctctataaatacccgctctggtatttggggttggcagctgttgctgccagggagatggttgggttgacatg

SEQ ID NO: 214 - DNA, 32 bp, Artificial Sequence
  Description of Artificial Sequence: Synthetic oligonucleotide
ctctataaatacccgctctggtatttggggtt

SEQ ID NO: 215 - DNA, 167 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagc

SEQ ID NO: 216 - DNA, 229 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 217 - DNA, 188 bp, Artificial Sequence
  Description of Artificial Sequence: Synthetic polynucleotide
acacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 218 - DNA, 344 bp, Artificial Sequence
  Description of Artificial Sequence: Synthetic polynucleotide
tccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacctgggaaatctagattccaggaaacccatctggaggg

SEQ ID NO: 219 - DNA, 152 bp, Artificial Sequence
  Description of Artificial Sequence: Synthetic polynucleotide
ccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaa

SEQ ID NO: 220 - DNA, 72 bp, Artificial Sequence
  Description of Artificial Sequence: Synthetic oligonucleotide
agggtcagtgtcctgccccaacccatgagatgacagactataatagccacaggattaacatagcaggcattg

SEQ ID NO: 221 - DNA, 430 bp, Artificial Sequence
  Description of Artificial Sequence: Synthetic polynucleotide
ctgaggggtgtcagagcacaggctgaggcctcttgcctgacgtgggaccccttggtctggcatttgtcagtgaggcaggctgggggcaggccccggagcttggcaggaggtgtaaaccggccttggaaggtagggccccacaatggggacagttggatctctgagggagacagggaggcatgatcactgccaaatgcccaccaaggacaaggcacatcccagggagacagacgcagacctggtgccctctggacactggcattcctggaggctgatgatggacagatgggcctggaggtggctcttcgccagctggtgtttcctttggacttcctcagtgtctttggagaagcagagccctaagaataagcagctgcccataaaatctaataccagccaagcatctcaggaattcatggattgtctccat

SEQ ID NO: 222 - DNA, 435 bp, Artificial Sequence
  Description of Artificial Sequence: Synthetic polynucleotide
ttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagg

SEQ ID NO: 223 - DNA, 108 bp, Artificial Sequence
  Description of Artificial Sequence: Synthetic polynucleotide
cctccctggcctggacccccgtggtaaccctataaggcgaggcagctgctgtctgaggcagggaggggctggtgtgggaggctaagggcagctgctaagtttagggtg

SEQ ID NO: 224 - DNA, 48 bp, Artificial Sequence
  Description of Artificial Sequence: Synthetic oligonucleotide
ttctcctctataaatacccgctctggtatttggggttggcagctgttg

SEQ ID NO: 225 - DNA, 378 bp, Artificial Sequence
  Description of Artificial Sequence: Synthetic polynucleotide
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggag

SEQ ID NO: 226 - DNA, 226 bp, Artificial Sequence
  Description of Artificial Sequence: Synthetic polynucleotide
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcgctctaaggtccctcactcccaactcagccccatgtcctgtcaattcacccgtcgccatatttgggtgtccgccct

SEQ ID NO: 227 - DNA, 226 bp, Artificial Sequence
  Description of Artificial Sequence: Synthetic polynucleotide
ctctaaggtccctcactcccaactcagccccatgtcctgtcaattcgacacccaaatatggcgacgggtgaggaatggtggggagttatttttagagcggtgaggaaggtgggcaggcagcaggtgttggcgctctaaaaataactcccgggagttatttttagagcgtaaggcgaggcagctgctgtctgaggcagacccgtcgccatatttgggtgtccgccct

SEQ ID NO: 228 - DNA, 240 bp, Artificial Sequence
  Description of Artificial Sequence: Synthetic polynucleotide
taaggcgaggcagctgctgtctgaggcagaggctaagggcagctgctaagtttagggtctctaaggtccctcactcccaactcagccccatgtcctgtcaattccgacacccaaatatggcgacgggtgaggaatggtggggagttatttttagagcaggcagcaggtgttggcgctctaaaaataactcccgggagttatttttagagcgacccgtcgccatatttgggtgtccgccct

SEQ ID NO: 229 - DNA, 46 bp, Artificial Sequence
  Description of Artificial Sequence: Synthetic oligonucleotide
tactaaaaatagaacgactatttttaggcttttctggcagctggcc

SEQ ID NO: 230 - DNA, 48 bp, Artificial Sequence
  Description of Artificial Sequence: Synthetic oligonucleotide
cgaggtactataaatacccttagaggtattttatcttggcagctaggt

SEQ ID NO: 231 - DNA, 70 bp, Artificial Sequence
  Description of Artificial Sequence: Synthetic oligonucleotide
tactaaaaatagaacgactatttttaggcttttctggcagctggccctgccagacagagttcctcagtaa

SEQ ID NO: 232 - DNA, 72 bp, Artificial Sequence
  Description of Artificial Sequence: Synthetic oligonucleotide
cgaggtactataaatacccttagaggtattttatcttggcagctaggtctgccagacagagttcctcagtaa

SEQ ID NO: 233 - DNA, 70 bp, Artificial Sequence
  Description of Artificial Sequence: Synthetic oligonucleotide
tactaaaaatagaacgactatttttaggcttttctggcagctggccctgccagacagataaacgagctat

SEQ ID NO: 234 - DNA, 72 bp, Artificial Sequence
  Description of Artificial Sequence: Synthetic oligonucleotide
cgaggtactataaatacccttagaggtattttatcttggcagctaggtctgccagacagataaacgagctat

SEQ ID NO: 235 - DNA, 73 bp, Artificial Sequence
  Description of Artificial Sequence: Synthetic oligonucleotide
ttaaacgagctattagttatgaggtccgtagattgaataaacgagctattagttatgaggtccgtagattgaa

SEQ ID NO: 236 - DNA, 229 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggtaaacgagctattagttatgaggtccgtagattgaacccgtcgccatatttgggtgtccgccct

SEQ ID NO: 237 - DNA, 252 bp, Artificial Sequence
  Description of Artificial Sequence: Synthetic polynucleotide
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggag

SEQ ID NO: 238 - DNA, 218 bp, Artificial Sequence
  Description of Artificial Sequence: Synthetic polynucleotide
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 239 - DNA, 218 bp, Artificial Sequence
  Description of Artificial Sequence: Synthetic polynucleotide
aggctaagggcagctgctaagtttagggtgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 240 - DNA, 111 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggag

SEQ ID NO: 241 - DNA, 199 bp, Artificial Sequence
  Description of Artificial Sequence: Synthetic polynucleotide
ctctgtctcctcaggtgcctggctgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggc

SEQ ID NO: 242 - DNA, 119 bp, Artificial Sequence
  Description of Artificial Sequence: Synthetic polynucleotide
agactggggcaggtgcaggctggattgggtttccagaggctatatatataaaggctgccgggagcccacattcctttccagaggccagctctccatttatagcccctgggcagagcagc

SEQ ID NO: 243 - DNA, 188 bp, Artificial Sequence
  Description of Artificial Sequence: Synthetic polynucleotide
acacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 244 - DNA, 229 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 245 - DNA, 229 bp, Artificial Sequence
  Description of Artificial Sequence: Synthetic polynucleotide
caccgcggtggcggccgtccgccctcggcaccatcctcacgacacccaaatatggcgacgggtgaggaatggtggggagctatttttagagcggtgaggaaggtgggcaggcagcaggtgttggcgctctaaaaatagctcccgggagctatttttagagcggaggaatggtggacacccaaatatggcgacggttcctcacccgtcgccatatttgggtgtccgccct

SEQ ID NO: 246 - DNA, 72 bp, Artificial Sequence
  Description of Artificial Sequence: Synthetic oligonucleotide
ttctcctctataaatacccgctctggtatttggggttggcagctgttgctgccagggagatggttgggttga

SEQ ID NO: 247 - DNA, 90 bp, Artificial Sequence
  Description of Artificial Sequence: Synthetic oligonucleotide
ccacagcagctgggggcatttatgggccttcctataaacttctgagagggtaactttatcctgcttctttcagccaagtatcctcctcca

SEQ ID NO: 248 - DNA, 121 bp, Artificial Sequence
  Description of Artificial Sequence: Synthetic polynucleotide
aaacccgtgactcacagcacagccagtgtgggggagggggtggctgcctccaatacgtggcgcccagagtcagctgttctggggccttctctggtttctccaactgagtcctgaggtttgg

SEQ ID NO: 249 - DNA, 229 bp, Artificial Sequence
  Description of Artificial Sequence: Synthetic polynucleotide
caccgcggtggcggccgtccgccctcggatagctcgtttagacacccaaatatggcgacggtaaacgagctattgggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccct

SEQ ID NO: 250 - DNA, 229 bp, Artificial Sequence
  Description of Artificial Sequence: Synthetic polynucleotide
caccgcggtggcggccgtccgccctcggatagctcgtttagacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 251 - DNA, 229 bp, Artificial Sequence
  Description of Artificial Sequence: Synthetic polynucleotide
caccgcggtggcggccgtccgccctcggcaccatcctcacgacacccaaatatggcgacggtaaacgagctattgggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 252 - DNA, 149 bp, Artificial Sequence
  Description of Artificial Sequence: Synthetic polynucleotide
acacccaaatatggcgacgggtgaggaatggtggggagttatttttagagcggtgaggaaggtgggcaggcagcaggtgttggcgctctaaaaataactcccgggagttatttttagagcgcccgtcgccatatttgggtgtccgccct

SEQ ID NO: 253 - DNA, 217 bp, Artificial Sequence
  Description of Artificial Sequence: Synthetic polynucleotide
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcggaggaatggtggacacccaaatatggcgacggttcctcacccgtcgccatatttgggtgtccgccct

SEQ ID NO: 254 - DNA, 217 bp, Artificial Sequence
  Description of Artificial Sequence: Synthetic polynucleotide
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcgagctctataaatacccgctctggtatttggggttttgaacccgtcgccatatttgggtgtccgccct

SEQ ID NO: 255 - DNA, 180 bp, Artificial Sequence
  Description of Artificial Sequence: Synthetic polynucleotide
ggccgtccgccctcgggacacccaaatatggcgacgggggagttatttttagagcgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 256 - DNA, 162 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcg

SEQ ID NO: 257 - DNA, 67 bp, Artificial Sequence
  Description of Artificial Sequence: Synthetic oligonucleotide
aggcagtgtatactcttccataaacgagctattagttatgaggtccgtagattgaaaagggtgacgg

SEQ ID NO: 258 - DNA, 229 bp, Artificial Sequence
  Description of Artificial Sequence: Synthetic polynucleotide
caccgcggtggcggccgtccgccctcggcaccatcctcacgacacccaaatatggcgacgggtgaggaatggtggggataaacgagctatgcggtgaggaaggtgggcaggcagcaggtgttggcgcatagctcgtttatcccgggataaacgagctatgcggaggaatggtggacacccaaatatggcgacggttcctcacccgtcgccatatttgggtgtccgccct

SEQ ID NO: 259 - DNA, 45 bp, Artificial Sequence
  Description of Artificial Sequence: Synthetic oligonucleotide
aggcagtgtatactcttccataaacgagctattagttatgaggtc

SEQ ID NO: 260 - DNA, 60 bp, Artificial Sequence
  Description of Artificial Sequence: Synthetic oligonucleotide
ccacagcagctgggggcatttctgagagggtaactttatcctgcttctttcagccaagta

SEQ ID NO: 261 - DNA, 106 bp, Artificial Sequence
  Description of Artificial Sequence: Synthetic polynucleotide
ctcacagcacagccagtgtgggggagggggtggctgcctccgtggcgcccagagtcagctgttctggggccttctctggtttctccaactgagtcctgaggtttgg

SEQ ID NO: 262 - DNA, 245 bp, Artificial Sequence
  Description of Artificial Sequence: Synthetic polynucleotide
actcgggggccaggcactggcgctgacgcaggctagcagggcgccactggctggtccccacccacctcggtgggttgggggatgggcgcaccagcccctcctgggtgagccctagcctggggcttcctatttcgggagccgggggcgtgggccacgtctcctcatgtgatgcgagggctatttaaagcggcagcccgggcagggagccgccgtcggagcccttgcacgcctgctctcttgtagct

SEQ ID NO: 263 - DNA, 205 bp, Artificial Sequence
  Description of Artificial Sequence: Synthetic polynucleotide
ctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccag

SEQ ID NO: 264 - DNA, 292 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 265 - DNA, 524 bp, Artificial Sequence
  Description of Artificial Sequence: Synthetic polynucleotide
gccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctcc

SEQ ID NO: 266 - DNA, 315 bp, Artificial Sequence
  Description of Artificial Sequence: Synthetic polynucleotide
ccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagc

SEQ ID NO: 267 - DNA, 170 bp, Artificial Sequence
  Description of Artificial Sequence: Synthetic polynucleotide
aggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtca

SEQ ID NO: 268 - DNA, 218 bp, Artificial Sequence
  Description of Artificial Sequence: Synthetic polynucleotide
cccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggag

SEQ ID NO: 269 - DNA, 249 bp, Artificial Sequence
  Description of Artificial Sequence: Synthetic polynucleotide
gactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 270 - DNA, 318 bp, Artificial Sequence
  Description of Artificial Sequence: Synthetic polynucleotide
catgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 271 - DNA, 70 bp, Artificial Sequence
  Description of Artificial Sequence: Synthetic oligonucleotide
ccaccgcctgctgccacggccggccgtataaatagaggcgaggagcagctgggctctcttggcagtcacc

SEQ ID NO: 272 - DNA, 451 bp, Artificial Sequence
  Description of Artificial Sequence: Synthetic polynucleotide
ccagctgcctgccccctgcctggcacagcccgtacctggccgcacgctccctcacaggtgaagctcgaaaactccgtccccgtaaggagccccgctgccccccgaggcctcctccctcacgcctcgctgcgctcccggctcccgcacggccctgggagaggcccccaccgcttcgtccttaacgggcccggcggtgccgggggattatttcggccccggccccgggggggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggcg

SEQ ID NO: 273 - DNA, 200 bp, Artificial Sequence
  Description of Artificial Sequence: Synthetic polynucleotide
tcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 274 - DNA, 251 bp, Artificial Sequence
  Description of Artificial Sequence: Synthetic polynucleotide
tcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttcc

SEQ ID NO: 275 - DNA, 80 bp, Artificial Sequence
  Description of Artificial Sequence: Synthetic oligonucleotide
cggccggggccgcattcctgggggccgggcggtgctcccgcccgcctcgataaaaggctccggggccggcggcggcccac

SEQ ID NO: 276 - DNA, 343 bp, Artificial Sequence
  Description of Artificial Sequence: Synthetic polynucleotide
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttcc

SEQ ID NO: 277 - DNA, 240 bp, Artificial Sequence
  Description of Artificial Sequence: Synthetic polynucleotide
ttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 278 - DNA, 168 bp, Artificial Sequence
  Description of Artificial Sequence: Synthetic polynucleotide
ataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgc

SEQ ID NO: 279 - DNA, 620 bp, Artificial Sequence
  Description of Artificial Sequence: Synthetic polynucleotide
caattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctg

SEQ ID NO: 280 - DNA, 168 bp, Artificial Sequence
  Description of Artificial Sequence: Synthetic polynucleotide
cgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 281 - DNA, 253 bp, Artificial Sequence
  Description of Artificial Sequence: Synthetic polynucleotide
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttc

SEQ ID NO: 282 - DNA, 26 bp, Artificial Sequence
  Description of Artificial Sequence: Synthetic oligonucleotide
ataaatacccgctctggtatttgggg

SEQ ID NO: 283 - DNA, 179 bp, Artificial Sequence
  Description of Artificial Sequence: Synthetic polynucleotide
tactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 284-289 - skipped (6 entries)

SEQ ID NO: 290 - DNA, 434 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggtactaaaaatagaacgactatttttaggcttttctggcagctggcccgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 291 - DNA, 436 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggcgaggtactataaatacccttagaggtattttatcttggcagctaggtcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 292 - DNA, 522 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttactaaaaatagaacgactatttttaggcttttctggcagctggccctgccagacagagttcctcagtaacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 293 - DNA, 524 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcgaggtactataaatacccttagaggtattttatcttggcagctaggtctgccagacagagttcctcagtaacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 294 - DNA, 522 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttactaaaaatagaacgactatttttaggcttttctggcagctggccctgccagacagataaacgagctatcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 295 - DNA, 524 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcgaggtactataaatacccttagaggtattttatcttggcagctaggtctgccagacagataaacgagctatcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 296 - DNA, 524 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttaaacgagctattagttatgaggtccgtagattgaataaacgagctattagttatgaggtccgtagattgaacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 297 - DNA, 240 bp, Artificial Sequence; identical to SEQ ID NO: 277
  Description of Artificial Sequence: Synthetic polynucleotide

SEQ ID NO: 298 - DNA, 335 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 299 - DNA, 340 bp, Artificial Sequence
  Description of Artificial Sequence: Synthetic polynucleotide
caccgcggtggcggccgtccgccctcggatagctcgtttagacacccaaatatggcgacggtaaacgagctattgggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 300 - DNA, 365 bp, Artificial Sequence
  Description of Artificial Sequence: Synthetic polynucleotide
caccgcggtggcggccgtccgccctcggatagctcgtttagacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 301 - DNA, 365 bp, Artificial Sequence
  Description of Artificial Sequence: Synthetic polynucleotide
caccgcggtggcggccgtccgccctcggcaccatcctcacgacacccaaatatggcgacggtaaacgagctattgggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 302 - DNA, 585 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccgccacc

SEQ ID NO: 303 - DNA, 546 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgcccgtcgccatatttgggtgtccgccctcggccggggccgcattcctgggggccgggcggtgctcccgcccgcctcgataaaaggctccggggccggcggcggcccacgagctacccggaggagcgggaggcgccgccacc

SEQ ID NO: 304 - DNA, 585 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcgccgccacc

SEQ ID NO: 305 - DNA, 328 bp, Artificial Sequence
  Description of Artificial Sequence: Synthetic polynucleotide
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 306 - DNA, 328 bp, Artificial Sequence
  Description of Artificial Sequence: Synthetic polynucleotide
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 307 - DNA, 436 bp, Artificial Sequence
  Description of Artificial Sequence: Synthetic polynucleotide
acacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 308 - DNA, 291 bp, Artificial Sequence
  Description of Artificial Sequence: Synthetic polynucleotide
ggccgtccgccctcgggacacccaaatatggcgacgggggagttatttttagagcgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 309 - DNA, 761 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 310 - DNA, 720 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 311 - DNA, 551 bp, Artificial Sequence
  Description of Artificial Sequence: Synthetic polynucleotide
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 312 - DNA, 688 bp, Artificial Sequence
  Description of Artificial Sequence: Synthetic polynucleotide
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 313 - DNA, 621 bp, Artificial Sequence
  Description of Artificial Sequence: Synthetic polynucleotide
ccccacagcagctgggggcatttatgggccttcctataaacttctgagagggtaactttatcctgcttctttcagccaagtatcctcctccaaggcagtgtatactcttccataaacgagctattagttatgaggtcaaacccgtgactcacagcacagccagtgtgggggagggggtggctgcctccaatacgtggcgcccagagtcagctgttctggggccttctctggtttctccaactgagtcctgaggtttggtgacggaattcggccgaacgggacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 314 - DNA, 759 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 315 - DNA, 837 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 316 - DNA, 298 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 317 - DNA, 365 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggcagtgtatactcttccataaacgagctattagttatgaggtccgtagattgaaaagggtgacggcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 318 - DNA, 356 bp, Artificial Sequence
  Description of Artificial Sequence: Synthetic polynucleotide
ctctataaatacccgctctggtatttggggttacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 319 - DNA, 772 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttcgccacc

SEQ ID NO: 320 - DNA, 837 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 321 - DNA, 772 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttcgccacc

SEQ ID NO: 322 - DNA, 764 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 323 - DNA, 671 bp, Artificial Sequence
  Description of Artificial Sequence: Synthetic polynucleotide
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 324 - DNA, 671 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 325 - DNA, 506 bp, Artificial Sequence
  Description of Artificial Sequence: Synthetic polynucleotide
ccacagcagctgggggcatttctgagagggtaactttatcctgcttctttcagccaagtactcacagcacagccagtgtgggggagggggtggctgcctccgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 326 - DNA, 365 bp, Artificial Sequence
  Description of Artificial Sequence: Synthetic polynucleotide
caccgcggtggcggccgtccgccctcggcaccatcctcacgacacccaaatatggcgacgggtgaggaatggtggggataaacgagctatgcggtgaggaaggtgggcaggcagcaggtgttggcgcatagctcgtttatcccgggataaacgagctatgcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 327 - DNA, 624 bp, Artificial Sequence
  Description of Artificial Sequence: Synthetic polynucleotide
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcggccggggccgcattcctgggggccgggcggtgctcccgcccgcctcgataaaaggctccggggccggcggcggcccacgagctacccggaggagcgggaggcggccacc

SEQ ID NO: 328 - DNA, 718 bp, Artificial Sequence
  Description of Artificial Sequence: Synthetic polynucleotide
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 329 - DNA, 465 bp, Artificial Sequence
  Description of Artificial Sequence: Synthetic polynucleotide
ccacagcagctgggggcatttctgagagggtaactttatcctgcttctttcagccaagtactcacagcacagccagtgtgggggagggggtggctgcctccgtggcgcccagagtcagctgttctggggccttctctggtttctccaactgagtcctgaggtttggacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 330 - DNA, 270 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttgcggccggggccgcattcctgggggccgggcggtgctcccgcccgcctcgataaaaggctccggggccggcggcggcccacgagctacccggaggagcgggaggcggccacc

SEQ ID NO: 331 - DNA, 258 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagagggtcagtgtcctgccccaacccatgagatgacagactataatagccacaggattaacatagcaggcattgcaccgcctgctgccacggccggccgtataaatagaggcgaggagcagctgggctctcttggcagtcaccgccacc

SEQ ID NO: 332 - DNA, 234 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttgcaccgcctgctgccacggccggccgtataaatagaggcgaggagcagctgggctctcttggcagtcaccgccacc

SEQ ID NO: 333 - DNA, 267 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccccagcccacctgtcccaatgctgacttagtgcaaggcgagccagcaaggagggaggacaggtggcagtggggggtgaggagcatctaaaaatagcccaccgcctgctgccacggccggccgtataaatagaggcgaggagcagctgggctctcttggcagtcaccgccacc

SEQ ID NO: 334 - DNA, 219 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttgcaccgcctgctgccacggccggccgtataaatagaggcgaggagcagctgggctctcttggcagtcaccgccacc

SEQ ID NO: 335 - DNA, 251 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttgcaccgcctgctgccacggccggccgtataaatagaggcgaggagcagctgggctctcttggcagtcaccgccacc

SEQ ID NO: 336 - DNA, 258 bp, Artificial Sequence
  Description of Artificial Sequence: Synthetic polynucleotide
tcgtcccctggctggcccatgtaatctgagcccagcattgtacatatcctgggaacagctgacaatgcagtggtcagacagctggtggggccagctagagctggcagggttggctgggaggggagtgtaggctgattctcctctataaatacccgctctggtatttggggttggcagctgttgcaccgcctgctgccacggccggccgtataaatagaggcgaggagcagctgggctctcttggcagtcaccgccacc

SEQ ID NO: 337 - DNA, 265 bp, Artificial Sequence
  Description of Artificial Sequence: Synthetic polynucleotide
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagcttctcctctataaatacccgctctggtatttggggttggcagctgttgcaccgcctgctgccacggccggccgtataaatagaggcgaggagcagctgggctctcttggcagtcaccgccacc

SEQ ID NO: 338 - DNA, 269 bp, Artificial Sequence
  Description of Artificial Sequence: Synthetic polynucleotide
ccctgccatcttgggtttcagggcagaggagtcttgctaattttgatgcctatttttggacacttcagctgccactggctccttataaacgcatgacaccccatgcaaacacactacccctccctccactgctgacaggtgtgtggttctcctctataaatacccgctctggtatttggggttggcagctgttgcaccgcctgctgccacggccggccgtataaatagaggcgaggagcagctgggctctcttggcagtcaccgccacc

SEQ ID NO: 339 - DNA, 274 bp, Artificial Sequence
  Description of Artificial Sequence: Synthetic polynucleotide
aattatttttaataacacttactggtaagagaaaggggagaaaccttagacaggcacttagatgtgactaaggcaggtttatctctgattccaaagcactggagtggaagtcacaccgtgactcagagcattgtgatgggccagctgtccattctcctctataaatacccgctctggtatttggggttggcagctgttgcaccgcctgctgccacggccggccgtataaatagaggcgaggagcagctgggctctcttggcagtcaccgccacc

SEQ ID NO: 340 - DNA, 274 bp, Artificial Sequence
  Description of Artificial Sequence: Synthetic polynucleotide
cagtgttcctatatttatcccaccatacagagcttcctttgcctcagaaggaccagcagtttcgctagcttaacaaaaccagccactcagggtattggtttacagtcaagcaactctgggagagggcagctgctctcagacatcatacagcttctcctctataaatacccgctctggtatttggggttggcagctgttgcaccgcctgctgccacggccggccgtataaatagaggcgaggagcagctgggctctcttggcagtcaccgccacc

SEQ ID NO: 341 - DNA, 240 bp, Artificial Sequence
  Description of Artificial Sequence: Synthetic polynucleotide
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcagggcaccgcctgctgccacggccggccgtataaatagaggcgaggagcagctgggctctcttggcagtcaccgccacc

SEQ ID NO: 342 - DNA, 275 bp, Artificial Sequence
  Description of Artificial Sequence: Synthetic polynucleotide
agggtcagtgtcctgccccaacccatgagatgacagactataatagccacaggattaacatagcaggcattgatttttaaagactgaggaattaggcacctgtcatttttgccagctggtgtagatgttaaaaattactgtcactcttccgcctgctactttattttgcacctgctgttacttgagttacaggcatttcacaccgcctgctgccacggccggccgtataaatagaggcgaggagcagctgggctctcttggcagtcaccgccacc

SEQ ID NO: 343 - DNA, 251 bp, Artificial Sequence
  Description of Artificial Sequence: Synthetic polynucleotide
atttttaaagactgaggaattaggcacctgtcatttttgccagctggtgtagatgttaaaaattactgtcactcttccgcctgctactttattttgcacctgctgttacttgagttacaggcatttcattctcctctataaatacccgctctggtatttggggttggcagctgttgcaccgcctgctgccacggccggccgtataaatagaggcgaggagcagctgggctctcttggcagtcaccgccacc

SEQ ID NO: 344 - DNA, 274 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcttctcctctataaatacccgctctggtatttggggttggcagctgttggggctgggcataaaagtcagggcagagccatctattgcttacatttgcttctggccacc

SEQ ID NO: 345 - DNA, 218 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttggggctgggcataaaagtcagggcagagccatctattgcttacatttgcttctggccacc

SEQ ID NO: 346 - DNA, 275 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccagggtcagtgtcctgccccaacccatgagatgacagactataatagccacaggattaacatagcaggcattgttctcctctataaatacccgctctggtatttggggttggcagctgttggggctgggcataaaagtcagggcagagccatctattgcttacatttgcttctggccacc

SEQ ID NO: 347 - DNA, 251 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttgttctcctctataaatacccgctctggtatttggggttggcagctgttggggctgggcataaaagtcagggcagagccatctattgcttacatttgcttctggccacc

SEQ ID NO: 348 - DNA, 235 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttggggctgggcataaaagtcagggcagagccatctattgcttacatttgcttctggccacc

SEQ ID NO: 349 - DNA, 242 bp, Artificial Sequence
  Description of Artificial Sequence: Synthetic polynucleotide
tcgtcccctggctggcccatgtaatctgagcccagcattgtacatatcctgggaacagctgacaatgcagtggtcagacagctggtggggccagctagagctggcagggttggctgggaggggagtgtaggctgattctcctctataaatacccgctctggtatttggggttggcagctgttggggctgggcataaaagtcagggcagagccatctattgcttacatttgcttctggccacc

SEQ ID NO: 350 - DNA, 249 bp, Artificial Sequence
  Description of Artificial Sequence: Synthetic polynucleotide
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagcttctcctctataaatacccgctctggtatttggggttggcagctgttggggctgggcataaaagtcagggcagagccatctattgcttacatttgcttctggccacc

SEQ ID NO: 351 - DNA, 253 bp, Artificial Sequence
  Description of Artificial Sequence: Synthetic polynucleotide
ccctgccatcttgggtttcagggcagaggagtcttgctaattttgatgcctatttttggacacttcagctgccactggctccttataaacgcatgacaccccatgcaaacacactacccctccctccactgctgacaggtgtgtggttctcctctataaatacccgctctggtatttggggttggcagctgttggggctgggcataaaagtcagggcagagccatctattgcttacatttgcttctggccacc

SEQ ID NO: 352 - DNA, 272 bp, Artificial Sequence
  Description of Artificial Sequence: Synthetic polynucleotide
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcagggttctcctctataaatacccgctctggtatttggggttggcagctgttggggctgggcataaaagtcagggcagagccatctattgcttacatttgcttctggccacc

SEQ ID NO: 353 - DNA, 259 bp, Artificial Sequence
  Description of Artificial Sequence: Synthetic polynucleotide
agggtcagtgtcctgccccaacccatgagatgacagactataatagccacaggattaacatagcaggcattgatttttaaagactgaggaattaggcacctgtcatttttgccagctggtgtagatgttaaaaattactgtcactcttccgcctgctactttattttgcacctgctgttacttgagttacaggcatttcagggctgggcataaaagtcagggcagagccatctattgcttacatttgcttctggccacc

SEQ ID NO: 354 - DNA, 263 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttggtacttatataagggggtgggggcgcgttcgtcctcagtcgcgatcgaacactcgagccgagcagacgtgcctacggaccggccacc

SEQ ID NO: 355 - DNA, 287 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttgcggccggggccgcattcctgggggccgggcggtgctcccgcccgcctcgataaaaggctccggggccggcggcggcccacgagctacccggaggagcgggaggcggccacc

SEQ ID NO: 356 - DNA, 237 bp, Artificial Sequence
  Description of Artificial Sequence: Synthetic polynucleotide
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttgcaccgcctgctgccacggccggccgtataaatagaggcgaggagcagctgggctctcttggcagtcaccgccacc

SEQ ID NO: 357 - DNA, 221 bp, Artificial Sequence
  Description of Artificial Sequence: Synthetic polynucleotide
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttggggctgggcataaaagtcagggcagagccatctattgcttacatttgcttctggccacc

SEQ ID NO: 358 - DNA, 249 bp, Artificial Sequence
  Description of Artificial Sequence: Synthetic polynucleotide
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttggtacttatataagggggtgggggcgcgttcgtcctcagtcgcgatcgaacactcgagccgagcagacgtgcctacggaccggccacc

SEQ ID NO: 359 - DNA, 159 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagttctcctctataaatacccgctctggtatttggggttggcagctgttg

SEQ ID NO: 360 - DNA, 183 bp, Artificial Sequence
  Description of Artificial Sequence: Synthetic polynucleotide
tctgagggagacagggaggcatgatcactgccaaatgcccaccaaggacaaggcacatcccagggagacagacgcagacctggtgccctctggacactggcattcctggagagggtcagtgtcctgccccaacccatgagatgacagactataatagccacaggattaacatagcaggcattg

SEQ ID NO: 361 - skipped

SEQ ID NO: 362 - DNA, 192 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccccagcccacctgtcccaatgctgacttagtgcaaggcgagccagcaaggagggaggacaggtggcagtggggggtgaggagcatctaaaaatagcc

SEQ ID NO: 363 - DNA, 144 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttg

SEQ ID NO: 364 - DNA, 176 bp, Artificial Sequence
  Description of Artificial Sequence: Synthetic polynucleotide
ctagactagcatgctgcccatgtaaggaggcaaggcctggggacacccgagatgcctggttataattaacccagacatgtggctgcccccccccccccaacacctgctgcctctaaaaataaccctgcttctcctctataaatacccgctctggtatttggggttggcagctgttg

SEQ ID NO: 365 - DNA, 183 bp, Artificial Sequence
  Description of Artificial Sequence: Synthetic polynucleotide
tcgtcccctggctggcccatgtaatctgagcccagcattgtacatatcctgggaacagctgacaatgcagtggtcagacagctggtggggccagctagagctggcagggttggctgggaggggagtgtaggctgattctcctctataaatacccgctctggtatttggggttggcagctgttg

SEQ ID NO: 366 - DNA, 190 bp, Artificial Sequence
  Description of Artificial Sequence: Synthetic polynucleotide
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagcttctcctctataaatacccgctctggtatttggggttggcagctgttg

SEQ ID NO: 367 - DNA, 194 bp, Artificial Sequence
  Description of Artificial Sequence: Synthetic polynucleotide
ccctgccatcttgggtttcagggcagaggagtcttgctaattttgatgcctatttttggacacttcagctgccactggctccttataaacgcatgacaccccatgcaaacacactacccctccctccactgctgacaggtgtgtggttctcctctataaatacccgctctggtatttggggttggcagctgttg

SEQ ID NO: 368 - DNA, 199 bp, Artificial Sequence
  Description of Artificial Sequence: Synthetic polynucleotide
aattatttttaataacacttactggtaagagaaaggggagaaaccttagacaggcacttagatgtgactaaggcaggtttatctctgattccaaagcactggagtggaagtcacaccgtgactcagagcattgtgatgggccagctgtccattctcctctataaatacccgctctggtatttggggttggcagctgttg

SEQ ID NO: 369 - DNA, 199 bp, Artificial Sequence
  Description of Artificial Sequence: Synthetic polynucleotide
cagtgttcctatatttatcccaccatacagagcttcctttgcctcagaaggaccagcagtttcgctagcttaacaaaaccagccactcagggtattggtttacagtcaagcaactctgggagagggcagctgctctcagacatcatacagcttctcctctataaatacccgctctggtatttggggttggcagctgttg

SEQ ID NO: 370 - DNA, 165 bp, Artificial Sequence
  Description of Artificial Sequence: Synthetic polynucleotide
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcaggg

SEQ ID NO: 371 - DNA, 200 bp, Artificial Sequence
  Description of Artificial Sequence: Synthetic polynucleotide
agggtcagtgtcctgccccaacccatgagatgacagactataatagccacaggattaacatagcaggcattgatttttaaagactgaggaattaggcacctgtcatttttgccagctggtgtagatgttaaaaattactgtcactcttccgcctgctactttattttgcacctgctgttacttgagttacaggcatttca

SEQ ID NO: 372 - DNA, 176 bp, Artificial Sequence
  Description of Artificial Sequence: Synthetic polynucleotide
atttttaaagactgaggaattaggcacctgtcatttttgccagctggtgtagatgttaaaaattactgtcactcttccgcctgctactttattttgcacctgctgttacttgagttacaggcatttcattctcctctataaatacccgctctggtatttggggttggcagctgttg

SEQ ID NO: 373 - DNA, 215 bp, Artificial Sequence
  Description of Artificial Sequence: Synthetic polynucleotide
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcttctcctctataaatacccgctctggtatttggggttggcagctgttg

SEQ ID NO: 374 - skipped

SEQ ID NO: 375 - DNA, 216 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccagggtcagtgtcctgccccaacccatgagatgacagactataatagccacaggattaacatagcaggcattgttctcctctataaatacccgctctggtatttggggttggcagctgttg

SEQ ID NO: 376 - DNA, 192 bp, Artificial Sequence
  Description of Artificial Sequence: Synthetic polynucleotide
ccagcccacctgtcccaatgctgacttagtgcaaggcgagccagcaaggagggaggacaggtggcagtggggggtgaggagcatctaaaaatagccttctcctctataaatacccgctctggtatttggggttggcagctgttgttctcctctataaatacccgctctggtatttggggttggcagctgttg

SEQ ID NO: 377-380 - skipped (4 entries)

SEQ ID NO: 381 - DNA, 213 bp, Artificial Sequence
  Description of Artificial Sequence: Synthetic polynucleotide
cttctcaagccaaaggagcaagagttaaaaataacaggctcaccctggcagccacctgtgctggccagccccaccccatccctccctcggggacagctgcagctcctcaggccccgcccgggacattttgggaacactttctcctcttacttctcatcttcagggttctcctctataaatacccgctctggtatttggggttggcagctgttg

SEQ ID NO: 382-384 - skipped (3 entries)

SEQ ID NO: 385 - DNA, 162 bp, Artificial Sequence
  Description of Artificial Sequence: Synthetic polynucleotide
gctgcccatgtaaggaggcaaggcctggggacacccgagatgcctggttataattaacccagacatgtggctgccccccccccccaacacctgctgcctctaaaaataaccctgttctcctctataaatacccgctctggtatttggggttggcagctgttg

SEQ ID NO: 386-387 - skipped (2 entries)

SEQ ID NO: 388 - DNA, 151 bp, Artificial Sequence
  Description of Artificial Sequence: Synthetic polynucleotide
aattatttttaataacacttactggtaagagaaaggggagaaaccttagacaggcacttagatgtgactaaggcaggtttatctctgattccaaagcactggagtggaagtcacaccgtgactcagagcattgtgatgggccagctgtcca

SEQ ID NO: 389 - DNA, 142 bp, Artificial Sequence
  Description of Artificial Sequence: Synthetic polynucleotide
ttggcccaggtcacactggggtgaggctagtgttcctgagccttgacaaggagacagcttgaaatagacgagtgtcacatttctgagcagctgtgtggcgacagcaggaggggtagggaatagacagtataaaagagaaagc

SEQ ID NO: 390 - DNA, 146 bp, Artificial Sequence
  Description of Artificial Sequence: Synthetic polynucleotide
ccctgccatcttgggtttcagggcagaggagtcttgctaattttgatgcctatttttggacacttcagctgccactggctccttataaacgcatgacaccccatgcaaacacactacccctccctccactgctgacaggtgtgtgg

SEQ ID NO: 391 - DNA, 165 bp, Artificial Sequence; identical to SEQ ID NO: 370
  Description of Artificial Sequence: Synthetic polynucleotide

SEQ ID NO: 392 - DNA, 151 bp, Artificial Sequence
  Description of Artificial Sequence: Synthetic polynucleotide
cagtgttcctatatttatcccaccatacagagcttcctttgcctcagaaggaccagcagtttcgctagcttaacaaaaccagccactcagggtattggtttacagtcaagcaactctgggagagggcagctgctctcagacatcatacagc

SEQ ID NO: 393 - DNA, 135 bp, Artificial Sequence
  Description of Artificial Sequence: Synthetic polynucleotide
tcgtcccctggctggcccatgtaatctgagcccagcattgtacatatcctgggaacagctgacaatgcagtggtcagacagctggtggggccagctagagctggcagggttggctgggaggggagtgtaggctga

SEQ ID NO: 394 - DNA, 114 bp, Artificial Sequence
  Description of Artificial Sequence: Synthetic polynucleotide
gctgcccatgtaaggaggcaaggcctggggacacccgagatgcctggttataattaacccagacatgtggctgccccccccccccaacacctgctgcctctaaaaataaccctg

SEQ ID NO: 395 - DNA, 111 bp, Artificial Sequence; identical to SEQ ID NO: 240
  Description of Artificial Sequence: Synthetic polynucleotide

SEQ ID NO: 396 - DNA, 48 bp, Artificial Sequence; identical to SEQ ID NO: 224
  Description of Artificial Sequence: Synthetic oligonucleotide

SEQ ID NO: 397 - DNA, 72 bp, Artificial Sequence; identical to SEQ ID NO: 220
  Description of Artificial Sequence: Synthetic oligonucleotide

SEQ ID NO: 398 - DNA, 96 bp, Artificial Sequence
  Description of Artificial Sequence: Synthetic oligonucleotide
ccagcccacctgtcccaatgctgacttagtgcaaggcgagccagcaaggagggaggacaggtggcagtggggggtgaggagcatctaaaaatagcc

SEQ ID NO: 399 - DNA, 128 bp, Artificial Sequence
  Description of Artificial Sequence: Synthetic polynucleotide
atttttaaagactgaggaattaggcacctgtcatttttgccagctggtgtagatgttaaaaattactgtcactcttccgcctgctactttattttgcacctgctgttacttgagttacaggcatttca

SEQ ID NO: 400 - DNA, 128 bp, Artificial Sequence; identical to SEQ ID NO: 211
  Description of Artificial Sequence: Synthetic polynucleotide

SEQ ID NO: 401 - DNA, 167 bp, Artificial Sequence; identical to SEQ ID NO: 215
  Description of Artificial Sequence: Synthetic polynucleotide

SEQ ID NO: 402 - DNA, 53 bp, Artificial Sequence
  Description of Artificial Sequence: Synthetic oligonucleotide
gggctgggcataaaagtcagggcagagccatctattgcttacatttgcttctg

SEQ ID NO: 403 - DNA, 81 bp, Artificial Sequence
  Description of Artificial Sequence: Synthetic oligonucleotide
gtacttatataagggggtgggggcgcgttcgtcctcagtcgcgatcgaacactcgagccgagcagacgtgcctacggaccg

SEQ ID NO: 404 - DNA, 105 bp, Artificial Sequence; identical to SEQ ID NO: 42
  Description of Artificial Sequence: Synthetic polynucleotide

SEQ ID NO: 405 - DNA, 69 bp, Artificial Sequence
  Description of Artificial Sequence: Synthetic oligonucleotide
caccgcctgctgccacggccggccgtataaatagaggcgaggagcagctgggctctcttggcagtcacc

SEQ ID NO: 406 - DNA, 6746 bp, Artificial Sequence
  Description of Artificial Sequence: Synthetic polynucleotide
cagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgtctagagctagcatatggatccatcgatttagggataacagggtaattatcagcacacaattgcccattatacgcgcgtataatggactattgtgtgctgatatctgtacacttaagggctagatcttagcttacgtcactagagggtccacgtttagtttttaagatccattgatctcctaaacgctgcaagattcgcaacctggtatacttagcctaggcgctaggtcctagtgcagcgggactttttttctaaagtcgttgagaggaggagtcgtcagaccagatagctttgatgtcctgatcggaaggatcgttggcccccctgcaggcagctgttaattaaccgattcattaatgcagcagctgcgcgctcgctcgctcactgaggccgcccgggcaaagcccgggcgtcgggcgacctttggtcgcccggcctcagtgagcgagcgagcgcgcagagagggagtggcgcgccacgcgtcggccgtccgccctcggcaccattcctcacgacaccgaaatatggcgacgggtgaggaatggtggggagttatttttagagcggtgaggaatggtgggcaggcagcaggtgttgggggagttatttttagagcggggagttatttttagagcggtgaggaatggtggacaccgaaatatggcgacgggtgaggaatggtgccgtcgccatatttgggtgtcccgtccgccctcggccggggccgcattcctgggggccgggcggtgctcccgcccgcctcgataaaaggctccggggccggcggcggcccacgagctacccggaggagcgggaggcgtctctgccagcggtccgacgcgcagtcagcaccaggtaggtgggcaccgcgccgtgccgtgccactagtatctaggtgagtatctcagggatccagacatggggatatgggaggtgcctctgatcccagggctcactgtgggtctctctgttcacagagaccgcgggccaccatgagactgacaagatgtcaagctgccctggctgctgccatcacactgaatctgctggtgctgttctatgtgtcctggctgcagcatcagcccagaaactccagagccagaggacccagaagggcctctgctgctggacctagagtgacagtgcttgtcagagagtttgaggcctttgacaatgctgtgcctgagctggtggacagcttcctgcagcaagatcctgctcagcctgtggtggtggctgctgatacactgccttatcctccactggctctgcctagaatccccaatgttagactggccctcctgcagcctgctctggatagacctgctgctgcttccagacctgagacatatgtggccactgagtttgtggccctggtgcctgatggtgccagagctgaagctcctggcctgctggaaagaatggttgaggccctgagagctggatctgccagactggttgctgctcctgtggctacagccaatcctgccagatgtctggccctgaatgtgtccctgagagaatggacagccagatatggtgctgccccagctgctcctagatgtgatgctcttgatggggatgctgtggtcctgctgagagccagggatctgttcaatctgtctgccccactggccagacctgtgggcacatctctgtttctgcagacagctctgagaggctgggctgtgcagctgctggatctcacctttgctgctgcaagacagcctcctctggccacagctcatgccagatggaaggctgagagagagggcagagctagaagggctgctctgctcagagcactgggcatcagactggtgtcttgggaaggtggcagacttgagtggtttggctgcaacaaagaaaccaccagatgctttggcacagttgtgggagacacccctgcctacctgtatgaggaaagatggaccccaccttgctgtctgagagccctgagggaaacagctagatatgttgttggagtgcttgaggctgctggtgtcagatactggctggaaggtggaagtctgctgggagctgctaggcatggggacatcatcccttgggactatgatgtggacctgggcatctacctggaagatgtgggcaattgtgaacagctgagaggggctgaagctggctctgttgtggatgagagaggctttgtctgggagaaagctgttgagggagacttcttcagagtgcagtactctgagagcaaccacctccatgtggatctgtggccattctaccccagaaatggggtcatgacaaaggacacctggctggaccacagacaggatgtggaattccctgagcactttctgcagcctctggtgccactgccttttgctggatttgtggctcaggcccctaacaactacagaagattcctggaactgaagtttggccctggggtcatagagaaccctcagtaccctaatcctgcactgctgagcctgactggatctggctgatgagtcgacaggcctaataaagagctcagatgcatcgatcagagtgtgttggttttttgtgtggtttaaacgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactggcgtaatagcgaagaggttaattaaggcgccctaggccgacccttagactctgtactcagttctataaacgagccattggatacgagatccgtagattgataagggacacggaatatccccggacgcaatagacaccggtggacagcttggtatcctgagcacagtcgcgcgtccgaatctagctctactttagaggccccggattctgatggtcgtagaccgcagaaccgattggggggatgagatctactagttatcagcacacaattgcccattatacgcgcgtataatggactattgtgtgctgatatagggataacagggtaattctagagctagcatatggatccatcgatttattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcagggtaccgggccccccctcgaggtcgacggtatcgataagcttgatatcgaattcctcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcctttttttcctgcagcccgggggatccaagttctagagcccgccaccgcggtggagctcgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 407 - DNA, 1491 bp, Artificial Sequence
  Description of Artificial Sequence: Synthetic polynucleotide
atgagactgacaagatgtcaagctgccctggctgctgccatcacactgaatctgctggtgctgttctatgtgtcctggctgcagcatcagcccagaaactccagagccagaggacccagaagggcctctgctgctggacctagagtgacagtgcttgtcagagagtttgaggcctttgacaatgctgtgcctgagctggtggacagcttcctgcagcaagatcctgctcagcctgtggtggtggctgctgatacactgccttatcctccactggctctgcctagaatccccaatgttagactggccctcctgcagcctgctctggatagacctgctgctgcttccagacctgagacatatgtggccactgagtttgtggccctggtgcctgatggtgccagagctgaagctcctggcctgctggaaagaatggttgaggccctgagagctggatctgccagactggttgctgctcctgtggctacagccaatcctgccagatgtctggccctgaatgtgtccctgagagaatggacagccagatatggtgctgccccagctgctcctagatgtgatgctcttgatggggatgctgtggtcctgctgagagccagggatctgttcaatctgtctgccccactggccagacctgtgggcacatctctgtttctgcagacagctctgagaggctgggctgtgcagctgctggatctcacctttgctgctgcaagacagcctcctctggccacagctcatgccagatggaaggctgagagagagggcagagctagaagggctgctctgctcagagcactgggcatcagactggtgtcttgggaaggtggcagacttgagtggtttggctgcaacaaagaaaccaccagatgctttggcacagttgtgggagacacccctgcctacctgtatgaggaaagatggaccccaccttgctgtctgagagccctgagggaaacagctagatatgttgttggagtgcttgaggctgctggtgtcagatactggctggaaggtggaagtctgctgggagctgctaggcatggggacatcatcccttgggactatgatgtggacctgggcatctacctggaagatgtgggcaattgtgaacagctgagaggggctgaagctggctctgttgtggatgagagaggctttgtctgggagaaagctgttgagggagacttcttcagagtgcagtactctgagagcaaccacctccatgtggatctgtggccattctaccccagaaatggggtcatgacaaaggacacctggctggaccacagacaggatgtggaattccctgagcactttctgcagcctctggtgccactgccttttgctggatttgtggctcaggcccctaacaactacagaagattcctggaactgaagtttggccctggggtcatagagaaccctcagtaccctaatcctgcactgctgagcctgactggatctggctgatga

SEQ ID NO: 408 - DNA, 1639 bp, Artificial Sequence
  Description of Artificial Sequence: Synthetic polynucleotide
cctggctgcagcatcagcccagaaactccagagccagaggacccagaagggcctctgctgctggacctagagtgacagtgcttgtcagagagtttgaggcctttgacaatgctgtgcctgagctggtggacagcttcctgcagcaagatcctgctcagcctgtggtggtggctgctgatacactgccttatcctccactggctctgcctagaatccccaatgttagactggccctcctgcagcctgctctggatagacctgctgctgcttccagacctgagacatatgtggccactgagtttgtggccctggtgcctgatggtgccagagctgaagctcctggcctgctggaaagaatggttgaggccctgagagctggatctgccagactggttgctgctcctgtggctacagccaatcctgccagatgtctggccctgaatgtgtccctgagagaatggacagccagatatggtgctgccccagctgctcctagatgtgatgctcttgatggggatgctgtggtcctgctgagagccagggatctgttcaatctgtctgccccactggccagacctgtgggcacatctctgtttctgcagacagctctgagaggctgggctgtgcagctgctggatctcacctttgctgctgcaagacagcctcctctggccacagctcatgccagatggaaggctgagagagagggcagagctagaagggctgctctgctcagagcactgggcatcagactggtgtcttgggaaggtggcagacttgagtggtttggctgcaacaaagaaaccaccagatgctttggcacagttgtgggagacacccctgcctacctgtatgaggaaagatggaccccaccttgctgtctgagagccctgagggaaacagctagatatgttgttggagtgcttgaggctgctggtgtcagatactggctggaaggtggaagtctgctgggagctgctaggcatggggacatcatcccttgggactatgatgtggacctgggcatctacctggaagatgtgggcaattgtgaacagctgagaggggctgaagctggctctgttgtggatgagagaggctttgtctgggagaaagctgttgagggagacttcttcagagtgcagtactctgagagcaaccacctccatgtggatctgtggccattctaccccagaaatggggtcatgacaaaggacacctggctggaccacagacaggatgtggaattccctgagcactttctgcagcctctggtgccactgccttttgctggatttgtggctcaggcccctaacaactacagaagattcctggaactgaagtttggccctggggtcatagagaaccctcagtaccctaatcctgcactgctgagcctgactggatctggctgatgagtcgacaggcctaataaagagctcagatgcatcgatcagagtgtgttggttttttgtgtggtttaaacgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 409 - DNA, 199 bp, Artificial Sequence
  Description of Artificial Sequence: Synthetic polynucleotide
aggaacccctagtgatggagttggccactccctctctgcgcgctcgctcgctcgggattgggattgggattgggattgggattgggattgataaaaatcaatcccaatcccaatcccaatcccaatcccaatcccgcgagcgagcgcgcaggagagggagtggccaactccatcactaggggttcctaagcttattata

SEQ ID NO: 410 - DNA, 154 bp, Artificial Sequence
  Description of Artificial Sequence: Synthetic polynucleotide
aggaacccctagtgatggagttggccactccctctctgcgcgctcgctcgctcactgagggcgcctataaagataaaaatccaggctttgcctgcctcagttagcgagcgagcgcgcagagagggagtggccaactccatcactaggggttcct

SEQ ID NO: 411 - DNA, 127 bp, Artificial Sequence
  Description of Artificial Sequence: Synthetic polynucleotide
ctagtgatggagttggccactccctctctgcgcgctcgctcgctcactgagggataaaaatccaggctttgcctgcctcagtgagcgagcgagcgcgcagagagggagtggccaactccatcactag